(19) United States
(12) Patent Application Publication
(10) Pub. No.: US 2005/0203072 A1
(43) Pub. Date: Sep. 15, 2005

(54) Compositions, combinations, and methods for treating cardiovascular conditions and other associated conditions

(76) Inventors: Amy E. Rudolph, Fairfield, CT (US); Ricardo Rocha, Flemington, NJ (US)

Correspondence Address: Julie M. Lappin;Pharmacia Corporation, Mail Zone MC5S, 575 Maryville Centre Drive, St. Louis, MO, 63141, US

(21) Appl. No.: 10/787,721

(22) Filed: Feb. 26, 2004

Related U.S. Application Data
(60) Provisional application No. 60/450,529, filed on Feb. 26, 2003.

(51) Int. Cl. A61K 31/56; A61K 31/415

(52) U.S. Cl. 514/171; 514/406; 514/223.5

(57) ABSTRACT

This invention is directed generally to a method for treating a pathological condition (particularly a cardiovascular condition (e.g., hypertension or heart failure) or a condition associated with a cardiovascular condition) using a p38-kinase inhibitor (e.g., a p38-kinase-inhibiting substituted pyrazole), and specifically a combination comprising a p38-kinase inhibitor with an aldosterone antagonist or diuretic for treating a cardiovascular condition. This invention also is directed generally to combinations comprising a p38-kinase inhibitor, and specifically to combinations comprising a p38-kinase inhibitor with an aldosterone antagonist or diuretic. This invention is further directed generally to pharmaceutical compositions comprising a p38-kinase inhibitor, and more specifically to compositions comprising the above-described combinations.

### PRIORITY CLAIM TO RELATED PATENT APPLICATION

This patent claims priority to U.S. Provisional Patent Application Ser. No. 60/450,529 (filed Feb. 26, 2003), which is incorporated by reference into this patent.

### FIELD OF THE INVENTION

This invention is directed generally to a method for treating a pathological condition (particularly a cardiovascular condition (e.g., hypertension or heart failure) or a condition associated with a cardiovascular condition) using a p38-kinase inhibitor (e.g., a p38-kinase-inhibiting substituted pyrazole), and specifically a combination comprising a p38-kinase inhibitor with an aldosterone antagonist or diuretic. This invention also is directed generally to combinations comprising a p38-kinase inhibitor, and specifically to combinations comprising a p38-kinase inhibitor with an aldosterone antagonist or diuretic for treating a cardiovascular condition. This invention is further directed generally to pharmaceutical compositions comprising a p38-kinase inhibitor, and more specifically to compositions comprising the above-described combinations.

### BACKGROUND OF THE INVENTION

Mitogen-activated protein kinases (MAPKs) are collectively a family of proline-directed serine/threonine kinases that transduce signals from the cell membrane to the cell nucleus in response to a variety of signals. These kinases activate their substrates by phosphorylation. Three major subgroups of MAPKs have been identified: extracellular signal-related kinases (“ERK”), p38 MAPKs, and c-jun-NH₂ kinases (JNK).

The p38 MAPKs are present in a variety of isoforms, including p38α, p38β, and p38γ. These kinases are responsible for phosphorylating and activating transcription factors (e.g., ATF2, CHOP, and MEF2C), as well as other kinases (e.g., MAPKAP-2 and MAPKAP-3). The p38 isoforms are activated by, for example, endotoxins (i.e., bacterial lipopolysaccharides), physical cellular stress, chemical cellular stress, cell proliferation, cell growth, cell death, and inflammation. The products of the p38 phosphorylation, in turn, mediate the production of inflammatory cytokines, such as tumor necrosis factors (“TNF”), IL-1, and cyclooxygenase-2.

It has been reported that p38α kinase can cause (or contribute to the effects of), for example, inflammation generally; arthritis; neuroinflammation; pain; fever; pulmonary disorders; cardiovascular diseases; cardiomyopathy; stroke; ischemia; reperfusion injury; renal reperfusion injury; brain edema; neurotrauma and brain trauma; neurodegenerative disorders; central nervous system disorders; liver disease and nephritis; gastrointestinal conditions; ulcerative diseases; ophthalmic diseases; ophthalmological conditions; glaucoma; acute injury to the eye tissue and ocular traumas; diabetes; diabetic nephropathy; skin-related conditions; viral and bacterial infections; myalgias due to infection; influenza; endotoxic shock; toxic shock syndrome; autoimmune disease; bone resorption diseases; multiple sclerosis; disorders of the female reproductive system; pathological (but non-malignant) conditions, such as hemaginomas, angiofibroma of the nasopharynx, and avascular necrosis of bone; benign and malignant tumors/neoplasia including cancer; leukemia; lymphoma; systemic lupus erthrematosis (SLE); angiogenesis including neoplasia; and metastasis. See, e.g., PCT Patent Publication No. WO 00/31063 or U.S. Pat. No. 6,525,059. See also, PCT Publication No. WO 98/52940. See also, U.S. Pat. No. 6,423,713.

Recently, increased cardiac p38 MAPK levels and activity have been reported to be associated with human heart failure secondary to ischaemic heart disease. See, e.g., Cook S. A., et al., “Activation of c-Jun N-terminal kinases and p38-mitogen-activated protein kinases in human heart failure secondary to ischemic heart disease”, J Mol Cell Cardiol., 31:1429-1434 (1999). See also, e.g., Adams, J. W., et al., “Enhanced Gαq signaling: a common pathway mediates cardiac hypertrophy and apoptotic heart failure”, Proc Natl Acad Sci USA., 95:10140-10145 (1998). See also, e.g., Liao, P, et al., “The in vivo role of p38 MAP kinases in cardiac remodeling and restrictive cardiomyopathy”, Proc Natl Acad Sci USA., 98:12283-12288 (2001). See also, e.g., Liao, P., et al., “p38 mitogen-activated protein kinase mediates a negative inotropic effect in cardiac myocytes”, Circ Res., 90, No. 2: 190-96 (2002). See also, e.g., Haq, S., et al., “Differential activation of signal transduction pathways in human hearts with hypertrophy versus advanced heart failure”, Circulation, 103:670-677 (2001). It has been reported that possible stimuli for these increases may include, for example, neurohormones, pro-inflammatory cytokines, and wall stress. See, e.g., Behr, T. M., et al., “Hypertensive end-organ damage and premature mortality are p38 mitogen-activated protein kinase-dependent in a rat model of cardiac hypertrophy and dysfunction”, Circulation, 104:1292-1298 (2001). See also, e.g., Sugden, P. H., et al., “Stress-responsive” mitogen-activated protein kinases (c-Jun N-terminal kinases and p38 mitogen-activated protein kinases) in the myocardium”, Circ Res., 83:345-352 (1998). It has been reported that the p38-α isoform is particularly associated with inducing cardiac hypertrophy, while the p38-β isoform is more associated with cardiomyocyte apoptosis, which occurs actively when compensated cardiac hypertrophy develops into decompensated heart failure. Wang, Y., et al., “Cardiac muscle cell hypertrophy and apoptosis induced by distinct members of the p38 mitogen-activated protein kinase family”, J. Biol. Chem., 273:2161-2168 (1998).

Inhibition of p38 MAPKs has been investigated as a possible method for treating various cardiovascular conditions. It has been reported, for example, that inhibition of p38 activity improved cardiac function after myocardial ischemia and reperfusion. See, e.g., Ma, X. L., et al., “Inhibition of p38 mitogen-activated protein kinase decreases cardiomyocyte apoptosis and improves cardiac function after myocardial ischemia and reperfusion”, Circulation, 99:1685-1691 (1999). It also has been reported that trans-1-(4-hydroxycyclohexyl)-4-(4-fluorophenyl methoxypyridimidin-4-yl)imidazole (reported to be a specific p38 inhibitor) protected against hypertensive end-organ damage, reduced plasma tumor necrosis factor (TNF-α), and improved survival in a rat model of cardiac hypertrophy and dysfunction. See, e.g., Behr T. M., et al. And it has been reported that p38 MAPKs are associated with myocardial apoptosis, and that p38 inhibition reduced post-ischemic myocardial apoptosis. See, e.g., Ma, X. L., et al. See also, Xia, Z., et al., “Opposing effects of ERK and JNK-p38 MAP kinases on apoptosis”, Science, 270:1326-1331 (1995).

In U.S. Pat. No. 6,093,742, Salituro et al. discuss generally the use of various oxo, thioxo, and imino compounds that purportedly inhibit p38 kinase to treat, inter alia, myocardial ischemia, heart attack, cardiac hypertrophy, and thrombin-induced platelet aggregation. And, in U.S. Pat. No. 6,130,235, Mavunkel et al. discuss generally the use of various piperidinyl and piperazinyl compounds that purportedly inhibit p38 kinase to treat, inter alia, coronary artery disease; congestive heart failure; cardiomyopathy; myocarditis; vasculitis; restinosis, such as restinosis that occurs following coronary angioplasty; valvular disease; atherosclerosis; heart failure characterized by ischemia and reperfusion injury; conditions associated with cardiopulmonary bypass; and coronary artery bypass graft.

Other patent references discuss use of substituted-pyrazole p38-kinase inhibitors to treat cardiovascular conditions. See, e.g., Anantanarayan et al., PCT Application No. PCT/US98/10807; and U.S. Pat. Nos. 5,932,576; 6,087,496; and 6,335,336. See also, e.g., Hanson, et al., PCT Application No. PCT/US98/11684; and U.S. Pat. Nos. 6,087,381 and 6,503,930. See also, e.g., Weier, et al., PCT Application No. PCT/US99/07036; and U.S. Pat. No. 6,509,361. See also, e.g., Anantanarayan, et al., PCT Application No. PCT/US98/10436. See also, e.g., Anantanarayan et al., U.S. Pat. Nos. 6,514,977 and 6,423,713. See also, e.g., Anantanarayan et al., PCT Application No. PCT/US99/26007; and U.S. Pat. No. 6,525,059. See also, e.g. Benson, et al., U.S. Patent Application Ser. No. 60/386,415 (filed Jun. 5, 2002).

Various combination therapies for treating cardiovascular diseases have been described in the literature.

For example, in PCT Application No. PCT/US99/27946, Keller et al. disclose combinations comprising ileal bile acid transport (“IBAT”) inhibitors or cholesteryl ester transport protein (“CTEP”) inhibitors with other agents to treat various cardiovascular conditions.

In PCT Application No. PCT/US00/31263, Williams et al. disclose combinations comprising epoxy-steroidal aldosterone antagonists with other agents to treat hypertension and other various cardiovascular conditions.

In U.S. Pat. No. 6,410,524, Perez et al. disclose combinations comprising ACE inhibitors, aldosterone antagonists, and diuretics to treat various circulatory disorders.

Combinations of IBAT inhibitors with HMG CoA reductase inhibitors useful for the treatment of cardiovascular disease are disclosed by Keller, et al. in U.S. Pat. No. 6,268,392 and Reitz et al. in PCT Patent Publication No. 98/40375.

A combination therapy of fluvastatin and niceritrol is described by J. Sasaki et al. (Int. J. Clin. Pharm. Ther., 33(7), 420-26 (1995)). Those researchers conclude that the combination of fluvastatin with niceritrol “at a dose of 750 mg/day dose does not appear to augment or attenuate beneficial effects of fluvastatin.”

Cashin-Hemphill et al. (J. Am. Med. Assoc., 264(23), 3013-17 (1990)) report beneficial effects of a combination therapy of colestipol and niacin on coronary atherosclerosis. The described effects include non-progression and regression in native coronary artery lesions.

A combination therapy of acipimox and simvastatin has been reported to show beneficial HDL effects in patients having high triglyceride levels (N. Hoogerbrugge et al., J. Internal Med., 241, 151-55 (1997)).

Sitostanol ester margarine and pravastatin combination therapy is described by H. Gylling et al. (J. Lipid Res., 37, 1776-85 (1996)). That therapy is reported to simultaneously inhibit cholesterol absorption and lower LDL cholesterol significantly in non-insulin-dependent diabetic men.

Brown et al. (New Eng. J. Med., 323(19), 1289-1339 (1990)) describe a combination therapy of lovastatin and colestipol which reportedly reduces atherosclerotic lesion progression and increase lesion regression relative to lovastatin alone.

In PCT Patent Publication No. WO 99/11260, Scott describes combinations of atorvastatin (an HMG CoA reductase inhibitor) with an antihypertensive agent for the treatment of angina pectoris, atherosclerosis, combined hypertension and hyperlipidemia, and symptoms of cardiac risk.

In PCT Patent Publication No. WO 96/40255, Egan et al. describe a combination therapy of an angiotensin II antagonist and an epoxy-steroidal aldosterone antagonist. The epoxy-steroidal aldosterone antagonists in the Egan application include eplerenone.

In PCT Patent Publication No. WO 02/09759, Rocha et al. describe a combination therapy of an aldosterone antagonist and cyclooxygenase-2 inhibitor for the treatment of inflammation-related cardiovascular disorders.

In PCT Patent Publication No. WO 02/09760, Alexander et al. describe a combination therapy of an epoxy-steroidal aldosterone antagonist and beta-adrenergic antagonist for treating congestive heart failure.

In PCT Patent Publication No. WO 02/09761, Schuh describes a combination therapy of an epoxy-steroidal aldosterone antagonist and calcium channel blocker for treating congestive heart failure.

In PCT Patent Publication No. WO 02/09683, Williams et al. describe, inter alia, combination therapies of an aldosterone antagonist and, for example, an ACE inhibitor or diuretic to treat inflammation-related disorders, including cardiovascular disorders.

In PCT Patent Publication No. WO 01/95893, Williams et al. describe, inter alia, combination therapies of an epoxy-steroidal aldosterone antagonist and, for example, an ACE inhibitor or diuretic to treat aldosterone-mediated pathogenic effects, including cardiovascular disorders.

Despite the foregoing, heart disease continues to be one of the leading causes of human healthcare costs and death in the world, and the leading cause of human death in the United States and other countries. Thus, there continues to be a need for effective methods and compositions to treat cardiovascular diseases. The following disclosure describes methods and compositions addressing this need.

### SUMMARY OF THE INVENTION

This invention is directed, in part, to a method for treating a pathological cardiovascular condition or a condition associated with a cardiovascular condition. Such a method is typically suitable for use with mammals, such as humans, other primates (e.g., monkeys, chimpanzees. etc.), companion animals (e.g., dogs, cats, horses. etc.), farm animals (e.g., goats, sheep, pigs, cattle, etc.), laboratory animals (e.g., mice, rats, etc.), and wild and zoo animals (e.g., wolves, bears, deer, etc.).

Briefly, therefore, this invention is directed, in part, to a method for treating a pathological condition in a mammal.

In some embodiments, the method comprises administering to the mammal a first amount of a compound that comprises a substituted-pyrazole that inhibits p38-kinase activity. The method also comprises administering to the mammal a second amount of a compound that comprises an aldosterone antagonist or diuretic. Here, the first and second amounts together comprise a therapeutically-effective amount of the compounds.

In some embodiments, the method comprises administering to the mammal a first amount of a compound that inhibits p38-kinase activity. The method also comprises administering to the mammal a second amount of a compound that comprises an aldosterone antagonist or a diuretic. The first and second amounts together comprise a therapeutically-effective amount of the compounds. Here, the pathological condition comprises a cardiovascular disease, glomerulosclerosis, end-stage renal disease, acute renal failure, diabetic nephropathy, reduced renal blood flow, increased glomerular filtration fraction, decreased glomerular filtration rate, decreased creatine clearance, renal arteriopathy, ischemic renal lesions, vascular damage in the kidney, vascular inflammation in the kidney, malignant nephrosclerosis, thrombotic vascular disease, proliferative arteriopathy, atherosclerosis, decreased vascular compliance, retinopathy, neuropathy, edema, or insulinopathy.

This invention also is directed, in part, to a composition (particularly a pharmaceutical composition or medicament). The composition comprises a first amount of a compound that comprises a compound that inhibits p38-kinase activity. The composition also comprises a second amount of a compound that comprises an aldosterone antagonist or diuretic.

This invention also is directed, in part, to a kit. The kit comprises a first dosage form comprising a compound that inhibits p38-kinase activity. The kit also comprises a second dosage form that comprises an aldosterone antagonist or diuretic.

This invention also is directed, in part, to a use of a p38-kinase inhibiting compound and a compound that comprises an aldosterone antagonist or diuretic for making a medicament to treat a pathological condition in a mammal. The medicament comprises a first amount of the p38-kinase inhibiting compound, and a second amount of the compound that comprises the aldosterone antagonist or diuretic. The first and second amounts of the compounds together comprise a therapeutically-effective amount of the compounds.

In some embodiments directed to making a medicament, the p38-kinase inhibiting compound comprises a substituted pyrazole.

In some embodiments directed to making a medicament, the pathological condition comprises a cardiovascular disease, glomerulosclerosis, end-stage renal disease, acute renal failure, diabetic nephropathy, reduced renal blood flow, increased glomerular filtration fraction, decreased glomerular filtration rate, decreased creatine clearance, renal arteriopathy, ischemic renal lesions, vascular damage in the kidney, vascular inflammation in the kidney, malignant nephrosclerosis, thrombotic vascular disease, proliferative arteriopathy, atherosclerosis, decreased vascular compliance, retinopathy, neuropathy, edema, or insulinopathy.

Further benefits of Applicants' invention will be apparent to one skilled in the art from reading this specification.

### DETAILED DESCRIPTION OF THE PREFERRED EMBODIMENTS

This detailed description of preferred embodiments is intended only to acquaint others skilled in the art with Applicants' invention, its principles, and its practical application so that others skilled in the art may adapt and apply the invention in its numerous forms, as they may be best suited to the requirements of a particular use. This detailed description and its specific examples, while indicating preferred embodiments of this invention, are intended for purposes of illustration only. This invention, therefore, is not limited to the preferred embodiments described in this specification, and may be variously modified.

It has been discovered that administration of one or more p38-kinase inhibitors (particularly in combination with aldosterone antagonists and/or diuretics) generally provides an effective treatment for a variety of cardiovascular conditions. Such effectiveness may be realized in, for example, efficacy, potency, dosing requirements, and/or reduced side effects. The term “cardiovascular condition” is used broadly in this application, and includes, for example, hypertension, heart failure (such as congestive heart failure (i.e., “CHF”), or heart failure following myocardial infarction), arrhythmia, diastolic dysfunction (such as left ventricular diastolic dysfunction, diastolic heart failure, or impaired diastolic filling), systolic dysfunction, ischemia (such as myocardial ischemia), cardiomyopathy (such as hypertrophic cardiomyopathy and dilated cardiomyopathy), sudden cardiac death, myocardial fibrosis, vascular fibrosis, impaired arterial compliance, myocardial necrotic lesions, vascular damage in the heart, vascular inflammation in the heart, myocardial infarction (“MI”) (including both acute post-MI and chronic post-MI conditions), coronary angioplasty, left ventricular hypertrophy, decreased ejection fraction, coronary thrombosis, cardiac lesions, vascular wall hypertrophy in the heart, endothelial thickening, myocarditis, and coronary artery disease (such as fibrinoid necrosis of coronary arteries).

It also has been discovered that administration of one or more p38-kinase inhibitors (particularly in combination with aldosterone antagonists and/or diuretics) generally provides an effective treatment for a variety of conditions that are associated (either directly or indirectly) with hypertension, heart failure, and/or other cardiovascular conditions. Such secondary conditions include, for example, renal dysfunctions, cerebrovascular diseases, vascular diseases generally, retinopathy, neuropathy (such as peripheral neuropathy), edema, endothelial dysfunction, and insulinopathy (including complications arising from insulinopathy). Examples of renal dysfunctions include glomerulosclerosis, end-stage renal disease, acute renal failure, diabetic nephropathy, reduced renal blood flow, increased glomerular filtration fraction, proteinuria, decreased glomerular filtration rate, decreased creatine clearance, microalbuminuria, renal arteriopathy, ischemic lesions, vascular damage in the kidney, vascular inflammation in the kidney, and malignant nephrosclerosis (such as ischemic retraction, thrombonecrosis of capillary tufts, arteriolar fibrinoid necrosis, and thrombotic microangiopathic lesions affecting glomeruli and microvessels). Examples of cerebrovascular diseases include stroke. Examples of vascular diseases include thrombotic vascular disease (such as mural fibrinoid necrosis, extravasation and fragmentation of red blood cells, and luminal and/or mural thrombosis), proliferative arteriopathy (such as swollen myointimal cells surrounded by mucinous extracellular matrix and nodular thickening), atherosclerosis, decreased vascular compliance (such as pathological vascular stiffness and/or reduced ventricular compliance), and endothelial dysfunction. Examples of edema include peripheral tissue edema and lung congestion. Examples of insulinopathies include insulin resistance, Type I diabetes mellitus, Type II diabetes mellitus, glucose sensitivity, pre- and diabetic syndrome X.

In some embodiments, the pathological condition comprises a cardiovascular disease, renal dysfunction, edema, a cerebrovascular disease, or an insulinopathy.

In some embodiments, the pathological condition comprises a cardiovascular disease, stroke, or type II diabetes.

In some embodiments, the pathological condition comprises hypertension, heart failure, left ventricular hypertrophy, or stroke.

In some embodiments, the pathological condition comprises a cardiovascular disease.

In some embodiments, the pathological condition comprises hypertension.

In some embodiments, the pathological condition comprises heart failure, arrhythmia, diastolic dysfunction, systolic dysfunction, ischemia, cardiomyopathy, sudden cardiac death, myocardial fibrosis, vascular fibrosis, impaired arterial compliance, myocardial necrotic lesions, vascular damage in the heart, myocardial infarction, left ventricular hypertrophy, decreased ejection fraction, vascular wall hypertrophy in the heart, or endothelial thickening.

In some embodiments, the pathological condition comprises heart failure.

In some embodiments, the pathological condition comprises acute heart failure.

In some embodiments, the pathological condition comprises acute post-myocardial-infarction heart failure.

In some embodiments, the pathological condition comprises chronic heart failure.

In some embodiments, the pathological condition comprises chronic post-myocardial-infarction heart failure.

In some embodiments, the pathological condition comprises hypertension-driven heart failure.

In some embodiments, the pathological condition comprises sudden cardiac death.

In some embodiments, the pathological condition comprises vascular inflammation in the heart.

In some embodiments, the pathological condition comprises coronary angioplasty.

In some embodiments, the pathological condition comprises coronary thrombosis.

In some embodiments, the pathological condition comprises cardiac lesions.

In some embodiments, the pathological condition comprises myocarditis.

In some embodiments, the pathological condition comprises coronary artery disease, such as fibrinoid necrosis of coronary arteries.

In some embodiments, the pathological condition comprises renal dysfunction.

In some embodiments, the pathological condition comprises a cerebrovascular disease.

In some embodiments, the pathological condition comprises an insulinopathy.

In some embodiments, the patient is a companion animal. In some such embodiments, for example, the companion animal is a dog (or “canine”), and the pathological condition comprises heart failure.

It should be recognized that a condition treatable by methods of this invention may exist as a continuous or intermittent condition in a subject. The condition also may be a chronic or acute condition.

### A. Examples of p38-Kinase Inhibitors

In some preferred embodiments, the p38-kinase inhibitor comprises a substituted pyrazole.

In some embodiments wherein the p38-kinase inhibitor comprises a substituted pyrazole, the p38-kinase inhibitor is selected from the group consisting of p38-kinase inhibitors disclosed by Anantanarayan et al. in WIPO Int'l Application No. PCT/US98/10807 (filed May 22, 1998; published Nov. 26, 1998 as Publ. No. WO 98/52937); U.S. Pat. No. 5,932,576 (issued Aug. 3, 1999; filed May 22, 1998 as U.S. application Ser. No. 09/083,923); U.S. Pat. No. 6,087,496 (issued Jul. 11, 2000; filed Apr. 1, 1999 as U.S. application Ser. No. 09/283,718); U.S. Pat. No. 6,335,336 (issued Jan. 1, 2002; filed Apr. 28, 2000 as U.S. application Ser. No. 09/561,423); and U.S. patent application Ser. No. 10/024,071 (filed Dec. 18, 2001) (all of which are incorporated by reference into this patent).

In some embodiments wherein the p38-kinase inhibitor comprises a substituted pyrazole, the p38-kinase inhibitor is selected from the group consisting of p38-kinase inhibitors disclosed by Hanson, et al. in WIPO Int'l Application No. PCT/US98/11684 (filed May 22, 1998; published Nov. 26, 1998 as Publ. No. WO 98/52941); U.S. Pat. No. 6,087,381 (issued Jul. 11, 2000; filed May 22, 1998 as U.S. application Ser. No. 09/083,724); U.S. Pat. No. 6,503,930 (issued Jan. 7, 2003; filed Mar. 31, 2000 as U.S. application Ser. No. 09/540,464); and U.S. patent application Ser. No. 10/267,650 (filed Oct. 9, 2002) (all of which are incorporated by reference into this patent).

In some embodiments wherein the p38-kinase inhibitor comprises a substituted pyrazole, the p38-kinase inhibitor is selected from the group consisting of p38-kinase inhibitors disclosed by Weier, et al. in WIPO Int'l Application No. PCT/US99/07036 (filed May 12, 1999; published Nov. 18, 1999 as Publ. No. WO 99/58523); U.S. Pat. No. 6,509,361 (issued Jan. 21, 2003; filed Feb. 21, 2001 as U.S. application Ser. No. 09/674,653); and U.S. patent application Ser. No. 10/322,039 (filed Dec. 17, 2002) (all of which are incorporated by reference into this patent).

In some embodiments wherein the p38-kinase inhibitor comprises a substituted pyrazole, the p38-kinase inhibitor is selected from the group consisting of p38-kinase inhibitors disclosed by Anantanarayan, et al. in WIPO Int'l Application No. PCT/US98/10436 (filed May 22, 1998; published Nov. 26, 1998 as Publ. No. WO 98/52940) (incorporated by reference into this patent).

In some embodiments wherein the p38-kinase inhibitor comprises a substituted pyrazole, the p38-kinase inhibitor is selected from the group consisting of p38-kinase inhibitors disclosed by Anantanarayan et al. in U.S. Pat. No. 6,514,977 (issued Feb. 4, 2003; filed May 22, 1998 as U.S. application Ser. No. 09/083,670); U.S. Pat. No. 6,423,713 (issued Jul. 23, 2002; filed Jul. 31, 2001 as U.S. application Ser. No. 09/918,481); and U.S. patent application Ser. No. 10/114,297 (filed Apr. 2, 2002) (all of which are incorporated by reference into this patent).

In some embodiments wherein the p38-kinase inhibitor comprises a substituted pyrazole, the p38-kinase inhibitor is selected from the group consisting of p38-kinase inhibitors disclosed by Anantanarayan et al. in WIPO Int'l Application No. PCT/US99/26007 (filed Nov. 17, 1999; published Jun. 2, 2000 as Publ. No. WO 00/31063); U.S. Pat. No. 6,525,059 (issued Feb. 25, 2003; filed Feb. 24, 2000 as U.S. application Ser. No. 09/513,351); and U.S. patent application Ser. No. 10/021,780 (filed Dec. 7, 2001) (all of which are incorporated by reference into this patent). Those p38-kinase inhibitors include, for example, the compounds shown in Table 1:

TABLE 1

| Compound | |
|---|---|
| Number | Compound |
| P-1 | 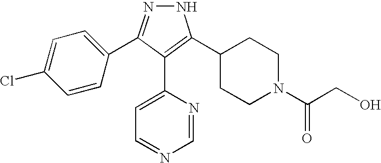 |
| P-2 | 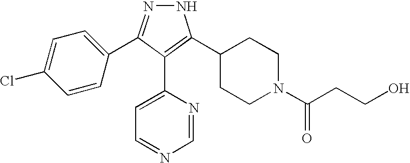 |
| P-3 | 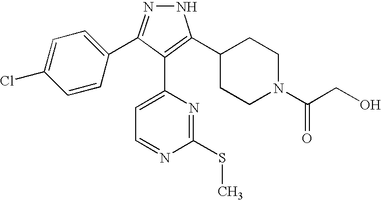 |
| P-4 | 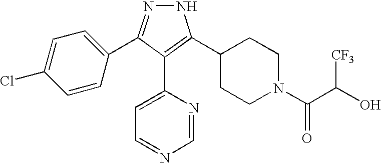 |
| P-5 | 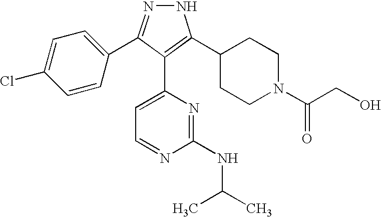 |
| P-6 | 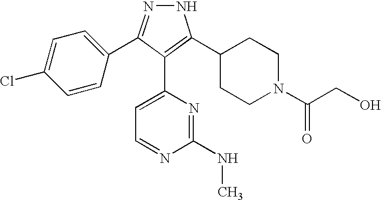 |
| P-7 | 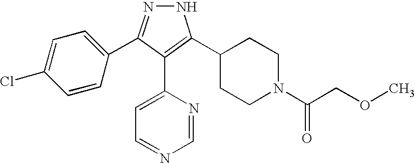 |
| P-8 | 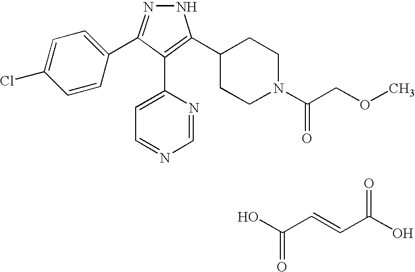 |
| P-9 | 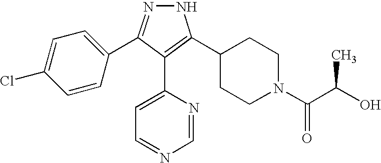 |
| P-10 | 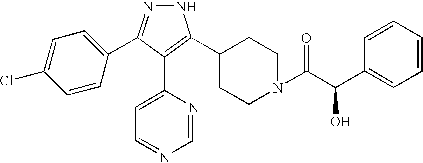 |
| P-11 | 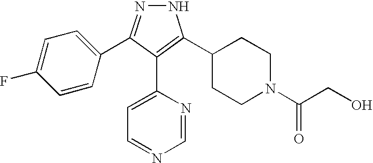 |
| P-12 | 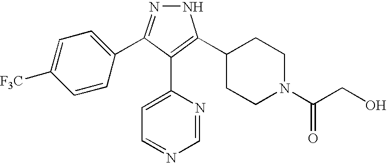 |
| P-13 | 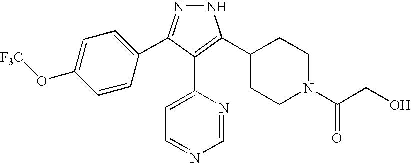 |
| P-14 | 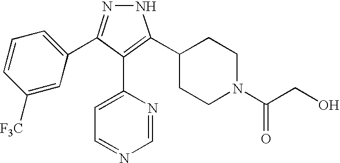 |
| P-15 | 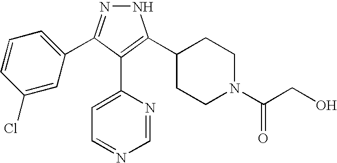 |
| P-16 | 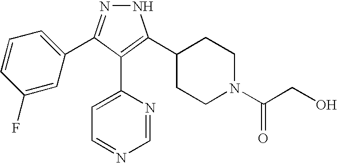 |
| P-17 | 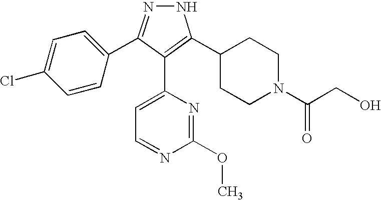 |
| P-18 | 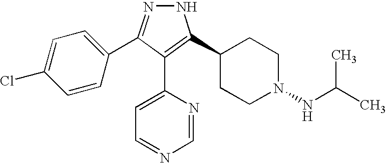 |
| P-19 | 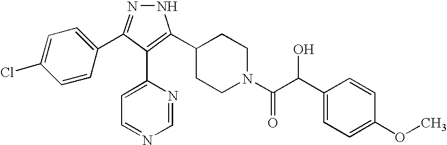 |
| P-20 | 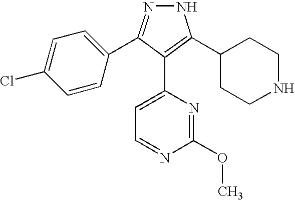 |
| P-21 | 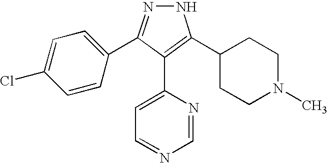 |

In some preferred embodiments, these compounds are prepared by a process disclosed by Allen et al. in U.S. patent application Ser. No. 10/254,445 (filed Sep. 25, 2002); and PCT Publication No. WO 03/026663 (both of which are incorporated by reference into this patent). See also, U.S. patent application Ser. No. 10/456,933 (filed Jun. 5, 2003); and PCT Patent Publication No. WO 03/104223 (both of which are incorporated by reference into this patent).

In some embodiments wherein the p38-kinase inhibitor comprises a substituted pyrazole, the p38-kinase inhibitor corresponds in structure to Formula P-1: 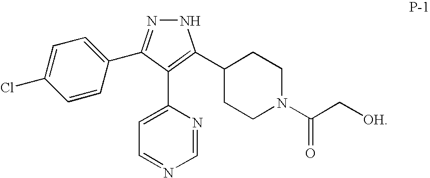
In some preferred embodiments, this compound comprises a crystalline form disclosed by Allen et al. in U.S. patent application Ser. No. 10/254,697 (filed Sep. 25, 2002); and PCT Application No. PCT/US02/30538 (filed Sep. 25, 2002) (both of which are incorporated by reference into this patent).

In some embodiments wherein the p38-kinase inhibitor comprises a substituted pyrazole the p38-kinase inhibitor corresponds in structure to Formula P-15:

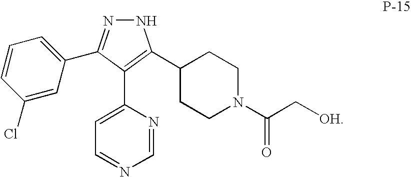

In some embodiments wherein the p38-kinase inhibitor comprises a substituted pyrazole the p38-kinase inhibitor corresponds in structure to Formula P-18:

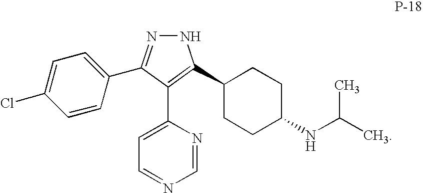

In some embodiments wherein the p38-kinase inhibitor comprises a substituted pyrazole the p38-kinase inhibitor corresponds in structure to Formula P-21:

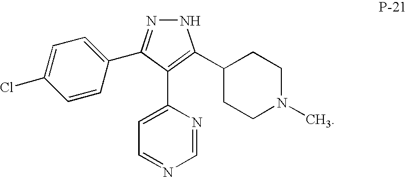

In some embodiments wherein the p38-kinase inhibitor comprises a substituted pyrazole, the p38-kinase inhibitor is selected from the group of p38-kinase inhibitors disclosed by Benson, et al. in U.S. Patent Application Ser. No. 60/386,415 (filed Jun. 5, 2002) (incorporated by referenced into this patent). Those p38-kinase inhibitors include, for example, the compounds shown in Table 2:

TABLE 2

| Compound | |
|---|---|
| Number | Compound |
| P-22 | 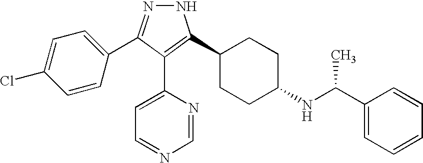 |
| P-23 | 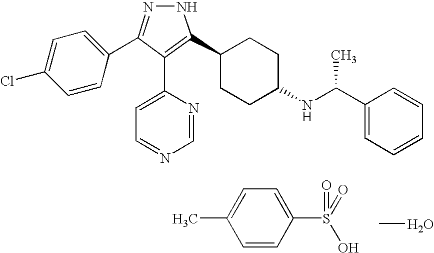 |
| P-24 | 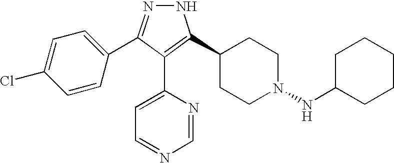 |
| P-25 | 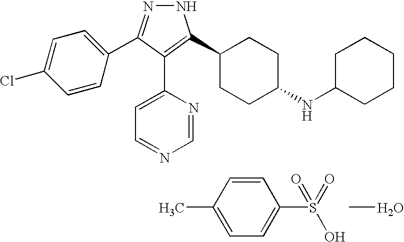 |
| P-26 | 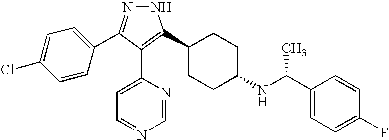 |
| P-27 | 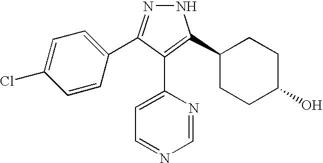 |
| P-28 | 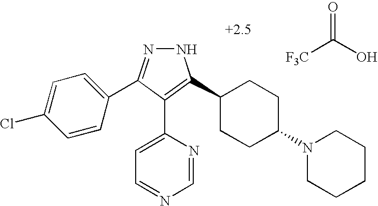 |
| P-29 | 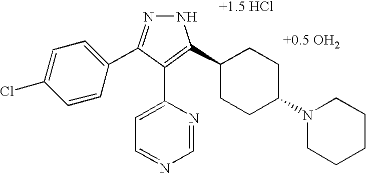 |
| P-30 | 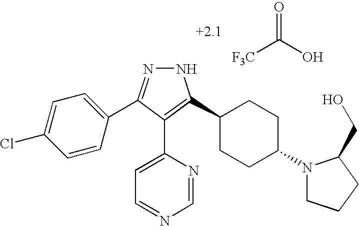 |
| P-31 | 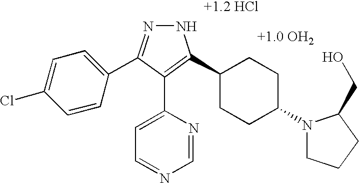 |
| P-32 | 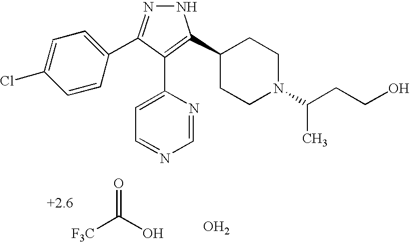 |
| P-33 | 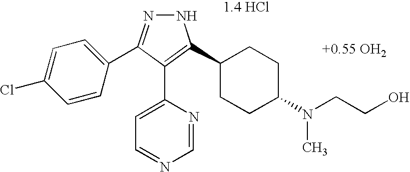 |
| P-34 | 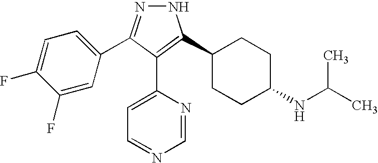 |
| P-35 | 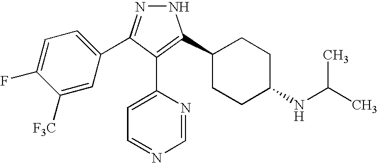 |
| P-36 | 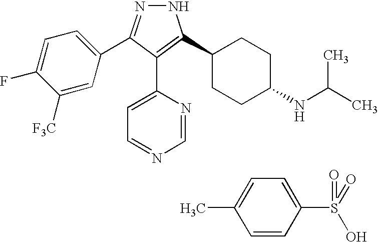 |
| P-37 | 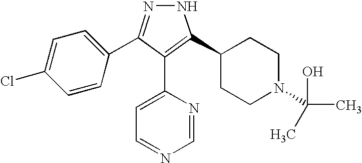 |
| P-38 | 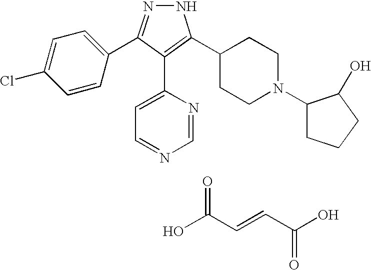 |
| P-39 | 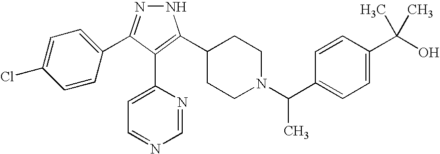 |
| P-40 | 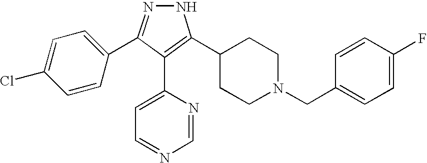 |
| P-41 | 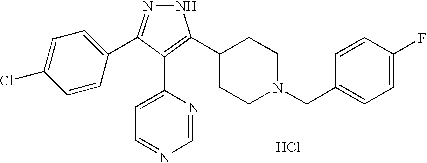 |
| P-42 | 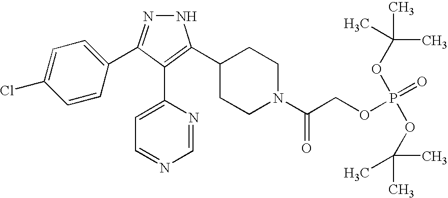 |
| P-43 | 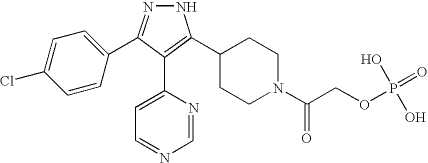 |
| P-44 | 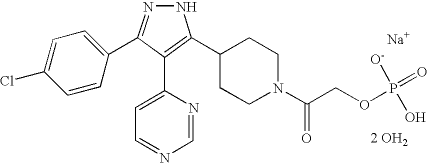 |
| P-45 | 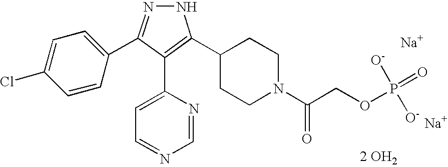 |
| P-46 | 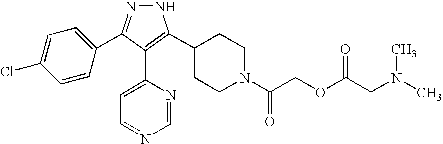 |
| P-47 | 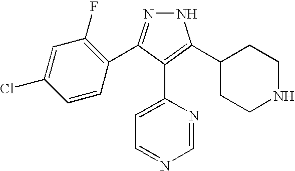 |
| P-48 | 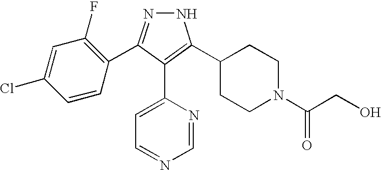 |
| P-49 | 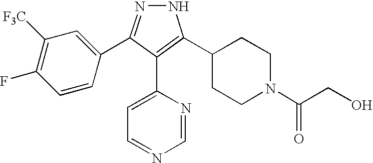 |
| P-50 | 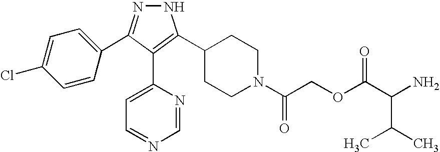 |
| P-51 | 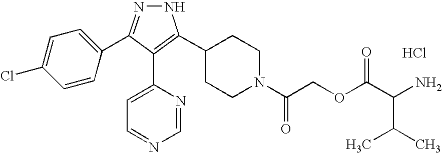 |
| P-52 | 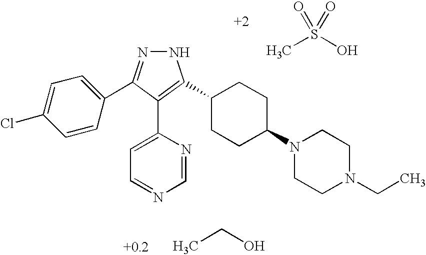 |
| P-53 | 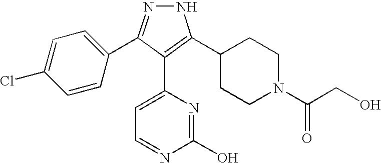 |
| P-54 | 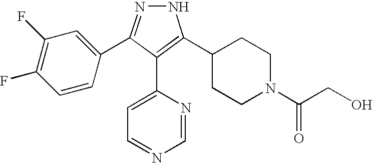 |
| P-55 | 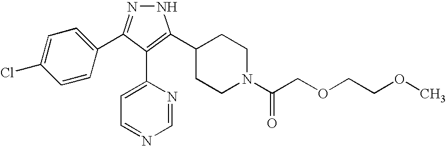 |
| P-56 | 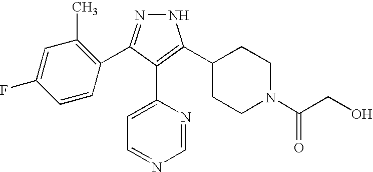 |
| P-57 | 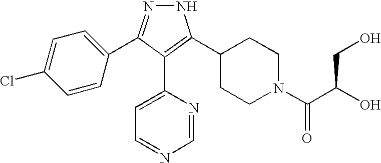 |
| P-58 | 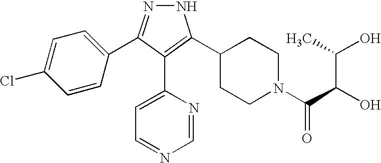 |
| P-59 | 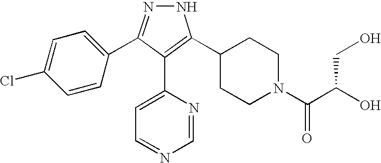 |
| P-60 | 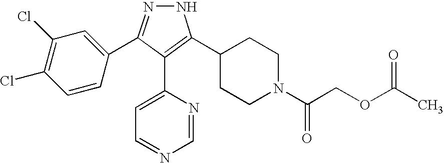 |
| P-61 | 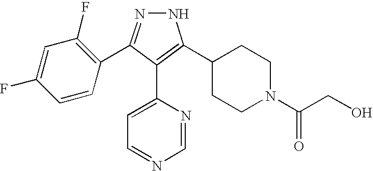 |
| P-62 | 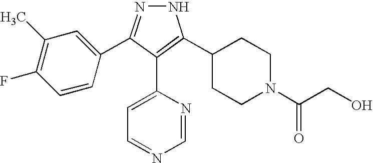 |
| P-63 | 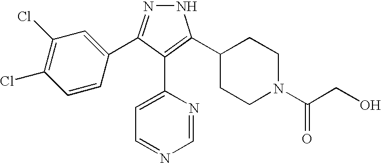 |
| P-64 | 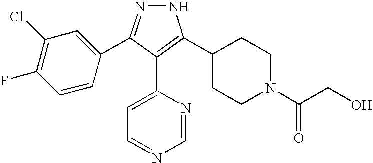 |
| P-65 | 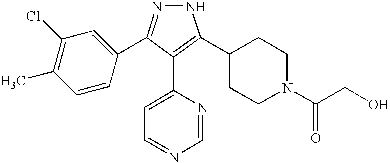 |
| P-66 | 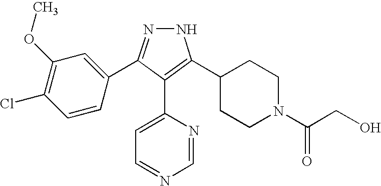 |
| P-67 | 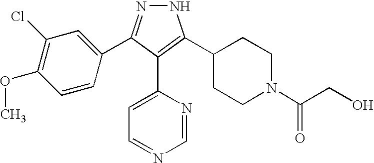 |
| P-68 | 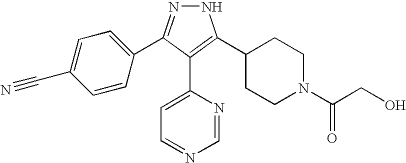 |
| P-69 | 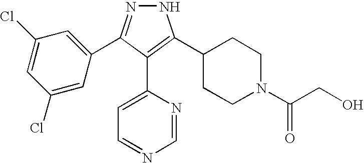 |
| P-70 | 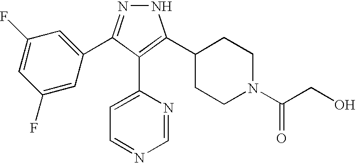 |
| P-71 | 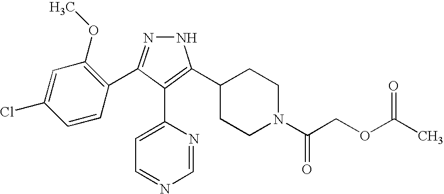 |
| P-72 | 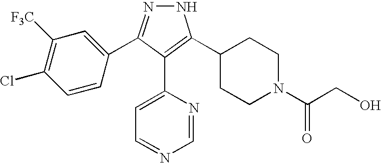 |
| P-73 | 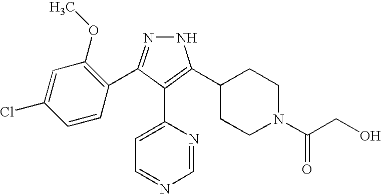 |
| P-74 | 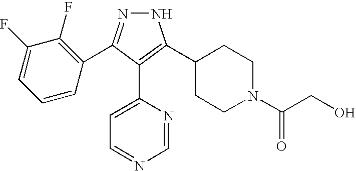 |
| P-75 | 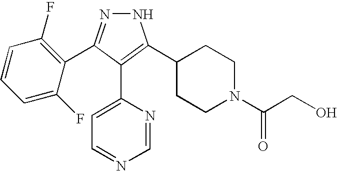 |
| P-76 | 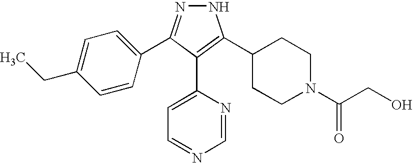 |
| P-77 | 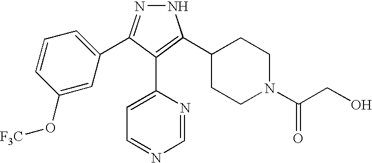 |
| P-78 | 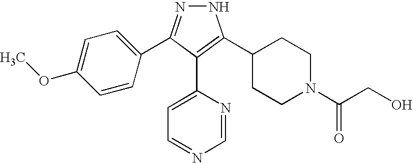 |
| P-79 | 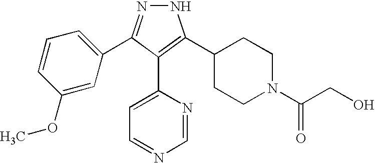 |
| P-80 | 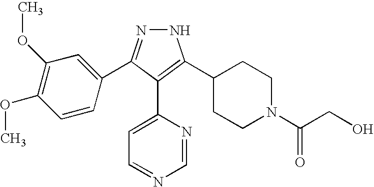 |
| P-81 | 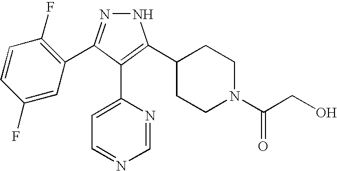 |
| P-82 | 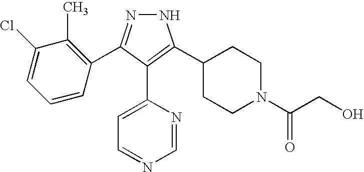 |
| P-83 | 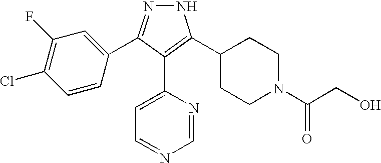 |
| P-84 | 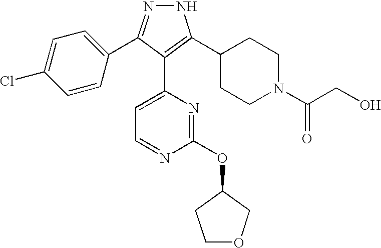 |
| P-85 | 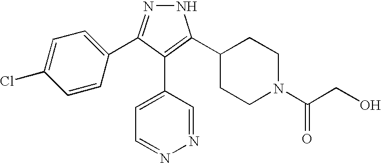 |
| P-86 | 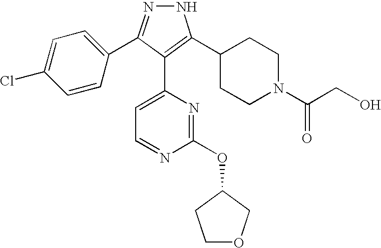 |
| P-87 | 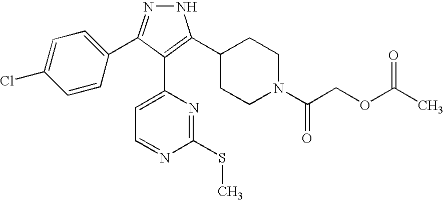 |
| P-88 | 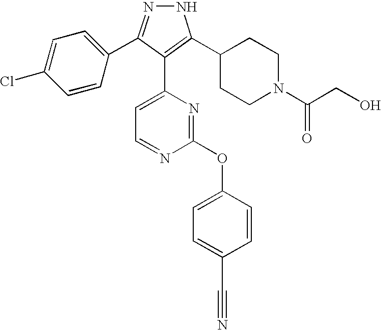 |
| P-89 | 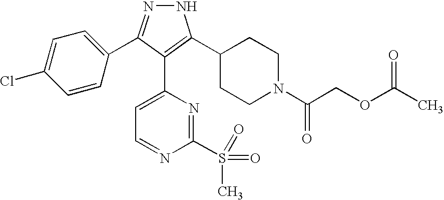 |
| P-90 | 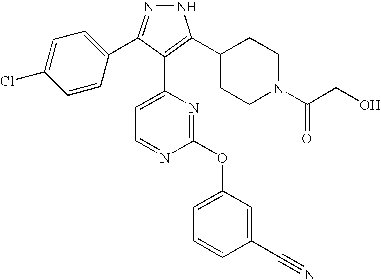 |
| P-91 | 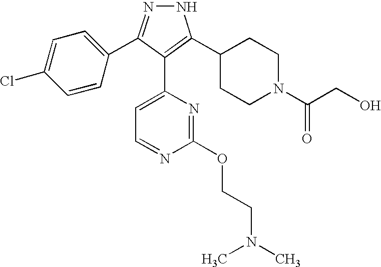 |
| P-92 | 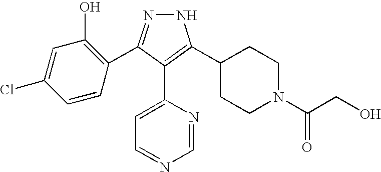 |
| P-93 | 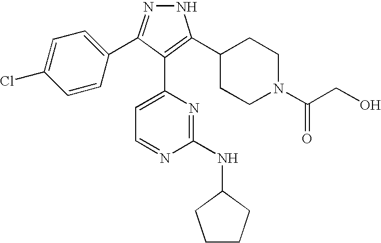 |
| P-94 | 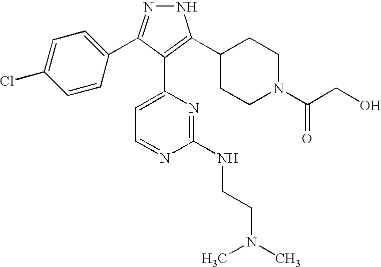 |
| P-95 | 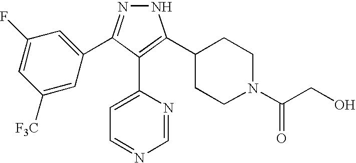 |
| P-96 | 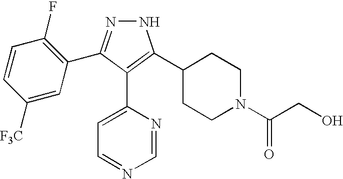 |
| P-97 | 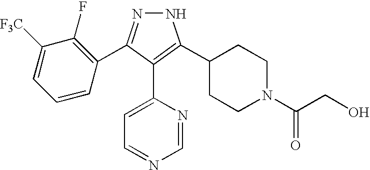 |
| P-98 | 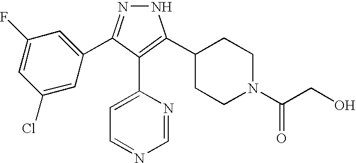 |
| P-99 | 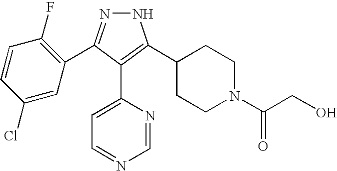 |
| P-100 | 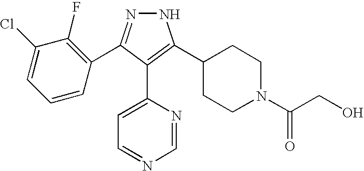 |
| P-101 | 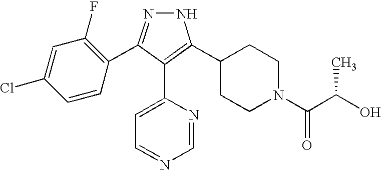 |
| P-102 | 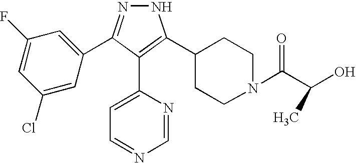 |
| P-103 | 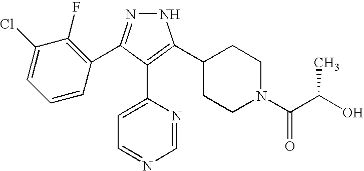 |
| P-104 | 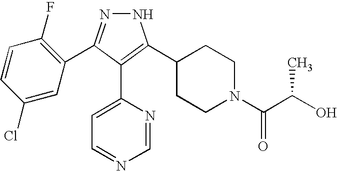 |
| P-105 | 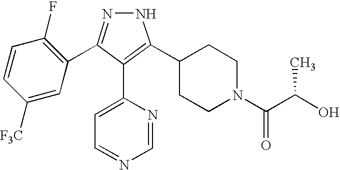 |
| P-106 | 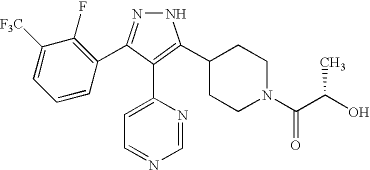 |
| P-107 | 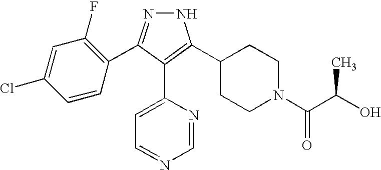 |
| P-108 | 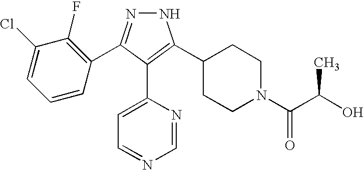 |
| P-109 | 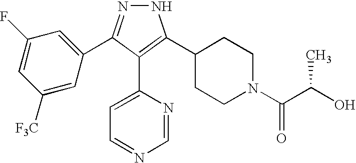 |
| P-110 | 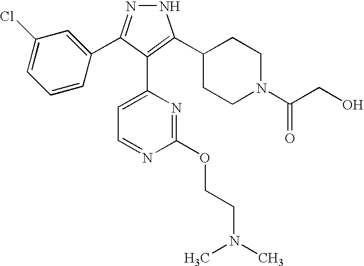 |
| P-111 | 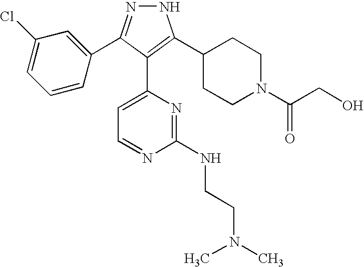 |
| P-112 | 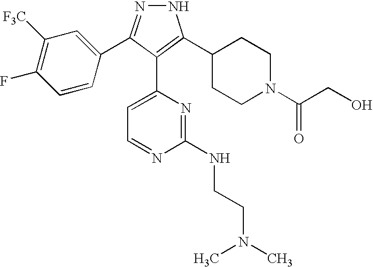 |
| P-113 | 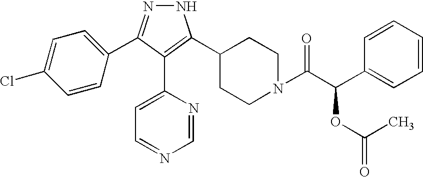 |
| P-114 | 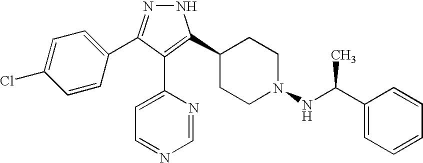 |
| P-115 | 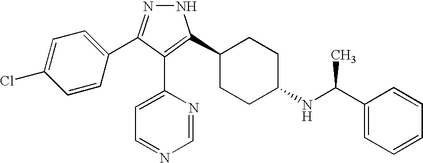 |
| P-116 | 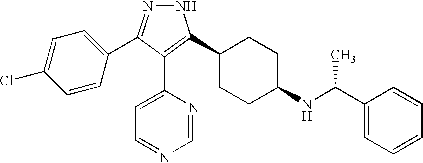 |
| P-117 | 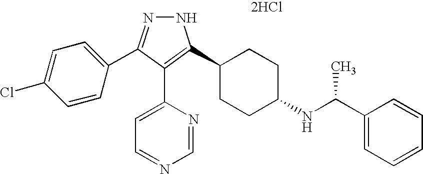 |
| P-118 | 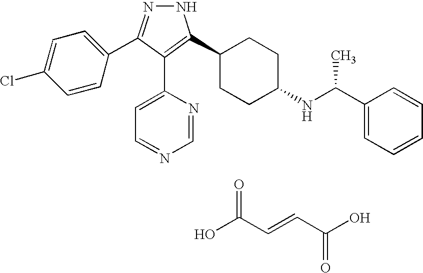 |
| P-119 | 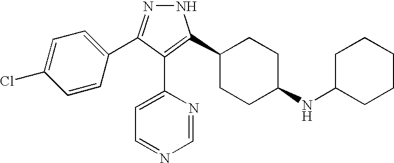 |
| P-120 | 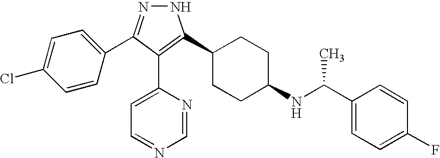 |
| P-121 | 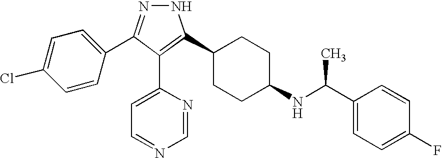 |
| P-122 | 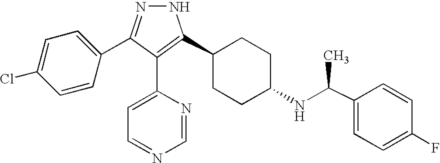 |
| P-123 | 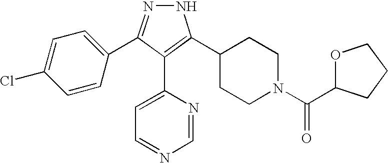 |
| P-124 | 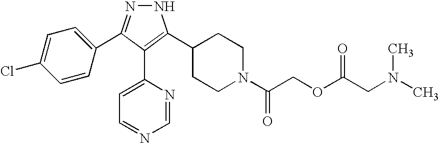 |
| P-125 | 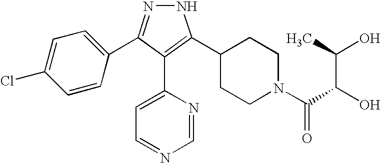 |
| P-126 | 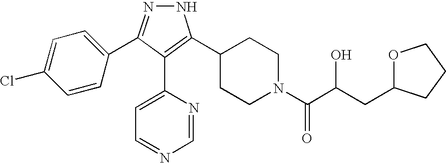 |
| P-127 | 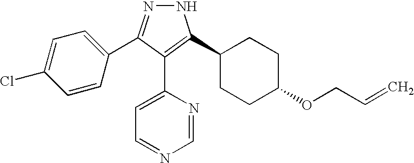 |
| P-128 | 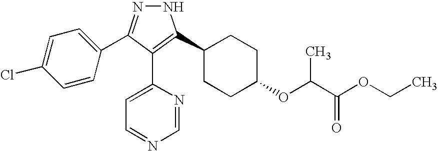 |

In some preferred embodiments, these compounds are prepared by a process disclosed by Allen et al. in U.S. patent application Ser. No. 10/254,445; and PCT Application No. PCT/US02/30409 (both of which are cited above incorporated by reference into this patent).

In some embodiments wherein the p38-kinase inhibitor comprises a substituted pyrazole, the p38-kinase inhibitor corresponds in structure to Formula P-48:

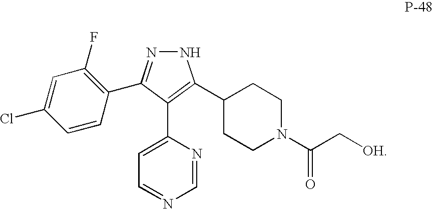

In some embodiments wherein the p38-kinase inhibitor comprises a substituted pyrazole, the p38-kinase inhibitor corresponds in structure to Formula P-49:

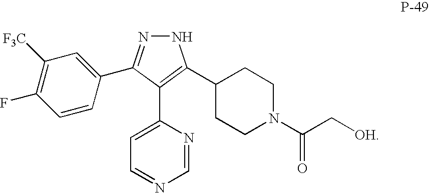

In some embodiments, the p38-kinase inhibitor comprises a substituted pyrazole corresponding in structure to an analogue of a compound in Table 1 or 2 wherein the pyrimidine at the 4-position of the pyrazole has been replaced with a pyridine.

In some embodiments wherein the p38-kinase inhibitor comprises a substituted pyrazole, the p38-kinase inhibitor comprises a compound selected from the group of reported p38-kinase inhibitors in Table 3:

TABLE 3

| | | | | Patent/ |
|---|---|---|---|---|
| | | | | Literature |
| Compound | | Compound | CAS Registry | Reference(s) for |
| Number | Compound | Identifier | Number | Compound |
| P-129 | 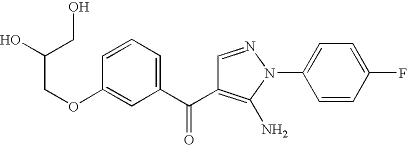 | | | |
| P-130 | 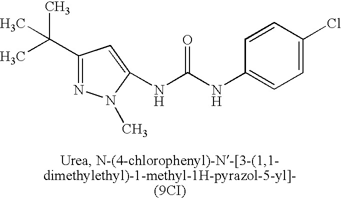 | | 432042-02-9 | Nature Structural Biology, 9(4), 268-272 (2002); Journal of Medicinal Chemistry, 45(14), 2994-3008 (2002). |
| P-131 | 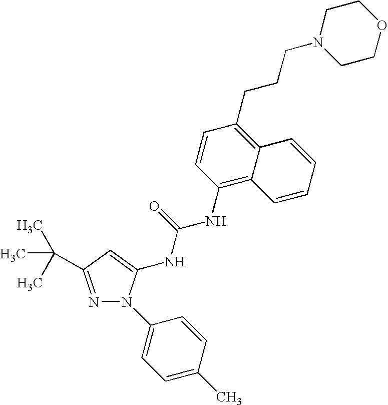 | BIRB 786 | | |
| P-132 | 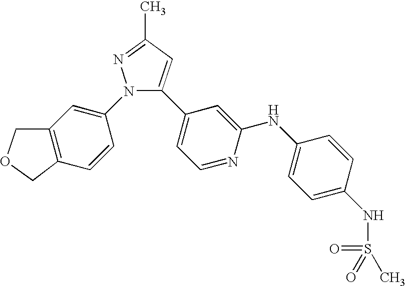 | | | WO 02/072571 |
| P-133 | 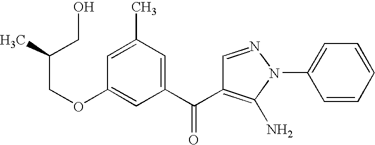 | | | |

The references cited in the above table generally disclose methods for making the corresponding compounds, and are incorporated by reference into this patent.

In some embodiments, the p38-kinase inhibitor comprises the reported p38-kinase inhibitor shown in Table 4:

TABLE 4

| | | | | Patent/ |
|---|---|---|---|---|
| | | | | Literature |
| Compound | | Compound | CAS Registry | Reference(s) for |
| Number | Compound | Identifier | Number | Compound |
| P-134 | 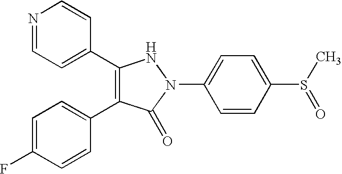 | | 219138-27-9 | Pharmacol Ther. 82: 389-397 (1999); Bioorganic & Medicinal Chemistry Letters, 8(19), 2689-2694 (1998). |

The references cited in the above table generally disclose methods for making the depicted compound, and are incorporated by reference into this patent.

In some embodiments, the p38-kinase inhibitor comprises a reported p38-kinase inhibitor shown in Table 5:

TABLE 5

| | | | | Patent/ |
|---|---|---|---|---|
| | | | | Literature |
| Compound | | Compound | CAS Registry | Reference(s) for |
| number | Compound | Identifier | Number | Compound |
| P-135 | 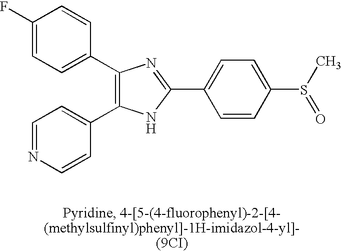 | SB203580 | 152121-47-6 | J. Pharmacol. Exp. Ther. 279: 1453-1461 (1996) WO 93/14081 WO 95/03297 |
| P-136 | 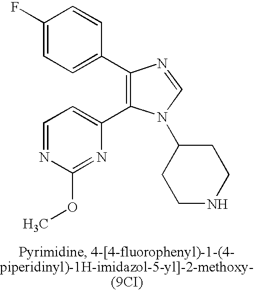 | SB242235 | 193746-75-7 | WO 97/25046 US 5,716,955 |
| P-137 | 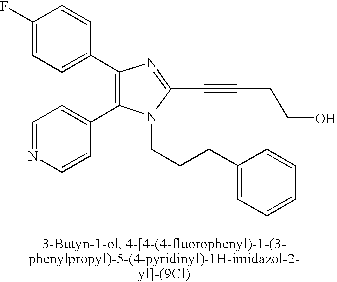 | RWJ67657 | 215303-72-3 | WO 98/47892 |
| P-138 | 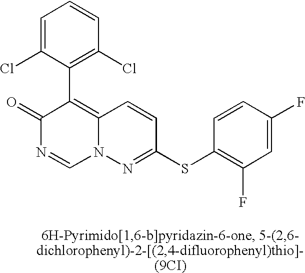 | VX-745 | 209410-46-8 | WO 98/27098 |
| P-139 | 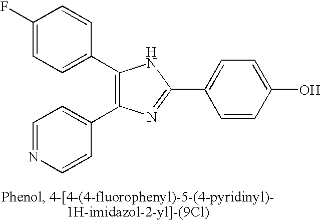 | SB202190 | 152121-30-7 | WO 93/14081 US 5,656,644 US 5,686,455 |
| P-140 | 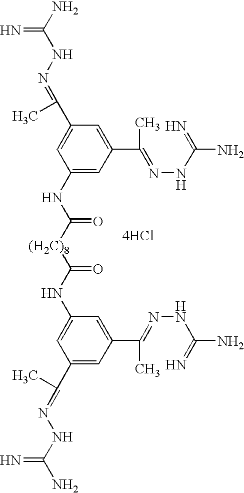 | CNI-1493 | 164301-51-3 | WO 9519767 WO9820868 US 5750573 |
| | decanediamide, N,N′-bis[3,5-bis[1- | | | |
| | [(aminoiminomethyl)hydrazono]ethyl]phenyl], | | | |
| | tetrahydrochloride (9CI) | | | |
| P-141 | 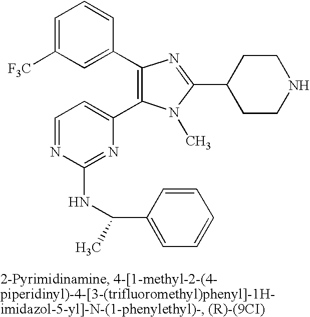 | | 200801-85-0 | Journal of Medicinal Chemistry 42(12): 2180-2190 (1999) WO 97/47618 |
| P-142 | 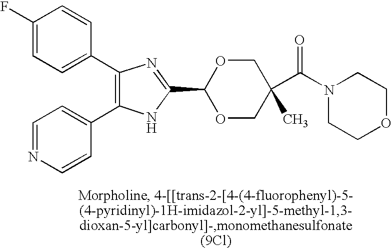 | RPR200765A | 218162-38-0 | WO 98/56788 |
| P-143 | 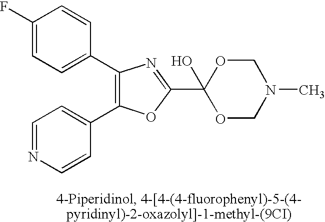 | | 290357-24-3 | Bioorganic & Medicinal Chemistry Letters 10(11): 1261-1264 (2000) |
| P-144 | 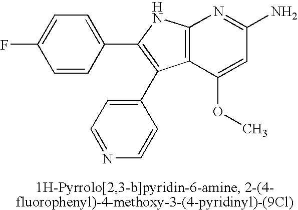 | RWJ68354 | 215306-39-1 | WO 98/47899 Tetrahedron Letters 39(48): 8763-8764 (1998) |
| P-145 | 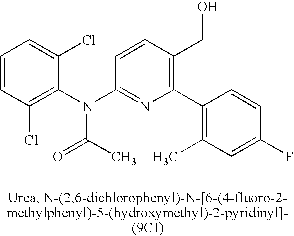 | | 250123-27-4 | WO99/58502 |
| P-146 | 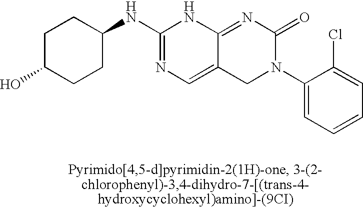 | | 335652-44-3 | WO 01/29042 |
| P-147 | 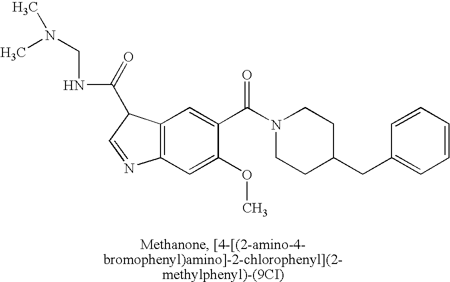 | | 321351-00-2 | WO 01/12074 |
| P-148 | 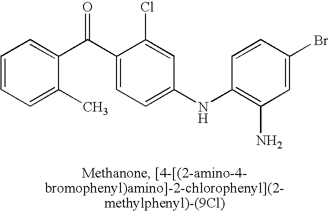 | EO1428 | 321351-00-2 | WO 0105744 WO 0105745 WO 0105746 WO 0105749 WO 0105751 |
| P-149 | 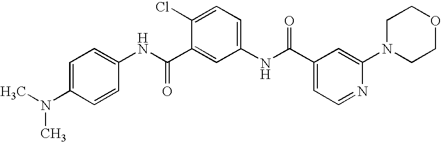 | | | Exp. Opin. Ther. Pat. 11: 1471-1473 (2001) |
| P-150 | 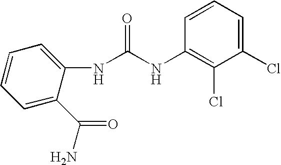 | Vertex | | |
| P151 | 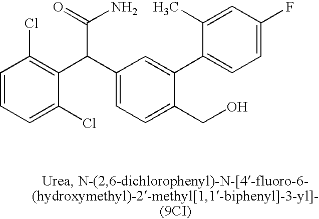 | Vertex | 304439-93-8 | Sibley et al., Bioorganic & Medicinal Chemistry Letters, 10(18): 2047-2050 (2000). |
| P-152 | 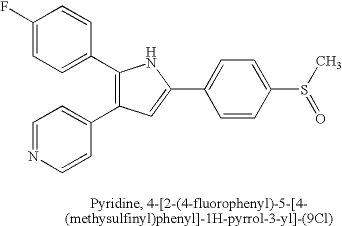 | L-167307 | 188352-45-6 | WO 9705878 WO 9716441 US 5837719 WO 0066124 |
| P-153 | 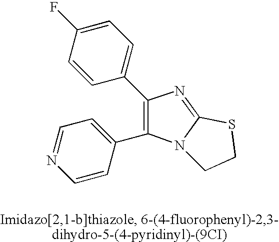 | SK&F 86002 | 72873-74-6 | Newton et al. Drug Metabolism & Disposition, 17(2): 174-9 (1989). US 4,175,127 |
| P-154 | 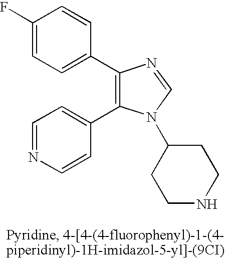 | HEP 689/ SB 235699 | 180869-32-3 | WO 9621452 US 5593992 US 5593991 |
| P-155 | 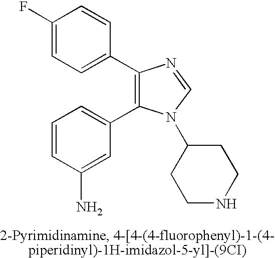 | SB 220025 | 165806-53-1 | WO 9502591 WO 9621452 US 5593992 WO 9723479 |
| P-156 | 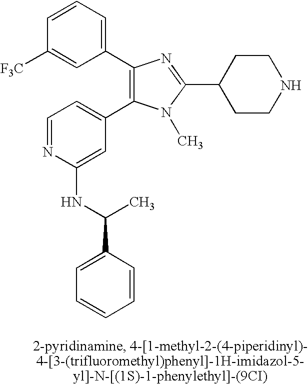 | | 189442-43-1 | WO 9712876 US 5717100 US 6083949 |
| P-157 | 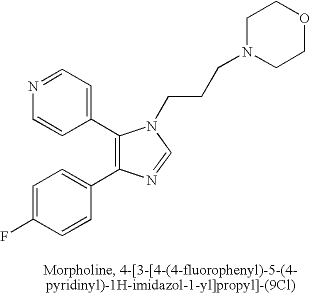 | SB 210313 | 165806-09-7 | WO 9502591 WO 9621452 US 5593992 US 5670527 |
| P-158 | 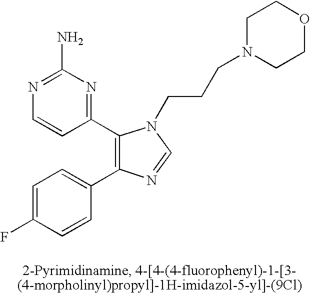 | SB 216385 | 165806-48-4 | WO 95/02591 WO 96/21452 US 5,593,992 |
| P-159 | 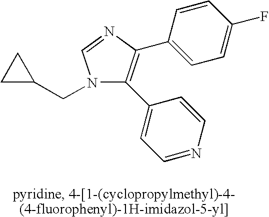 | SB 216995 | 165806-34-8 | WO 9502591 US 5,593,991 US 5,593,992 US 5670527 |
| P-160 | 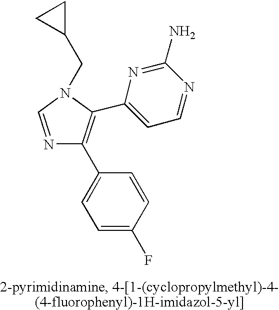 | SB 218655 | 165806-51-9 | WO 9502591 US 5,593,991 US 5,593,992 US 5670527 |
| P-161 | 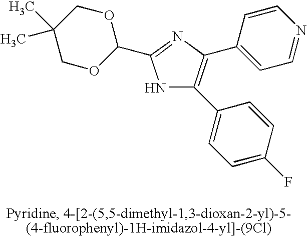 | RPR-132331 | 218145-98-3 | WO 9856788 |
| P-162 | 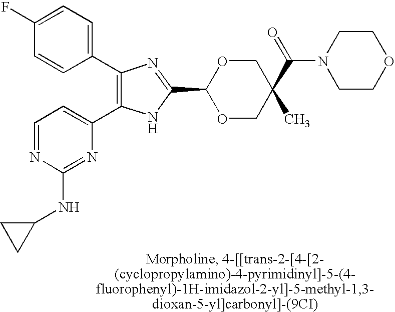 | RPR-203494 | 218160-26-0 | WO 9856788; Bioorganic & Medicinal Chemistry Letters 11(5) 693-696 (2001) |
| P-163 | 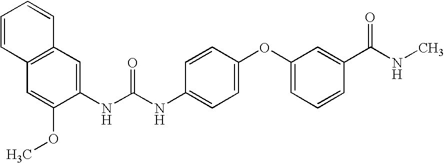 | | | |
| P-164 | 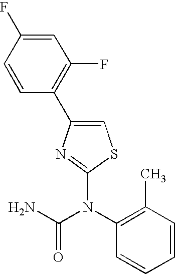 | | | WO 00/17175 |
| P-165 | 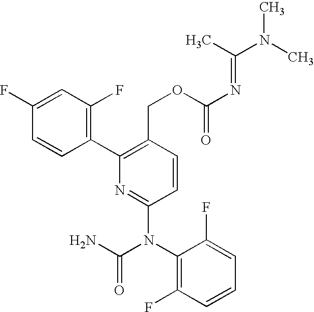 | | | WO 01/70695 WO 02/14281 |
| P-166 | 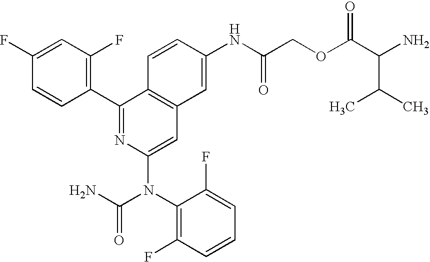 | | | WO 02/100405 |
| P-167 | 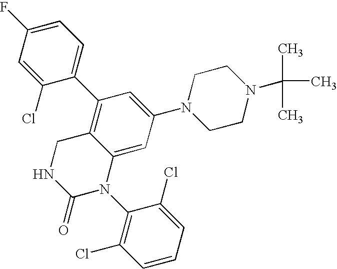 | | | WO 02/058695 |
| P-168 | 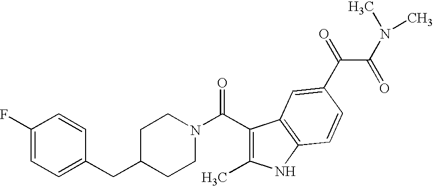 | | | WO 02/42292 |
| P-169 | 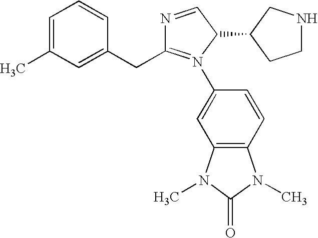 | | | |
| P-170 | 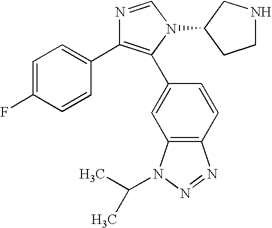 | | | EP 02-252153 |

The references cited in the above table generally disclose methods for making the corresponding compounds, and are incorporated by reference into this patent.

In some embodiments, the p38-kinase inhibitor comprises the reported p38- or kinase inhibitor corresponding in structure to Formula P-135:

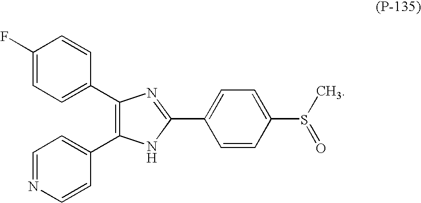

In some embodiments, the p38-kinase inhibitor comprises the reported p38-kinase inhibitor corresponding in structure to Formula P-136:

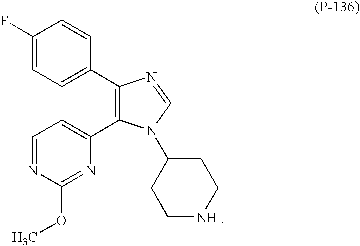

In some embodiments, the p38-kinase inhibitor comprises the reported p38-kinase inhibitor corresponding in structure to Formula P-137:

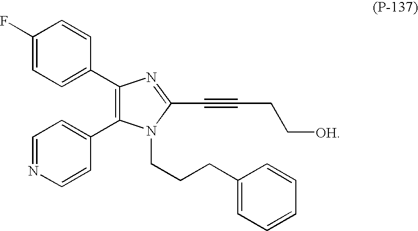

In some embodiments, the p38-kinase inhibitor comprises the reported p38-kinase inhibitor corresponding in structure to Formula P-138:

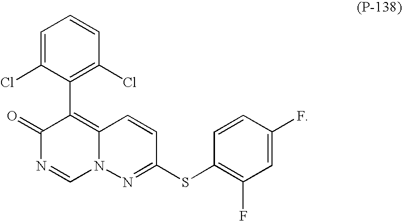

In some embodiments, the p38-kinase inhibitor comprises the reported p38-kinase inhibitor corresponding in structure to Formula P-139:

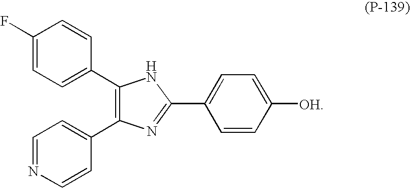

In some embodiments, the p38-kinase inhibitor comprises the reported p38-kinase inhibitor corresponding in structure to Formula P-140:

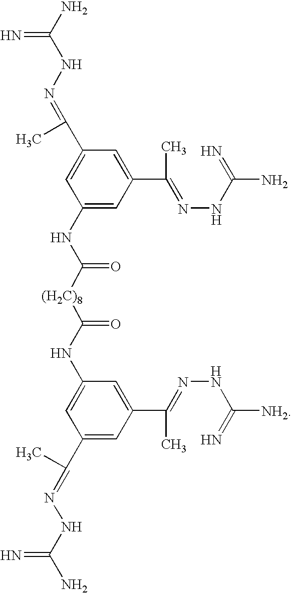

In many preferred embodiments, the p38-kinase inhibitor comprises a substituted imidazole.

Other contemplated p38-kinase inhibitors include diastomers, enantiomers, racemates, salts, conjugate acids, and pro-drugs of the above-described compounds. The present invention further contemplates any tautomeric forms of the above-described compounds. For example, pyrazoles of Formula I and I′ are magnetically and structurally equivalent because of the prototropic tautomeric nature of the hydrogen:

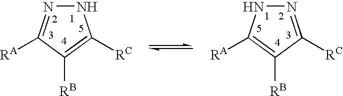

The typically preferred mode for this invention is to administer a p38-kinase inhibitor in combination with one or more aldosterone antagonists and/or diuretics to treat the above-described diseases. It should be recognized, however, that this invention also embraces the use of one or more p38-kinase inhibitors (particularly substituted-pyrazole p38-kinase inhibitors, and even more particularly substituted-pyrazole p38-kinase inhibitors described above) alone to treat the above-described diseases.

### B. Examples of Aldosterone Antagonists

The phrase “aldosterone antagonist” embraces an agent or compound, or a combination of two or more of such agents or compounds, which counteract the effect of aldosterone. Such agents and compounds, such as mespirenone, may antagonize the action of aldosterone through a pre-receptor mechanism. Other agents and compounds, such as spironolactone and eplerenone, fall generally within a class known as aldosterone receptor antagonists, which bind to mineralocorticoid receptors to prevent natural ligand activation of post-receptor events. Many suitable aldosterone antagonists are described by, for example, Perez et al. in U.S. Pat. No. 6,410,524 (issued Jun. 25, 2002; filed Nov. 5, 1999 as U.S. patent application Ser. No. 09/434,685) (incorporated by reference into this patent).

The aldosterone antagonists used in the methods of the present invention generally are spirolactone-type steroidal compounds. The term “spirolactone-type” is intended to characterize a structure comprising a lactone moiety attached to a steroid nucleus, typically at the steroid “D” ring, through a spiro bond configuration. A subclass of spirolactone-type aldosterone antagonist compounds consists of epoxy-steroidal aldosterone antagonist compounds such as eplerenone. Another subclass of spirolactone-type antagonist compounds consists of non-epoxy-steroidal aldosterone antagonist compounds such as spironolactone.

The epoxy-steroidal aldosterone antagonist compounds used in the method of the present invention generally have a steroidal nucleus substituted with an epoxy-type moiety. The term “epoxy-type” moiety is intended to embrace any moiety characterized in having an oxygen atom as a bridge between two carbon atoms, examples of which include the following moieties: 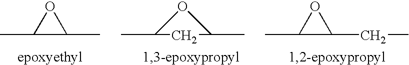
The term “steroidal”, as used in the phrase “epoxy-steroidal”, denotes a nucleus provided by a cyclopenteno-phenanthrene moiety, having the conventional “A”, “B”, “C” and “D” rings. The epoxy-type moiety may be attached to the cyclopentenophenanthrene nucleus at any attachable or substitutable positions, that is, fused to one of the rings of the steroidal nucleus or the moiety may be substituted on a ring member of the ring system. The phrase “epoxy-steroidal” is intended to embrace a steroidal nucleus having one or a plurality of epoxy-type moieties attached thereto

Epoxy-steroidal aldosterone antagonists suitable for use in the present methods include a family of compounds having an epoxy moiety fused to the “C” ring of the steroidal nucleus. Especially preferred are 20-spiroxane compounds characterized by the presence of a 9α,11α-substituted epoxy moiety. Compounds 1 through 11 in Table 6 below are illustrative 9α,11α-epoxy-steroidal compounds that may be used in the present invention. These epoxy steroids may be prepared by procedures described in Grob et al., U.S. Pat. No. 4,559,332 (incorporated by reference into this patent). Additional processes for the preparation of 9,11-epoxy steroidal compounds and their salts are disclosed in Ng et al., WO 97/21720 and Ng et al., WO 98/25948 (both of which are incorporated by reference into this patent).

TABLE 6

| Compound | | |
|---|---|---|
| No. | Structure | Name |
| A-1 | 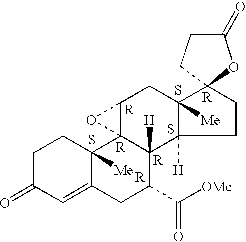 | Pregn-4-ene-7, 21-dicarboxylic acid, 9, 11-epoxy-17-hydroxy-3- oxo-, γ-lactone, methyl ester, (7α, 11α, 17β)- |
| A-2 | 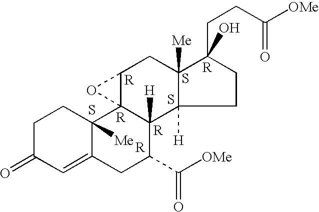 | Pregn-4-ene-7, 21-dicarboxylic acid, 9, 11-epoxy-17-hydroxy-3- oxo-, dimethyl ester, (7α, 11α, 17β)- |
| A-3 | 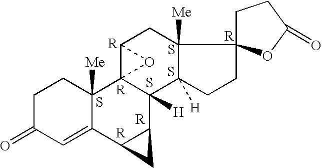 | 3′H-cyclopropa[6, 7]pregna-4, 6- diene-21-carboxylic acid, 9, 11- epoxy-6, 7-dihydro-17-hydroxy-3- oxo-, γ-lactone, (6β, 7β, 11α, 17β)- |
| A-4 | 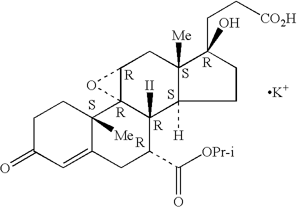 | Pregn-4-ene-7, 21-dicarboxylic acid, 9, 11-epoxy-17-hydroxy-3- oxo-, 7-(1-methylethyl) ester, monopotassium salt, (7α, 11α, 17β)- |
| A-5 | 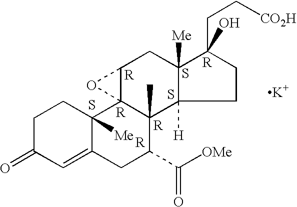 | Pregn-4-ene-7, 21-dicarboxylic acid, 9, 11-epoxy-17-hydroxy-3- oxo-, 7-methylethyl) ester, monopotassium salt, (7α, 11α, 17β)- |
| A-6 | 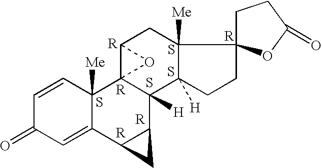 | 3′H-cyclopropa[6, 7]pregna-1, 4, 6- triene-21-carboxylic acid, 9, 11- epoxy-6, 7-dihydro-17-hydroxy-3- oxo-, γ-lactone(6β, 7β, 11α)- |
| A-7 | 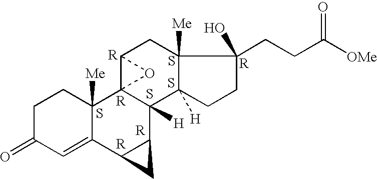 | 3′H-cyclopropa[6, 7]pregna-4, 6- diene-21-carboxylic acid, 9, 11- epoxy-6, 7-dihydro-17-hydroxy-3- oxo-, methyl ester, (6β, 7β, 11α, 17β)- |
| A-8 | 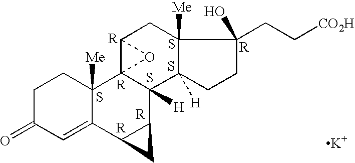 | 3′H-cyclopropa[6, 7]pregna-4, 6- diene-21-carboxylic acid, 9, 11- epoxy-6, 7-dihydro-17-hydroxy-3- oxo-, monopotassium salt, (6β, 7β, 11α, 17β)- |
| A-9 | 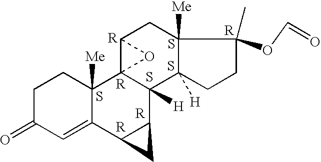 | 3′H-cyclopropa[6, 7]pregna-1, 4, 6- triene-21-carboxylic acid, 9, 11- epoxy-6, 7-dihydro-17-hydroxy-3- oxo-, γ-lactone(6β, 7β, 11α, 17β)- |
| A-10 | 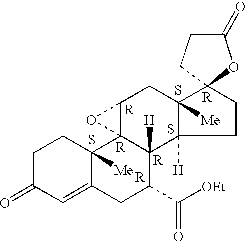 | Pregn-4-ene-7, 21-dicarboxylic acid, 9, 11-epoxy-17-hydroxy-3- oxo-, γ-lactone, ethyl ester, (7α, 11α, 17β)- |
| A-11 | 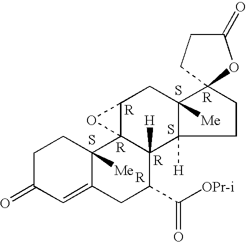 | Pregn-4-ene-7, 21-dicarboxylic acid, 9, 11-epoxy-17-hydroxy-3- oxo-, γ-lactone, 1-methylethyl ester (7α, 11α, 17β)- |

Of particular interest is the compound eplerenone (also known as epoxymexrenone or “CGP 30 083”), illustrated above as compound A-1 in Table 6. The chemical name for eplerenone is pregn-4-ene-7,21-dicarboxylic acid, 9,11-epoxy-17-hydroxy-3-oxo, γ-lactone, methyl ester, (7α, 11α, 17α)-. This chemical name corresponds to the CAS registry name for eplerenone (the CAS registry number for eplerenone is 107724-20-9). U.S. Pat. No. 4,559,332 identifies eplerenone by the alternative name of 9α,11α-epoxy-7α-methoxycarbonyl-20-spirox-4-ene-3,21-dione. Such “spiroxane” nomenclature is further described in, for example, U.S. Pat. No. 4,559,332 at col. 2, line 16 to col. 4, line 48.

Eplerenone is an aldosterone receptor antagonist, and has a greater specificity for aldosterone receptors than does, for example, spironolactone. Selection of eplerenone as the aldosterone antagonist in the present method would generally tend to be beneficial for reducing certain side-effects, such as, for example, gynecomastia (which tends to occur when less-specific aldosterone antagonists are used).

Non-epoxy-steroidal aldosterone antagonists suitable for use in the present methods include a family of spirolactone-type compounds defined by Formula I: 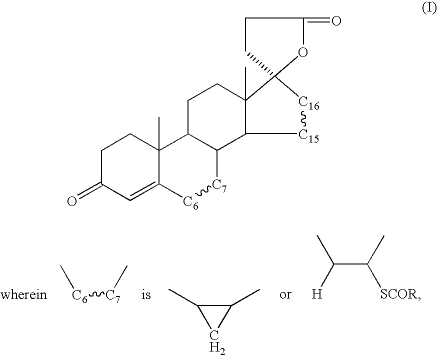
wherein R is lower alkyl having up to 5 carbon atoms, and

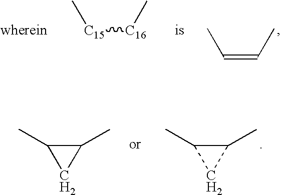

Lower alkyl residues include branched and un-branched groups, preferably methyl, ethyl, or n-propyl.

Preferred examples of such compounds include the following:
7α-acetylthio-3-oxo-4,15-androstadiene-[17(β-1′)-spiro-5′]perhydrofuran-2′-one;
3-oxo-7α-propionylthio-4, 15-androstadiene-[17((β-1′)-spiro-5′]perhydrofuran-2′-one;
6β,7β-methylene-3-oxo4,15-androstadiene-[17((β-1′)-spiro-5′]perhydrofuran-2′-one;
15α,16α-methylene-3-oxo-4,7α-propionylthio-4-androstene[17(β-1′)-spiro-5′]perhydrofuran-2′-one;
6β,7β,15α,16α-dimethylene-3-oxo-4-androstene[17(β-1′)-spiro-5′]-perhydrofuran-2′-one;
7α-acetylthio-15β, 16β-Methylene-3-oxo-4-androstene-[17(β-1′)-spiro-5′]perhydrofuran-2′-one;
15β,16β-methylene-3-oxo-7β-propionylthio-4-androstene-[17(β-1′)-spiro-5′]perhydrofuran-2′-one; and
6β,7β,15β,16β-dimethylene-3-oxo-4-androstene-[17(−1′)-spiro-5′]perhydrofuran-2′-one.

Methods to make compounds of Formula I are described by Wiechart et al. in U.S. Pat. No. 4,129,564 (issued Dec. 12, 1978) (incorporated by reference into this patent).

Another family of non-epoxy-steroidal compounds of interest is defined by Formula II: 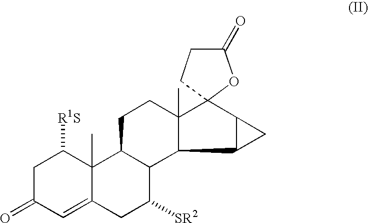
wherein R¹ is C₁₋₃-alkyl or C₁₋₃ acyl and R² is H or C₁₋₃-alkyl.

Preferred examples of such compounds include the following:
1α-acetylthio-15β,16β-methylene-7α-methylthio-3-oxo-17α-pregn-4-ene-21,17-carbolactone; and
15β,16β-methylene-1α,7α-dimethylthio-3-oxo-17α-pregn-4-ene-21,17-carbolactone.

Methods to make the compounds of Formula II are described by Nickisch et al. in U.S. Pat. No. 4,789,668 (issued Dec. 6, 1988) (incorporated by reference into this patent).

Yet another family of non-epoxy-steroidal compounds of interest is defined by a structure of Formula III: 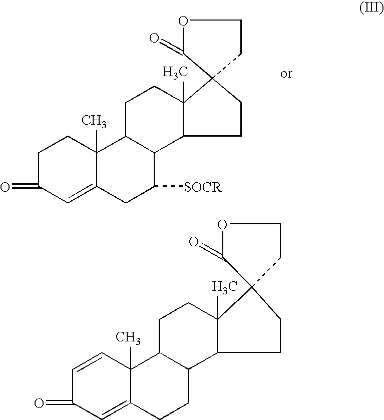
wherein R is lower alkyl, with preferred lower alkyl groups being methyl, ethyl, propyl and butyl.

Preferred examples of such compounds include:
3β,21-dihydroxy-17α-pregna-5,15-diene-17-carboxylic acid (-lactone;
3β,21-dihydroxy-17α-pregna-5,15-diene-17-carboxylic acid (-lactone 3-acetate;
3β,21-dihydroxy-17α-pregn-5-ene-17-carboxylic acid (-lactone;
3β,21-dihydroxy-17α-pregn-5-ene-17-carboxylic acid (-lactone 3-acetate;
21-hydroxy-3-oxo-17α-pregn-4-ene-17-carboxylic acid (-lactone;
21-hydroxy-3-oxo-17α-pregna-4,6-diene-17-carboxylic acid (-lactone;
21-hydroxy-3-oxo-17α-pregna-1,4-diene-17-carboxylic acid (-lactone;
7α-acylthio-21-hydroxy-3-oxo-17α-pregn-4-ene-17-carboxylic acid (lactone; and
7α-acetylthio-21-hydroxy-3-oxo-17α-pregn-4-ene-17-carboxylic acid (-lactone.

Methods to make the compounds of Formula III are described by Patchett in U.S. Pat. No. 3,257,390 (issued Jun. 21, 1966) (incorporated by reference into this patent).

Still another family of non-epoxy-steroidal compounds of interest is represented by Formula IV: 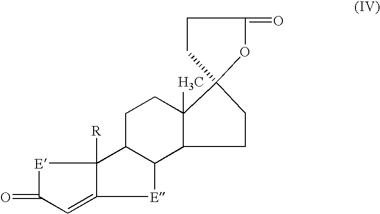
wherein E′ is ethylene, vinylene, or a (lower alkanoyl)thioethylene; E″ is ethylene, vinylene, (lower alkanoyl)thioethylene, or (lower alkanoyl)thiopropylene; R is methyl except when E′ and E″ are ethylene and (lower alkanoyl)thioethylene, respectively, in which case R is hydrogen or methyl; and the selection of E′ and E″ is such that at least one (lower alkanoyl)thio radical is present.

A preferred family of non-epoxy-steroidal compounds within Formula IV is represented by Formula V:

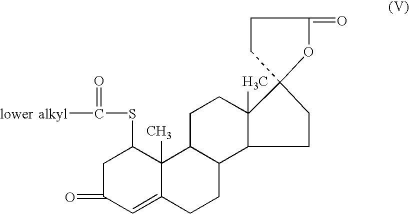

A more preferred compound of Formula V is 1-acetylthio-17α-(2-carboxyethyl)-17β-hydroxy-androst-4-en-3-one lactone.

Another preferred family of non-epoxy-steroidal compounds within Formula IV is represented by Formula VI:

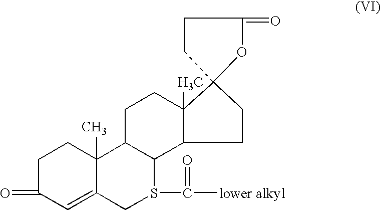

Preferred examples of compounds falling within Formula VI include the following:
7α-acetylthio-17α-(2-carboxyethyl)-17β-hydroxy-androst-4-en-3-one lactone;
7β-acetylthio-17α-(2-carboxyethyl)-17β-hydroxy-androst-4-en-3-one lactone;
1α,7α-diacetylthio-17α-(2-carboxyethyl)-17β-hydroxy-androsta-4,6-dien-3-one lactone;
7α-acetylthio-17α-(2-carboxyethyl)-17β-hydroxy-androsta-1,4-dien-3-one lactone;
7α-acetylthio-17α-(2-carboxyethyl)-17β-hydroxy-19-norandrost-4-en-3-one lactone; and
7α-acetylthio-17α-(2-carboxyethyl)-17β-hydroxy-6α-methylandrost-4-en-3-one lactone.

In Formulae IV-VI, the term “alkyl” is intended to embrace linear and branched alkyl radicals containing from 1 to about 8 carbons. The term “(lower alkanoyl)thio” embraces radicals of the formula lower alkyl

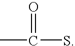

Of particular interest is the compound spironolactone, which has the following structure:

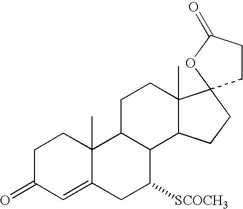

The chemical name for “spironolactone” is 17-hydroxy-7α-mercapto-3-oxo-17α-pregn-4-ene-21-carboxylic acid γ-lactone acetate.

Methods to make compounds of Formulae IV-VI are described by Cella et al. in U.S. Pat. No. 3,013,012 (issued Dec. 12, 1961) (incorporated by reference into this patent).

Another family of steroidal aldosterone antagonists is exemplified by drospirenone, i.e., [6R-6α,7α,8β,9α,10β,13β, 14α,15α,16α,17β)]-1,3′,4′,6,7,8,9,10,11,12,13,14,15,16,20,21-hexadecahydro-10,13-dimethylspiro[17H-dicyclopropa[6,7:15,16]cyclopenta[a]phenanthrene-17,2′(5H)-furan]-3,5′(2H)-dione (CAS Reg. No. 67392-87-4). Methods that may be used to make and use drospirenone are described in patent GB 1550568 (1979), which claims priority to DE 2652761 (1976) (both of which are incorporated by reference into this patent).

### C. Examples of Diuretics

The term “diuretic” includes, for example, diuretic benzothiadiazine derivatives, diuretic organomercurials, diuretic purines, diuretic steroids (including diuretic steroids having no substantial activity as an aldosterone receptor antagonist), diuretic sulfonamide derivatives, diuretic uracils, etc.

In some embodiments, the diuretic comprises a diuretic selected from the group shown in Table 7:

TABLE 7

| Compound | | |
|---|---|---|
| Number | Compound Name | Reference |
| D-1 | amanozine | Austrian Patent No. 168,063 |
| D-2 | amiloride | Belgian Patent No. 639,386 |
| D-3 | arbutin | Tschb&habln, Annalen, 1930, 479, 303 |
| D-4 | chlorazanil | Austrian Patent No. 168,063 |
| D-5 | ethacrynic acid | U.S. Pat. No. 3,255,241 |
| D-6 | etozolin | U.S. Pat. No. 3,072,653 |
| D-7 | hydracarbazine | British Patent No. 856,409 |
| D-8 | isosorbide | U.S. Pat. No. 3,160,641 |
| D-9 | mannitol | U.S. Pat. No. 2,642,462; or 2,749,371; |
| | | or 2,759,024 |
| D-10 | metochalcone | Freudenberg et al., Ber., 1957, 90, 957 |
| D-11 | muzolimine | U.S. Pat. No. 4,018,890 |
| D-12 | perhexiline | British Patent No. 1,025,578 |
| D-13 | ticrynafen | U.S. Pat. No. 3,758,506 |
| D-14 | triamterene | U.S. Pat. No. 3,081,230 |
| D-15 | urea | can be purchased from commercial |
| | | sources |

The references cited in the above table generally disclose methods for making the corresponding compounds, and are incorporated by reference into this patent.

In some embodiments, the diuretic comprises a benzothiadiazine derivative. Examples of such diuretics include, for example, those shown in Table 8:

TABLE 8

| Com- | | |
|---|---|---|
| pound | | |
| Number | Compound Name | Reference |
| D-16 | althiazide | British Patent No. 902,658 |
| D-17 | bendroflumethiazide | U.S. Pat. No. 3,265,573 |
| D-18 | benzthiazide | McManus et al., 136th Am. Soc. |
| | | Meeting (Atlantic City, September |
| | | 1959). Abstract of Papers, pp 13-O |
| D-19 | benzylhydrochlorothiazide | U.S. Pat. No. 3,108,097 |
| D-20 | buthiazide | British Patent Nos. 861,367 and |
| | | 885,078 |
| D-21 | chlorothiazide | U.S. Pat. Nos. 2,809,194 and |
| | | 2,937,169 |
| D-22 | chlorthalidone | U.S. Pat. No. 3,055,904 |
| D-23 | cyclopenthiazide | Belgian Patent No. 587,225 |
| D-24 | cyclothiazide | Whitehead et al., Journal of |
| | | Organic Chemistry, 1961, 26, 2814 |
| D-25 | epithiazide | U.S. Pat. No. 3,009,911 |
| D-26 | ethiazide | British Patent No. 861,367 |
| D-27 | fenquizone | U.S. Pat. No. 3,870,720 |
| D-28 | hydrochlorothiazide | U.S. Pat. No. 3,164,588 |
| D-29 | hydroflumethiazide | U.S. Pat. No. 3,254,076 |
| D-30 | indapamide | U.S. Pat. No. 3,565,911 |
| D-31 | methyclothiazide | Close et al., Journal of |
| | | the American |
| | | Chemical Society, 1960, 82, 1132 |
| D-32 | meticrane | French Patent Nos. M2790 and |
| | | 1,365,504 |
| D-33 | metolazone | U.S. Pat. No. 3,360,518 |
| D-34 | paraflutizide | Belgian Patent No. 620,829 |
| D-35 | polythiazide | U.S. Pat. No. 3,009,911 |
| D-36 | quinethazone | U.S. Pat. No. 2,976,289 |
| D-37 | teclothiazide | Close et al., Journal of the |
| | | American Chemical Society, |
| | | 1960, 82, 1132 |
| D-38 | trichlormethiazide | DeStevens et al., Experientia, |
| | | 1960, 16, 113 |

The references cited in the above table generally disclose methods for making the corresponding compounds, and are incorporated by reference into this patent.

In some embodiments, the diuretic comprises a sulfonamide derivative. Examples of such diuretics include, for example, those shown in Table 9:

TABLE 9

| Compound | | |
|---|---|---|
| Number | Compound Name | Reference |
| D-39 | acetazolamide | U.S. Pat. No. 2,980,679 |
| D-40 | ambuside | U.S. Pat. No. 3,188,329 |
| D-41 | azosemide | U.S. Pat. No. 3,665,002 |
| D-42 | bumetanide | U.S. Pat. No. 3,634,583 |
| D-43 | butazolamide | British Patent No. 769,757 |
| D-44 | chloraminophenamide | U.S. Pat. Nos. 2,809,194, |
| | | 2,965,655 and 2,965,656 |
| D-45 | clofenamide | Olivier, Rec. Trav. Chim., |
| | | 1918, 37, 307 |
| D-46 | clopamide | U.S. Pat. No. 3,459,756 |
| D-47 | clorexolone | U.S. Pat. No. 3,183,243 |
| D-48 | disulfamide | British Patent No. 851,287 |
| D-49 | ethoxolamide | British Patent No. 795,174 |
| D-50 | furosemide | U.S. Pat. No. 3,058,882 |
| D-51 | mefruside | U.S. Pat. No. 3,356,692 |
| D-52 | methazolamide | U.S. Pat. No. 2,783,241 |
| D-53 | piretanide | U.S. Pat. No. 4,010,273 |
| D-54 | torasemide | U.S. Pat. No. 4,018,929 |
| D-55 | tripamide | Japanese Patent No. 73 05,585 |
| D-56 | xipamide | U.S. Pat. No. 3,567,777 |

The references cited in the above table generally disclose methods for making the corresponding compounds, and are incorporated by reference into this patent.

In some embodiments, the diuretic comprises an organic mercurial diuretic. Examples of organic mercurial diuretics include mercaptomerin sodium, merethoxylline, procaine, and mersalyl with theophylline.

In some embodiments, the diuretic comprises amiloride, ethacrynic acid, triamterene, hydrochlorothiazide, chlorothiazide, bumetamide, or furosemide.

In some embodiments, the diuretic comprises hydrochlorothiazide.

In some embodiments, the diuretic comprises a loop diuretic. Examples of such diuretics include bumetamide, ethacrynic acid, and furosemide.

In some embodiments, the diuretic comprises a potassium-sparing diuretic. Examples of such diuretics include amiloride and triamterene.

### D. Definitions

The phrase “treating a condition” means ameliorating, suppressing, eradicating, reducing the severity of, decreasing the frequency of incidence of, preventing, reducing the risk of, and/or delaying the onset of the condition.

The term “combination therapy” means the administration of two or more therapeutic agents to treat a pathological condition. In this specification, the pathological condition generally comprises a cardiovascular condition or a condition associated with a cardiovascular condition. The therapeutic agents of the combination generally may be co-administered in a substantially simultaneous manner, such as, for example, (a) in a single formulation (e.g., a single capsule) having a fixed ratio of active ingredients, or (b) in multiple, separate formulations (e.g., multiple capsules) for each agent. The therapeutic agents of the combination may alternatively (or additionally) be administered at different times. In either case, the chosen treatment regimen preferably provides beneficial effects of the drug combination in treating the condition.

The phrase “therapeutically-effective” qualifies the amount of each therapeutic agent that will achieve the goal of ameliorating, suppressing, eradicating, reducing the severity of, decreasing the frequency of incidence of, preventing, reducing the risk of, and/or delaying the onset of a pathological condition.

The term “pharmaceutically-acceptable” is used adjectivally to mean that the modified noun is appropriate for use in a pharmaceutical product. When it is used, for example, to describe a carrier in a pharmaceutical composition, it characterizes the carrier as being compatible with the other ingredients of the composition and not deleterious to the recipient. Pharmaceutically acceptable cations include metallic ions and organic ions. More preferred metallic ions include, for example, appropriate alkali metal salts, alkaline earth metal salts, and other physiologically acceptable metal ions. Exemplary ions include aluminum, calcium, lithium, magnesium, potassium, sodium, and zinc in their usual valences. Preferred organic ions include protonated amines and quaternary ammonium cations, including, in part, trimethylamine, diethylamine, N,N′-dibenzylethylenediamine, chloroprocaine, choline, diethanolamine, ethylenediamine, meglumine (N-methylglucamine), and procaine. Exemplary pharmaceutically acceptable acids include, for example, hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid, oxalacetic acid, fumaric acid, propionic acid, aspartic acid, glutamic acid, benzoic acid, and the like.

With reference to the use of the words “comprise” or “comprises” or “comprising” in this patent (including the claims), Applicants note that unless the context requires otherwise, those words are used on the basis and clear understanding that they are to be interpreted inclusively, rather than exclusively.

### E. Contemplated Advantages Of The Combination Therapies Of This Invention

Benefits from the combination therapies contemplated in this patent (relative to mono-therapies using a p38-kinase inhibitor, aldosterone antagonist, or diuretic alone) may include, for example, reduced dosing requirements, greater dosing flexibility, fewer and/or less-severe side effects (particularly where there is a reduction in dosage), greater therapeutic effect(s), quicker onset of the therapeutic effect(s), and/or longer duration of the therapeutic effect(s).

### F. Preferred Dosages and Treatment Regimen

This invention is directed, in part, to a method for preventing or treating a cardiovascular condition, and/or a condition associated with a cardiovascular condition in a subject (particularly a mammal, such as a human, companion animal, farm animal, laboratory animal, zoo animal, or wild animal) having or disposed to having such a condition(s).

A contemplated combination therapy of this invention comprises dosing a first amount of a p38-kinase inhibitor and a second amount of an aldosterone antagonist or diuretic such that the first and second amounts together form a therapeutically-effective treatment for the targeted condition(s). It should be recognized that the specific dose level and frequency of dosing for the p38-kinase inhibitor and other therapeutic agents will depend on a variety of factors including, for example, the particular combination of agents selected; the activity, efficacy, pharmacokinetic, and toxicology profiles of the particular therapeutic agents used (including such profiles when the agents are used in combination); the age, weight, general health, sex, and diet of the patient; the frequency of administration; the rate of excretion; the condition(s) being treated; the severity of the condition(s) being treated; whether a drug delivery system is used; the form, route, and frequency of administration; and whether other pharmaceutically-active compounds also are being administered. Thus, the dosage regimen actually employed may vary widely, and therefore may deviate from the preferred dosage regimens set forth in this patent.

The total daily dose of each drug generally may be administered to the patient in a single dose, or in proportionate multiple subdoses. Subdoses typically are administered from 2 to about 6 times per day, and more typically from 2 to about 4 times per day. Doses may be in an immediate-release form or sustained-release form effective to obtain desired results. It should be recognized that, although the dosing frequency for the therapeutic agents in this invention is typically daily or multiple times per day, this invention also contemplates dosing regimens wherein the preferred period between administration of one or more of the therapeutic agents is greater than 24 hours. In such embodiments, the dosing frequency may be, for example, every 36 hours, every 48 hours, every 72 hours, weekly, or monthly.

In combination therapies comprising a p38-kinase inhibitor and an aldosterone antagonist or diuretic, the administration may comprise administering the p38-kinase inhibitor and the aldosterone antagonist or diuretic in a substantially simultaneous manner using either a single formulation (e.g., a single capsule) having a fixed ratio of the therapeutic agents, or separate formulations (e.g., multiple capsules) that each comprise at least one of the therapeutic agents. Such administration also may comprise administering the p38-kinase inhibitor and other therapeutic agent at different times in separate formulations. This may include, for example, administering the components of the combination in a sequential manner. Or it may include administering one component multiple times between the administration of another component. Or it may include administering two components at the same time, while also separately administering another portion at least one of those components at a different time as well. Or it may include administering the two components sequentially for a two-step effect. Where the components of the combination are dosed separately, the time period between the dosing of each component may range from a few minutes to several hours or days, and will depend on, for example, the properties of each component (e.g., potency, solubility, bioavailability, half-life, and kinetic profile), as well as the condition of the patient.

The following describes typical dosages and frequencies for p38-kinase inhibitors, and particularly for combinations comprising p38-kinase inhibitors with aldosterone antagonists and diuretics. Further dosage and dosage-frequency optimization (to the extent desirable) may be determined in trials. It should be recognized that multiple doses per day typically may be used to increase the total daily dose, if desired.

The preferred total daily dose of the p38-kinase inhibitor is typically from about 0.01 to about 100 mg/kg, more typically from about 0.1 to about 50 mg/kg, and even more typically from about 0.5 to about 30 mg/kg (i.e., mg p38-kinase inhibitor per kg body weight). A p38-kinase inhibitor typically is administered as a single daily dose, or split into from 2 to about 4 sub-doses per day.

The preferred daily dosage of aldosterone antagonist will typically be from about 0.001 to 300 mg/kg, more typically from about 0.005 and about 200 mg/kg, still more typically from about 0.01 and about 150 mg/kg. In some embodiments, the preferred dosage is from about 0.05 and about 10 mg/kg. In other embodiments, the preferred dosage is from about 0.01 to 5 mg/kg (i.e., mg p38-kinase inhibitor per kg body weight). The daily dose of aldosterone antagonist administered to a human subject typically will range from about 1 to about 400 mg. In another embodiment of the present invention, the daily dose range is from about 1 to about 200 mg. In a further embodiment of the present invention, the daily dose range is from about 1 to about 100 mg. In another embodiment of the present invention, the daily dose range is from about 10 to about 100 mg. In a further embodiment of the present invention, the daily dose range is from about 25 to about 100 mg. In another embodiment of the present invention, the daily dose is 5, 10, 12.5, 25, 50, 75, or 100 mg. In a further embodiment of the present invention, the daily dose is 25, 50, or 100 mg. A daily dose of aldosterone antagonist that produces no substantial diuretic and/or anti-hypertensive effect in a subject is specifically embraced by the present method.

Dosing of the aldosterone antagonist can be determined and adjusted based on measurement of parameters that would be known to one skilled in the art. Non-limiting examples of such parameters generally include blood pressure or appropriate surrogate markers (such as natriuretic peptides, endothelins, and other surrogate markers). Blood pressure and/or surrogate marker levels after administration of the aldosterone antagonist can be compared against the corresponding baseline levels before administering the aldosterone antagonist to determine efficacy of the present method and titrated as needed. Non-limiting examples of surrogate markers useful in the method are surrogate markers for renal and cardiovascular disease.

The dosage level for a diuretic generally will depend on the particular potency and therapeutic mechanism of the particular diuretic used (in addition to, for example, the other factors outlined above for dosage levels in general).

In some embodiments, for example, the diuretic comprises bendroflumethiazide, and the preferred dosage range is from about 2.5 to about 5 mg/day for an average-size human. Bendroflumethiazide typically is administered as a single daily dose.

In other embodiments, the diuretic comprises benzthiazide, and the preferred dosage range is from about 12.5 to about 50 mg/day. Benzthiazide typically is administered as a single daily dose.

In other embodiments, the diuretic comprises chlorothiazide, and the preferred dosage range is from about 500 to about 6000 mg/day. In other embodiments, the preferred dosage range is from about 250 to about 1000 mg/day. The chlorothiazide dosage typically is split into 2 or 3 (more typically 2) sub-doses per day.

In other embodiments, the diuretic comprises chlorthalidone, and the preferred dosage range is from about 12.5 to about 50 mg/day. Chlorthalidone typically is administered as a single daily dose.

In other embodiments, the diuretic comprises cyclothiazide, and the preferred dosage range is from about 1 to about 2 mg/day. Cyclothiazide typically is administered as a single daily dose.

In other embodiments, the diuretic comprises hydrochlorothiazide, and the preferred dosage range is from about 5 to about 100 mg/day. In other embodiments, the preferred hydrochlorothiazide dosage range is from about 5 to about 50 mg/day, and, in some embodiments, is from about 12.5 to about 50 mg/day. Hydrochlorothiazide typically is administered as a single daily dose (e.g., 12.5 or 25 mg).

In other embodiments, the diuretic comprises hydroflumethiazide, and the dosage range is from about 12.5 to about 50 mg/day. Hydroflumethiazide typically is administered as a single daily dose.

In other embodiments, the diuretic comprises indapamide, and the preferred dosage range is from about 2.5 to about 5 mg/day. Indapamide typically is administered as a single daily dose.

In other embodiments, the diuretic comprises methylcyclothiazide, and the preferred dosage range is from about 2.5 to about 5 mg/day. Methylcyclothiazide typically administered as a single daily dose.

In other embodiments, the diuretic comprises metolazone, and the preferred dosage range is from about 0.5 to about 5 mg/day. Metolazone typically is administered as a single daily dose.

In other embodiments, the diuretic comprises polythiazide, and the preferred dosage range is from about 1 to about 4 mg/day. Polythiazide typically is administered as a single daily dose.

In other embodiments, the diuretic comprises quinethiazone, and the preferred dosage range is from about 25 to about 100 mg/day. Quinethiazone typically is is administered as a single daily dose.

In other embodiments, the diuretic comprises trichloromethiazide, and the preferred dosage range is from about 1 to about 4 mg/day. Trichloromethiazide typically is administered as a single daily dose.

In other embodiments, the diuretic comprises bumetamide, and the preferred dosage range is from about 0.5 to about 5 mg/day. Bumetamide typically is administered as a single daily dose, or split into 2 or 3 sub-doses per day.

In other embodiments, the diuretic comprises ethacrynic acid, and the preferred dosage range is from about 20 to about 400 mg/day. In other embodiments, the preferred dosage range is from about 25 to about 100 mg/day. Ethacrynic acid typically is administered as a single daily dose, or split into 2 or 3 sub-doses per day.

In other embodiments, the diuretic comprises furosernide, and the preferred dosage range is from about 5 to about 1000 mg/day. In other embodiments, the preferred dosage range is from about 20 to about 320 mg/day. In embodiments wherein the furosemide comprises slow-release furosemide, the preferred dosage range is from about 30 to about 120 mg/day. Furosemide typically is administered as a single daily dose, or split into 2 or 3 sub-doses per day.

In other embodiments, the diuretic comprises amiloride, and the preferred dosage range is from about 1 to about 20 mg/day. In other embodiments, the preferred dosage range is from about 5 to about 10 mg/day. Amiloride typically is administered as a single daily dose.

In other embodiments, the diuretic comprises triamterene, and the preferred dosage range is from about 50 to about 150 mg/day. Triamterene typically is administered as a single daily dose.

It should be recognized that it is often preferred to start dosing the therapeutic agents of the combination at an intermediate levels (particularly an intermediate levels falling within the above-described preferred dosage ranges), and then titrate up or down, depending on observed efficacy and side-effects. In many embodiments, treatment is continued as necessary over a period of several weeks to several months or years until the condition(s) has been controlled or eliminated. Patients undergoing treatment with the p38-kinase inhibitors (and combinations comprising p38-kinase inhibitors) disclosed herein can be routinely monitored by a wide variety of methods known in the art for determining the effectiveness of a treatment for the particular condition being treated. This may include, for example, blood pressure, echocardiography; MRI; monitoring C-reactive protein, brain natriuretic peptides (“BNP”), fibrinogen levels, and pro-inflammatory molecule (e.g., TNF-α, MMP-2, MMP-3, MMP-13, etc.) levels in the bloodstream; and, for kidney-related diseases, it also may include, for example, monitoring the urea appearance rate (“UAR”). Continuous analysis of such data permits modification of the treatment regimen during therapy so that optimal effective amounts of each type of therapeutic agent are administered at any time, and so that the duration of treatment can be determined as well. In this way, the treatment regimen/dosing schedule can be rationally modified over the course of therapy so that the lowest amount of each therapeutic agent that together exhibit satisfactory effectiveness is administered, and so that administration is continued only so long as is necessary to successfully treat the condition.

### F-1A., Prophylactic Dosing

The combinations of this invention may be administered prophylactically, before a diagnosis of a cardiovascular condition (or associated condition), and to continue administration of the combination during the period of time the subject is susceptible to the condition. Individuals with no remarkable clinical presentation, but that are nonetheless susceptible to pathologic effects, therefore can be placed on a prophylactic dose of the combination. Such prophylactic doses may, but need not, be lower than the doses used to treat the specific pathogenic effect of interest.

### F-1B. Cardiovascular Pathology Dosing

In some embodiments of this invention, cardiac pathologies are identified, and an effective dosing and frequency determined, based on blood concentrations of natriuretic peptides. Natriuretic peptides are a group of structurally similar, but genetically distinct, peptides that have diverse actions in cardiovascular, renal, and endocrine homeostasis. Atrial natriuretic peptide (“ANP”) and brain natriuretic peptide (“BNP”) are of myocardial cell origin and C-type natriuretic peptide (“CNP”) is of endothelial origin. ANP and BNP bind to the natriuretic peptide-A receptor (“NPR-A”), which, via 3′,5′-cyclic guanosine monophosphate (cGMP), mediates natriuresis, vasodilation, renin inhibition, antimitogenesis, and lusitropic properties. Elevated natriuretic peptide levels in the blood, particularly blood BNP levels, generally are observed in subjects under conditions of blood volume expansion and after vascular injury such as acute myocardial infarction and remain elevated for an extended period of time after the infarction. (Uusimaa et al., Int. J. Cardiol, vol 69, pp. 5-14 (1999). A decrease in natriuretic peptide level relative to the baseline level measured before administration of a combination of this invention indicates a decrease in the pathologic effect of the combination, and, therefore, provides a correlation with inhibition of the pathologic effect. Blood levels of the desired natriuretic peptide level therefore can be compared against the corresponding baseline level before administration of the combination to determine efficacy of the present method in treating the pathologic effect. Based on such natriuretic peptide level measurements, dosing of the combination can be adjusted to reduce the cardiovascular pathologic effect. Cardiac pathologies also can be identified, and the appropriate dosing determined, based on circulating and urinary cGMP Levels. An increased plasma level of cGMP parallels a fall in mean arterial pressure. Increased urinary excretion of cGMP is correlated with the natriuresis.

In some embodiments, a combination of this invention is administered at a dosage and frequency effective to cause a statistically-significant decrease in tissue or circulating C-reactive protein (CRP) levels.

In some embodiments, a combination of this invention is administered at a dosage and frequency effective to cause a statistically-significant decrease in circulating pro-inflammatory molecule (e.g., TNF-α, MMP-2, MMP-9, and/or MMP-13) levels.

In some embodiments a combination of this invention is administered at a dosage and frequency effective to cause a statistically-significant decrease in circulating fibrinogen levels.

In some embodiments, a combination of this invention is administered to a patient having an ejection fraction of less than about 45%, particularly less than about 40%, and even more particularly less than about 30%. In such embodiments, the combination preferably is administered at a dosage and frequency effective to cause a statistically-significant increase (or preserve, or at least partially preserve) left ventricular ejection fraction.

In some embodiments, a combination of this invention is administered at a dosage and frequency effective to cause a statistically-significant increase (or preserve, or at least partially preserve) stroke volume.

In some embodiments, a combination of this invention is administered at a dosage and frequency effective to cause a statistically-significant decrease in left ventricular end systolic area, end diastolic area, end systolic volume, or end diastolic volume.

In some embodiments, a combination of this invention is administered at a dosage and frequency effective to cause a statistically-significant decrease in left ventricular mass.

In some embodiments, a combination of this invention is administered at a dosage and frequency effective to cause a statistically-significant decrease in interstitial collagen fraction in the heart (which can be monitored by, for example, measuring collagen markers or measuring the stiffness of the heart using, for example, an echocardiogram).

In some embodiments, a combination of this invention is administered based on the presence of myocardial infarction or heart failure or left ventricular hypertrophy. Left ventricular hypertrophy can be identified by echo-cardiogram or magnetic resonance imaging and used to monitor the progress of the treatment and appropriateness of the dosing.

### F-1C. Hypertension Dosing

For the treatment of hypertension, the subject is typically first identified as normotensive, borderline hypertensive, or hypertensive based on blood pressure determinations. For humans, in particular, such a determination may be achieved using a seated cuff mercury sphygmomanometer. Individuals may be deemed normotensive when systolic blood pressure and diastolic blood pressure are less than about 125 mm Hg and less than about 80 mm Hg, respectively; borderline hypertensive when systolic blood pressure and diastolic blood pressure are in the range of from about 125 to about 140 mm Hg and from about 80 to about 90 mm Hg, respectively; and hypertensive when systolic blood pressure and diastolic blood pressure are greater than about 140 mm Hg and 90 mm Hg, respectively. As the severity of the hypertensive condition increases, the preferred dose of at least one component of the combination typically increases. Based on post-administration blood pressure measurement, the doses of the components of the combination may be titrated. After an initial evaluation of the subject's response to the treatment, the doses may be increased or decreased accordingly to achieve the desired blood pressure lowering effect.

### F-1D. Renal Pathology Dosing

Dosing and frequency to treat pathologies of renal function can be determined and adjusted based on, for example, measurement of proteinuria, microalbuminuria, decreased glomerular filtration rate (GFR), or decreased creatinine clearance. Proteinuria is identified by the presence of greater than about 0.3 g of urinary protein in a 24 hour urine collection. Microalbuminuria is identified by an increase in assayable urinary albumin. Based upon such measurements, dosing of the dosing and frequency of a combination of this invention can be adjusted to ameliorate a renal pathologic effect.

### F-1E. Neuropathy Pathology Dosing

Neuropathy, especially peripheral neuropathy, can be identified by, and dosing and frequency adjustments based on, neurologic exam of sensory deficit or sensory motor ability.

### F-1F. Retinopathy Pathology Dosing

Retinopathy can be identified by, and dosing and frequency adjustments based on, ophthalmologic exam.

### F-2A. Example Combinations Comprising

### A p38-Kinase Inhibitors With An Aldosterone Antagonist

Table 10 illustrates examples of some of the combinations of the present invention wherein the combination comprises a first amount of a substituted-pyrazole p38-kinase inhibitor and a second amount of an aldosterone antagonist:

TABLE 10

| Example | | |
|---|---|---|
| Combination No. | p38-kinase inhibitor | aldosterone antagonist |
| 1 | P-1 | eplerenone |
| | | (A-1 in Table 6) |
| 2 | P-1 | spironolactone |
| 3 | P-2 | eplerenone |
| 4 | P-2 | spironolactone |
| 5 | P-3 | eplerenone |
| 6 | P-3 | spironolactone |
| 7 | P-4 | eplerenone |
| 8 | P-4 | spironolactone |
| 9 | P-5 | eplerenone |
| 10 | P-5 | spironolactone |
| 11 | P-6 | eplerenone |
| 12 | P-6 | spironolactone |
| 13 | P-7 | eplerenone |
| 14 | P-7 | spironolactone |
| 15 | P-8 | eplerenone |
| 16 | P-8 | spironolactone |
| 17 | P-9 | eplerenone |
| 18 | P-9 | spironolactone |
| 19 | P-10 | eplerenone |
| 20 | P-10 | spironolactone |
| 21 | P-11 | eplerenone |
| 22 | P-11 | spironolactone |
| 23 | P-12 | eplerenone |
| 24 | P-12 | spironolactone |
| 25 | P-13 | eplerenone |
| 26 | P-13 | spironolactone |
| 27 | P-14 | eplerenone |
| 28 | P-14 | spironolactone |
| 29 | P-15 | eplerenone |
| 30 | P-15 | spironolactone |
| 31 | P-16 | eplerenone |
| 32 | P-16 | spironolactone |
| 33 | P-17 | eplerenone |
| 34 | P-17 | spironolactone |
| 35 | P-18 | eplerenone |
| 36 | P-18 | spironolactone |
| 37 | P-19 | eplerenone |
| 38 | P-19 | spironolactone |
| 39 | P-20 | eplerenone |
| 40 | P-20 | spironolactone |
| 41 | P-21 | eplerenone |
| 42 | P-21 | spironolactone |
| 43 | P-22 | eplerenone |
| 44 | P-22 | spironolactone |
| 45 | P-23 | eplerenone |
| 46 | P-23 | spironolactone |
| 47 | P-24 | eplerenone |
| 48 | P-24 | spironolactone |
| 49 | P-25 | eplerenone |
| 50 | P-25 | spironolactone |
| 51 | P-26 | eplerenone |
| 52 | P-26 | spironolactone |
| 53 | P-27 | eplerenone |
| 54 | P-27 | spironolactone |
| 55 | P-28 | eplerenone |
| 56 | P-28 | spironolactone |
| 57 | P-29 | eplerenone |
| 58 | P-29 | spironolactone |
| 59 | P-30 | eplerenone |
| 60 | P-30 | spironolactone |
| 61 | P-31 | eplerenone |
| 62 | P-31 | spironolactone |
| 63 | P-32 | eplerenone |
| 64 | P-32 | spironolactone |
| 65 | P-33 | eplerenone |
| 66 | P-33 | spironolactone |
| 67 | P-34 | eplerenone |
| 68 | P-34 | spironolactone |
| 69 | P-35 | eplerenone |
| 70 | P-35 | spironolactone |
| 71 | P-36 | eplerenone |
| 72 | P-36 | spironolactone |
| 73 | P-37 | eplerenone |
| 74 | P-37 | spironolactone |
| 75 | P-38 | eplerenone |
| 76 | P-38 | spironolactone |
| 77 | P-39 | eplerenone |
| 78 | P-39 | spironolactone |
| 79 | P-40 | eplerenone |
| 80 | P-40 | spironolactone |
| 81 | P-41 | eplerenone |
| 82 | P-41 | spironolactone |
| 83 | P-42 | eplerenone |
| 84 | P-42 | spironolactone |
| 85 | P-43 | eplerenone |
| 86 | P-43 | spironolactone |
| 87 | P-44 | eplerenone |
| 88 | P-44 | spironolactone |
| 89 | P-45 | eplerenone |
| 90 | P-45 | spironolactone |
| 91 | P-46 | eplerenone |
| 92 | P-46 | spironolactone |
| 93 | P-47 | eplerenone |
| 94 | P-47 | spironolactone |
| 95 | P-48 | eplerenone |
| 96 | P-48 | spironolactone |
| 97 | P-49 | eplerenone |
| 98 | P-49 | spironolactone |
| 99 | P-50 | eplerenone |
| 100 | P-50 | spironolactone |
| 101 | P-51 | eplerenone |
| 102 | P-51 | spironolactone |
| 103 | P-52 | eplerenone |
| 104 | P-52 | spironolactone |
| 105 | P-53 | eplerenone |
| 106 | P-53 | spironolactone |
| 107 | P-54 | eplerenone |
| 108 | P-54 | spironolactone |
| 109 | P-55 | eplerenone |
| 110 | P-55 | spironolactone |
| 111 | P-56 | eplerenone |
| 112 | P-56 | spironolactone |
| 113 | P-57 | eplerenone |
| 114 | P-57 | spironolactone |
| 115 | P-58 | eplerenone |
| 116 | P-58 | spironolactone |
| 117 | P-59 | eplerenone |
| 118 | P-59 | spironolactone |
| 119 | P-60 | eplerenone |
| 120 | P-60 | spironolactone |
| 121 | P-61 | eplerenone |
| 122 | P-61 | spironolactone |
| 123 | P-62 | eplerenone |
| 124 | P-62 | spironolactone |
| 125 | P-63 | eplerenone |
| 126 | P-63 | spironolactone |
| 127 | P-64 | eplerenone |
| 128 | P-64 | spironolactone |
| 129 | P-65 | eplerenone |
| 130 | P-65 | spironolactone |
| 131 | P-66 | eplerenone |
| 132 | P-66 | spironolactone |
| 133 | P-67 | eplerenone |
| 134 | P-67 | spironolactone |
| 135 | P-68 | eplerenone |
| 136 | P-68 | spironolactone |
| 137 | P-69 | eplerenone |
| 138 | P-69 | spironolactone |
| 139 | P-70 | eplerenone |
| 140 | P-70 | spironolactone |
| 141 | P-71 | eplerenone |
| 142 | P-71 | spironolactone |
| 143 | P-72 | eplerenone |
| 144 | P-72 | spironolactone |
| 145 | P-73 | eplerenone |
| 146 | P-73 | spironolactone |
| 147 | P-74 | eplerenone |
| 148 | P-74 | spironolactone |
| 149 | P-75 | eplerenone |
| 150 | P-75 | spironolactone |
| 151 | P-76 | eplerenone |
| 152 | P-76 | spironolactone |
| 153 | P-77 | eplerenone |
| 154 | P-77 | spironolactone |
| 155 | P-78 | eplerenone |
| 156 | P-78 | spironolactone |
| 157 | P-79 | eplerenone |
| 158 | P-79 | spironolactone |
| 159 | P-80 | eplerenone |
| 160 | P-80 | spironolactone |
| 161 | P-81 | eplerenone |
| 162 | P-81 | spironolactone |
| 163 | P-82 | eplerenone |
| 164 | P-82 | spironolactone |
| 165 | P-83 | eplerenone |
| 166 | P-83 | spironolactone |
| 167 | P-84 | eplerenone |
| 168 | P-84 | spironolactone |
| 169 | P-85 | eplerenone |
| 170 | P-85 | spironolactone |
| 171 | P-86 | eplerenone |
| 172 | P-86 | spironolactone |
| 173 | P-87 | eplerenone |
| 174 | P-87 | spironolactone |
| 175 | P-88 | eplerenone |
| 176 | P-88 | spironolactone |
| 177 | P-89 | eplerenone |
| 178 | P-89 | spironolactone |
| 179 | P-90 | eplerenone |
| 180 | P-90 | spironolactone |
| 181 | P-91 | eplerenone |
| 182 | P-91 | spironolactone |
| 183 | P-92 | eplerenone |
| 184 | P-92 | spironolactone |
| 185 | P-93 | eplerenone |
| 186 | P-93 | spironolactone |
| 187 | P-94 | eplerenone |
| 188 | P-94 | spironolactone |
| 189 | P-95 | eplerenone |
| 190 | P-95 | spironolactone |
| 191 | P-96 | eplerenone |
| 192 | P-96 | spironolactone |
| 193 | P-97 | eplerenone |
| 194 | P-97 | spironolactone |
| 195 | P-98 | eplerenone |
| 196 | P-98 | spironolactone |
| 197 | P-99 | eplerenone |
| 198 | P-99 | spironolactone |
| 199 | P-100 | eplerenone |
| 200 | P-100 | spironolactone |
| 201 | P-101 | eplerenone |
| 202 | P-101 | spironolactone |
| 203 | P-102 | eplerenone |
| 204 | P-102 | spironolactone |
| 205 | P-103 | eplerenone |
| 206 | P-103 | spironolactone |
| 207 | P-104 | eplerenone |
| 208 | P-104 | spironolactone |
| 209 | P-105 | eplerenone |
| 210 | P-105 | spironolactone |
| 211 | P-106 | eplerenone |
| 212 | P-106 | spironolactone |
| 213 | P-107 | eplerenone |
| 214 | P-107 | spironolactone |
| 215 | P-108 | eplerenone |
| 216 | P-108 | spironolactone |
| 217 | P-109 | eplerenone |
| 218 | P-109 | spironolactone |
| 219 | P-110 | eplerenone |
| 220 | P-110 | spironolactone |
| 221 | P-111 | eplerenone |
| 222 | P-111 | spironolactone |
| 223 | P-112 | eplerenone |
| 224 | P-112 | spironolactone |
| 225 | P-113 | eplerenone |
| 226 | P-113 | spironolactone |
| 227 | P-114 | eplerenone |
| 228 | P-114 | spironolactone |
| 229 | P-115 | eplerenone |
| 230 | P-115 | spironolactone |
| 231 | P-116 | eplerenone |
| 232 | P-116 | spironolactone |
| 233 | P-117 | eplerenone |
| 234 | P-117 | spironolactone |
| 235 | P-118 | eplerenone |
| 236 | P-118 | spironolactone |
| 237 | P-119 | eplerenone |
| 238 | P-119 | spironolactone |
| 239 | P-120 | eplerenone |
| 240 | P-120 | spironolactone |
| 241 | P-121 | eplerenone |
| 242 | P-121 | spironolactone |
| 243 | P-122 | eplerenone |
| 244 | P-122 | spironolactone |
| 245 | P-123 | eplerenone |
| 246 | P-123 | spironolactone |
| 247 | P-124 | eplerenone |
| 248 | P-124 | spironolactone |
| 249 | P-125 | eplerenone |
| 250 | P-125 | spironolactone |
| 251 | P-126 | eplerenone |
| 252 | P-126 | spironolactone |
| 253 | P-127 | eplerenone |
| 254 | P-127 | spironolactone |
| 255 | P-128 | eplerenone |
| 256 | P-128 | spironolactone |
| 257 | P-129 | eplerenone |
| 258 | P-129 | spironolactone |
| 259 | P-130 | eplerenone |
| 260 | P-130 | spironolactone |
| 261 | P-131 | eplerenone |
| 262 | P-131 | spironolactone |
| 263 | P-132 | eplerenone |
| 264 | P-132 | spironolactone |
| 265 | P-133 | eplerenone |
| 266 | P-133 | spironolactone |
| 267 | P-134 | eplerenone |
| 268 | P-134 | spironolactone |
| 269 | P-135 | eplerenone |
| 270 | P-135 | spironolactone |
| 271 | P-136 | eplerenone |
| 272 | P-136 | spironolactone |
| 273 | P-137 | eplerenone |
| 274 | P-137 | spironolactone |
| 275 | P-138 | eplerenone |
| 276 | P-138 | spironolactone |
| 277 | P-139 | eplerenone |
| 278 | P-139 | spironolactone |
| 279 | P-140 | eplerenone |
| 280 | P-140 | spironolactone |
| 281 | P-141 | eplerenone |
| 282 | P-141 | spironolactone |
| 283 | P-142 | eplerenone |
| 284 | P-142 | spironolactone |
| 285 | P-143 | eplerenone |
| 286 | P-143 | spironolactone |
| 287 | P-144 | eplerenone |
| 288 | P-144 | spironolactone |
| 289 | P-145 | eplerenone |
| 290 | P-145 | spironolactone |
| 291 | P-146 | eplerenone |
| 292 | P-146 | spironolactone |
| 293 | P-147 | eplerenone |
| 294 | P-147 | spironolactone |
| 295 | P-148 | eplerenone |
| 296 | P-148 | spironolactone |
| 297 | P-149 | eplerenone |
| 298 | P-149 | spironolactone |
| 299 | P-150 | eplerenone |
| 300 | P-150 | spironolactone |
| 301 | P-151 | eplerenone |
| 302 | P-151 | spironolactone |
| 303 | P-152 | eplerenone |
| 304 | P-152 | spironolactone |
| 305 | P-153 | eplerenone |
| 306 | P-153 | spironolactone |
| 307 | P-154 | eplerenone |
| 308 | P-154 | spironolactone |
| 309 | P-155 | eplerenone |
| 310 | P-155 | spironolactone |
| 311 | P-156 | eplerenone |
| 312 | P-156 | spironolactone |
| 313 | P-157 | eplerenone |
| 314 | P-157 | spironolactone |
| 315 | P-158 | eplerenone |
| 316 | P-158 | spironolactone |
| 317 | P-159 | eplerenone |
| 318 | P-159 | spironolactone |
| 319 | P-160 | eplerenone |
| 320 | P-160 | spironolactone |
| 321 | P-161 | eplerenone |
| 322 | P-161 | spironolactone |
| 323 | P-162 | eplerenone |
| 324 | P-162 | spironolactone |
| 325 | P-163 | eplerenone |
| 326 | P-163 | spironolactone |
| 327 | P-164 | eplerenone |
| 328 | P-164 | spironolactone |
| 329 | P-165 | eplerenone |
| 330 | P-165 | spironolactone |
| 331 | P-166 | eplerenone |
| 332 | P-166 | spironolactone |
| 333 | P-167 | eplerenone |
| 334 | P-167 | spironolactone |
| 335 | P-168 | eplerenone |
| 336 | P-168 | spironolactone |
| 337 | P-169 | eplerenone |
| 338 | P-169 | spironolactone |
| 339 | P-170 | eplerenone |
| 340 | P-170 | spironolactone |

Table 11 illustrates examples of some of the combinations of the present invention comprises a first amount of a reported substituted-pyrazole p38-kinase inhibitor and a second amount of an aldosterone antagonist:

TABLE 11

| Example | | |
|---|---|---|
| Combination No. | p38-kinase inhibitor | aldosterone antagonist |
| 341 | P-1 | A-2 |
| 342 | P-1 | A-3 |
| 343 | P-1 | A-4 |
| 344 | P-1 | A-5 |
| 345 | P-1 | A-6 |
| 346 | P-1 | A-7 |
| 347 | P-1 | A-8 |
| 348 | P-1 | A-9 |
| 349 | P-1 | A-10 |
| 350 | P-1 | A-11 |
| 351 | P-15 | A-2 |
| 352 | P-15 | A-3 |
| 353 | P-15 | A-4 |
| 354 | P-15 | A-5 |
| 355 | P-15 | A-6 |
| 356 | P-15 | A-7 |
| 357 | P-15 | A-8 |
| 358 | P-15 | A-9 |
| 359 | P-15 | A-10 |
| 360 | P-15 | A-11 |
| 361 | P-18 | A-2 |
| 362 | P-18 | A-3 |
| 363 | P-18 | A-4 |
| 364 | P-18 | A-5 |
| 365 | P-18 | A-6 |
| 366 | P-18 | A-7 |
| 367 | P-18 | A-8 |
| 368 | P-18 | A-9 |
| 369 | P-18 | A-10 |
| 370 | P-18 | A-11 |
| 371 | P-21 | A-2 |
| 372 | P-21 | A-3 |
| 373 | P-21 | A-4 |
| 374 | P-21 | A-5 |
| 375 | P-21 | A-6 |
| 376 | P-21 | A-7 |
| 377 | P-21 | A-8 |
| 378 | P-21 | A-9 |
| 379 | P-21 | A-10 |
| 380 | P-21 | A-11 |
| 381 | P-48 | A-2 |
| 382 | P-48 | A-3 |
| 383 | P-48 | A-4 |
| 384 | P-48 | A-5 |
| 385 | P-48 | A-6 |
| 386 | P-48 | A-7 |
| 387 | P-48 | A-8 |
| 388 | P-48 | A-9 |
| 389 | P-48 | A-10 |
| 390 | P-48 | A-11 |
| 391 | P-49 | A-2 |
| 392 | P-49 | A-3 |
| 393 | P-49 | A-4 |
| 394 | P-49 | A-5 |
| 395 | P-49 | A-6 |
| 396 | P-49 | A-7 |
| 397 | P-49 | A-8 |
| 398 | P-49 | A-9 |
| 399 | P-49 | A-10 |
| 400 | P-49 | A-11 |

The “A” numbers identifying the aldosterone antagonists in Table II correspond to the compounds numbers in the tables above. The same is true for the remaining combination table that follow.

Table 12 illustrates examples of some of the combinations of the present invention wherein the combination comprises a first amount of a reported substituted-pyrazole p38-kinase inhibitor and a second amount of an aldosterone antagonist:

TABLE 12

| Example | | |
|---|---|---|
| Combination No. | p38-kinase inhibitor | aldosterone antagonist |
| 401 | P-129 | A-2 |
| 402 | P-129 | A-3 |
| 403 | P-129 | A-4 |
| 404 | P-129 | A-5 |
| 405 | P-129 | A-6 |
| 406 | P-129 | A-7 |
| 407 | P-129 | A-8 |
| 408 | P-129 | A-9 |
| 409 | P-129 | A-10 |
| 410 | P-129 | A-11 |
| 411 | P-130 | A-2 |
| 412 | P-130 | A-3 |
| 413 | P-130 | A-4 |
| 414 | P-130 | A-5 |
| 415 | P-130 | A-6 |
| 416 | P-130 | A-7 |
| 417 | P-130 | A-8 |
| 418 | P-130 | A-9 |
| 419 | P-130 | A-10 |
| 420 | P-130 | A-11 |
| 421 | P-131 | A-2 |
| 422 | P-131 | A-3 |
| 423 | P-131 | A-4 |
| 424 | P-131 | A-5 |
| 425 | P-131 | A-6 |
| 426 | P-131 | A-7 |
| 427 | P-131 | A-8 |
| 428 | P-131 | A-9 |
| 429 | P-131 | A-10 |
| 430 | P-131 | A-11 |
| 431 | P-132 | A-2 |
| 432 | P-132 | A-3 |
| 433 | P-132 | A-4 |
| 434 | P-132 | A-5 |
| 435 | P-132 | A-6 |
| 436 | P-132 | A-7 |
| 437 | P-132 | A-8 |
| 438 | P-132 | A-9 |
| 439 | P-132 | A-10 |
| 440 | P-132 | A-11 |
| 441 | P-133 | A-2 |
| 442 | P-133 | A-3 |
| 443 | P-133 | A-4 |
| 444 | P-133 | A-5 |
| 445 | P-133 | A-6 |
| 446 | P-133 | A-7 |
| 447 | P-133 | A-8 |
| 448 | P-133 | A-9 |
| 449 | P-133 | A-10 |
| 450 | P-133 | A-11 |

Table 13 illustrates additional examples of some of the combinations of the present invention wherein the combination comprises a first amount of a reported p38-kinase inhibitor and a second amount of an aldosterone antagonist:

TABLE 13

| Example | | |
|---|---|---|
| Combination No. | p38-kinase inhibitor | aldosterone antagonist |
| 451 | P-134 | A-2 |
| 452 | P-134 | A-3 |
| 453 | P-134 | A-4 |
| 454 | P-134 | A-5 |
| 455 | P-134 | A-6 |
| 456 | P-134 | A-7 |
| 457 | P-134 | A-8 |
| 458 | P-134 | A-9 |
| 459 | P-134 | A-10 |
| 460 | P-134 | A-11 |

Table 14 illustrates additional examples of some of the combinations of the present invention wherein the combination comprises a first amount of a reported p38-kinase inhibitor and a second amount of an aldosterone antagonist:

TABLE 14

| Example | | |
|---|---|---|
| Combination No. | p38-kinase inhibitor | aldosterone antagonist |
| 461 | P-135 | A-2 |
| 462 | P-135 | A-3 |
| 463 | P-135 | A-4 |
| 464 | P-135 | A-5 |
| 465 | P-135 | A-6 |
| 466 | P-135 | A-7 |
| 467 | P-135 | A-8 |
| 468 | P-135 | A-9 |
| 469 | P-135 | A-10 |
| 470 | P-135 | A-11 |
| 471 | P-136 | A-2 |
| 472 | P-136 | A-3 |
| 473 | P-136 | A-4 |
| 474 | P-136 | A-5 |
| 475 | P-136 | A-6 |
| 476 | P-136 | A-7 |
| 477 | P-136 | A-8 |
| 478 | P-136 | A-9 |
| 479 | P-136 | A-10 |
| 480 | P-136 | A-11 |
| 481 | P-137 | A-2 |
| 482 | P-137 | A-3 |
| 483 | P-137 | A-4 |
| 484 | P-137 | A-5 |
| 485 | P-137 | A-6 |
| 486 | P-137 | A-7 |
| 487 | P-137 | A-8 |
| 488 | P-137 | A-9 |
| 489 | P-137 | A-10 |
| 490 | P-137 | A-11 |
| 491 | P-138 | A-2 |
| 492 | P-138 | A-3 |
| 493 | P-138 | A-4 |
| 494 | P-138 | A-5 |
| 495 | P-138 | A-6 |
| 496 | P-138 | A-7 |
| 497 | P-138 | A-8 |
| 498 | P-138 | A-9 |
| 499 | P-138 | A-10 |
| 500 | P-138 | A-11 |
| 501 | P-139 | A-2 |
| 502 | P-139 | A-3 |
| 503 | P-139 | A-4 |
| 504 | P-139 | A-5 |
| 505 | P-139 | A-6 |
| 506 | P-139 | A-7 |
| 507 | P-139 | A-8 |
| 508 | P-139 | A-9 |
| 509 | P-139 | A-10 |
| 510 | P-139 | A-11 |
| 511 | P-140 | A-2 |
| 512 | P-140 | A-3 |
| 513 | P-140 | A-4 |
| 514 | P-140 | A-5 |
| 515 | P-140 | A-6 |
| 516 | P-140 | A-7 |
| 517 | P-140 | A-8 |
| 518 | P-140 | A-9 |
| 519 | P-140 | A-10 |
| 520 | P-140 | A-11 |

Table 15 illustrates additional examples of some of the combinations of the present invention wherein the combination comprises a first amount of a reported p38-kinase inhibitor and a second amount of an aldosterone antagonist:

TABLE 15

| Example | | |
|---|---|---|
| Combination No. | p38-kinase inhibitor | aldosterone antagonist |
| 521 | P-141 | A-2 |
| 522 | P-141 | A-3 |
| 523 | P-141 | A-4 |
| 524 | P-141 | A-5 |
| 525 | P-141 | A-6 |
| 526 | P-141 | A-7 |
| 527 | P-141 | A-8 |
| 528 | P-141 | A-9 |
| 529 | P-141 | A-10 |
| 530 | P-141 | A-11 |
| 531 | P-142 | A-2 |
| 532 | P-142 | A-3 |
| 533 | P-142 | A-4 |
| 534 | P-142 | A-5 |
| 535 | P-142 | A-6 |
| 536 | P-142 | A-7 |
| 537 | P-142 | A-8 |
| 538 | P-142 | A-9 |
| 539 | P-142 | A-10 |
| 540 | P-142 | A-11 |
| 541 | P-143 | A-2 |
| 542 | P-143 | A-3 |
| 543 | P-143 | A-4 |
| 544 | P-143 | A-5 |
| 545 | P-143 | A-6 |
| 546 | P-143 | A-7 |
| 547 | P-143 | A-8 |
| 548 | P-143 | A-9 |
| 549 | P-143 | A-10 |
| 550 | P-143 | A-11 |
| 551 | P-144 | A-2 |
| 552 | P-144 | A-3 |
| 553 | P-144 | A-4 |
| 554 | P-144 | A-5 |
| 555 | P-144 | A-6 |
| 556 | P-144 | A-7 |
| 557 | P-144 | A-8 |
| 558 | P-144 | A-9 |
| 559 | P-144 | A-10 |
| 560 | P-144 | A-11 |
| 561 | P-145 | A-2 |
| 562 | P-145 | A-3 |
| 563 | P-145 | A-4 |
| 564 | P-145 | A-5 |
| 565 | P-145 | A-6 |
| 566 | P-145 | A-7 |
| 567 | P-145 | A-8 |
| 568 | P-145 | A-9 |
| 569 | P-145 | A-10 |
| 570 | P-145 | A-11 |
| 571 | P-146 | A-2 |
| 572 | P-146 | A-3 |
| 573 | P-146 | A-4 |
| 574 | P-146 | A-5 |
| 575 | P-146 | A-6 |
| 576 | P-146 | A-7 |
| 577 | P-146 | A-8 |
| 578 | P-146 | A-9 |
| 579 | P-146 | A-10 |
| 580 | P-146 | A-11 |
| 581 | P-147 | A-2 |
| 582 | P-147 | A-3 |
| 583 | P-147 | A-4 |
| 584 | P-147 | A-5 |
| 585 | P-147 | A-6 |
| 586 | P-147 | A-7 |
| 587 | P-147 | A-8 |
| 588 | P-147 | A-9 |
| 589 | P-147 | A-10 |
| 590 | P-147 | A-11 |
| 591 | P-148 | A-2 |
| 592 | P-148 | A-3 |
| 593 | P-148 | A-4 |
| 594 | P-148 | A-5 |
| 595 | P-148 | A-6 |
| 596 | P-148 | A-7 |
| 597 | P-148 | A-8 |
| 598 | P-148 | A-9 |
| 599 | P-148 | A-10 |
| 600 | P-148 | A-11 |
| 601 | P-149 | A-2 |
| 602 | P-149 | A-3 |
| 603 | P-149 | A-4 |
| 604 | P-149 | A-5 |
| 605 | P-149 | A-6 |
| 606 | P-149 | A-7 |
| 607 | P-149 | A-8 |
| 608 | P-149 | A-9 |
| 609 | P-149 | A-10 |
| 610 | P-149 | A-11 |
| 611 | P-150 | A-2 |
| 612 | P-150 | A-3 |
| 613 | P-150 | A-4 |
| 614 | P-150 | A-5 |
| 615 | P-150 | A-6 |
| 616 | P-150 | A-7 |
| 617 | P-150 | A-8 |
| 618 | P-150 | A-9 |
| 619 | P-150 | A-10 |
| 620 | P-150 | A-11 |
| 621 | P-151 | A-2 |
| 622 | P-151 | A-3 |
| 623 | P-151 | A-4 |
| 624 | P-151 | A-5 |
| 625 | P-151 | A-6 |
| 626 | P-151 | A-7 |
| 627 | P-151 | A-8 |
| 628 | P-151 | A-9 |
| 629 | P-151 | A-10 |
| 630 | P-151 | A-11 |
| 631 | P-152 | A-2 |
| 632 | P-152 | A-3 |
| 633 | P-152 | A-4 |
| 634 | P-152 | A-5 |
| 635 | P-152 | A-6 |
| 636 | P-152 | A-7 |
| 637 | P-152 | A-8 |
| 638 | P-152 | A-9 |
| 639 | P-152 | A-10 |
| 640 | P-152 | A-11 |
| 641 | P-153 | A-2 |
| 642 | P-153 | A-3 |
| 643 | P-153 | A-4 |
| 644 | P-153 | A-5 |
| 645 | P-153 | A-6 |
| 646 | P-153 | A-7 |
| 647 | P-153 | A-8 |
| 648 | P-153 | A-9 |
| 649 | P-153 | A-10 |
| 650 | P-153 | A-11 |
| 651 | P-154 | A-2 |
| 652 | P-154 | A-3 |
| 653 | P-154 | A-4 |
| 654 | P-154 | A-5 |
| 655 | P-154 | A-6 |
| 656 | P-154 | A-7 |
| 657 | P-154 | A-8 |
| 658 | P-154 | A-9 |
| 659 | P-154 | A-10 |
| 660 | P-154 | A-11 |
| 661 | P-155 | A-2 |
| 662 | P-155 | A-3 |
| 663 | P-155 | A-4 |
| 664 | P-155 | A-5 |
| 665 | P-155 | A-6 |
| 666 | P-155 | A-7 |
| 667 | P-155 | A-8 |
| 668 | P-155 | A-9 |
| 669 | P-155 | A-10 |
| 670 | P-155 | A-11 |
| 671 | P-156 | A-2 |
| 672 | P-156 | A-3 |
| 673 | P-156 | A-4 |
| 674 | P-156 | A-5 |
| 675 | P-156 | A-6 |
| 676 | P-156 | A-7 |
| 677 | P-156 | A-8 |
| 678 | P-156 | A-9 |
| 679 | P-156 | A-10 |
| 680 | P-156 | A-11 |
| 681 | P-157 | A-2 |
| 682 | P-157 | A-3 |
| 683 | P-157 | A-4 |
| 684 | P-157 | A-5 |
| 685 | P-157 | A-6 |
| 686 | P-157 | A-7 |
| 687 | P-157 | A-8 |
| 688 | P-157 | A-9 |
| 689 | P-157 | A-10 |
| 690 | P-157 | A-11 |
| 691 | P-158 | A-2 |
| 692 | P-158 | A-3 |
| 693 | P-158 | A-4 |
| 694 | P-158 | A-5 |
| 695 | P-158 | A-6 |
| 696 | P-158 | A-7 |
| 697 | P-158 | A-8 |
| 698 | P-158 | A-9 |
| 699 | P-158 | A-10 |
| 700 | P-158 | A-11 |
| 701 | P-159 | A-2 |
| 702 | P-159 | A-3 |
| 703 | P-159 | A-4 |
| 704 | P-159 | A-5 |
| 705 | P-159 | A-6 |
| 706 | P-159 | A-7 |
| 707 | P-159 | A-8 |
| 708 | P-159 | A-9 |
| 709 | P-159 | A-10 |
| 710 | P-159 | A-11 |
| 711 | P-160 | A-2 |
| 712 | P-160 | A-3 |
| 713 | P-160 | A-4 |
| 714 | P-160 | A-5 |
| 715 | P-160 | A-6 |
| 716 | P-160 | A-7 |
| 717 | P-160 | A-8 |
| 718 | P-160 | A-9 |
| 719 | P-160 | A-10 |
| 720 | P-160 | A-11 |
| 721 | P-161 | A-2 |
| 722 | P-161 | A-3 |
| 723 | P-161 | A-4 |
| 724 | P-161 | A-5 |
| 725 | P-161 | A-6 |
| 726 | P-161 | A-7 |
| 727 | P-161 | A-8 |
| 728 | P-161 | A-9 |
| 729 | P-161 | A-10 |
| 730 | P-161 | A-11 |
| 731 | P-162 | A-2 |
| 732 | P-162 | A-3 |
| 733 | P-162 | A-4 |
| 734 | P-162 | A-5 |
| 735 | P-162 | A-6 |
| 736 | P-162 | A-7 |
| 737 | P-162 | A-8 |
| 738 | P-162 | A-9 |
| 739 | P-162 | A-10 |
| 740 | P-162 | A-11 |
| 741 | P-163 | A-2 |
| 742 | P-163 | A-3 |
| 743 | P-163 | A-4 |
| 744 | P-163 | A-5 |
| 745 | P-163 | A-6 |
| 746 | P-163 | A-7 |
| 747 | P-163 | A-8 |
| 748 | P-163 | A-9 |
| 749 | P-163 | A-10 |
| 750 | P-163 | A-11 |
| 751 | P-164 | A-2 |
| 752 | P-164 | A-3 |
| 753 | P-164 | A-4 |
| 754 | P-164 | A-5 |
| 755 | P-164 | A-6 |
| 756 | P-164 | A-7 |
| 757 | P-164 | A-8 |
| 758 | P-164 | A-9 |
| 759 | P-164 | A-10 |
| 760 | P-164 | A-11 |
| 761 | P-165 | A-2 |
| 762 | P-165 | A-3 |
| 763 | P-165 | A-4 |
| 764 | P-165 | A-5 |
| 765 | P-165 | A-6 |
| 766 | P-165 | A-7 |
| 767 | P-165 | A-8 |
| 768 | P-165 | A-9 |
| 769 | P-165 | A-10 |
| 770 | P-165 | A-11 |
| 771 | P-166 | A-2 |
| 772 | P-166 | A-3 |
| 773 | P-166 | A-4 |
| 774 | P-166 | A-5 |
| 775 | P-166 | A-6 |
| 776 | P-166 | A-7 |
| 777 | P-166 | A-8 |
| 778 | P-166 | A-9 |
| 779 | P-166 | A-10 |
| 780 | P-166 | A-11 |
| 781 | P-167 | A-1 |
| 782 | P-167 | A-2 |
| 783 | P-167 | A-3 |
| 784 | P-167 | A-4 |
| 785 | P-167 | A-5 |
| 786 | P-167 | A-6 |
| 787 | P-167 | A-7 |
| 788 | P-167 | A-8 |
| 789 | P-167 | A-9 |
| 790 | P-167 | A-10 |
| 791 | P-167 | A-11 |
| 792 | P-168 | A-2 |
| 793 | P-168 | A-3 |
| 794 | P-168 | A-4 |
| 795 | P-168 | A-5 |
| 796 | P-168 | A-6 |
| 797 | P-168 | A-7 |
| 798 | P-168 | A-8 |
| 799 | P-168 | A-9 |
| 800 | P-168 | A-10 |
| 801 | P-168 | A-11 |
| 802 | P-169 | A-2 |
| 803 | P-169 | A-3 |
| 804 | P-169 | A-4 |
| 805 | P-169 | A-5 |
| 806 | P-169 | A-6 |
| 807 | P-169 | A-7 |
| 808 | P-169 | A-8 |
| 809 | P-169 | A-9 |
| 810 | P-169 | A-10 |
| 811 | P-169 | A-11 |
| 812 | P-170 | A-2 |
| 813 | P-170 | A-3 |
| 814 | P-170 | A-4 |
| 815 | P-170 | A-5 |
| 816 | P-170 | A-6 |
| 817 | P-170 | A-7 |
| 818 | P-170 | A-8 |
| 819 | P-170 | A-9 |
| 820 | P-170 | A-10 |
| 821 | P-170 | A-11 |

It should be recognized that the above tables simply illustrate examples of various combinations of 8-kinase inhibitors with various aldosterone antagonists. This invention therefore should not be limited to those combinations.

It should also be recognized that this invention contemplates combinations comprising more than one p38-kinase inhibitor with an aldosterone antagonist, as well as a combinations comprising a p38-kinase inhibitor with more than one aldosterone antagonist, as well as combinations comprising more than one p38-kinase inhibitor with more than one aldosterone antagonist. Further, any such combination (or any combination comprising only one p38-kinase inhibitor and only one aldosterone antagonist) may further comprise one or more ACE inhibitors, one or more diuretics, and/or one or more other therapeutic agents. Such other therapeutic agents may include, for example, one or more IBAT inhibitors, CETP inhibitors, fibrates, digoxin, calcium channel blockers, endothelin antagonists, inhibitors of microsomal triglyceride transfer protein, cholesterol absorption antagonists, phytosterols, bile acid sequestrants, vasodilators, adrenergic blockers, adrenergic stimulants, and/or inhibitors of HMG-CoA reductase activity. Such other therapeutic agents may also comprise, for example, one or more conventional anti-inflammatories, such as steroids, cyclooxygenase-2 inhibitors, DMARDs, immunosuppressive agents, NSAIDs, 5-lipoxygenase inhibitors, LTB4 antagonists, and LTA4 hydrolase inhibitors.

### F-2B. Example Combinations Comprising A p38-Kinase Inhibitors With A Diuretic

Table 16 illustrates examples of some of the combinations of the present invention wherein the combination comprises a first amount of a substituted-pyrazole p38-kinase inhibitor and a second amount of a diuretic:

TABLE 16

| Example | | |
|---|---|---|
| Combination No. | p38-kinase inhibitor | diuretic |
| 822 | P-1 | amanozine |
| 823 | P-1 | amiloride |
| 824 | P-1 | arbutin |
| 825 | P-1 | chlorazanil |
| 826 | P-1 | ethacrynic acid |
| 827 | P-1 | etozolin |
| 828 | P-1 | hydracarbazine |
| 829 | P-1 | isosorbide |
| 830 | P-1 | mannitol |
| 831 | P-1 | metochalcone |
| 832 | P-1 | muzolimine |
| 833 | P-1 | perhexiline |
| 834 | P-1 | ticrynafen |
| 835 | P-1 | triamterene |
| 836 | P-1 | urea |
| 837 | P-1 | althiazide |
| 838 | P-1 | bendroflumethiazide |
| 839 | P-1 | benzthiazide |
| 840 | P-1 | benzylhydrochlorothiazide |
| 841 | P-1 | buthiazide |
| 842 | P-1 | chlorothiazide |
| 843 | P-1 | chlorthalidone |
| 844 | P-1 | cyclopenthiazide |
| 845 | P-1 | cyclothiazide |
| 846 | P-1 | epithiazide |
| 847 | P-1 | ethiazide |
| 848 | P-1 | fenquizone |
| 849 | P-1 | hydrochlorothiazide |
| 850 | P-1 | hydroflumethiazide |
| 851 | P-1 | indapamide |
| 852 | P-1 | methyclothiazide |
| 853 | P-1 | meticrane |
| 854 | P-1 | metolazone |
| 855 | P-1 | paraflutizide |
| 856 | P-1 | polythiazide |
| 857 | P-1 | quinethazone |
| 858 | P-1 | teclothiazide |
| 859 | P-1 | trichlormethiazide |
| 860 | P-1 | acetazolamide |
| 861 | P-1 | ambuside |
| 862 | P-1 | azosemide |
| 863 | P-1 | bumetanide |
| 864 | P-1 | butazolamide |
| 865 | P-1 | chloraminophenamide |
| 866 | P-1 | clofenamide |
| 867 | P-1 | clopamide |
| 868 | P-1 | clorexolone |
| 869 | P-1 | disulfamide |
| 870 | P-1 | ethoxolamide |
| 871 | P-1 | furosemide |
| 872 | P-1 | mefruside |
| 873 | P-1 | methazolamide |
| 874 | P-1 | piretanide |
| 875 | P-1 | torasemide |
| 876 | P-1 | tripamide |
| 877 | P-1 | xipamide |
| 878 | P-1 | mercaptomerin sodium |
| 879 | P-1 | merethoxylline |
| 880 | P-1 | procaine |
| 881 | P-1 | mersalyl with thiophylline |
| 882 | P-15 | amanozine |
| 883 | P-15 | amiloride |
| 884 | P-15 | arbutin |
| 885 | P-15 | chlorazanil |
| 886 | P-15 | ethacrynic acid |
| 887 | P-15 | etozolin |
| 888 | P-15 | hydracarbazine |
| 889 | P-15 | isosorbide |
| 890 | P-15 | mannitol |
| 891 | P-1 5 | metochalcone |
| 892 | P-15 | muzolimine |
| 893 | P-15 | perhexiline |
| 894 | P-15 | ticrynafen |
| 895 | P-15 | triamterene |
| 896 | P-15 | urea |
| 897 | P-15 | althiazide |
| 898 | P-15 | bendroflumethiazide |
| 899 | P-15 | benzthiazide |
| 900 | P-15 | benzylhydrochlorothiazide |
| 901 | P-15 | buthiazide |
| 902 | P-15 | chlorothiazide |
| 903 | P-15 | chlorthalidone |
| 904 | P-15 | cyclopenthiazide |
| 905 | P-15 | cyclothiazide |
| 906 | P-15 | epithiazide |
| 907 | P-15 | ethiazide |
| 908 | P-15 | fenquizone |
| 909 | P-15 | hydrochlorothiazide |
| 910 | P-15 | hydroflumethiazide |
| 911 | P-15 | indapamide |
| 912 | P-15 | methyclothiazide |
| 913 | P-15 | meticrane |
| 914 | P-15 | metolazone |
| 915 | P-15 | paraflutizide |
| 916 | P-15 | polythiazide |
| 917 | P-15 | quinethazone |
| 918 | P-15 | teclothiazide |
| 919 | P-15 | trichlormethiazide |
| 920 | P-15 | acetazolamide |
| 921 | P-15 | ambuside |
| 922 | P-15 | azosemide |
| 923 | P-15 | bumetanide |
| 924 | P-15 | butazolamide |
| 925 | P-15 | chloraminophenamide |
| 926 | P-15 | clofenamide |
| 927 | P-15 | clopamide |
| 928 | P-15 | clorexolone |
| 929 | P-15 | disulfamide |
| 930 | P-15 | ethoxolamide |
| 931 | P-15 | furosemide |
| 932 | P-15 | mefruside |
| 933 | P-15 | methazolamide |
| 934 | P-15 | piretanide |
| 935 | P-15 | torasemide |
| 936 | P-15 | tripamide |
| 937 | P-15 | xipamide |
| 938 | P-15 | mercaptomerin sodium |
| 939 | P-15 | merethoxylline |
| 940 | P-15 | procaine |
| 941 | P-15 | mersalyl with thiophylline |
| 942 | P-18 | amanozine |
| 943 | P-18 | amiloride |
| 944 | P-18 | arbutin |
| 945 | p-18 | chlorazanil |
| 946 | P-18 | ethacrynic acid |
| 947 | P-18 | etozolin |
| 948 | P-18 | hydracarbazine |
| 949 | P-18 | isosorbide |
| 950 | P-18 | mannitol |
| 951 | P-18 | metochalcone |
| 952 | P-18 | muzolimine |
| 953 | P-18 | perhexiline |
| 954 | P-18 | ticrynafen |
| 955 | P-18 | triamterene |
| 956 | p-18 | urea |
| 957 | p-18 | althiazide |
| 958 | p-18 | bendroflumethiazide |
| 959 | P-18 | benzthiazide |
| 960 | P-18 | benzylhydrochlorothiazide |
| 961 | P-18 | buthiazide |
| 962 | P-18 | chlorothiazide |
| 963 | p-18 | chlorthalidone |
| 964 | P-18 | cyclopenthiazide |
| 965 | P-18 | cyclothiazide |
| 966 | P-18 | epithiazide |
| 967 | P-18 | ethiazide |
| 968 | P-18 | fenquizone |
| 969 | P-18 | hydrochlorothiazide |
| 970 | P-18 | hydroflumethiazide |
| 971 | P-18 | indapamide |
| 972 | P-18 | methyclothiazide |
| 973 | P-18 | meticrane |
| 974 | P-18 | metolazone |
| 975 | P-18 | paraflutizide |
| 976 | P-18 | polythiazide |
| 977 | P-18 | quinethazone |
| 978 | P-18 | teclothiazide |
| 979 | P-18 | trichlormethiazide |
| 980 | P-18 | acetazolamide |
| 981 | P-18 | ambuside |
| 982 | P-18 | azosemide |
| 983 | P-18 | bumetanide |
| 984 | P-18 | butazolamide |
| 985 | P-18 | chloraminophenamide |
| 986 | P-18 | clofenamide |
| 987 | P-18 | clopamide |
| 988 | P-18 | clorexolone |
| 989 | P-18 | disulfamide |
| 990 | P-18 | ethoxolamide |
| 991 | P-18 | furosemide |
| 992 | P-18 | mefruside |
| 993 | P-18 | methazolamide |
| 994 | P-18 | piretanide |
| 995 | P-18 | torasemide |
| 996 | P-18 | tripamide |
| 997 | P-18 | xipamide |
| 998 | P-18 | mercaptomerin sodium |
| 999 | P-18 | merethoxylline |
| 1000 | P-18 | procaine |
| 1001 | P-18 | mersalyl with thiophylline |
| 1002 | P-21 | amanozine |
| 1003 | P-21 | amiloride |
| 1004 | P-21 | arbutin |
| 1005 | P-21 | chlorazanil |
| 1006 | P-21 | ethacrynic acid |
| 1007 | P-21 | etozolin |
| 1008 | P-21 | hydracarbazine |
| 1009 | P-21 | isosorbide |
| 1010 | P-21 | mannitol |
| 1011 | P-21 | metochalcone |
| 1012 | P-21 | muzolimine |
| 1013 | P-21 | perhexiline |
| 1014 | P-21 | ticrynafen |
| 1015 | P-21 | triamterene |
| 1016 | P-21 | urea |
| 1017 | P-21 | althiazide |
| 1018 | P-21 | bendroflumethiazide |
| 1019 | P-21 | benzthiazide |
| 1020 | P-21 | benzylhydrochlorothiazide |
| 1021 | P-21 | buthiazide |
| 1022 | P-21 | chlorothiazide |
| 1023 | P-21 | chlorthalidone |
| 1024 | P-21 | cyclopenthiazide |
| 1025 | P-21 | cyclothiazide |
| 1026 | P-21 | epithiazide |
| 1027 | P-21 | ethiazide |
| 1028 | P-21 | fenquizone |
| 1029 | P-21 | hydrochlorothiazide |
| 1030 | P-21 | hydroflumethiazide |
| 1031 | P-21 | indapamide |
| 1032 | P-21 | methyclothiazide |
| 1033 | P-21 | meticrane |
| 1034 | P-21 | metolazone |
| 1035 | P-21 | paraflutizide |
| 1036 | P-21 | polythiazide |
| 1037 | P-21 | quinethazone |
| 1038 | P-21 | teclothiazide |
| 1039 | P-21 | trichlormethiazide |
| 1040 | P-21 | acetazolamide |
| 1041 | P-21 | ambuside |
| 1042 | P-21 | azosemide |
| 1043 | P-21 | bumetanide |
| 1044 | P-21 | butazolamide |
| 1045 | P-21 | chloraminophenamide |
| 1046 | P-21 | clofenamide |
| 1047 | P-21 | clopamide |
| 1048 | P-21 | clorexolone |
| 1049 | P-21 | disulfamide |
| 1050 | P-21 | ethoxolamide |
| 1051 | P-21 | furosemide |
| 1052 | P-21 | mefruside |
| 1053 | P-21 | methazolamide |
| 1054 | P-21 | piretanide |
| 1055 | P-21 | torasemide |
| 1056 | P-21 | tripamide |
| 1057 | P-21 | xipamide |
| 1058 | P-21 | mercaptomerin sodium |
| 1059 | P-21 | merethoxylline |
| 1060 | P-21 | procaine |
| 1061 | P-21 | mersalyl with thiophylline |
| 1062 | P-48 | amanozine |
| 1063 | P-48 | amiloride |
| 1064 | P-48 | arbutin |
| 1065 | P-48 | chlorazanil |
| 1066 | P-48 | ethacrynic acid |
| 1067 | P-48 | etozolin |
| 1068 | P-48 | hydracarbazine |
| 1069 | P-48 | isosorbide |
| 1070 | P-48 | mannitol |
| 1071 | P-48 | metochalcone |
| 1072 | P-48 | muzolimine |
| 1073 | P-48 | perhexiline |
| 1074 | P-48 | ticrynafen |
| 1075 | P-48 | triamterene |
| 1076 | P-48 | urea |
| 1077 | P-48 | althiazide |
| 1078 | P-48 | bendroflumethiazide |
| 1079 | P-48 | benzthiazide |
| 1080 | P-48 | benzylhydrochlorothiazide |
| 1081 | P-48 | buthiazide |
| 1082 | P-48 | chlorothiazide |
| 1083 | P-48 | chlorthalidone |
| 1084 | P-48 | cyclopenthiazide |
| 1085 | P-48 | cyclothiazide |
| 1086 | P-48 | epithiazide |
| 1087 | P-48 | ethiazide |
| 1088 | P-48 | fenquizone |
| 1089 | P-48 | hydrochlorothiazide |
| 1090 | P-48 | hydroflumethiazide |
| 1091 | P-48 | indapamide |
| 1092 | P-48 | methyclothiazide |
| 1093 | P-48 | meticrane |
| 1094 | P-48 | metolazone |
| 1095 | P-48 | paraflutizide |
| 1096 | P-48 | polythiazide |
| 1097 | P-48 | quinethazone |
| 1098 | P-48 | teclothiazide |
| 1099 | P-48 | trichlormethiazide |
| 1100 | P-48 | acetazolamide |
| 1101 | P-48 | ambuside |
| 1102 | P-48 | azosemide |
| 1103 | P-48 | bumetanide |
| 1104 | P-48 | butazolamide |
| 1105 | P-48 | chloraminophenamide |
| 1106 | P-48 | clofenamide |
| 1107 | P-48 | clopamide |
| 1108 | P-48 | clorexolone |
| 1109 | P-48 | disulfamide |
| 1110 | P-48 | ethoxolamide |
| 1111 | P-48 | furosemide |
| 1112 | P-48 | mefruside |
| 1113 | P-48 | methazolamide |
| 1114 | P-48 | piretanide |
| 1115 | P-48 | torasemide |
| 1116 | P-48 | tripamide |
| 1117 | P-48 | xipamide |
| 1118 | P-48 | mercaptomerin sodium |
| 1119 | P-48 | merethoxylline |
| 1120 | P-48 | procaine |
| 1121 | P-48 | mersalyl with thiophylline |
| 1122 | P-49 | amanozine |
| 1123 | P-49 | amiloride |
| 1124 | P-49 | arbutin |
| 1125 | P-49 | chlorazanil |
| 1126 | P-49 | ethacrynic acid |
| 1127 | P-49 | etozolin |
| 1128 | P-49 | hydracarbazine |
| 1129 | P-49 | isosorbide |
| 1130 | P-49 | mannitol |
| 1131 | P-49 | metochalcone |
| 1132 | P-49 | muzolimine |
| 1133 | P-49 | perhexiline |
| 1134 | P-49 | ticrynafen |
| 1135 | P-49 | triamterene |
| 1136 | P-49 | urea |
| 1137 | P-49 | althiazide |
| 1138 | P-49 | bendroflumethiazide |
| 1139 | P-49 | benzthiazide |
| 1140 | P-49 | benzylhydrochlorothiazide |
| 1141 | P-49 | buthiazide |
| 1142 | P-49 | chlorothiazide |
| 1143 | P-49 | chlorthalidone |
| 1144 | P-49 | cyclopenthiazide |
| 1145 | P-49 | cyclothiazide |
| 1146 | P-49 | epithiazide |
| 1147 | P-49 | ethiazide |
| 1148 | P-49 | fenquizone |
| 1149 | P-49 | hydrochlorothiazide |
| 1150 | P-49 | hydroflumethiazide |
| 1151 | P-49 | indapamide |
| 1152 | P-49 | methyclothiazide |
| 1153 | P-49 | meticrane |
| 1154 | P-49 | metolazone |
| 1155 | P-49 | paraflutizide |
| 1156 | P-49 | polythiazide |
| 1157 | P-49 | quinethazone |
| 1158 | P-49 | teclothiazide |
| 1159 | P-49 | trichlormethiazide |
| 1160 | P-49 | acetazolamide |
| 1161 | P-49 | ambuside |
| 1162 | P-49 | azosemide |
| 1163 | P-49 | bumetanide |
| 1164 | P-49 | butazolamide |
| 1165 | P-49 | chloraminophenamide |
| 1166 | P-49 | clofenamide |
| 1167 | P-49 | clopamide |
| 1168 | P-49 | clorexolone |
| 1169 | P-49 | disulfamide |
| 1170 | P-49 | ethoxolamide |
| 1171 | P-49 | furosemide |
| 1172 | P-49 | mefruside |
| 1173 | P-49 | methazolamide |
| 1174 | P-49 | piretanide |
| 1175 | P-49 | torasemide |
| 1176 | P-49 | tripamide |
| 1177 | P-49 | xipamide |
| 1178 | P-49 | mercaptomerin sodium |
| 1179 | P-49 | merethoxylline |
| 1180 | P-49 | procaine |
| 1181 | P-49 | mersalyl with thiophylline |

Table 17 illustrates examples of some of the combinations of the present invention wherein the combination comprises a first amount of a reported substituted-pyrazole p38-kinase inhibitor and a second amount of a diuretic:

TABLE 17

| Example | | |
|---|---|---|
| Combination No. | p38-kinase inhibitor | diuretic |
| 1182 | P-129 | amanozine |
| 1183 | P-129 | amiloride |
| 1184 | P-129 | arbutin |
| 1185 | P-129 | chlorazanil |
| 1186 | P-129 | ethacrynic acid |
| 1187 | P-129 | etozolin |
| 1188 | P-129 | hydracarbazine |
| 1189 | P-129 | isosorbide |
| 1190 | P-129 | mannitol |
| 1191 | P-129 | metochalcone |
| 1192 | P-129 | muzolimine |
| 1193 | P-129 | perhexiline |
| 1194 | P-129 | ticrynafen |
| 1195 | P-129 | triamterene |
| 1196 | P-129 | urea |
| 1197 | P-129 | althiazide |
| 1198 | P-129 | bendroflumethiazide |
| 1199 | P-129 | benzthiazide |
| 1200 | P-129 | benzylhydrochlorothiazide |
| 1201 | P-129 | buthiazide |
| 1202 | P-129 | chlorothiazide |
| 1203 | P-129 | chlorthalidone |
| 1204 | P-129 | cyclopenthiazide |
| 1205 | P-129 | cyclothiazide |
| 1206 | P-129 | epithiazide |
| 1207 | P-129 | ethiazide |
| 1208 | P-129 | fenquizone |
| 1209 | P-129 | hydrochlorothiazide |
| 1210 | P-129 | hydroflumethiazide |
| 1211 | P-129 | indapamide |
| 1212 | P-129 | methyclothiazide |
| 1213 | P-129 | meticrane |
| 1214 | P-129 | metolazone |
| 1215 | P-129 | paraflutizide |
| 1216 | P-129 | polythiazide |
| 1217 | P-129 | quinethazone |
| 1218 | P-129 | teclothiazide |
| 1219 | P-129 | trichlormethiazide |
| 1220 | P-129 | acetazolamide |
| 1221 | P-129 | ambuside |
| 1222 | P-129 | azosemide |
| 1223 | P-129 | bumetanide |
| 1224 | P-129 | butazolamide |
| 1225 | P-129 | chloraminophenamide |
| 1226 | P-129 | clofenamide |
| 1227 | P-129 | clopamide |
| 1228 | P-129 | clorexolone |
| 1229 | P-129 | disulfamide |
| 1230 | P-129 | ethoxolamide |
| 1231 | P-129 | furosemide |
| 1232 | P-129 | mefruside |
| 1233 | P-129 | methazolamide |
| 1234 | P-129 | piretanide |
| 1235 | P-129 | torasemide |
| 1236 | P-129 | tripamide |
| 1237 | P-129 | xipamide |
| 1238 | P-129 | mercaptomerin sodium |
| 1239 | P-129 | merethoxylline |
| 1240 | P-129 | procaine |
| 1241 | P-129 | mersalyl with thiophylline |
| 1242 | P-130 | amanozine |
| 1243 | P-130 | amiloride |
| 1244 | P-130 | arbutin |
| 1245 | P-130 | chlorazanil |
| 1246 | P-130 | ethacrynic acid |
| 1247 | P-130 | etozolin |
| 1248 | P-130 | hydracarbazine |
| 1249 | P-130 | isosorbide |
| 1250 | P-130 | mannitol |
| 1251 | P-130 | metochalcone |
| 1252 | P-130 | muzolimine |
| 1253 | P-130 | perhexiline |
| 1254 | P-130 | ticrynafen |
| 1255 | P-130 | triamterene |
| 1256 | P-130 | urea |
| 1257 | P-130 | althiazide |
| 1258 | P-130 | bendroflumethiazide |
| 1259 | P-130 | benzthiazide |
| 1260 | P-130 | benzylhydrochlorothiazide |
| 1261 | P-130 | buthiazide |
| 1262 | P-130 | chlorothiazide |
| 1263 | P-130 | chlorthalidone |
| 1264 | P-130 | cyclopenthiazide |
| 1265 | P-130 | cyclothiazide |
| 1266 | P-130 | epithiazide |
| 1267 | P-130 | ethiazide |
| 1268 | P-130 | fenquizone |
| 1269 | P-130 | hydrochlorothiazide |
| 1270 | P-130 | hydroflumethiazide |
| 1271 | P-130 | indapamide |
| 1272 | P-130 | methyclothiazide |
| 1273 | P-130 | meticrane |
| 1274 | P-130 | metolazone |
| 1275 | P-130 | paraflutizide |
| 1276 | P-130 | polythiazide |
| 1277 | P-130 | quinethazone |
| 1278 | P-130 | teclothiazide |
| 1279 | P-130 | trichlormethiazide |
| 1280 | P-130 | acetazolamide |
| 1281 | P-130 | ambuside |
| 1282 | P-130 | azosemide |
| 1283 | P-130 | bumetanide |
| 1284 | P-130 | butazolamide |
| 1285 | P-130 | chloraminophenamide |
| 1286 | P-130 | clofenamide |
| 1287 | P-130 | clopamide |
| 1288 | P-130 | clorexolone |
| 1289 | P-130 | disulfamide |
| 1290 | P-130 | ethoxolamide |
| 1291 | P-130 | furosemide |
| 1292 | P-130 | mefruside |
| 1293 | P-130 | methazolamide |
| 1294 | P-130 | piretanide |
| 1295 | P-130 | torasemide |
| 1296 | P-130 | tripamide |
| 1297 | P-130 | xipamide |
| 1298 | P-130 | mercaptomerin sodium |
| 1299 | P-130 | merethoxylline |
| 1300 | P-130 | procaine |
| 1301 | P-130 | mersalyl with thiophylline |
| 1302 | P-131 | amanozine |
| 1303 | P-131 | amiloride |
| 1304 | P-131 | arbutin |
| 1305 | P-131 | chlorazanil |
| 1306 | P-131 | ethacrynic acid |
| 1307 | P-131 | etozolin |
| 1308 | P-131 | hydracarbazine |
| 1309 | P-131 | isosorbide |
| 1310 | P-131 | mannitol |
| 1311 | P-131 | metochalcone |
| 1312 | P-131 | muzolimine |
| 1313 | P-131 | perhexiline |
| 1314 | P-131 | ticrynafen |
| 1315 | P-131 | triamterene |
| 1316 | P-131 | urea |
| 1317 | P-131 | althiazide |
| 1318 | P-131 | bendroflumethiazide |
| 1319 | P-131 | benzthiazide |
| 1320 | P-131 | benzylhydrochlorothiazide |
| 1321 | P-131 | buthiazide |
| 1322 | P-131 | chlorothiazide |
| 1323 | P-131 | chlorthalidone |
| 1324 | P-131 | cyclopenthiazide |
| 1325 | P-131 | cyclothiazide |
| 1326 | P-131 | epithiazide |
| 1327 | P-131 | ethiazide |
| 1328 | P-131 | fenquizone |
| 1329 | P-131 | hydrochlorothiazide |
| 1330 | P-131 | hydroflumethiazide |
| 1331 | P-131 | indapamide |
| 1332 | P-131 | methyclothiazide |
| 1333 | P-131 | meticrane |
| 1334 | P-131 | metolazone |
| 1335 | P-131 | paraflutizide |
| 1336 | P-131 | polythiazide |
| 1337 | P-131 | quinethazone |
| 1338 | P-131 | teclothiazide |
| 1339 | P-131 | trichlormethiazide |
| 1340 | P-131 | acetazolamide |
| 1341 | P-131 | ambuside |
| 1342 | P-131 | azosemide |
| 1343 | P-131 | bumetanide |
| 1344 | P-131 | butazolamide |
| 1345 | P-131 | chloraminophenamide |
| 1346 | P-131 | clofenamide |
| 1347 | P-131 | clopamide |
| 1348 | P-131 | clorexolone |
| 1349 | P-131 | disulfamide |
| 1350 | P-131 | ethoxolamide |
| 1351 | P-131 | furosemide |
| 1352 | P-131 | mefruside |
| 1353 | P-131 | methazolamide |
| 1354 | P-131 | piretanide |
| 1355 | P-131 | torasemide |
| 1356 | P-131 | tripamide |
| 1357 | P-131 | xipamide |
| 1358 | P-131 | mercaptomerin sodium |
| 1359 | P-131 | merethoxylline |
| 1360 | P-131 | procaine |
| 1361 | P-131 | mersalyl with thiophylline |
| 1362 | P-132 | amanozine |
| 1363 | P-132 | amiloride |
| 1364 | P-132 | arbutin |
| 1365 | P-132 | chlorazanil |
| 1366 | P-132 | ethacrynic acid |
| 1367 | P-132 | etozolin |
| 1368 | P-132 | hydracarbazine |
| 1369 | P-132 | isosorbide |
| 1370 | P-132 | mannitol |
| 1371 | P-132 | metochalcone |
| 1372 | P-132 | muzolimine |
| 1373 | P-132 | perhexiline |
| 1374 | P-132 | ticrynafen |
| 1375 | P-132 | triamterene |
| 1376 | P-132 | urea |
| 1377 | P-132 | althiazide |
| 1378 | P-132 | bendroflumethiazide |
| 1379 | P-132 | benzthiazide |
| 1380 | P-132 | benzylhydrochlorothiazide |
| 1381 | P-132 | buthiazide |
| 1382 | P-132 | chlorothiazide |
| 1383 | P-132 | chlorthalidone |
| 1384 | P-132 | cyclopenthiazide |
| 1385 | P-132 | cyclothiazide |
| 1386 | P-132 | epithiazide |
| 1387 | P-132 | ethiazide |
| 1388 | P-132 | fenquizone |
| 1389 | P-132 | hydrochlorothiazide |
| 1390 | P-132 | hydroflumethiazide |
| 1391 | P-132 | indapamide |
| 1392 | P-132 | methyclothiazide |
| 1393 | P-132 | meticrane |
| 1394 | P-132 | metolazone |
| 1395 | P-132 | paraflutizide |
| 1396 | P-132 | polythiazide |
| 1397 | P-132 | quinethazone |
| 1398 | P-132 | teclothiazide |
| 1399 | P-132 | trichlormethiazide |
| 1400 | P-132 | acetazolamide |
| 1401 | P-132 | ambuside |
| 1402 | P-132 | azosemide |
| 1403 | P-132 | bumetanide |
| 1404 | P-132 | butazolamide |
| 1405 | P-132 | chloraminophenamide |
| 1406 | P-132 | clofenamide |
| 1407 | P-132 | clopamide |
| 1408 | P-132 | clorexolone |
| 1409 | P-132 | disulfamide |
| 1410 | P-132 | ethoxolamide |
| 1411 | P-132 | furosemide |
| 1412 | P-132 | mefruside |
| 1413 | P-132 | methazolamide |
| 1414 | P-132 | piretanide |
| 1415 | P-132 | torasemide |
| 1416 | P-132 | tripamide |
| 1417 | P-132 | xipamide |
| 1418 | P-132 | mercaptomerin sodium |
| 1419 | P-132 | merethoxylline |
| 1420 | P-132 | procaine |
| 1421 | P-132 | mersalyl with thiophylline |
| 1422 | P-133 | amanozine |
| 1423 | P-133 | amiloride |
| 1424 | P-133 | arbutin |
| 1425 | P-133 | chlorazanil |
| 1426 | P-133 | ethacrynic acid |
| 1427 | P-133 | etozolin |
| 1428 | P-133 | hydracarbazine |
| 1429 | P-133 | isosorbide |
| 1430 | P-133 | mannitol |
| 1431 | P-133 | metochalcone |
| 1432 | P-133 | muzolimine |
| 1433 | P-133 | perhexiline |
| 1434 | P-133 | ticrynafen |
| 1435 | P-133 | triamterene |
| 1436 | P-133 | urea |
| 1437 | P-133 | althiazide |
| 1438 | P-133 | bendroflumethiazide |
| 1439 | P-133 | benzthiazide |
| 1440 | P-133 | benzylhydrochlorothiazide |
| 1441 | P-133 | buthiazide |
| 1442 | P-133 | chlorothiazide |
| 1443 | P-133 | chlorthalidone |
| 1444 | P-133 | cyclopenthiazide |
| 1445 | P-133 | cyclothiazide |
| 1446 | P-133 | epithiazide |
| 1447 | P-133 | ethiazide |
| 1448 | P-133 | fenquizone |
| 1449 | P-133 | hydrochlorothiazide |
| 1450 | P-133 | hydroflumethiazide |
| 1451 | P-133 | indapamide |
| 1452 | P-133 | methyclothiazide |
| 1453 | P-133 | meticrane |
| 1454 | P-133 | metolazone |
| 1455 | P-133 | paraflutizide |
| 1456 | P-133 | polythiazide |
| 1457 | P-133 | quinethazone |
| 1458 | P-133 | teclothiazide |
| 1459 | P-133 | trichlormethiazide |
| 1460 | P-133 | acetazolamide |
| 1461 | P-133 | ambuside |
| 1462 | P-133 | azosemide |
| 1463 | P-133 | bumetanide |
| 1464 | P-133 | butazolamide |
| 1465 | P-133 | chloraminophenamide |
| 1466 | P-133 | clofenamide |
| 1467 | P-133 | clopamide |
| 1468 | P-133 | clorexolone |
| 1469 | P-133 | disulfamide |
| 1470 | P-133 | ethoxolamide |
| 1471 | P-133 | furosemide |
| 1472 | P-133 | mefruside |
| 1473 | P-133 | methazolamide |
| 1474 | P-133 | piretanide |
| 1475 | P-133 | torasemide |
| 1476 | P-133 | tripamide |
| 1477 | P-133 | xipamide |
| 1478 | P-133 | mercaptomerin sodium |
| 1479 | P-133 | merethoxylline |
| 1480 | P-133 | procaine |
| 1481 | P-133 | mersalyl with thiophylline |

Table 18 illustrates additional examples of some of the combinations of the present invention wherein the combination comprises a first amount of a reported p38-kinase inhibitor and a second amount of a diuretic:

TABLE 18

| Example | | |
|---|---|---|
| Combination No. | p38-kinase inhibitor | diuretic |
| 1482 | P-134 | amanozine |
| 1483 | P-134 | amiloride |
| 1484 | P-134 | arbutin |
| 1485 | P-134 | chlorazanil |
| 1486 | P-134 | ethacrynic acid |
| 1487 | P-134 | etozolin |
| 1488 | P-134 | hydracarbazine |
| 1489 | P-134 | isosorbide |
| 1490 | P-134 | mannitol |
| 1491 | P-134 | metochalcone |
| 1492 | P-134 | muzolimine |
| 1493 | P-134 | perhexiline |
| 1494 | P-134 | ticrynafen |
| 1495 | P-134 | triamterene |
| 1496 | P-134 | urea |
| 1497 | P-134 | althiazide |
| 1498 | P-134 | bendroflumethiazide |
| 1499 | P-134 | benzthiazide |
| 1500 | P-134 | benzylhydrochlorothiazide |
| 1501 | P-134 | buthiazide |
| 1502 | P-134 | chlorothiazide |
| 1503 | P-134 | chlorthalidone |
| 1504 | P-134 | cyclopenthiazide |
| 1505 | P-134 | cyclothiazide |
| 1506 | P-134 | epithiazide |
| 1507 | P-134 | ethiazide |
| 1508 | P-134 | fenquizone |
| 1509 | P-134 | hydrochlorothiazide |
| 1510 | P-134 | hydroflumethiazide |
| 1511 | P-134 | indapamide |
| 1512 | P-134 | methyclothiazide |
| 1513 | P-134 | meticrane |
| 1514 | P-134 | metolazone |
| 1515 | P-134 | paraflutizide |
| 1516 | P-134 | polythiazide |
| 1517 | P-134 | quinethazone |
| 1518 | P-134 | teclothiazide |
| 1519 | P-134 | trichlormethiazide |
| 1520 | P-134 | acetazolamide |
| 1521 | P-134 | ambuside |
| 1522 | P-134 | azosemide |
| 1523 | P-134 | bumetanide |
| 1524 | P-134 | butazolamide |
| 1525 | P-134 | chloraminophenamide |
| 1526 | P-134 | clofenamide |
| 1527 | P-134 | clopamide |
| 1528 | P-134 | clorexolone |
| 1529 | P-134 | disulfamide |
| 1530 | P-134 | ethoxolamide |
| 1531 | P-134 | furosemide |
| 1532 | P-134 | mefruside |
| 1533 | P-134 | methazolamide |
| 1534 | P-134 | piretanide |
| 1535 | P-134 | torasemide |
| 1536 | P-134 | tripamide |
| 1537 | P-134 | xipamide |
| 1538 | P-134 | mercaptomerin sodium |
| 1539 | P-134 | merethoxylline |
| 1540 | P-134 | procaine |
| 1541 | P-134 | mersalyl with thiophylline |

Table 19 illustrates additional examples of some of the combinations of the present invention wherein the combination comprises a first amount of a reported p38-kinase inhibitor and a second amount of a diuretic:

TABLE 19

| Example | | |
|---|---|---|
| Combination No. | p38-kinase inhibitor | diuretic |
| 1542 | P-135 | amanozine |
| 1543 | P-135 | amiloride |
| 1544 | P-135 | arbutin |
| 1545 | P-135 | chlorazanil |
| 1546 | P-135 | ethacrynic acid |
| 1547 | P-135 | etozolin |
| 1548 | P-135 | hydracarbazine |
| 1549 | P-135 | isosorbide |
| 1550 | P-135 | mannitol |
| 1551 | P-135 | metochalcone |
| 1552 | P-135 | muzolimine |
| 1553 | P-135 | perhexiline |
| 1554 | P-135 | ticrynafen |
| 1555 | P-135 | triamterene |
| 1556 | P-135 | urea |
| 1557 | P-135 | althiazide |
| 1558 | P-135 | bendroflumethiazide |
| 1559 | P-135 | benzthiazide |
| 1560 | P-135 | benzylhydrochlorothiazide |
| 1561 | P-135 | buthiazide |
| 1562 | P-135 | chlorothiazide |
| 1563 | P-135 | chlorthalidone |
| 1564 | P-135 | cyclopenthiazide |
| 1565 | P-135 | cyclothiazide |
| 1566 | P-135 | epithiazide |
| 1567 | P-135 | ethiazide |
| 1568 | P-135 | fenquizone |
| 1569 | P-135 | hydrochlorothiazide |
| 1570 | P-135 | hydroflumethiazide |
| 1571 | P-135 | indapamide |
| 1572 | P-135 | methyclothiazide |
| 1573 | P-135 | meticrane |
| 1574 | P-135 | metolazone |
| 1575 | P-135 | paraflutizide |
| 1576 | P-135 | polythiazide |
| 1577 | P-135 | quinethazone |
| 1578 | P-135 | teclothiazide |
| 1579 | P-135 | trichlormethiazide |
| 1580 | P-135 | acetazolamide |
| 1581 | P-135 | ambuside |
| 1582 | P-135 | azosemide |
| 1583 | P-135 | bumetanide |
| 1584 | P-135 | butazolamide |
| 1585 | P-135 | chloraminophenamide |
| 1586 | P-135 | clofenamide |
| 1587 | P-135 | clopamide |
| 1588 | P-135 | clorexolone |
| 1589 | P-135 | disulfamide |
| 1590 | P-135 | ethoxolamide |
| 1591 | P-135 | furosemide |
| 1592 | P-135 | mefruside |
| 1593 | P-135 | methazolamide |
| 1594 | P-135 | piretanide |
| 1595 | P-135 | torasemide |
| 1596 | P-135 | tripamide |
| 1597 | P-135 | xipamide |
| 1598 | P-135 | mercaptomerin sodium |
| 1599 | P-135 | merethoxylline |
| 1600 | P-135 | procaine |
| 1601 | P-135 | mersalyl with thiophylline |
| 1602 | P-136 | amanozine |
| 1603 | P-136 | amiloride |
| 1604 | P-136 | arbutin |
| 1605 | P-136 | chlorazanil |
| 1606 | P-136 | ethacrynic acid |
| 1607 | P-136 | etozolin |
| 1608 | P-136 | hydracarbazine |
| 1609 | P-136 | isosorbide |
| 1610 | P-136 | mannitol |
| 1611 | P-136 | metochalcone |
| 1612 | P-136 | muzolimine |
| 1613 | P-136 | perhexiline |
| 1614 | P-136 | ticrynafen |
| 1615 | P-136 | triamterene |
| 1616 | P-136 | urea |
| 1617 | P-136 | althiazide |
| 1618 | P-136 | bendroflumethiazide |
| 1619 | P-136 | benzthiazide |
| 1620 | P-136 | benzylhydrochlorothiazide |
| 1621 | P-136 | buthiazide |
| 1622 | P-136 | chlorothiazide |
| 1623 | P-136 | chlorthalidone |
| 1624 | P-136 | cyclopenthiazide |
| 1625 | P-136 | cyclothiazide |
| 1626 | P-136 | epithiazide |
| 1627 | P-136 | ethiazide |
| 1628 | P-136 | fenquizone |
| 1629 | P-136 | hydrochlorothiazide |
| 1630 | P-136 | hydroflumethiazide |
| 1631 | P-136 | indapamide |
| 1632 | P-136 | methyclothiazide |
| 1633 | P-136 | meticrane |
| 1634 | P-136 | metolazone |
| 1635 | P-136 | paraflutizide |
| 1636 | P-136 | polythiazide |
| 1637 | P-136 | quinethazone |
| 1638 | P-136 | teclothiazide |
| 1639 | P-136 | trichlormethiazide |
| 1640 | P-136 | acetazolamide |
| 1641 | P-136 | ambuside |
| 1642 | P-136 | azosemide |
| 1643 | P-136 | bumetanide |
| 1644 | P-136 | butazolamide |
| 1645 | P-136 | chloraminophenamide |
| 1646 | P-136 | clofenamide |
| 1647 | P-136 | clopamide |
| 1648 | P-136 | clorexolone |
| 1649 | P-136 | disulfamide |
| 1650 | P-136 | ethoxolamide |
| 1651 | P-136 | furosemide |
| 1652 | P-136 | mefruside |
| 1653 | P-136 | methazolamide |
| 1654 | P-136 | piretanide |
| 1655 | P-136 | torasemide |
| 1656 | P-136 | tripamide |
| 1657 | P-136 | xipamide |
| 1658 | P-136 | mercaptomerin sodium |
| 1659 | P-136 | merethoxylline |
| 1660 | P-136 | procaine |
| 1661 | P-136 | mersalyl with thiophylline |
| 1662 | P-137 | amanozine |
| 1663 | P-137 | amiloride |
| 1664 | P-137 | arbutin |
| 1665 | P-137 | chlorazanil |
| 1666 | P-137 | ethacrynic acid |
| 1667 | P-137 | etozolin |
| 1668 | P-137 | hydrcarbazine |
| 1669 | P-137 | isosorbide |
| 1670 | P-137 | mannitol |
| 1671 | P-137 | metochalcone |
| 1672 | P-137 | muzolimine |
| 1673 | P-137 | perhexiline |
| 1674 | P-137 | ticrynafen |
| 1675 | P-137 | triamterene |
| 1676 | P-137 | urea |
| 1677 | P-137 | althiazide |
| 1678 | P-137 | bendroflumethiazide |
| 1679 | P-137 | benzthiazide |
| 1680 | P-137 | benzylhydrochlorothiazide |
| 1681 | P-137 | buthiazide |
| 1682 | P-137 | chlorothiazide |
| 1683 | P-137 | chlorthalidone |
| 1684 | P-137 | cyclopenthiazide |
| 1685 | P-137 | cyclothiazide |
| 1686 | P-137 | epithiazide |
| 1687 | P-137 | ethiazide |
| 1688 | P-137 | fenquizone |
| 1689 | P-137 | hydrochlorothiazide |
| 1690 | P-137 | hydroflumethiazide |
| 1691 | P-137 | indapamide |
| 1692 | P-137 | methyclothiazide |
| 1693 | P-137 | meticrane |
| 1694 | P-137 | metolazone |
| 1695 | P-137 | paraflutizide |
| 1696 | P-137 | polythiazide |
| 1697 | P-137 | quinethazone |
| 1698 | P-137 | teclothiazide |
| 1699 | P-137 | trichlormethiazide |
| 1700 | P-137 | acetazolamide |
| 1701 | P-137 | ambuside |
| 1702 | P-137 | azosemide |
| 1703 | P-137 | bumetanide |
| 1704 | P-137 | butazolamide |
| 1705 | P-137 | chloraminophenamide |
| 1706 | P-137 | clofenamide |
| 1707 | P-137 | clopamide |
| 1708 | P-137 | clorexolone |
| 1709 | P-137 | disulfamide |
| 1710 | P-137 | ethoxolamide |
| 1711 | P-137 | furosemide |
| 1712 | P-137 | mefruside |
| 1713 | P-137 | methazolamide |
| 1714 | P-137 | piretanide |
| 1715 | P-137 | torasemide |
| 1716 | P-137 | tripamide |
| 1717 | P-137 | xipamide |
| 1718 | P-137 | mercaptomerin sodium |
| 1719 | P-137 | merethoxylline |
| 1720 | P-137 | procaine |
| 1721 | P-137 | mersalyl with thiophylline |
| 1722 | P-138 | amanozine |
| 1723 | P-138 | amiloride |
| 1724 | P-138 | arbutin |
| 1725 | P-138 | chlorazanil |
| 1726 | P-138 | ethacrynic acid |
| 1727 | P-138 | etozolin |
| 1728 | P-138 | hydracarbazine |
| 1729 | P-138 | isosorbide |
| 1730 | P-138 | mannitol |
| 1731 | P-138 | metochalcone |
| 1732 | P-138 | muzolimine |
| 1733 | P-138 | perhexiline |
| 1734 | P-138 | ticrynafen |
| 1735 | P-138 | triamterene |
| 1736 | P-138 | urea |
| 1737 | P-138 | althiazide |
| 1738 | P-138 | bendroflumethiazide |
| 1739 | P-138 | benzthiazide |
| 1740 | P-138 | benzylhydrochlorothiazide |
| 1741 | P-138 | buthiazide |
| 1742 | P-138 | chlorothiazide |
| 1743 | P-138 | chlorthalidone |
| 1744 | P-138 | cyclopenthiazide |
| 1745 | P-138 | cyclothiazide |
| 1746 | P-138 | epithiazide |
| 1747 | P-138 | ethiazide |
| 1748 | P-138 | fenquizone |
| 1749 | P-138 | hydrochlorothiazide |
| 1750 | P-138 | hydroflumethiazide |
| 1751 | P-138 | indapamide |
| 1752 | P-138 | methyclothiazide |
| 1753 | P-138 | meticrane |
| 1754 | P-138 | metolazone |
| 1755 | P-138 | paraflutizide |
| 1756 | P-138 | polythiazide |
| 1757 | P-138 | quinethazone |
| 1758 | P-138 | teclothiazide |
| 1759 | P-138 | trichlormethiazide |
| 1760 | P-138 | acetazolamide |
| 1761 | P-138 | ambuside |
| 1762 | P-138 | azosemide |
| 1763 | P-138 | bumetanide |
| 1764 | P-138 | butazolamide |
| 1765 | P-138 | chloraminophenamide |
| 1766 | P-138 | clofenamide |
| 1767 | P-138 | clopamide |
| 1768 | P-138 | clorexolone |
| 1769 | P-138 | disulfamide |
| 1770 | P-138 | ethoxolamide |
| 1771 | P-138 | furosemide |
| 1772 | P-138 | mefruside |
| 1773 | P-138 | methazolamide |
| 1774 | P-138 | piretanide |
| 1775 | P-138 | torasemide |
| 1776 | P-138 | tripamide |
| 1777 | P-138 | xipamide |
| 1778 | P-138 | mercaptomerin sodium |
| 1779 | P-138 | merethoxylline |
| 1780 | P-138 | procaine |
| 1781 | P-138 | mersalyl with thiophylline |
| 1782 | P-139 | amanozine |
| 1783 | P-139 | amiloride |
| 1784 | P-139 | arbutin |
| 1785 | P-139 | chlorazanil |
| 1786 | P-139 | ethacrynic acid |
| 1787 | P-139 | etozolin |
| 1788 | P-139 | hydracarbazine |
| 1789 | P-139 | isosorbide |
| 1790 | P-139 | mannitol |
| 1791 | P-139 | metochalcone |
| 1792 | P-139 | muzolimine |
| 1793 | P-139 | perhexiline |
| 1794 | P-139 | ticrynafen |
| 1795 | P-139 | triamterene |
| 1796 | P-139 | urea |
| 1797 | P-139 | althiazide |
| 1798 | P-139 | bendroflumethiazide |
| 1799 | P-139 | benzthiazide |
| 1800 | P-139 | benzylhydrochlorothiazide |
| 1801 | P-139 | buthiazide |
| 1802 | P-139 | chlorothiazide |
| 1803 | P-139 | chlorthalidone |
| 1804 | P-139 | cyclopenthiazide |
| 1805 | P-139 | cyclothiazide |
| 1806 | P-139 | epithiazide |
| 1807 | P-139 | ethiazide |
| 1808 | P-139 | fenquizone |
| 1809 | P-139 | hydrochlorothiazide |
| 1810 | P-139 | hydroflumethiazide |
| 1811 | P-139 | indapamide |
| 1812 | P-139 | methyclothiazide |
| 1813 | P-139 | meticrane |
| 1814 | P-139 | metolazone |
| 1815 | P-139 | paraflutizide |
| 1816 | P-139 | polythiazide |
| 1817 | P-139 | quinethazone |
| 1818 | P-139 | teclothiazide |
| 1819 | P-139 | trichlormethiazide |
| 1820 | P-139 | acetazolamide |
| 1821 | P-139 | ambuside |
| 1822 | P-139 | azosemide |
| 1823 | P-139 | bumetanide |
| 1824 | P-139 | butazolamide |
| 1825 | P-139 | chloraminophenamide |
| 1826 | P-139 | clofenamide |
| 1827 | P-139 | clopamide |
| 1828 | P-139 | clorexolone |
| 1829 | P-139 | disulfamide |
| 1830 | P-139 | ethoxolamide |
| 1831 | P-139 | furosemide |
| 1832 | P-139 | mefruside |
| 1833 | P-139 | methazolamide |
| 1834 | P-139 | piretanide |
| 1835 | P-139 | torasemide |
| 1836 | P-139 | tripamide |
| 1837 | P-139 | xipamide |
| 1838 | P-139 | mercaptomerin sodium |
| 1839 | P-139 | merethoxylline |
| 1840 | P-139 | procaine |
| 1841 | P-139 | mersalyl with thiophylline |
| 1842 | P-140 | amanozine |
| 1843 | P-140 | amiloride |
| 1844 | P-140 | arbutin |
| 1845 | P-140 | chlorazanil |
| 1846 | P-140 | ethacrynic acid |
| 1847 | P-140 | etozolin |
| 1848 | P-140 | hydracarbazine |
| 1849 | P-140 | isosorbide |
| 1850 | P-140 | mannitol |
| 1851 | P-140 | metochalcone |
| 1852 | P-140 | muzolimine |
| 1853 | P-140 | perhexiline |
| 1854 | P-140 | ticrynafen |
| 1855 | P-140 | triamterene |
| 1856 | P-140 | urea |
| 1857 | P-140 | althiazide |
| 1858 | P-140 | bendroflumethiazide |
| 1859 | P-140 | benzthiazide |
| 1860 | P-140 | benzylhydrochlorothiazide |
| 1861 | P-140 | buthiazide |
| 1862 | P-140 | chlorothiazide |
| 1863 | P-140 | chlorthalidone |
| 1864 | P-140 | cyclopenthiazide |
| 1865 | P-140 | cyclothiazide |
| 1866 | P-140 | epithiazide |
| 1867 | P-140 | ethiazide |
| 1868 | P-140 | fenquizone |
| 1869 | P-140 | hydrochlorothiazide |
| 1870 | P-140 | hydroflumethiazide |
| 1871 | P-140 | indapamide |
| 1872 | P-140 | methyclothiazide |
| 1873 | P-140 | meticrane |
| 1874 | P-140 | metolazone |
| 1875 | P-140 | paraflutizide |
| 1876 | P-140 | polythiazide |
| 1877 | P-140 | quinethazone |
| 1878 | P-140 | teclothiazide |
| 1879 | P-140 | trichlormethiazide |
| 1880 | P-140 | acetazolamide |
| 1881 | P-140 | ambuside |
| 1882 | P-140 | azosemide |
| 1883 | P-140 | bumetanide |
| 1884 | P-140 | butazolamide |
| 1885 | P-140 | chloraminophenamide |
| 1886 | P-140 | clofenamide |
| 1887 | P-140 | clopamide |
| 1888 | P-140 | clorexolone |
| 1889 | P-140 | disulfamide |
| 1890 | P-140 | ethoxolamide |
| 1891 | P-140 | furosemide |
| 1892 | P-140 | mefruside |
| 1893 | P-140 | methazolamide |
| 1894 | P-140 | piretanide |
| 1895 | P-140 | torasemide |
| 1896 | P-140 | tripamide |
| 1897 | P-140 | xipamide |
| 1898 | P-140 | mercaptomerin sodium |
| 1899 | P-140 | merethoxylline |
| 1900 | P-140 | procaine |
| 1901 | P-140 | mersalyl with thiophylline |

Table 20 illustrates additional examples of some of the combinations of the present invention wherein the combination comprises a first amount of a reported p38-kinase inhibitor and a second amount of a diuretic:

TABLE 20

| Example | | |
|---|---|---|
| Combination No. | p38-kinase inhibitor | diuretic |
| 1902 | P-141 | amanozine |
| 1903 | P-141 | amiloride |
| 1904 | P-141 | arbutin |
| 1905 | P-141 | chlorazanil |
| 1906 | P-141 | ethacrynic acid |
| 1907 | P-141 | etozolin |
| 1908 | P-141 | hydracarbazine |
| 1909 | P-141 | isosorbide |
| 1910 | P-141 | mannitol |
| 1911 | P-141 | metochalcone |
| 1912 | P-141 | muzolimine |
| 1913 | P-141 | perhexiline |
| 1914 | P-141 | ticrynafen |
| 1915 | P-141 | triamterene |
| 1916 | P-141 | urea |
| 1917 | P-141 | althiazide |
| 1918 | P-141 | bendroflumethiazide |
| 1919 | P-141 | benzthiazide |
| 1920 | P-141 | benzylhydrochlorothiazide |
| 1921 | P-141 | buthiazide |
| 1922 | P-141 | chlorothiazide |
| 1923 | P-141 | chlorthalidone |
| 1924 | P-141 | cyclopenthiazide |
| 1925 | P-141 | cyclothiazide |
| 1926 | P-141 | epithiazide |
| 1927 | P-141 | ethiazide |
| 1928 | P-141 | fenquizone |
| 1929 | P-141 | hydrochlorothiazide |
| 1930 | P-141 | hydroflumethiazide |
| 1931 | P-141 | indapamide |
| 1932 | P-141 | methyclothiazide |
| 1933 | P-141 | meticrane |
| 1934 | P-141 | metolazone |
| 1935 | P-141 | paraflutizide |
| 1936 | P-141 | polythiazide |
| 1937 | P-141 | quinethazone |
| 1938 | P-141 | teclothiazide |
| 1939 | P-141 | trichlormethiazide |
| 1940 | P-141 | acetazolamide |
| 1941 | P-141 | ambuside |
| 1942 | P-141 | azosemide |
| 1943 | P-141 | bumetanide |
| 1944 | P-141 | butazolamide |
| 1945 | P-141 | chloraminophenamide |
| 1946 | P-141 | clofenamide |
| 1947 | P-141 | clopamide |
| 1948 | P-141 | clorexolone |
| 1949 | P-141 | disulfamide |
| 1950 | P-141 | ethoxolamide |
| 1951 | P-141 | furosemide |
| 1952 | P-141 | mefruside |
| 1953 | P-141 | methazolamide |
| 1954 | P-141 | piretanide |
| 1955 | P-141 | torasemide |
| 1956 | P-141 | tripamide |
| 1957 | P-141 | xipamide |
| 1958 | P-141 | mercaptomerin sodium |
| 1959 | P-141 | merethoxylline |
| 1960 | P-141 | procaine |
| 1961 | P-141 | mersalyl with thiophylline |
| 1962 | P-142 | amanozine |
| 1963 | P-142 | amiloride |
| 1964 | P-142 | arbutin |
| 1965 | P-142 | chlorazanil |
| 1966 | P-142 | ethacrynic acid |
| 1967 | P-142 | etozolin |
| 1968 | P-142 | hydracarbazine |
| 1969 | P-142 | isosorbide |
| 1970 | P-142 | mannitol |
| 1971 | P-142 | metochalcone |
| 1972 | P-142 | muzolimine |
| 1973 | P-142 | perhexiline |
| 1974 | P-142 | ticrynafen |
| 1975 | P-142 | triamterene |
| 1976 | P-142 | urea |
| 1977 | P-142 | althiazide |
| 1978 | P-142 | bendroflumethiazide |
| 1979 | P-142 | benzthiazide |
| 1980 | P-142 | benzylhydrochlorothiazide |
| 1981 | P-142 | buthiazide |
| 1982 | P-142 | chlorothiazide |
| 1983 | P-142 | chlorthalidone |
| 1984 | P-142 | cyclopenthiazide |
| 1985 | P-142 | cyclothiazide |
| 1986 | P-142 | epithiazide |
| 1987 | P-142 | ethiazide |
| 1988 | P-142 | fenquizone |
| 1989 | P-142 | hydrochlorothiazide |
| 1990 | P-142 | hydroflumethiazide |
| 1991 | P-142 | indapamide |
| 1992 | P-142 | methyclothiazide |
| 1993 | P-142 | meticrane |
| 1994 | P-142 | metolazone |
| 1995 | P-142 | paraflutizide |
| 1996 | P-142 | polythiazide |
| 1997 | P-142 | quinethazone |
| 1998 | P-142 | teclothiazide |
| 1999 | P-142 | trichlormethiazide |
| 2000 | P-142 | acetazolamide |
| 2001 | P-142 | ambuside |
| 2002 | P-142 | azosemide |
| 2003 | P-142 | bumetanide |
| 2004 | P-142 | butazolamide |
| 2005 | P-142 | chloraminophenamide |
| 2006 | P-142 | clofenamide |
| 2007 | P-142 | clopamide |
| 2008 | P-142 | clorexolone |
| 2009 | P-142 | disulfamide |
| 2010 | P-142 | ethoxolamide |
| 2011 | P-142 | furosemide |
| 2012 | P-142 | mefruside |
| 2013 | P-142 | methazolamide |
| 2014 | P-142 | piretanide |
| 2015 | P-142 | torasemide |
| 2016 | P-142 | tripamide |
| 2017 | P-142 | xipamide |
| 2018 | P-142 | mercaptomerin sodium |
| 2019 | P-142 | merethoxylline |
| 2020 | P-142 | procaine |
| 2021 | P-142 | mersalyl with thiophylline |
| 2022 | P-143 | amanozine |
| 2023 | P-143 | amiloride |
| 2024 | P-143 | arbutin |
| 2025 | P-143 | chlorazanil |
| 2026 | P-143 | ethacrynic acid |
| 2027 | P-143 | etozolin |
| 2028 | P-143 | hydracarbazine |
| 2029 | P-143 | isosorbide |
| 2030 | P-143 | mannitol |
| 2031 | P-143 | metochalcone |
| 2032 | P-143 | muzolimine |
| 2033 | P-143 | perhexiline |
| 2034 | P-143 | ticrynafen |
| 2035 | P-143 | triamterene |
| 2036 | P-143 | urea |
| 2037 | P-143 | althiazide |
| 2038 | P-143 | bendroflumethiazide |
| 2039 | P-143 | benzthiazide |
| 2040 | P-143 | benzylhydrochlorothiazide |
| 2041 | P-143 | buthiazide |
| 2042 | P-143 | chlorothiazide |
| 2043 | P-143 | chlorthalidone |
| 2044 | P-143 | cyclopenthiazide |
| 2045 | P-143 | cyclothiazide |
| 2046 | P-143 | epithiazide |
| 2047 | P-143 | ethiazide |
| 2048 | P-143 | fenquizone |
| 2049 | P-143 | hydrochlorothiazide |
| 2050 | P-143 | hydroflumethiazide |
| 2051 | P-143 | indapamide |
| 2052 | P-143 | methyclothiazide |
| 2053 | P-143 | meticrane |
| 2054 | P-143 | metolazone |
| 2055 | P-143 | paraflutizide |
| 2056 | P-143 | polythiazide |
| 2057 | P-143 | quinethazone |
| 2058 | P-143 | teclothiazide |
| 2059 | P-143 | trichlormethiazide |
| 2060 | P-143 | acetazolamide |
| 2061 | P-143 | ambuside |
| 2062 | P-143 | azosemide |
| 2063 | P-143 | bumetanide |
| 2064 | P-143 | butazolamide |
| 2065 | P-143 | chloraminophenamide |
| 2066 | P-143 | clofenamide |
| 2067 | P-143 | clopamide |
| 2068 | P-143 | clorexolone |
| 2069 | P-143 | disulfamide |
| 2070 | P-143 | ethoxolamide |
| 2071 | P-143 | furosemide |
| 2072 | P-143 | mefruside |
| 2073 | P-143 | methazolamide |
| 2074 | P-143 | piretanide |
| 2075 | P-143 | torasemide |
| 2076 | P-143 | tripamide |
| 2077 | P-143 | xipamide |
| 2078 | P-143 | mercaptomerin sodium |
| 2079 | P-143 | merethoxylline |
| 2080 | P-143 | procaine |
| 2081 | P-143 | mersalyl with thiophylline |
| 2082 | P-144 | amanozine |
| 2083 | P-144 | amiloride |
| 2084 | P-144 | arbutin |
| 2085 | P-144 | chlorazanil |
| 2086 | P-144 | ethacrynic acid |
| 2087 | P-144 | etozolin |
| 2088 | P-144 | hydracarbazine |
| 2089 | P-144 | isosorbide |
| 2090 | P-144 | mannitol |
| 2091 | P-144 | metochalcone |
| 2092 | P-144 | muzolimine |
| 2093 | P-144 | perhexiline |
| 2094 | P-144 | ticrynafen |
| 2095 | P-144 | triamterene |
| 2096 | P-144 | urea |
| 2097 | P-144 | althiazide |
| 2098 | P-144 | bendroflumethiazide |
| 2099 | P-144 | benzthiazide |
| 2100 | P-144 | benzylhydrochlorothiazide |
| 2101 | P-144 | buthiazide |
| 2102 | P-144 | chlorothiazide |
| 2103 | P-144 | chlorthalidone |
| 2104 | P-144 | cyclopenthiazide |
| 2105 | P-144 | cyclothiazide |
| 2106 | P-144 | epithiazide |
| 2107 | P-144 | ethiazide |
| 2108 | P-144 | fenquizone |
| 2109 | P-144 | hydrochlorothiazide |
| 2110 | P-144 | hydroflumethiazide |
| 2111 | P-144 | indapamide |
| 2112 | P-144 | methyclothiazide |
| 2113 | P-144 | meticrane |
| 2114 | P-144 | metolazone |
| 2115 | P-144 | paraflutizide |
| 2116 | P-144 | polythiazide |
| 2117 | P-144 | quinethazone |
| 2118 | P-144 | teclothiazide |
| 2119 | P-144 | trichlormethiazide |
| 2120 | P-144 | acetazolamide |
| 2121 | P-144 | ambuside |
| 2122 | P-144 | azosemide |
| 2123 | P-144 | bumetanide |
| 2124 | P-144 | butazolamide |
| 2125 | P-144 | chloraminophenamide |
| 2126 | P-144 | clofenamide |
| 2127 | P-144 | clopamide |
| 2128 | P-144 | clorexolone |
| 2129 | P-144 | disulfamide |
| 2130 | P-144 | ethoxolamide |
| 2131 | P-144 | furosemide |
| 2132 | P-144 | mefruside |
| 2133 | P-144 | methazolamide |
| 2134 | P-144 | piretanide |
| 2135 | P-144 | torasemide |
| 2136 | P-144 | tripamide |
| 2137 | P-144 | xipamide |
| 2138 | P-144 | mercaptomerin sodium |
| 2139 | P-144 | merethoxylline |
| 2140 | P-144 | procaine |
| 2141 | P-144 | mersalyl with thiophylline |
| 2142 | P-145 | amanozine |
| 2143 | P-145 | amiloride |
| 2144 | P-145 | arbutin |
| 2145 | P-145 | chlorazanil |
| 2146 | P-145 | ethacrynic acid |
| 2147 | P-145 | etozolin |
| 2148 | P-145 | hydracarbazine |
| 2149 | P-145 | isosorbide |
| 2150 | P-145 | mannitol |
| 2151 | P-145 | metochalcone |
| 2152 | P-145 | muzolimine |
| 2153 | P-145 | perhexiline |
| 2154 | P-145 | ticrynafen |
| 2155 | P-145 | triamterene |
| 2156 | P-145 | urea |
| 2157 | P-145 | althiazide |
| 2158 | P-145 | bendroflumethiazide |
| 2159 | P-145 | benzthiazide |
| 2160 | P-145 | benzylhydrochlorothiazide |
| 2161 | P-145 | buthiazide |
| 2162 | P-145 | chlorothiazide |
| 2163 | P-145 | chlorthalidone |
| 2164 | P-145 | cyclopenthiazide |
| 2165 | P-145 | cyclothiazide |
| 2166 | P-145 | epithiazide |
| 2167 | P-145 | ethiazide |
| 2168 | P-145 | fenquizone |
| 2169 | P-145 | hydrochlorothiazide |
| 2170 | P-145 | hydroflumethiazide |
| 2171 | P-145 | indapamide |
| 2172 | P-145 | methyclothiazide |
| 2173 | P-145 | meticrane |
| 2174 | P-145 | metolazone |
| 2175 | P-145 | paraflutizide |
| 2176 | P-145 | polythiazide |
| 2177 | P-145 | quinethazone |
| 2178 | P-145 | teclothiazide |
| 2179 | P-145 | trichlormethiazide |
| 2180 | P-145 | acetazolamide |
| 2181 | P-145 | ambuside |
| 2182 | P-145 | azosemide |
| 2183 | P-145 | bumetanide |
| 2184 | P-145 | butazolamide |
| 2185 | P-145 | chloraminophenamide |
| 2186 | P-145 | clofenamide |
| 2187 | P-145 | clopamide |
| 2188 | P-145 | clorexolone |
| 2189 | P-145 | disulfamide |
| 2190 | P-145 | ethoxolamide |
| 2191 | P-145 | furosemide |
| 2192 | P-145 | mefruside |
| 2193 | P-145 | methazolamide |
| 2194 | P-145 | piretanide |
| 2195 | P-145 | torasemide |
| 2196 | P-145 | tripamide |
| 2197 | P-145 | xipamide |
| 2198 | P-145 | mercaptomerin sodium |
| 2199 | P-145 | merethoxylline |
| 2200 | P-145 | procaine |
| 2201 | P-145 | mersalyl with thioplline |
| 2202 | P-146 | amanozine |
| 2203 | P-146 | amiloride |
| 2204 | P-146 | arbutin |
| 2205 | P-146 | chlorazanil |
| 2206 | P-146 | ethacrynic acid |
| 2207 | P-146 | etozolin |
| 2208 | P-146 | hydracarbazine |
| 2209 | P-146 | isosorbide |
| 2210 | P-146 | mannitol |
| 2211 | P-146 | metochalcone |
| 2212 | P-146 | muzolimine |
| 2213 | P-146 | perhexiline |
| 2214 | P-146 | ticrynafen |
| 2215 | P-146 | triamterene |
| 2216 | P-146 | urea |
| 2217 | P-146 | althiazide |
| 2218 | P-146 | bendroflumethiazide |
| 2219 | P-146 | benzthiazide |
| 2220 | P-146 | benzylhydrochlorothiazide |
| 2221 | P-146 | buthiazide |
| 2222 | P-146 | chlorothiazide |
| 2223 | P-146 | chlorthalidone |
| 2224 | P-146 | cyclopenthiazide |
| 2225 | P-146 | cyclothiazide |
| 2226 | P-146 | epithiazide |
| 2227 | P-146 | ethiazide |
| 2228 | P-146 | fenquizone |
| 2229 | P-146 | hydrochlorothiazide |
| 2230 | P-146 | hydroflumethiazide |
| 2231 | P-146 | indapamide |
| 2232 | P-146 | methyclothiazide |
| 2233 | P-146 | meticrane |
| 2234 | P-146 | metolazone |
| 2235 | P-146 | paraflutizide |
| 2236 | P-146 | polythiazide |
| 2237 | P-146 | quinethazone |
| 2238 | P-146 | teclothiazide |
| 2239 | P-146 | trichlormethiazide |
| 2240 | P-146 | acetazolamide |
| 2241 | P-146 | ambuside |
| 2242 | P-146 | azosemide |
| 2243 | P-146 | bumetanide |
| 2244 | P-146 | butazolamide |
| 2245 | P-146 | chloraminophenamide |
| 2246 | P-146 | clofenamide |
| 2247 | P-146 | clopamide |
| 2248 | P-146 | clorexolone |
| 2249 | P-146 | disulfamide |
| 2250 | P-146 | ethoxolamide |
| 2251 | P-146 | furosemide |
| 2252 | P-146 | mefruside |
| 2253 | P-146 | methazolamide |
| 2254 | P-146 | piretanide |
| 2255 | P-146 | torasemide |
| 2256 | P-146 | tripamide |
| 2257 | P-146 | xipamide |
| 2258 | P-146 | mercaptomerin sodium |
| 2259 | P-146 | merethoxylline |
| 2260 | P-146 | procaine |
| 2261 | P-146 | mersalyl with thiophylline |
| 2262 | P-147 | amanozine |
| 2263 | P-147 | amiloride |
| 2264 | P-147 | arbutin |
| 2265 | P-147 | chlorazanil |
| 2266 | P-147 | ethacrynic acid |
| 2267 | P-147 | etozolin |
| 2268 | P-147 | hydracarbazine |
| 2269 | P-147 | isosorbide |
| 2270 | P-147 | mannitol |
| 2271 | P-147 | metochalcone |
| 2272 | P-147 | muzolimine |
| 2273 | P-147 | perhexiline |
| 2274 | P-147 | ticrynafen |
| 2275 | P-147 | triamterene |
| 2276 | P-147 | urea |
| 2277 | P-147 | althiazide |
| 2278 | P-147 | bendroflumethiazide |
| 2279 | P-147 | benzthiazide |
| 2280 | P-147 | benzylhydrochlorothiazide |
| 2281 | P-147 | buthiazide |
| 2282 | P-147 | chlorothiazide |
| 2283 | P-147 | chlorthalidone |
| 2284 | P-147 | cyclopenthiazide |
| 2285 | P-147 | cyclothiazide |
| 2286 | P-147 | epithiazide |
| 2287 | P-147 | ethiazide |
| 2288 | P-147 | fenquizone |
| 2289 | P-147 | hydrochlorothiazide |
| 2290 | P-147 | hydroflumethiazide |
| 2291 | P-147 | indapamide |
| 2292 | P-147 | methyclothiazide |
| 2293 | P-147 | meticrane |
| 2294 | P-147 | metolazone |
| 2295 | P-147 | paraflutizide |
| 2296 | P-147 | polythiazide |
| 2297 | P-147 | quinethazone |
| 2298 | P-147 | teclothiazide |
| 2299 | P-147 | trichlormethiazide |
| 2300 | P-147 | acetazolamide |
| 2301 | P-147 | ambuside |
| 2302 | P-147 | azosemide |
| 2303 | P-147 | bumetanide |
| 2304 | P-147 | butazolamide |
| 2305 | P-147 | chloraminophenamide |
| 2306 | P-147 | clofenamide |
| 2307 | P-147 | clopamide |
| 2308 | P-147 | clorexolone |
| 2309 | P-147 | disulfamide |
| 2310 | P-147 | ethoxolamide |
| 2311 | P-147 | furosemide |
| 2312 | P-147 | mefruside |
| 2313 | P-147 | methazolamide |
| 2314 | P-147 | piretanide |
| 2315 | P-147 | torasemide |
| 2316 | P-147 | tripamide |
| 2317 | P-147 | xipamide |
| 2318 | P-147 | mercaptomerin sodium |
| 2319 | P-147 | merethoxylline |
| 2320 | P-147 | procaine |
| 2321 | P-147 | mersalyl with thiophylline |
| 2322 | P-148 | amanozine |
| 2323 | P-148 | amiloride |
| 2324 | P-148 | arbutin |
| 2325 | P-148 | chlorazanil |
| 2326 | P-148 | ethacrynic acid |
| 2327 | P-148 | etozolin |
| 2328 | P-148 | hydracarbazine |
| 2329 | P-148 | isosorbide |
| 2330 | P-148 | mannitol |
| 2331 | P-148 | metochalcone |
| 2332 | P-148 | muzolimine |
| 2333 | P-148 | perhexiline |
| 2334 | P-148 | ticrynafen |
| 2335 | P-148 | triamterene |
| 2336 | P-148 | urea |
| 2337 | P-148 | althiazide |
| 2338 | P-148 | bendroflumethiazide |
| 2339 | P-148 | benzthiazide |
| 2340 | P-148 | benzylhydrochlorothiazide |
| 2341 | P-148 | buthiazide |
| 2342 | P-148 | chlorothiazide |
| 2343 | P-148 | chlorthalidone |
| 2344 | P-148 | cyclopenthiazide |
| 2345 | P-148 | cyclothiazide |
| 2346 | P-148 | epithiazide |
| 2347 | P-148 | ethiazide |
| 2348 | P-148 | fenquizone |
| 2349 | P-148 | hydrochlorothiazide |
| 2350 | P-148 | hydroflumethiazide |
| 2351 | P-148 | indapamide |
| 2352 | P-148 | methyclothiazide |
| 2353 | P-148 | meticrane |
| 2354 | P-148 | metolazone |
| 2355 | P-148 | paraflutizide |
| 2356 | P-148 | polythiazide |
| 2357 | P-148 | quinethazone |
| 2358 | P-148 | teclothiazide |
| 2359 | P-148 | trichlormethiazide |
| 2360 | P-148 | acetazolamide |
| 2361 | P-148 | ambuside |
| 2362 | P-148 | azosemide |
| 2363 | P-148 | bumetanide |
| 2364 | P-148 | butazolamide |
| 2365 | P-148 | chloraminophenamide |
| 2366 | P-148 | clofenamide |
| 2367 | P-148 | clopamide |
| 2368 | P-148 | clorexolone |
| 2369 | P-148 | disulfamide |
| 2370 | P-148 | ethoxolamide |
| 2371 | P-148 | furosemide |
| 2372 | P-148 | mefruside |
| 2373 | P-148 | methazolamide |
| 2374 | P-148 | piretanide |
| 2375 | P-148 | torasemide |
| 2376 | P-148 | tripamide |
| 2377 | P-148 | xipamide |
| 2378 | P-148 | mercaptomerin sodium |
| 2379 | P-148 | merethoxylline |
| 2380 | P-148 | procaine |
| 2381 | P-148 | mersalyl with thiophylline |
| 2382 | P-149 | amanozine |
| 2383 | P-149 | amiloride |
| 2384 | P-149 | arbutin |
| 2385 | P-149 | chlorazanil |
| 2386 | P-149 | ethacrynic acid |
| 2387 | P-149 | etozolin |
| 2388 | P-149 | hydracarbazine |
| 2389 | P-149 | isosorbide |
| 2390 | P-149 | mannitol |
| 2391 | P-149 | metochalcone |
| 2392 | P-149 | muzolimine |
| 2393 | P-149 | perhexiline |
| 2394 | P-149 | ticrynafen |
| 2395 | P-149 | triamterene |
| 2396 | P-149 | urea |
| 2397 | P-149 | althiazide |
| 2398 | P-149 | bendroflumethiazide |
| 2399 | P-149 | benzthiazide |
| 2400 | P-149 | benzylhydrochlorothiazide |
| 2401 | P-149 | buthiazide |
| 2402 | P-149 | chlorothiazide |
| 2403 | P-149 | chlorthalidone |
| 2404 | P-149 | cyclopenthiazide |
| 2405 | P-149 | cyclothiazide |
| 2406 | P-149 | epithiazide |
| 2407 | P-149 | ethiazide |
| 2408 | P-149 | fenquizone |
| 2409 | P-149 | hydrochlorothiazide |
| 2410 | P-149 | hydroflumethiazide |
| 2411 | P-149 | indapamide |
| 2412 | P-149 | methyclothiazide |
| 2413 | P-149 | meticrane |
| 2414 | P-149 | metolazone |
| 2415 | P-149 | paraflutizide |
| 2416 | P-149 | polythiazide |
| 2417 | P-149 | quinethazone |
| 2418 | P-149 | teclothiazide |
| 2419 | P-149 | trichlormethiazide |
| 2420 | P-149 | acetazolamide |
| 2421 | P-149 | ambuside |
| 2422 | P-149 | azosemide |
| 2423 | P-149 | bumetanide |
| 2424 | P-149 | butazolamide |
| 2425 | P-149 | chloraminophenamide |
| 2426 | P-149 | clofenamide |
| 2427 | P-149 | clopamide |
| 2428 | P-149 | clorexolone |
| 2429 | P-149 | disulfamide |
| 2430 | P-149 | ethoxolamide |
| 2431 | P-149 | furosemide |
| 2432 | P-149 | mefruside |
| 2433 | P-149 | methazolamide |
| 2434 | P-149 | piretanide |
| 2435 | P-149 | torasemide |
| 2436 | P-149 | tripamide |
| 2437 | P-149 | xipamide |
| 2438 | P-149 | mercaptomerin sodium |
| 2439 | P-149 | merethoxylline |
| 2440 | P-149 | procaine |
| 2441 | P-149 | mersalyl with thiophylline |
| 2442 | P-150 | amanozine |
| 2443 | P-150 | amiloride |
| 2444 | P-150 | arbutin |
| 2445 | P-150 | chlorazanil |
| 2446 | P-150 | ethacrynic acid |
| 2447 | P-150 | etozolin |
| 2448 | P-150 | hydracarbazine |
| 2449 | P-150 | isosorbide |
| 2450 | P-150 | mannitol |
| 2451 | P-150 | metochalcone |
| 2452 | P-150 | muzolimine |
| 2453 | P-150 | perhexiline |
| 2454 | P-150 | ticrynafen |
| 2455 | P-150 | triamterene |
| 2456 | P-150 | urea |
| 2457 | P-150 | althiazide |
| 2458 | P-150 | bendroflumethiazide |
| 2459 | P-150 | benzthiazide |
| 2460 | P-150 | benzylhydrochlorothiazide |
| 2461 | P-150 | buthiazide |
| 2462 | P-150 | chlorothiazide |
| 2463 | P-150 | chlorthalidone |
| 2464 | P-150 | cyclopenthiazide |
| 2465 | P-150 | cyclothiazide |
| 2466 | P-150 | epithiazide |
| 2467 | P-150 | ethiazide |
| 2468 | P-150 | fenquizone |
| 2469 | P-150 | hydrochlorothiazide |
| 2470 | P-150 | hydroflumethiazide |
| 2471 | P-150 | indapamide |
| 2472 | P-150 | methyclothiazide |
| 2473 | P-150 | meticrane |
| 2474 | P-150 | metolazone |
| 2475 | P-150 | paraflutizide |
| 2476 | P-150 | polythiazide |
| 2477 | P-150 | quinethazone |
| 2478 | P-150 | teclothiazide |
| 2479 | P-150 | trichlormethiazide |
| 2480 | P-150 | acetazolamide |
| 2481 | P-150 | ambuside |
| 2482 | P-150 | azosemide |
| 2483 | P-150 | bumetanide |
| 2484 | P-150 | butazolamide |
| 2485 | P-150 | chloraminophenamide |
| 2486 | P-150 | clofenamide |
| 2487 | P-150 | clopamide |
| 2488 | P-150 | clorexolone |
| 2489 | P-150 | disulfamide |
| 2490 | P-150 | ethoxolamide |
| 2491 | P-150 | furosemide |
| 2492 | P-150 | mefruside |
| 2493 | P-150 | methazolamide |
| 2494 | P-150 | piretanide |
| 2495 | P-150 | torasemide |
| 2496 | P-150 | tripamide |
| 2497 | P-150 | xipamide |
| 2498 | P-150 | mercaptomerin sodium |
| 2499 | P-150 | merethoxylline |
| 2500 | P-150 | procaine |
| 2501 | P-150 | mersalyl with thiophylline |
| 2502 | P-151 | amanozine |
| 2503 | P-151 | amiloride |
| 2504 | P-151 | arbutin |
| 2505 | P-151 | chlorazanil |
| 2506 | P-151 | ethacrynic acid |
| 2507 | P-151 | etozolin |
| 2508 | P-151 | hydracarbazine |
| 2509 | P-151 | isosorbide |
| 2510 | P-151 | mannitol |
| 2511 | P-151 | metochalcone |
| 2512 | P-151 | muzolimine |
| 2513 | P-151 | perhexiline |
| 2514 | P-151 | ticrynafen |
| 2515 | P-151 | triamterene |
| 2516 | P-151 | urea |
| 2517 | P-151 | althiazide |
| 2518 | P-151 | bendroflumethiazide |
| 2519 | P-151 | benzthiazide |
| 2520 | P-151 | benzylhydrochlorothiazide |
| 2521 | P-151 | buthiazide |
| 2522 | P-151 | chlorothiazide |
| 2523 | P-151 | chlorthalidone |
| 2524 | P-151 | cyclopenthiazide |
| 2525 | P-151 | cyclothiazide |
| 2526 | P-151 | epithiazide |
| 2527 | P-151 | ethiazide |
| 2528 | P-151 | fenquizone |
| 2529 | P-151 | hydrochlorothiazide |
| 2530 | P-151 | hydroflumethiazide |
| 2531 | P-151 | indapamide |
| 2532 | P-151 | methyclothiazide |
| 2533 | P-151 | meticrane |
| 2534 | P-151 | metolazone |
| 2535 | P-151 | paraflutizide |
| 2536 | P-151 | polythiazide |
| 2537 | P-151 | quinethazone |
| 2538 | P-151 | teclothiazide |
| 2539 | P-151 | trichlormethiazide |
| 2540 | P-151 | acetazolamide |
| 2541 | P-151 | ambuside |
| 2542 | P-151 | azosemide |
| 2543 | P-151 | bumetanide |
| 2544 | P-151 | butazolamide |
| 2545 | P-151 | chloraminophenamide |
| 2546 | P-151 | clofenamide |
| 2547 | P-151 | clopamide |
| 2548 | P-151 | clorexolone |
| 2549 | P-151 | disulfamide |
| 2550 | P-151 | ethoxolamide |
| 2551 | P-151 | furosemide |
| 2552 | P-151 | mefruside |
| 2553 | P-151 | methazolamide |
| 2554 | P-151 | piretanide |
| 2555 | P-151 | torasemide |
| 2556 | P-151 | tripamide |
| 2557 | P-151 | xipamide |
| 2558 | P-151 | mercaptomerin sodium |
| 2559 | P-151 | merethoxylline |
| 2560 | P-151 | procaine |
| 2561 | P-151 | mersalyl with thiophylline |
| 2562 | P-152 | amanozine |
| 2563 | P-152 | amiloride |
| 2564 | P-152 | arbutin |
| 2565 | P-152 | chlorazanil |
| 2566 | P-152 | ethacrynic acid |
| 2567 | P-152 | etozolin |
| 2568 | P-152 | hydracarbazine |
| 2569 | P-152 | isosorbide |
| 2570 | P-152 | mannitol |
| 2571 | P-152 | metochalcone |
| 2572 | P-152 | muzolimine |
| 2573 | P-152 | perhexiline |
| 2574 | P-152 | ticrynafen |
| 2575 | P-152 | triamterene |
| 2576 | P-152 | urea |
| 2577 | P-152 | althiazide |
| 2578 | P-152 | bendroflumethiazide |
| 2579 | P-152 | benzthiazide |
| 2580 | P-152 | benzylhydrochlorothiazide |
| 2581 | P-152 | buthiazide |
| 2582 | P-152 | chlorothiazide |
| 2583 | P-152 | chlorthalidone |
| 2584 | P-152 | cyclopenthiazide |
| 2585 | P-152 | cyclothiazide |
| 2586 | P-152 | epithiazide |
| 2587 | P-152 | ethiazide |
| 2588 | P-152 | fenquizone |
| 2589 | P-152 | hydrochlorothiazide |
| 2590 | P-152 | hydroflumethiazide |
| 2591 | P-152 | indapamide |
| 2592 | P-152 | methyclothiazide |
| 2593 | P-152 | meticrane |
| 2594 | P-152 | metolazone |
| 2595 | P-152 | paraflutizide |
| 2596 | P-152 | polythiazide |
| 2597 | P-152 | quinethazone |
| 2598 | P-152 | teclothiazide |
| 2599 | P-152 | trichlormethiazide |
| 2600 | P-152 | acetazolamide |
| 2601 | P-152 | ambuside |
| 2602 | P-152 | azosemide |
| 2603 | P-152 | bumetanide |
| 2604 | P-152 | butazolamide |
| 2605 | P-152 | chloraminophenamide |
| 2606 | P-152 | clofenamide |
| 2607 | P-152 | clopamide |
| 2608 | P-152 | clorexolone |
| 2609 | P-152 | disulfamide |
| 2610 | P-152 | ethoxolamide |
| 2611 | P-152 | furosemide |
| 2612 | P-152 | mefruside |
| 2613 | P-152 | methazolamide |
| 2614 | P-152 | piretanide |
| 2615 | P-152 | torasemide |
| 2616 | P-152 | tripamide |
| 2617 | P-152 | xipamide |
| 2618 | P-152 | mercaptomerin sodium |
| 2619 | P-152 | merethoxylline |
| 2620 | P-152 | procaine |
| 2621 | P-152 | mersalyl with thiophylline |
| 2622 | P-153 | amanozine |
| 2623 | P-153 | amiloride |
| 2624 | P-153 | arbutin |
| 2625 | P-153 | chlorazanil |
| 2626 | P-153 | ethacrynic acid |
| 2627 | P-153 | etozolin |
| 2628 | P-153 | hydracarbazine |
| 2629 | P-153 | isosorbide |
| 2630 | P-153 | mannitol |
| 2631 | P-153 | metochalcone |
| 2632 | P-153 | muzolimine |
| 2633 | P-153 | perhexiline |
| 2634 | P-153 | ticrynafen |
| 2635 | P-153 | triamterene |
| 2636 | P-153 | urea |
| 2637 | P-153 | althiazide |
| 2638 | P-153 | bendroflumethiazide |
| 2639 | P-153 | benzthiazide |
| 2640 | P-153 | benzylhydrochlorothiazide |
| 2641 | P-153 | buthiazide |
| 2642 | P-153 | chlorothiazide |
| 2643 | P-153 | chlorthalidone |
| 2644 | P-153 | cyclopenthiazide |
| 2645 | P-153 | cyclothiazide |
| 2646 | P-153 | epithiazide |
| 2647 | P-153 | ethiazide |
| 2648 | P-153 | fenquizone |
| 2649 | P-153 | hydrochlorothiazide |
| 2650 | P-153 | hydroflumethiazide |
| 2651 | P-153 | indapamide |
| 2652 | P-153 | methyclothiazide |
| 2653 | P-153 | meticrane |
| 2654 | P-153 | metolazone |
| 2655 | P-153 | paraflutizide |
| 2656 | P-153 | polythiazide |
| 2657 | P-153 | quinethazone |
| 2658 | P-153 | teclothiazide |
| 2659 | P-153 | trichlormethiazide |
| 2660 | P-153 | acetazolamide |
| 2661 | P-153 | ambuside |
| 2662 | P-153 | azosemide |
| 2663 | P-153 | bumetanide |
| 2664 | P-153 | butazolamide |
| 2665 | P-153 | chloraminophenamide |
| 2666 | P-153 | clofenamide |
| 2667 | P-153 | clopamide |
| 2668 | P-153 | clorexolone |
| 2669 | P-153 | disulfamide |
| 2670 | P-153 | ethoxolamide |
| 2671 | P-153 | furosemide |
| 2672 | P-153 | mefruside |
| 2673 | P-153 | methazolamide |
| 2674 | P-153 | piretanide |
| 2675 | P-153 | torasemide |
| 2676 | P-153 | tripamide |
| 2677 | P-153 | xipamide |
| 2678 | P-153 | mercaptomerin sodium |
| 2679 | P-153 | merethoxylline |
| 2680 | P-153 | procaine |
| 2681 | P-153 | mersalyl with thiophylline |
| 2682 | P-154 | amanozine |
| 2683 | P-154 | amiloride |
| 2684 | P-154 | arbutin |
| 2685 | P-154 | chlorazanil |
| 2686 | P-154 | ethacrynic acid |
| 2687 | P-154 | etozolin |
| 2688 | P-154 | hydracarbazine |
| 2689 | P-154 | isosorbide |
| 2690 | P-154 | mannitol |
| 2691 | P-154 | metochalcone |
| 2692 | P-154 | muzolimine |
| 2693 | P-154 | perhexiline |
| 2694 | P-154 | ticrynafen |
| 2695 | P-154 | triamterene |
| 2696 | P-154 | urea |
| 2697 | P-154 | althiazide |
| 2698 | P-154 | bendroflumethiazide |
| 2699 | P-154 | benzthiazide |
| 2700 | P-154 | benzylhydrochlorothiazide |
| 2701 | P-154 | buthiazide |
| 2702 | P-154 | chlorothiazide |
| 2703 | P-154 | chlorthalidone |
| 2704 | P-154 | cyclopenthiazide |
| 2705 | P-154 | cyclothiazide |
| 2706 | P-154 | epithiazide |
| 2707 | P-154 | ethiazide |
| 2708 | P-154 | fenquizone |
| 2709 | P-154 | hydrochlorothiazide |
| 2710 | P-154 | hydroflumethiazide |
| 2711 | P-154 | indapamide |
| 2712 | P-154 | methyclothiazide |
| 2713 | P-154 | meticrane |
| 2714 | P-154 | metolazone |
| 2715 | P-154 | paraflutizide |
| 2716 | P-154 | polythiazide |
| 2717 | P-154 | quinethazone |
| 2718 | P-154 | teclothiazide |
| 2719 | P-154 | trichlormethiazide |
| 2720 | P-154 | acetazolamide |
| 2721 | P-154 | ambuside |
| 2722 | P-154 | azosemide |
| 2723 | P-154 | bumetanide |
| 2724 | P-154 | butazolamide |
| 2725 | P-154 | chloraminophenamide |
| 2726 | P-154 | clofenamide |
| 2727 | P-154 | clopamide |
| 2728 | P-154 | clorexolone |
| 2729 | P-154 | disulfamide |
| 2730 | P-154 | ethoxolamide |
| 2731 | P-154 | furosemide |
| 2732 | P-154 | mefruside |
| 2733 | P-154 | methazolamide |
| 2734 | P-154 | piretanide |
| 2735 | P-154 | torasemide |
| 2736 | P-154 | tripamide |
| 2737 | P-154 | xipamide |
| 2738 | P-154 | mercaptomerin sodium |
| 2739 | P-154 | merethoxylline |
| 2740 | P-154 | procaine |
| 2741 | P-154 | mersalyl with thiophylline |
| 2742 | P-155 | amanozine |
| 2743 | P-155 | amiloride |
| 2744 | P-155 | arbutin |
| 2745 | P-155 | chlorazanil |
| 2746 | P-155 | ethacrynic acid |
| 2747 | P-155 | etozolin |
| 2748 | P-155 | hydracarbazine |
| 2749 | P-155 | isosorbide |
| 2750 | P-155 | mannitol |
| 2751 | P-155 | metochalcone |
| 2752 | P-155 | muzolimine |
| 2753 | P-155 | perhexiline |
| 2754 | P-155 | ticrynafen |
| 2755 | P-155 | triamterene |
| 2756 | P-155 | urea |
| 2757 | P-155 | althiazide |
| 2758 | P-155 | bendroflumethiazide |
| 2759 | P-155 | benzthiazide |
| 2760 | P-155 | benzylhydrochlorothiazide |
| 2761 | P-155 | buthiazide |
| 2762 | P-155 | chlorothiazide |
| 2763 | P-155 | chlorthalidone |
| 2764 | P-155 | cyclopenthiazide |
| 2765 | P-155 | cyclothiazide |
| 2766 | P-155 | epithiazide |
| 2767 | P-155 | ethiazide |
| 2768 | P-155 | fenquizone |
| 2769 | P-155 | hydrochlorothiazide |
| 2770 | P-155 | hydroflumethiazide |
| 2771 | P-155 | indapamide |
| 2772 | P-155 | methyclothiazide |
| 2773 | P-155 | meticrane |
| 2774 | P-155 | metolazone |
| 2775 | P-155 | paraflutizide |
| 2776 | P-155 | polythiazide |
| 2777 | P-155 | quinethazone |
| 2778 | P-155 | teclothiazide |
| 2779 | P-155 | trichlormethiazide |
| 2780 | P-155 | acetazolamide |
| 2781 | P-155 | ambuside |
| 2782 | P-155 | azosemide |
| 2783 | P-155 | bumetanide |
| 2784 | P-155 | butazolamide |
| 2785 | P-155 | chloraminophenamide |
| 2786 | P-155 | clofenamide |
| 2787 | P-155 | clopamide |
| 2788 | P-155 | clorexolone |
| 2789 | P-155 | disulfamide |
| 2790 | P-155 | ethoxolamide |
| 2791 | P-155 | furosemide |
| 2792 | P-155 | mefruside |
| 2793 | P-155 | methazolamide |
| 2794 | P-155 | piretanide |
| 2795 | P-155 | torasemide |
| 2796 | P-155 | tripamide |
| 2797 | P-155 | xipamide |
| 2798 | P-155 | mercaptomerin sodium |
| 2799 | P-155 | merethoxylline |
| 2800 | P-155 | procaine |
| 2801 | P-155 | mersalyl with thiophylline |
| 2802 | P-156 | amanozine |
| 2803 | P-156 | amiloride |
| 2804 | P-156 | arbutin |
| 2805 | P-156 | chlorazanil |
| 2806 | P-156 | ethacrynic acid |
| 2807 | P-156 | etozolin |
| 2808 | P-156 | hydracarbazine |
| 2809 | P-156 | isosorbide |
| 2810 | P-156 | mannitol |
| 2811 | P-156 | metochalcone |
| 2812 | P-156 | muzolimine |
| 2813 | P-156 | perhexiline |
| 2814 | P-156 | ticrynafen |
| 2815 | P-156 | triamterene |
| 2816 | P-156 | urea |
| 2817 | P-156 | althiazide |
| 2818 | P-156 | bendroflumethiazide |
| 2819 | P-156 | benzthiazide |
| 2820 | P-156 | benzylhydrochlorothiazide |
| 2821 | P-156 | buthiazide |
| 2822 | P-156 | chlorothiazide |
| 2823 | P-156 | chlorthalidone |
| 2824 | P-156 | cyclopenthiazide |
| 2825 | P-156 | cyclothiazide |
| 2826 | P-156 | epithiazide |
| 2827 | P-156 | ethiazide |
| 2828 | P-156 | fenquizone |
| 2829 | P-156 | hydrochlorothiazide |
| 2830 | P-156 | hydroflumethiazide |
| 2831 | P-156 | indapamide |
| 2832 | P-156 | methyclothiazide |
| 2833 | P-156 | meticrane |
| 2834 | P-156 | metolazone |
| 2835 | P-156 | paraflutizide |
| 2836 | P-156 | polythiazide |
| 2837 | P-156 | quinethazone |
| 2838 | P-156 | teclothiazide |
| 2839 | P-156 | trichlormethiazide |
| 2840 | P-156 | acetazolamide |
| 2841 | P-156 | ambuside |
| 2842 | P-156 | azosemide |
| 2843 | P-156 | bumetanide |
| 2844 | P-156 | butazolamide |
| 2845 | P-156 | chloraminophenamide |
| 2846 | P-156 | clofenamide |
| 2847 | P-156 | clopamide |
| 2848 | P-156 | clorexolone |
| 2849 | P-156 | disulfamide |
| 2850 | P-156 | ethoxolamide |
| 2851 | P-156 | furosemide |
| 2852 | P-156 | mefruside |
| 2853 | P-156 | methazolamide |
| 2854 | P-156 | piretanide |
| 2855 | P-156 | torasemide |
| 2856 | P-156 | tripamide |
| 2857 | P-156 | xipamide |
| 2858 | P-156 | mercaptomerin sodium |
| 2859 | P-156 | merethoxylline |
| 2860 | P-156 | procaine |
| 2861 | P-156 | mersalyl with thiophylline |
| 2862 | P-157 | amanozine |
| 2863 | P-157 | amiloride |
| 2864 | P-157 | arbutin |
| 2865 | P-157 | chlorazanil |
| 2866 | P-157 | ethacrynic acid |
| 2867 | P-157 | etozolin |
| 2868 | P-157 | hydracarbazine |
| 2869 | P-157 | isosorbide |
| 2870 | P-157 | mannitol |
| 2871 | P-157 | metochalcone |
| 2872 | P-157 | muzolimine |
| 2873 | P-157 | perhexiline |
| 2874 | P-157 | ticrynafen |
| 2875 | P-157 | triamterene |
| 2876 | P-157 | urea |
| 2877 | P-157 | althiazide |
| 2878 | P-157 | bendroflumethiazide |
| 2879 | P-157 | benzthiazide |
| 2880 | P-157 | benzylhydrochlorothiazide |
| 2881 | P-157 | buthiazide |
| 2882 | P-157 | chlorothiazide |
| 2883 | P-157 | chlorthalidone |
| 2884 | P-157 | cyclopenthiazide |
| 2885 | P-157 | cyclothiazide |
| 2886 | P-157 | epithiazide |
| 2887 | P-157 | ethiazide |
| 2888 | P-157 | fenquizone |
| 2889 | P-157 | hydrochlorothiazide |
| 2890 | P-157 | hydroflumethiazide |
| 2891 | P-157 | indapamide |
| 2892 | P-157 | methyclothiazide |
| 2893 | P-157 | meticrane |
| 2894 | P-157 | metolazone |
| 2895 | P-157 | paraflutizide |
| 2896 | P-157 | polythiazide |
| 2897 | P-157 | quinethazone |
| 2898 | P-157 | teclothiazide |
| 2899 | P-157 | trichlormethiazide |
| 2900 | P-157 | acetazolamide |
| 2901 | P-157 | ambuside |
| 2902 | P-157 | azosemide |
| 2903 | P-157 | bumetanide |
| 2904 | P-157 | butazolamide |
| 2905 | P-157 | chloraminophenamide |
| 2906 | P-157 | clofenamide |
| 2907 | P-157 | clopamide |
| 2908 | P-157 | clorexolone |
| 2909 | P-157 | disulfamide |
| 2910 | P-157 | ethoxolamide |
| 2911 | P-157 | furosemide |
| 2912 | P-157 | mefruside |
| 2913 | P-157 | methazolamide |
| 2914 | P-157 | piretanide |
| 2915 | P-157 | torasemide |
| 2916 | P-157 | tripamide |
| 2917 | P-157 | xipamide |
| 2918 | P-157 | mercaptomerin sodium |
| 2919 | P-157 | merethoxylline |
| 2920 | P-157 | procaine |
| 2921 | P-157 | mersalyl with thiophylline |
| 2922 | P-158 | amanozine |
| 2923 | P-158 | amiloride |
| 2924 | P-158 | arbutin |
| 2925 | P-158 | chlorazanil |
| 2926 | P-158 | ethacrynic acid |
| 2927 | P-158 | etozolin |
| 2928 | P-158 | hydracarbazine |
| 2929 | P-158 | isosorbide |
| 2930 | P-158 | mannitol |
| 2931 | P-158 | metochalcone |
| 2932 | P-158 | muzolimine |
| 2933 | P-158 | perhexiline |
| 2934 | P-158 | ticrynafen |
| 2935 | P-158 | triamterene |
| 2936 | P-158 | urea |
| 2937 | P-158 | althiazide |
| 2938 | P-158 | bendroflumethiazide |
| 2939 | P-158 | benzthiazide |
| 2940 | P-158 | benzylhydrochlorothiazide |
| 2941 | P-158 | buthiazide |
| 2942 | P-158 | chlorothiazide |
| 2943 | P-158 | chlorthalidone |
| 2944 | P-158 | cyclopenthiazide |
| 2945 | P-158 | cyclothiazide |
| 2946 | P-158 | epithiazide |
| 2947 | P-158 | ethiazide |
| 2948 | P-158 | fenquizone |
| 2949 | P-158 | hydrochlorothiazide |
| 2950 | P-158 | hydroflumethiazide |
| 2951 | P-158 | indapamide |
| 2952 | P-158 | methyclothiazide |
| 2953 | P-158 | meticrane |
| 2954 | P-158 | metolazone |
| 2955 | P-158 | paraflutizide |
| 2956 | P-158 | polythiazide |
| 2957 | P-158 | quinethazone |
| 2958 | P-158 | teclothiazide |
| 2959 | P-158 | trichlormethiazide |
| 2960 | P-158 | acetazolamide |
| 2961 | P-158 | ambuside |
| 2962 | P-158 | azosemide |
| 2963 | P-158 | bumetanide |
| 2964 | P-158 | butazolamide |
| 2965 | P-158 | chloraminophenamide |
| 2966 | P-158 | clofenamide |
| 2967 | P-158 | clopamide |
| 2968 | P-158 | clorexolone |
| 2969 | P-158 | disulfamide |
| 2970 | P-158 | ethoxolamide |
| 2971 | P-158 | furosemide |
| 2972 | P-158 | mefruside |
| 2973 | P-158 | methazolamide |
| 2974 | P-158 | piretanide |
| 2975 | P-158 | torasemide |
| 2976 | P-158 | tripamide |
| 2977 | P-158 | xipamide |
| 2978 | P-158 | mercaptomerin sodium |
| 2979 | P-158 | merethoxylline |
| 2980 | P-158 | procaine |
| 2981 | P-158 | mersalyl with thiophylline |
| 2982 | P-159 | amanozine |
| 2983 | P-159 | amiloride |
| 2984 | P-159 | arbutin |
| 2985 | P-159 | chlorazanil |
| 2986 | P-159 | ethacrynic acid |
| 2987 | P-159 | etozolin |
| 2988 | P-159 | hydracarbazine |
| 2989 | P-159 | isosorbide |
| 2990 | P-159 | mannitol |
| 2991 | P-159 | metochalcone |
| 2992 | P-159 | muzolimine |
| 2993 | P-159 | perhexiline |
| 2994 | P-159 | ticrynafen |
| 2995 | P-159 | triamterene |
| 2996 | P-159 | urea |
| 2997 | P-159 | althiazide |
| 2998 | P-159 | bendroflumethiazide |
| 2999 | P-159 | benzthiazide |
| 3000 | P-159 | benzylhydrochlorothiazide |
| 3001 | P-159 | buthiazide |
| 3002 | P-159 | chlorothiazide |
| 3003 | P-159 | chlorthalidone |
| 3004 | P-159 | cyclopenthiazide |
| 3005 | P-159 | cyclothiazide |
| 3006 | P-159 | epithiazide |
| 3007 | P-159 | ethiazide |
| 3008 | P-159 | fenquizone |
| 3009 | P-159 | hydrochlorothiazide |
| 3010 | P-159 | hydroflumethiazide |
| 3011 | P-159 | indapamide |
| 3012 | P-159 | methyclothiazide |
| 3013 | P-159 | meticrane |
| 3014 | P-159 | metolazone |
| 3015 | P-159 | paraflutizide |
| 3016 | P-159 | polythiazide |
| 3017 | P-159 | quinethazone |
| 3018 | P-159 | teclothiazide |
| 3019 | P-159 | trichlormethiazide |
| 3020 | P-159 | acetazolamide |
| 3021 | P-159 | ambuside |
| 3022 | P-159 | azosemide |
| 3023 | P-159 | bumetanide |
| 3024 | P-159 | butazolamide |
| 3025 | P-159 | chloraminophenamide |
| 3026 | P-159 | clofenamide |
| 3027 | P-159 | clopamide |
| 3028 | P-159 | clorexolone |
| 3029 | P-159 | disulfamide |
| 3030 | P-159 | ethoxolamide |
| 3031 | P-159 | furosemide |
| 3032 | P-159 | mefruside |
| 3033 | P-159 | methazolamide |
| 3034 | P-159 | piretanide |
| 3035 | P-159 | torasemide |
| 3036 | P-159 | tripamide |
| 3037 | P-159 | xipamide |
| 3038 | P-159 | mercaptomerin sodium |
| 3039 | P-159 | merethoxylline |
| 3040 | P-159 | procaine |
| 3041 | P-159 | mersalyl with thiophylline |
| 3042 | P-160 | amanozine |
| 3043 | P-160 | amiloride |
| 3044 | P-160 | arbutin |
| 3045 | P-160 | chlorazanil |
| 3046 | P-160 | ethacrynic acid |
| 3047 | P-160 | etozolin |
| 3048 | P-160 | hydracarbazine |
| 3049 | P-160 | isosorbide |
| 3050 | P-160 | mannitol |
| 3051 | P-160 | metochalcone |
| 3052 | P-160 | muzolimine |
| 3053 | P-160 | perhexiline |
| 3054 | P-160 | ticrynafen |
| 3055 | P-160 | triamterene |
| 3056 | P-160 | urea |
| 3057 | P-160 | althiazide |
| 3058 | P-160 | bendroflumethiazide |
| 3059 | P-160 | benzthiazide |
| 3060 | P-160 | benzylhydrochlorothiazide |
| 3061 | P-160 | buthiazide |
| 3062 | P-160 | chlorothiazide |
| 3063 | P-160 | chlorthalidone |
| 3064 | P-160 | cyclopenthiazide |
| 3065 | P-160 | cyclothiazide |
| 3066 | P-160 | epithiazide |
| 3067 | P-160 | ethiazide |
| 3068 | P-160 | fenquizone |
| 3069 | P-160 | hydrochlorothiazide |
| 3070 | P-160 | hydroflumethiazide |
| 3071 | P-160 | indapamide |
| 3072 | P-160 | methyclothiazide |
| 3073 | P-160 | meticrane |
| 3074 | P-160 | metolazone |
| 3075 | P-160 | paraflutizide |
| 3076 | P-160 | polythiazide |
| 3077 | P-160 | quinethazone |
| 3078 | P-160 | teclothiazide |
| 3079 | P-160 | trichlormethiazide |
| 3080 | P-160 | acetazolamide |
| 3081 | P-160 | ambuside |
| 3082 | P-160 | azosemide |
| 3083 | P-160 | bumetanide |
| 3084 | P-160 | butazolamide |
| 3085 | P-160 | chloraminophenamide |
| 3086 | P-160 | clofenamide |
| 3087 | P-160 | clopamide |
| 3088 | P-160 | clorexolone |
| 3089 | P-160 | disulfamide |
| 3090 | P-160 | ethoxolamide |
| 3091 | P-160 | furosemide |
| 3092 | P-160 | mefruside |
| 3093 | P-160 | methazolamide |
| 3094 | P-160 | piretanide |
| 3095 | P-160 | torasemide |
| 3096 | P-160 | tripamide |
| 3097 | P-160 | xipamide |
| 3098 | P-160 | mercaptomerin sodium |
| 3099 | P-160 | merethoxylline |
| 3100 | P-160 | procaine |
| 3101 | P-160 | mersalyl with thiophylline |
| 3102 | P-161 | amanozine |
| 3103 | P-161 | amiloride |
| 3104 | P-161 | arbutin |
| 3105 | P-161 | chlorazanil |
| 3106 | P-161 | ethacrynic acid |
| 3107 | P-161 | etozolin |
| 3108 | P-161 | hydracarbazine |
| 3109 | P-161 | isosorbide |
| 3110 | P-161 | mannitol |
| 3111 | P-161 | metochalcone |
| 3112 | P-161 | muzolimine |
| 3113 | P-161 | perhexiline |
| 3114 | P-161 | ticrynafen |
| 3115 | P-161 | triamterene |
| 3116 | P-161 | urea |
| 3117 | P-161 | althiazide |
| 3118 | P-161 | bendroflumethiazide |
| 3119 | P-161 | benzthiazide |
| 3120 | P-161 | benzylhydrochlorothiazide |
| 3121 | P-161 | buthiazide |
| 3122 | P-161 | chlorothiazide |
| 3123 | P-161 | chlorthalidone |
| 3124 | P-161 | cyclopenthiazide |
| 3125 | P-161 | cyclothiazide |
| 3126 | P-161 | epithiazide |
| 3127 | P-161 | ethiazide |
| 3128 | P-161 | fenquizone |
| 3129 | P-161 | hydrochlorothiazide |
| 3130 | P-161 | hydroflumethiazide |
| 3131 | P-161 | indapamide |
| 3132 | P-161 | methyclothiazide |
| 3133 | P-161 | meticrane |
| 3134 | P-161 | metolazone |
| 3135 | P-161 | paraflutizide |
| 3136 | P-161 | polythiazide |
| 3137 | P-161 | quinethazone |
| 3138 | P-161 | teclothiazide |
| 3139 | P-161 | trichlormethiazide |
| 3140 | P-161 | acetazolamide |
| 3141 | P-161 | ambuside |
| 3142 | P-161 | azosemide |
| 3143 | P-161 | bumetanide |
| 3144 | P-161 | butazolamide |
| 3145 | P-161 | chloraminophenamide |
| 3146 | P-161 | clofenamide |
| 3147 | P-161 | clopamide |
| 3148 | P-161 | clorexolone |
| 3149 | P-161 | disulfamide |
| 3150 | P-161 | ethoxolamide |
| 3151 | P-161 | furosemide |
| 3152 | P-161 | mefruside |
| 3153 | P-161 | methazolamide |
| 3154 | P-161 | piretanide |
| 3155 | P-161 | torasemide |
| 3156 | P-161 | tripamide |
| 3157 | P-161 | xipamide |
| 3158 | P-161 | mercaptomerin sodium |
| 3159 | P-161 | merethoxylline |
| 3160 | P-161 | procaine |
| 3161 | P-161 | mersalyl with thiophylline |
| 3162 | P-162 | amanozine |
| 3163 | P-162 | amiloride |
| 3164 | P-162 | arbutin |
| 3165 | P-162 | chlorazanil |
| 3166 | P-162 | ethacrynic acid |
| 3167 | P-162 | etozolin |
| 3168 | P-162 | hydracarbazine |
| 3169 | P-162 | isosorbide |
| 3170 | P-162 | mannitol |
| 3171 | P-162 | metochalcone |
| 3172 | P-162 | muzolimine |
| 3173 | P-162 | perhexiline |
| 3174 | P-162 | ticrynafen |
| 3175 | P-162 | triamterene |
| 3176 | P-162 | urea |
| 3177 | P-162 | althiazide |
| 3178 | P-162 | bendroflumethiazide |
| 3179 | P-162 | benzthiazide |
| 3180 | P-162 | benzylhydrochlorothiazide |
| 3181 | P-162 | buthiazide |
| 3182 | P-162 | chlorothiazide |
| 3183 | P-162 | chlorthalidone |
| 3184 | P-162 | cyclopenthiazide |
| 3185 | P-162 | cyclothiazide |
| 3186 | P-162 | epithiazide |
| 3187 | P-162 | ethiazide |
| 3188 | P-162 | fenquizone |
| 3189 | P-162 | hydrochlorothiazide |
| 3190 | P-162 | hydroflumethiazide |
| 3191 | P-162 | indapamide |
| 3192 | P-162 | methyclothiazide |
| 3193 | P-162 | meticrane |
| 3194 | P-162 | metolazone |
| 3195 | P-162 | paraflutizide |
| 3196 | P-162 | polythiazide |
| 3197 | P-162 | quinethazone |
| 3198 | P-162 | teclothiazide |
| 3199 | P-162 | trichlormethiazide |
| 3200 | P-162 | acetazolamide |
| 3201 | P-162 | ambuside |
| 3202 | P-162 | azosemide |
| 3203 | P-162 | bumetanide |
| 3204 | P-162 | butazolamide |
| 3205 | P-162 | chloraminophenamide |
| 3206 | P-162 | clofenamide |
| 3207 | P-162 | clopamide |
| 3208 | P-162 | clorexolone |
| 3209 | P-162 | disulfamide |
| 3210 | P-162 | ethoxolamide |
| 3211 | P-162 | furosemide |
| 3212 | P-162 | mefruside |
| 3213 | P-162 | methazolamide |
| 3214 | P-162 | piretanide |
| 3215 | P-162 | torasemide |
| 3216 | P-162 | tripamide |
| 3217 | P-162 | xipamide |
| 3218 | P-162 | mercaptomerin sodium |
| 3219 | P-162 | merethoxylline |
| 3220 | P-162 | procaine |
| 3221 | P-162 | mersalyl with thiophylline |
| 3222 | P-163 | amanozine |
| 3223 | P-163 | amiloride |
| 3224 | P-163 | arbutin |
| 3225 | P-163 | chlorazanil |
| 3226 | P-163 | ethacrynic acid |
| 3227 | P-163 | etozolin |
| 3228 | P-163 | hydracarbazine |
| 3229 | P-163 | isosorbide |
| 3230 | P-163 | mannitol |
| 3231 | P-163 | metochalcone |
| 3232 | P-163 | muzolimine |
| 3233 | P-163 | perhexiline |
| 3234 | P-163 | ticrynafen |
| 3235 | P-163 | triamterene |
| 3236 | P-163 | urea |
| 3237 | P-163 | althiazide |
| 3238 | P-163 | bendroflumethiazide |
| 3239 | P-163 | benzthiazide |
| 3240 | P-163 | benzylhydrochlorothiazide |
| 3241 | P-163 | buthiazide |
| 3242 | P-163 | chlorothiazide |
| 3243 | P-163 | chlorthalidone |
| 3244 | P-163 | cyclopenthiazide |
| 3245 | P-163 | cyclothiazide |
| 3246 | P-163 | epithiazide |
| 3247 | P-163 | ethiazide |
| 3248 | P-163 | fenquizone |
| 3249 | P-163 | hydrochlorothiazide |
| 3250 | P-163 | hydroflumethiazide |
| 3251 | P-163 | indapamide |
| 3252 | P-163 | methyclothiazide |
| 3253 | P-163 | meticrane |
| 3254 | P-163 | metolazone |
| 3255 | P-163 | paraflutizide |
| 3256 | P-163 | polythiazide |
| 3257 | P-163 | quinethazone |
| 3258 | P-163 | teclothiazide |
| 3259 | P-163 | trichlormethiazide |
| 3260 | P-163 | acetazolamide |
| 3261 | P-163 | ambuside |
| 3262 | P-163 | azosemide |
| 3263 | P-163 | bumetanide |
| 3264 | P-163 | butazolamide |
| 3265 | P-163 | chloraminophenamide |
| 3266 | P-163 | clofenamide |
| 3267 | P-163 | clopamide |
| 3268 | P-163 | clorexolone |
| 3269 | P-163 | disulfamide |
| 3270 | P-163 | ethoxolamide |
| 3271 | P-163 | furosemide |
| 3272 | P-163 | mefruside |
| 3273 | P-163 | methazolamide |
| 3274 | P-163 | piretanide |
| 3275 | P-163 | torasemide |
| 3276 | P-163 | tripamide |
| 3277 | P-163 | xipamide |
| 3278 | P-163 | mercaptomerin sodium |
| 3279 | P-163 | merethoxylline |
| 3280 | P-163 | procaine |
| 3281 | P-163 | mersalyl with thiophylline |
| 3282 | P-164 | amanozine |
| 3283 | P-164 | amiloride |
| 3284 | P-164 | arbutin |
| 3285 | P-164 | chlorazanil |
| 3286 | P-164 | ethacrynic acid |
| 3287 | P-164 | etozolin |
| 3288 | P-164 | hydracarbazine |
| 3289 | P-164 | isosorbide |
| 3290 | P-164 | mannitol |
| 3291 | P-164 | metochalcone |
| 3292 | P-164 | muzolimine |
| 3293 | P-164 | perhexiline |
| 3294 | P-164 | ticrynafen |
| 3295 | P-164 | triamterene |
| 3296 | P-164 | urea |
| 3297 | P-164 | althiazide |
| 3298 | P-164 | bendroflumethiazide |
| 3299 | P-164 | benzthiazide |
| 3300 | P-164 | benzylhydrochlorothiazide |
| 3301 | P-164 | buthiazide |
| 3302 | P-164 | chlorothiazide |
| 3303 | P-164 | chlorthalidone |
| 3304 | P-164 | cyclopenthiazide |
| 3305 | P-164 | cyclothiazide |
| 3306 | P-164 | epithiazide |
| 3307 | P-164 | ethiazide |
| 3308 | P-164 | fenquizone |
| 3309 | P-164 | hydrochlorothiazide |
| 3310 | P-164 | hydroflumethiazide |
| 3311 | P-164 | indapamide |
| 3312 | P-164 | methyclothiazide |
| 3313 | P-164 | meticrane |
| 3314 | P-164 | metolazone |
| 3315 | P-164 | paraflutizide |
| 3316 | P-164 | polythiazide |
| 3317 | P-164 | quinethazone |
| 3318 | P-164 | teclothiazide |
| 3319 | P-164 | trichlormethiazide |
| 3320 | P-164 | acetazolamide |
| 3321 | P-164 | ambuside |
| 3322 | P-164 | azosemide |
| 3323 | P-164 | bumetanide |
| 3324 | P-164 | butazolamide |
| 3325 | P-164 | chloraminophenamide |
| 3326 | P-164 | clofenamide |
| 3327 | P-164 | clopamide |
| 3328 | P-164 | clorexolone |
| 3329 | P-164 | disulfamide |
| 3330 | P-164 | ethoxolamide |
| 3331 | P-164 | furosemide |
| 3332 | P-164 | mefruside |
| 3333 | P-164 | methazolamide |
| 3334 | P-164 | piretanide |
| 3335 | P-164 | torasemide |
| 3336 | P-164 | tripamide |
| 3337 | P-164 | xipamide |
| 3338 | P-164 | mercaptomerin sodium |
| 3339 | P-164 | merethoxylline |
| 3340 | P-164 | procaine |
| 3341 | P-164 | mersalyl with thiophylline |
| 3342 | P-165 | amanozine |
| 3343 | P-165 | amiloride |
| 3344 | P-165 | arbutin |
| 3345 | P-165 | chlorazanil |
| 3346 | P-165 | ethacrynic acid |
| 3347 | P-165 | etozolin |
| 3348 | P-165 | hydracarbazine |
| 3349 | P-165 | isosorbide |
| 3350 | P-165 | mannitol |
| 3351 | P-165 | metochalcone |
| 3352 | P-165 | muzolimine |
| 3353 | P-165 | perhexiline |
| 3354 | P-165 | ticrynafen |
| 3355 | P-165 | triamterene |
| 3356 | P-165 | urea |
| 3357 | P-165 | althiazide |
| 3358 | P-165 | bendroflumethiazide |
| 3359 | P-165 | benzthiazide |
| 3360 | P-165 | benzylhydrochlorothiazide |
| 3361 | P-165 | buthiazide |
| 3362 | P-165 | chlorothiazide |
| 3363 | P-165 | chlorthalidone |
| 3364 | P-165 | cyclopenthiazide |
| 3365 | P-165 | cyclothiazide |
| 3366 | P-165 | epithiazide |
| 3367 | P-165 | ethiazide |
| 3368 | P-165 | fenquizone |
| 3369 | P-165 | hydrochlorothiazide |
| 3370 | P-165 | hydroflumethiazide |
| 3371 | P-165 | indapamide |
| 3372 | P-165 | methyclothiazide |
| 3373 | P-165 | meticrane |
| 3374 | P-165 | metolazone |
| 3375 | P-165 | paraflutizide |
| 3376 | P-165 | polythiazide |
| 3377 | P-165 | quinethazone |
| 3378 | P-165 | teclothiazide |
| 3379 | P-165 | trichlormethiazide |
| 3380 | P-165 | acetazolamide |
| 3381 | P-165 | ambuside |
| 3382 | P-165 | azosemide |
| 3383 | P-165 | bumetanide |
| 3384 | P-165 | butazolamide |
| 3385 | P-165 | chloraminophenamide |
| 3386 | P-165 | clofenamide |
| 3387 | P-165 | clopamide |
| 3388 | P-165 | clorexolone |
| 3389 | P-165 | disulfamide |
| 3390 | P-165 | ethoxolamide |
| 3391 | P-165 | furosemide |
| 3392 | P-165 | mefruside |
| 3393 | P-165 | methazolamide |
| 3394 | P-165 | piretanide |
| 3395 | P-165 | torasemide |
| 3396 | P-165 | tripamide |
| 3397 | P-165 | xipamide |
| 3398 | P-165 | mercaptomerin sodium |
| 3399 | P-165 | merethoxylline |
| 3400 | P-165 | procaine |
| 3401 | P-165 | mersalyl with thiophylline |
| 3402 | P-166 | amanozine |
| 3403 | P-166 | amiloride |
| 3404 | P-166 | arbutin |
| 3405 | P-166 | chlorazanil |
| 3406 | P-166 | ethacrynic acid |
| 3407 | P-166 | etozolin |
| 3408 | P-166 | hydracarbazine |
| 3409 | P-166 | isosorbide |
| 3410 | P-166 | mannitol |
| 3411 | P-166 | metochalcone |
| 3412 | P-166 | muzolimine |
| 3413 | P-166 | perhexiline |
| 3414 | P-166 | ticrynafen |
| 3415 | P-166 | triamterene |
| 3416 | P-166 | urea |
| 3417 | P-166 | althiazide |
| 3418 | P-166 | bendroflumethiazide |
| 3419 | P-166 | benzthiazide |
| 3420 | P-166 | benzylhydrochlorothiazide |
| 3421 | P-166 | buthiazide |
| 3422 | P-166 | chlorothiazide |
| 3423 | P-166 | chlorthalidone |
| 3424 | P-166 | cyclopenthiazide |
| 3425 | P-166 | cyclothiazide |
| 3426 | P-166 | epithiazide |
| 3427 | P-166 | ethiazide |
| 3428 | P-166 | fenquizone |
| 3429 | P-166 | hydrochlorothiazide |
| 3430 | P-166 | hydroflumethiazide |
| 3431 | P-166 | indapamide |
| 3432 | P-166 | methyclothiazide |
| 3433 | P-166 | meticrane |
| 3434 | P-166 | metolazone |
| 3435 | P-166 | paraflutizide |
| 3436 | P-166 | polythiazide |
| 3437 | P-166 | quinethazone |
| 3438 | P-166 | teclothiazide |
| 3439 | P-166 | trichlormethiazide |
| 3440 | P-166 | acetazolamide |
| 3441 | P-166 | ambuside |
| 3442 | P-166 | azosemide |
| 3443 | P-166 | bumetanide |
| 3444 | P-166 | butazolamide |
| 3445 | P-166 | chloraminophenamide |
| 3446 | P-166 | clofenamide |
| 3447 | P-166 | clopamide |
| 3448 | P-166 | clorexolone |
| 3449 | P-166 | disulfamide |
| 3450 | P-166 | ethoxolamide |
| 3451 | P-166 | furosemide |
| 3452 | P-166 | mefruside |
| 3453 | P-166 | methazolamide |
| 3454 | P-166 | piretanide |
| 3455 | P-166 | torasemide |
| 3456 | P-166 | tripamide |
| 3457 | P-166 | xipamide |
| 3458 | P-166 | mercaptomerin sodium |
| 3459 | P-166 | merethoxylline |
| 3460 | P-166 | procaine |
| 3461 | P-166 | mersalyl with thiophylline |
| 3462 | P-167 | amanozine |
| 3463 | P-167 | amiloride |
| 3464 | P-167 | arbutin |
| 3465 | P-167 | chlorazanil |
| 3466 | P-167 | ethacrynic acid |
| 3467 | P-167 | etozolin |
| 3468 | P-167 | hydracarbazine |
| 3469 | P-167 | isosorbide |
| 3470 | P-167 | mannitol |
| 3471 | P-167 | metochalcone |
| 3472 | P-167 | muzolimine |
| 3473 | P-167 | perhexiline |
| 3474 | P-167 | ticrynafen |
| 3475 | P-167 | triamterene |
| 3476 | P-167 | urea |
| 3477 | P-167 | althiazide |
| 3478 | P-167 | bendroflumethiazide |
| 3479 | P-167 | benzthiazide |
| 3480 | P-167 | benzylhydrochlorothiazide |
| 3481 | P-167 | buthiazide |
| 3482 | P-167 | chlorothiazide |
| 3483 | P-167 | chlorthalidone |
| 3484 | P-167 | cyclopenthiazide |
| 3485 | P-167 | cyclothiazide |
| 3486 | P-167 | epithiazide |
| 3487 | P-167 | ethiazide |
| 3488 | P-167 | fenquizone |
| 3489 | P-167 | hydrochlorothiazide |
| 3490 | P-167 | hydroflumethiazide |
| 3491 | P-167 | indapamide |
| 3492 | P-167 | methyclothiazide |
| 3493 | P-167 | meticrane |
| 3494 | P-167 | metolazone |
| 3495 | P-167 | paraflutizide |
| 3496 | P-167 | polythiazide |
| 3497 | P-167 | quinethazone |
| 3498 | P-167 | teclothiazide |
| 3499 | P-167 | trichlormethiazide |
| 3500 | P-167 | acetazolamide |
| 3501 | P-167 | ambuside |
| 3502 | P-167 | azosemide |
| 3503 | P-167 | bumetanide |
| 3504 | P-167 | butazolamide |
| 3505 | P-167 | chloraminophenamide |
| 3506 | P-167 | clofenamide |
| 3507 | P-167 | clopamide |
| 3508 | P-167 | clorexolone |
| 3509 | P-167 | disulfamide |
| 3510 | P-167 | ethoxolamide |
| 3511 | P-167 | furosemide |
| 3512 | P-167 | mefruside |
| 3513 | P-167 | methazolamide |
| 3514 | P-167 | piretanide |
| 3515 | P-167 | torasemide |
| 3516 | P-167 | tripamide |
| 3517 | P-167 | xipamide |
| 3518 | P-167 | mercaptomerin sodium |
| 3519 | P-167 | merethoxylline |
| 3520 | P-167 | procaine |
| 3521 | P-167 | mersalyl with thiophylline |
| 3522 | P-168 | amanozine |
| 3523 | P-168 | amiloride |
| 3524 | P-168 | arbutin |
| 3525 | P-168 | chlorazanil |
| 3526 | P-168 | ethacrynic acid |
| 3527 | P-168 | etozolin |
| 3528 | P-168 | hydracarbazine |
| 3529 | P-168 | isosorbide |
| 3530 | P-168 | mannitol |
| 3531 | P-168 | metochalcone |
| 3532 | P-168 | muzolimine |
| 3533 | P-168 | perhexiline |
| 3534 | P-168 | ticrynafen |
| 3535 | P-168 | triamterene |
| 3536 | P-168 | urea |
| 3537 | P-168 | althiazide |
| 3538 | P-168 | bendroflumethiazide |
| 3539 | P-168 | benzthiazide |
| 3540 | P-168 | benzylhydrochlorothiazide |
| 3541 | P-168 | buthiazide |
| 3542 | P-168 | chlorothiazide |
| 3543 | P-168 | chlorthalidone |
| 3544 | P-168 | cyclopenthiazide |
| 3545 | P-168 | cyclothiazide |
| 3546 | P-168 | epithiazide |
| 3547 | P-168 | ethiazide |
| 3548 | P-168 | fenquizone |
| 3549 | P-168 | hydrochlorothiazide |
| 3550 | P-168 | hydroflumethiazide |
| 3551 | P-168 | indapamide |
| 3552 | P-168 | methyclothiazide |
| 3553 | P-168 | meticrane |
| 3554 | P-168 | metolazone |
| 3555 | P-168 | paraflutizide |
| 3556 | P-168 | polythiazide |
| 3557 | P-168 | quinethazone |
| 3558 | P-168 | teclothiazide |
| 3559 | P-168 | trichlormethiazide |
| 3560 | P-168 | acetazolamide |
| 3561 | P-168 | ambuside |
| 3562 | P-168 | azosemide |
| 3563 | P-168 | bumetanide |
| 3564 | P-168 | butazolamide |
| 3565 | P-168 | chloraminophenamide |
| 3566 | P-168 | clofenamide |
| 3567 | P-168 | clopamide |
| 3568 | P-168 | clorexolone |
| 3569 | P-168 | disulfamide |
| 3570 | P-168 | ethoxolamide |
| 3571 | P-168 | furosemide |
| 3572 | P-168 | mefruside |
| 3573 | P-168 | methazolamide |
| 3574 | P-168 | piretanide |
| 3575 | P-168 | torasemide |
| 3576 | P-168 | tripamide |
| 3577 | P-168 | xipamide |
| 3578 | P-168 | mercaptomerin sodium |
| 3579 | P-168 | merethoxylline |
| 3580 | P-168 | procaine |
| 3581 | P-168 | mersalyl with thiophylline |
| 3582 | P-169 | amanozine |
| 3583 | P-169 | amiloride |
| 3584 | P-169 | arbutin |
| 3585 | P-169 | chlorazanil |
| 3586 | P-169 | ethacrynic acid |
| 3587 | P-169 | etozolin |
| 3588 | P-169 | hydracarbazine |
| 3589 | P-169 | isosorbide |
| 3590 | P-169 | mannitol |
| 3591 | P-169 | metochalcone |
| 3592 | P-169 | muzolimine |
| 3593 | P-169 | perhexiline |
| 3594 | P-169 | ticrynafen |
| 3595 | P-169 | triamterene |
| 3596 | P-169 | urea |
| 3597 | P-169 | althiazide |
| 3598 | P-169 | bendroflumethiazide |
| 3599 | P-169 | benzthiazide |
| 3600 | P-169 | benzylhydrochlorothiazide |
| 3601 | P-169 | buthiazide |
| 3602 | P-169 | chlorothiazide |
| 3603 | P-169 | chlorthalidone |
| 3604 | P-169 | cyclopenthiazide |
| 3605 | P-169 | cyclothiazide |
| 3606 | P-169 | epithiazide |
| 3607 | P-169 | ethiazide |
| 3608 | P-169 | fenquizone |
| 3609 | P-169 | hydrochlorothiazide |
| 3610 | P-169 | hydroflumethiazide |
| 3611 | P-169 | indapamide |
| 3612 | P-169 | methyclothiazide |
| 3613 | P-169 | meticrane |
| 3614 | P-169 | metolazone |
| 3615 | P-169 | paraflutizide |
| 3616 | P-169 | polythiazide |
| 3617 | P-169 | quinethazone |
| 3618 | P-169 | teclothiazide |
| 3619 | P-169 | trichlormethiazide |
| 3620 | P-169 | acetazolamide |
| 3621 | P-169 | ambuside |
| 3622 | P-169 | azosemide |
| 3623 | P-169 | bumetanide |
| 3624 | P-169 | butazolamide |
| 3625 | P-169 | chloraminophenamide |
| 3626 | P-169 | clofenamide |
| 3627 | P-169 | clopamide |
| 3628 | P-169 | clorexolone |
| 3629 | P-169 | disulfamide |
| 3630 | P-169 | ethoxolamide |
| 3631 | P-169 | furosemide |
| 3632 | P-169 | mefruside |
| 3633 | P-169 | methazolamide |
| 3634 | P-169 | piretanide |
| 3635 | P-169 | torasemide |
| 3636 | P-169 | tripamide |
| 3637 | P-169 | xipamide |
| 3638 | P-169 | mercaptomerin sodium |
| 3639 | P-169 | merethoxylline |
| 3640 | P-169 | procaine |
| 3641 | P-169 | mersalyl with thiophylline |
| 3642 | P-170 | amanozine |
| 3643 | P-170 | amiloride |
| 3644 | P-170 | arbutin |
| 3645 | P-170 | chlorazanil |
| 3646 | P-170 | ethacrynic acid |
| 3647 | P-170 | etozolin |
| 3648 | P-170 | hydracarbazine |
| 3649 | P-170 | isosorbide |
| 3650 | P-170 | mannitol |
| 3651 | P-170 | metochalcone |
| 3652 | P-170 | muzolimine |
| 3653 | P-170 | perhexiline |
| 3654 | P-170 | ticrynafen |
| 3655 | P-170 | triamterene |
| 3656 | P-170 | urea |
| 3657 | P-170 | althiazide |
| 3658 | P-170 | bendroflumethiazide |
| 3659 | P-170 | benzthiazide |
| 3660 | P-170 | benzylhydrochlorothiazide |
| 3661 | P-170 | buthiazide |
| 3662 | P-170 | chlorothiazide |
| 3663 | P-170 | chlorthalidone |
| 3664 | P-170 | cyclopenthiazide |
| 3665 | P-170 | cyclothiazide |
| 3666 | P-170 | epithiazide |
| 3667 | P-170 | ethiazide |
| 3668 | P-170 | fenquizone |
| 3669 | P-170 | hydrochlorothiazide |
| 3670 | P-170 | hydroflumethiazide |
| 3671 | P-170 | indapamide |
| 3672 | P-170 | methyclothiazide |
| 3673 | P-170 | meticrane |
| 3674 | P-170 | metolazone |
| 3675 | P-170 | paraflutizide |
| 3676 | P-170 | polythiazide |
| 3677 | P-170 | quinethazone |
| 3678 | P-170 | teclothiazide |
| 3679 | P-170 | trichlormethiazide |
| 3680 | P-170 | acetazolamide |
| 3681 | P-170 | ambuside |
| 3682 | P-170 | azosemide |
| 3683 | P-170 | bumetanide |
| 3684 | P-170 | butazolamide |
| 3685 | P-170 | chloraminophenamide |
| 3686 | P-170 | clofenamide |
| 3687 | P-170 | clopamide |
| 3688 | P-170 | clorexolone |
| 3689 | P-170 | disulfamide |
| 3690 | P-170 | ethoxolamide |
| 3691 | P-170 | furosemide |
| 3692 | P-170 | mefruside |
| 3693 | P-170 | methazolamide |
| 3694 | P-170 | piretanide |
| 3695 | P-170 | torasemide |
| 3696 | P-170 | tripamide |
| 3697 | P-170 | xipamide |
| 3698 | P-170 | mercaptomerin sodium |
| 3699 | P-170 | merethoxylline |
| 3700 | P-170 | procaine |
| 3701 | P-170 | mersalyl with thiophylline |

It should be recognized that the above tables simply illustrate examples of various combinations of p38-kinase inhibitors with various diuretics. This invention therefore should not be limited to those combinations.

It should also be recognized that this invention contemplates combinations comprising more than one p38-kinase inhibitor with a diuretic, as well as combinations comprising a p38-kinase inhibitor with more than one diuretic, as well as combinations comprising more than one p38-kinase inhibitor with more than one diuretic. Further, any such combination (or any combination comprising only one p38-kinase inhibitor and only one diuretic) may further comprise one or more ACE inhibitor, one or more aldosterone antagonists, and/or one or more other therapeutic agents. Such other therapeutic agents may include, for example, one or more IBAT inhibitors, CETP inhibitors, fibrates, digoxin, calcium channel blockers, endothelin antagonists, inhibitors of microsomal triglyceride transfer protein, cholesterol absorption antagonists, phytosterols, bile acid sequestrants, vasodilators, adrenergic blockers, adrenergic stimulants, and/or inhibitors of HMG-CoA reductase activity. Such other therapeutic agents may also comprise, for example, one or more conventional anti-inflammatories, such as steroids, cyclooxygenase-2 inhibitors, DMARDs, immunosuppressive agents, NSAIDs, 5-lipoxygenase inhibitors, LTB4 antagonists, and LTA4 hydrolase inhibitors.

### G. Preferred Modes of Administration

The therapeutic agents used in this invention may be administered by any means that produces contact of each agent with its site of action in the body. Each therapeutic agent may each be administered as, for example, a compound per se or a pharmaceutically-acceptable salt thereof. Pharmaceutically-acceptable salts are often particularly suitable for medical applications because of their greater aqueous solubility relative to the compounds themselves. Typically, all the therapeutic agents are preferably administered orally. This invention, however, also contemplates methods wherein at least one of the therapeutic agents is administered by another means, such as parenterally.

In many embodiments, a therapeutic agent used in this invention is administered as part of a pharmaceutical composition (or medicament) that further comprises one or more pharmaceutically-acceptable carriers, diluents, wetting or suspending agents, vehicles, and/or adjuvants (the carriers, diluents, wetting or suspending agents, vehicles, and adjuvants sometimes being collectively referred to in this specification as “carrier materials”); and/or other active ingredients. Where the agent is administered as part of a combination therapy, the other agent(s) of the combination may also be contained in the same pharmaceutical composition or as a part of a separate pharmaceutical composition or both.

In many preferred embodiments, the pharmaceutical composition is in the form of a dosage unit containing a particular amount of the active ingredient(s). For example, a pharmaceutical composition comprising a p38-kinase inhibitor preferably comprises a dosage form containing from about 0.1 to 1000 mg of the p38-kinase inhibitor, and more typically from about 7.0 to about 350 mg of the p38-kinase inhibitor. Illustrating further, spironolactone is sold by Pharmacia Corporation under the trademark “ALDACTONE” in tablet dosage form at doses of 25, 50, or 100 mg per tablet.

In many embodiments, from about 0.05 to about 95% by weight of a pharmaceutical composition consists of an active therapeutic agent(s). The preferred composition depends on the method of administration. Pharmaceutical compositions suitable for this invention may be prepared by a variety of well-known techniques of pharmacy that include the step of bringing into association the therapeutic agent(s) with the carrier material(s). In general, the compositions are prepared by uniformly and intimately admixing the therapeutic agent(s) with a liquid or finely divided solid carrier material (or both), and then, if desirable, shaping the product. For example, a tablet may be prepared by compressing or molding a powder or granules of the therapeutic agent, optionally with one or more carrier materials and/or other active ingredients. Compressed tablets can be prepared by compressing, in a suitable machine, the therapeutic agent in a free-flowing form, such as a powder or granules optionally mixed with a binder, lubricant, inert diluent and/or surface active/dispersing agent(s). Molded tablets can be made, for example, by molding the powdered compound in a suitable machine. Formulation of drugs is generally discussed in, for example, Hoover, John E., Remington 's Pharmaceutical Sciences (Mack Publishing Co., Easton, Pa.: 1975) (incorporated by reference into this patent). See also, Liberman, H. A., Lachman, L., eds., Pharmaceutical Dosage Forms (Marcel Decker, New York, N.Y., 1980) (incorporated by reference into this patent). See also, Kibbe et al., eds., Handbook of Pharmaceutical Excipients, 3rd Ed., (American Pharmaceutical Association, Washington, D.C. 1999) (incorporated by reference into this patent).

Therapeutic agents (and combinations thereof) suitable for oral administration can be administered in discrete units comprising, for example, solid dosage forms. Such solid dosage forms include, for example, hard or soft capsules, cachets, lozenges, tablets, pills, powders, or granules, each containing a pre-determined amount of the therapeutic agent(s). In such solid dosage forms, the therapeutic agents are ordinarily combined with one or more adjuvants. If administered per os, the therapeutic agents may be mixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Pharmaceutical compositions particularly suitable for buccal (sub-lingual) administration include, for example, lozenges comprising the therapeutic agent(s) in a flavored base, usually sucrose, and acacia or tragacanth; or pastilles comprising the therapeutic agent(s) in an inert base, such as gelatin and glycerin or sucrose and acacia.

Therapeutic agents (and combinations thereof) suitable for oral administration also can be administered in discrete units comprising, for example, a liquid dosage forms. Such liquid dosage forms include, for example, pharmaceutically acceptable emulsions (including both oil-in-water and water-in-oil emulsions), solutions (including both aqueous and non-aqueous solutions), suspensions (including both aqueous and non-aqueous suspensions), syrups, and elixirs containing inert diluents commonly used in the art (e.g., water). Such compositions also may comprise adjuvants, such as wetting, emulsifying, suspending, flavoring (e.g., sweetening), and/or perfuming agents.

Oral delivery of the therapeutic agents in the present invention may include formulations that provide immediate delivery, or, alternatively, sustained (or prolonged) delivery of the agent by a variety of mechanisms. Immediate delivery formulations include, for example, oral solutions, oral suspensions, fast-dissolving tablets or capsules, disintegrating tablets, etc. Sustained-delivery formulations include, for example, pH-sensitive release from the dosage form based on the changing pH of the gastrointestinal tract, slow erosion of a tablet or capsule, retention in the stomach based on the physical properties of the formulation, bio-adhesion of the dosage form to the mucosal lining of the intestinal tract, or enzymatic release of the active drug from the dosage form. The intended effect is to extend the time period over which the active drug molecule is delivered to the site of action by manipulation of the dosage form. Thus, in the case of capsules, tablets, and pills, the dosage forms may comprise buffering agents, such as sodium citrate, or magnesium or calcium carbonate or bicarbonate. Tablets and pills additionally may be prepared with enteric coatings. Suitable enteric coatings include, for example, cellulose acetate phthalate, polyvinylacetate phthalate, hydroxypropylmethyl-cellulose phthalate, and anionic polymers of methacrylic acid and methacrylic acid methyl ester.

“Parenteral administration” includes subcutaneous injections, intravenous injections, intramuscular injections, intrasternal injections, and infusion. Injectable preparations (e.g., sterile injectable aqueous or oleaginous suspensions) may be formulated according to the known art using suitable dispersing, wetting agents, and/or suspending agents. Acceptable carrier materials include, for example, water, 1,3-butanediol, Ringer's solution, isotonic sodium chloride solution, bland fixed oils (e.g., synthetic mono- or diglycerides), dextrose, mannitol, fatty acids (e.g., oleic acid), dimethyl acetamide, surfactants (e.g., ionic and non-ionic detergents), and/or polyethylene glycols (e.g., PEG 400).

Formulations for parenteral administration may, for example, be prepared from sterile powders or granules having one or more of the carriers materials mentioned for use in the formulations for oral administration. The therapeutic agent(s) may be dissolved in water, polyethylene glycol, propylene glycol, ethanol, corn oil, cottonseed oil, peanut oil, sesame oil, benzyl alcohol, sodium chloride, and/or various buffers. The pH may be adjusted, if necessary, with a suitable acid, base, or buffer.

This invention also contemplates administering one or more therapeutic agents via a transdermal device. Here, administration may be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane. The transdermal patch may include the compound in a suitable solvent system with an adhesive system, such as an acrylic emulsion, and a polyester patch. The oily phase of the emulsions of this invention may be constituted from known ingredients in a known manner. While the phase may comprise merely an emulsifier, it may comprise, for example, a mixture of at least one emulsifier with a fat or an oil or with both a fat and an oil. Preferably, a hydrophilic emulsifier is included together with a lipophilic emulsifier which acts as a stabilizer. It is also preferable to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. Emulsifiers and emulsion stabilizers suitable for use in the formulation of the present invention include Tween 60, Span 80, cetostearyl alcohol, myristyl alcohol, glyceryl monostearate, and sodium lauryl sulfate, among others. The choice of suitable oils or fats for the formulation is based on achieving the desired cosmetic properties, given that the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters, for example, may be used. These may be used alone or in combination depending on the properties required. Alternatively, high melting point lipids such as white soft paraffin and/or liquid paraffin or other mineral oils may be used.

Other carrier materials and modes of administration known in the pharmaceutical art may also be used.

### H. Kits

The present invention further comprises kits that are suitable for use in performing the methods of treatment described above. In one embodiment, the kit comprises a first dosage form comprising a p38-kinase inhibitor and a second dosage form comprising an aldosterone antagonist or diuretic for a pathological condition (e.g., a cardiovascular condition or a condition associated with a cardiovascular condition) in quantities sufficient to carry out the methods of the present invention. Preferably, the first dosage form and the second dosage form together comprise a therapeutically-effective amount of the agents for the treatment of the targeted condition(s).

### EXAMPLES

The following examples are merely illustrative, and not limiting to the remainder of this disclosure in any way.

### Example 1

### In Vitro p38 Kinase Inhibition Analysis

Several p38-kinase inhibiting compounds disclosed in this application were analyzed in the in vitro assays described below to determine their ability to inhibit p38α kinase.

### Cloning of Human p38α

The coding region of the human p38α cDNA was obtained by PCR-amplification from RNA isolated from the human monocyte cell line THP.1. First strand cDNA was synthesized from total RNA as follows: 2 μg of RNA was annealed to 100 ng of random hexamer primers in a 10 μl reaction by heating to 70° C. for 10 min, followed by 2 min on ice. cDNA was then synthesized by adding 1 μl of RNAsin (Promega, Madison Wis.), 2 μl of 50 mM dNTP's, 4 μl of 5× buffer, 2 μl of 100 mM DTT and 1 μl (200 U) of Superscript II™ AMV reverse transcriptase. Random primer, dNTP's and Superscript™ reagents were all purchased from Life-Technologies, Gaithersburg, Mass. The reaction was incubated at 42° C. for 1 hr. Amplification of p38 cDNA was performed by aliquoting 5 μl of the reverse transcriptase reaction into a 100 μl PCR reaction containing the following: 80 μl dH₂O, 2 μl 50 mM dNTP's, 1 μl each of forward and reverse primers (50 pmol/μl), 10 μl of 10× buffer, and 1 μl Expand™ polymerase (Boehringer Mannheim). The PCR primers incorporated Bam HI sites onto the 5′ and 3′ end of the amplified fragment, and were purchased from Genosys. The sequences of the forward and reverse primers were 5′-GATCGAGGATTCATGTCTCAGGAGAGGCCCA-3′ and 5′GATCGAGGATTCTCAGGACTCCATCTCTTC-3′, respectively. The PCR amplification was carried out in a DNA Thermal Cycler (Perkin Elmer) by repeating 30 cycles of 94° C. for 1 min, 60° C. for 1 min, and 68° C. for 2 min. After amplification, excess primers and unincorporated dNTP's were removed from the amplified fragment with a Wizard™ PCR prep (Promega), and digested with Bam HI (New England Biolabs). The Bam HI digested fragment was ligated into BamHI digested pGEX 2T plasmid DNA (PharmaciaBiotech) using T-4 DNA ligase (New England Biolabs) as described by T. Maniatis, Molecular Cloning: A Laboratory Manual, 2nd ed. (1989). The ligation reaction was transformed into chemically competent E. coli DH10B cells purchased from Life-Technologies following the manufacturer's instructions. Plasmid DNA was isolated from the resulting bacterial colonies using a Promega Wizard™ miniprep kit. Plasmids containing the appropriate Bam HI fragment were sequenced in a DNA Thermal Cycler (Perkin Elmer) with Prism™ (Applied Biosystems Inc.). cDNA clones were identified that coded for both human p38a isoforms (Lee et al. Nature 372, 739). One of the clones which contained the cDNA for p38a-2 (CSBP-2) inserted in the cloning site of pGEX 2T, 3′ of the GST coding region was designated pMON 35802. The sequence obtained for this clone is an exact match of the cDNA clone reported by Lee et al. This expression plasmid allows for the production of a GST-p38a fusion protein.

### Expression of Human p38α

GST/p38α fusion protein was expressed from the plasmid pMON 35802 in E. coli, stain DH10B (Life Technologies, Gibco-BRL). Overnight cultures were grown in Luria Broth (LB) containing 100 mg/ml ampicillin. The next day, 500 ml of fresh LB was inoculated with 10 ml of overnight culture, and grown in a 2 liter flask at 37° C. with constant shaking until the culture reached an absorbance of 0.8 at 600 nm. Expression of the fusion protein was induced by addition of isopropyl b-D-thiogalactosidse (IPTG) to a final concentration of 0.05 mM. The cultures were shaken for three hr at room temperature, and the cells were harvested by centrifugation. The cell pellets were stored frozen until protein purification.

### Purification of p38α Kinase

All chemicals were from Sigma Chemical Co. unless noted. Twenty grams of E. coli cell pellet collected from five 1 L shake flask fermentations were re-suspended in a volume of PBS (140 mM NaCl, 2.7 mM KCl, 10 mM Na₂HPO₄, 1.8 mM KH₂PO₄, pH 7.3) up to 200 ml. The cell suspension was adjusted to 5 mM DTT with 2 M DTT and then split equally into five 50 ml Falcon conical tubes. The cells were sonicated (Ultrasonics model W375) with a 1 cm probe for 3×1 min (pulsed) on ice. Lysed cell material was removed by centrifugation (12,000×g, 15 min), and the clarified supernatant applied to glutathione-sepharose resin (Pharmacia).

### Glutathione-Sepharose Affinity Chromatography

Twelve ml of a 50% glutathione sepharose-PBS suspension was added to 200 ml clarified supernatant, and then incubated batchwise for 30 min at room temperature. The resin was collected by centrifugation (600×g, 5 min) and washed with 2×150 ml PBS/1% Triton X-100, followed by 4×40 ml PBS. To cleave the p38 kinase from the GST-p38 fusion protein, the glutathione-sepharose resin was re-suspended in 6 ml PBS containing 250 units thrombin protease (Pharmacia, specific activity >7500 units/mg), and then mixed gently for 4 hr at room temperature. The glutathione-sepharose resin was removed by centrifugation (600×g, 5 min) and washed 2×6 ml with PBS. The PBS wash fractions and digest supernatant containing p38 kinase protein were pooled and adjusted to 0.3 mM PMSF.

### Mono Q Anion Exchange Chromatography

The thrombin-cleaved p38 kinase was further purified by FPLC-anion exchange chromatography. Thrombin-cleaved sample was diluted 2-fold with Buffer A (25 mM HEPES, pH 7.5, 25 mM beta-glycerophosphate, 2 mM DTT, 5% glycerol) and injected onto a Mono Q HR 10/10 (Pharmacia) anion exchange column equilibrated with Buffer A. The column was eluted with a 160 ml 0.1 M-0.6 M NaCl/Buffer A gradient (2 ml/min flowrate). The p38 kinase peak eluting at 200 mM NaCl was collected and concentrated to 3-4 ml with a Filtron 10 concentrator (Filtron Corp.).

### Sephacryl S100 Gel Filtration Chromatography

The concentrated Mono Q-p38 kinase purified sample was purified by gel filtration chromatography (Pharmacia HiPrep 26/60 Sephacryl S100 column equilibrated with Buffer B (50 mM HEPES, pH 7.5, 50 mM NaCl, 2 mM DTT, 5% glycerol)). Protein was eluted from the column with Buffer B at a 0.5 ml/min flowrate and protein was detected by absorbance at 280 μm. Fractions containing p38 kinase (detected by SDS-polyacrylamide gel electrophoresis) were pooled and frozen at −80° C. Typical purified protein yields from 5 L E. coli shake flasks fermentations were 35 mg p38 kinase.

### In Vitro Assay

The ability of compounds to inhibit human p38 kinase alpha was evaluated using one of two in vitro assay methods. In the first method, activated human p38 kinase alpha phosphorylates a biotinylated substrate, PHAS-I (phosphorylated heat and acid stable protein-insulin inducible), in the presence of gamma ³²P-ATP (³²P-ATP). PHAS-I was biotinylated before the assay, and provided a means of capturing the substrate which was phosphorylated during the assay. p38 Kinase was activated by MKK6. Compounds were tested in 10 fold serial dilutions over the range of 100 μM to 0.001 μM using 1% DMSO. Each concentration of inhibitor was tested in triplicate.

All reactions were carried out in 96 well polypropylene plates. Each reaction well contained 25 mM HEPES pH 7.5, 10 mM magnesium acetate, and 50 μM unlabeled ATP. Activation of p38 was required to achieve sufficient signal in the assay. Biotinylated PHAS-I was used at 1-2 μg per 50 μl reaction volume, with a final concentration of 1.5 μM. Activated human p38 kinase alpha was used at 1 μg per 50 μl reaction volume, representing a final concentration of 0.3 μM. Gamma ³²P-ATP was used to follow the phosphorylation of PHAS-I. ³²P-ATP has a specific activity of 3000 Ci/mmol, and was used at 1.2 μCi per 50 μl reaction volume. The reaction proceeded either for one hr or overnight at 30° C.

Following incubation, 20 μl of reaction mixture was transferred to a high capacity streptavidin coated filter plate (SAM-streptavidin-matrix, Promega) prewetted with phosphate buffered saline. The transferred reaction mix was allowed to contact the streptavidin membrane of the Promega plate for 1-2 min. Following capture of biotinylated PHAS-I with ³²P incorporated, each well was washed to remove unincorporated ³²P-ATP three times with 2M NaCl, three washes of 2M NaCl with 1% phosphoric, three washes of distilled water, and finally a single wash of 95% ethanol. Filter plates were air dried and 20 μl of scintillant was added. The plates were sealed and counted.

A second assay format was alternatively employed. This assay is based on p38 kinase alpha being induced phosphorylation of EGFRP (epidermal growth factor receptor peptide, a 21 mer) in the presence of ³³P-ATP. Compounds were tested in 10 fold serial dilutions over the range of 100 μM to 0.001 μM in 10% DMSO. Each concentration of inhibitor was tested in triplicate. Compounds were evaluated in 50 μl reaction volumes in the presence of 25 mM HEPES pH 7.5, 10 mM magnesium acetate, 4% glycerol, 0.4% bovine serum albumin, 0.4 mM DTT, 50 μM unlabeled ATP, 25 μg EGFRP (200M), and 0.05 uCi gamma ³³P-ATP. Reactions were initiated by addition of 0.09 μg of activated, purified human GST-p38 kinase alpha. Activation was carried out using GST-MKK6 (5:1,p38:MKK6) for one hr at 30° C. in the presence of 50 μM ATP. Following incubation for 60 min at room temperature, the reaction was stopped by addition of 150 μl of AG 1X8 resin in 900 mM sodium formate buffer, pH 3.0 (I volume resin to 2 volumes buffer). The mixture was mixed three times with pipetting. Afterward, the resin was allowed to settle. A total of 50 μl of clarified solution head volume was transferred from the reaction wells to Microlite-2 plates. 150 μL of Microscint 40 was then added to each well of the Microlite plate, and the plate was sealed, mixed, and counted.

### Example 2

### Spontaneously Hypertensive Heart Failure (SHHF) Rat Model to Evaluate a Combination Therapy of a p38 Kinase Inhibitor with an Aldosterone Antagonist

The SHHF rat model has been described in the art. Heyen, J. R. R., et al., “Structural, functional, and molecular characterization of the SHHF rat model of heart failure”, Am. J. Physiol., vol. 283, pp. H1775-H1784 (2002) (incorporated by reference into this patent). This model may be used as described below to evaluate a combination therapy of a p38 kinase inhibitor with an aldosterone antagonist.

I. Experimental Protocol

This study is conducted using lean, male SHHF rats (Genetic Models Inc., Indianapolis, Ind.), and age-matched Sprague-Dawley (SD) rats (Charles River Labs, Raleigh, N.C.) as controls. All the animals are housed in a room lighted 12 hours per day at an ambient temperature of 22+1° C. The animals are allowed 3 weeks to adjust after arrival, and are given free access to rodent diet (Purina 5002; Ralston Purina, St. Louis, Mo.) and tap water ad libitum. At the initiation of the study, all the animals are 15 months of age.

The study is conducted over 12 weeks, with measurements and samples taken at baseline, and after 4, 8, and 12 weeks of treatment (termination of study). Following acclimation, baseline measurements are performed, and 1 week later, the rats are assigned to one of the following treatment groups after being randomized based on genotype: (1) rats receiving no treatment; (2) rats receiving an aldosterone antagonist of interest at a dose of interest, (3) rats receiving a p38 kinase inhibitor at a dose of interest, and (4) rats receiving a co-administration of the aldosterone antagonist at a dosing of interest and the p38 inhibitor at a dosing of interest.

II. Assays and Analyses

A. Genotyping

To determine homozygous and heterozygous lean male rats, genotyping is performed. Each tail snip is minced into 1 mm fragments, and placed into a 1.5 ml microfuge tube. DNA is isolated using the PureGene Genomic DNA Isolation Kit (Gentra Systems, Minneapolis, Minn.). One ml of the isolated DNA is added to a Ready-To-Go PCR bead (Amersham Pharmacia Biotech Inc., Piscataway, N.J.), followed by primers: Sense: 5′-ATG-AAT-GCT-GTG-CAG-TC-3′; Antisense: 5′-AAG-GTT-CTT-CCA-TTC-AAT-3′ (Invitrogen GibcoBRL/Life Technologies, Carlsbad, Calif.). Reaction tubes are placed into the PTC-100 Programmable Thermal Controller (MJ Research, Inc., Watertown, Mass.) using the following protocol: 94° C., 30 seconds; 55° C., 30 seconds; 72° C., 30 seconds; 30 cycles 4° C. post run dwell. After PCR, samples are digested with Tru9I (Promega, Madison, Wis.). Products are run on a 5% agarose gel, along with a 50 base pair DNA ladder (Promega, catalog # G4521). Band sizes indicated genotype: Homozygous Lean: One band at 121 bp. Heterozygous Lean: Three bands at 121, 82 and 39 bp.

B. Echocardiography

Transthoracic echocardiography examinations are performed using the method described in Heyen, J. R. R., et al. The examinations are performed at baseline, and after 4, 9, and 13 weeks of treatment during the progression of heart failure. During these examinations, each animal is lightly anesthetized with 1-2% isofluorane gas, the chest is shaved, and echocardiograms are obtained with a SONOS 5500 system (Alilent Technologies, Andover, Mass.) utilizing a 15 megahertz linear array probe. Parasternal long axis, parasternal short axis, and apical 2 and 4-chamber views are acquired using a 2-D mode. Doppler and m-mode images are also captured at the level of the mitral valve and papillary muscles, respectively. Data is analyzed from the resulting 2-D mode and Doppler images that are acquired and saved using software provided with the SONOS 5500 system.

Measurements and calculations used are as follows: percent LV fractional shortening (FS) is calculated as follows: FS=(LVIDd−LVIDs)/LVIDd×100, where LVIDd and LVIDs are end-diastolic and end-systolic LV internal dimensions, respectively. Relative wall thickness (RWT) is calculated as (PWd+IVSd)/LVIDd, where PWd and IVSd are end-diastolic posterior wall and interventricular septal thickness, respectively. End-diastolic (EDV) and end-systolic volumes (ESV) are calculated from LV systolic (LVAs) and diastolic (LVAd) areas via the method of discs. See Schiller, N. B., “Recommendations for quantitation of the left ventricle by two-dimensional echocardiography. American Society of Echocardiography Committee on Standards, Subcommittee on Quantitation of Two-Dimensional Echocardiograms”, J. Am. Soc. Echocardiogr., vol. 2, pp. 358-367 (1989) (incorporated by reference into this patent). EF is calculated from systolic and diastolic volumes with the following formula: EF=(EDV−ESV)/EDV×100. Other measurements taken include early filling velocity (E-velocity; E-vel), late filling velocity (A-velocity; A-vel), mitral valve deceleration time (Decel T), LV mass (area length method), heart rate (HR; m-mode R—R interval), stroke volume (SV; SV=EDV−ESV) and cardiac output (CO=SV×HR).

C. Systolic Blood Pressure

Intra-ventricular systolic blood pressure is measured following 12 weeks of treatment. During this analysis, each animal is anesthetized with 5% isoflurane, followed by 2-3% isoflurane. The right common carotid artery is cannulated with a Millar catheter transducer (Millar, Houston, Tex.) passed under constant pressure into the left ventricle. Data is collected every 10 seconds for 3 minutes and analyzed using a HPA-210 heart performance analyzer (Micro-Med, Louisville, Ky.).

Alternatively, tail-cuff systolic blood pressure is analyzed non-invasively at baseline, and after 6 and 12 weeks of treatment using the Visitech BP-2000 Blood Pressure Analysis System (Visitech Systems, Apex, N.C.). Six measures are taken for each animal and averaged for a mean SBP reading.

D. Inflammatory Marker Analysis

Inflammatory markers include, for example, circulating TNFR1, TNFR2, osteopontin, and TNF-α These markers may be quantitated using, for example, established immunoassay techniques. The following techniques are used according to their respective manufacturers' instructions: TNFR1, catalog #MRT10, and TNFR2, catalog #MRT20 (R&D Systems, Minneapolis, Minn.); osteopontin, catalog #17360 (Immuno-Biological Laboratories Co., LTD, Fijioka-Shi, Gunma, Japan); and TNF-α, catalog #KRC3013 (Biosource Int'l, Inc., Camarillo, Calif.). Plasma aldosterone levels are determined using an aldosterone enzyme immunoassay kit (Cayman Chemical, Ann Arbor, Mich.).

E. Electrolytes

Serum electrolytes are analyzed using a Hitachi 912 automated diagnostic clinical chemistry analyzer (Roche Diagnostics Corp., Indianapolis, Ind.) according to standard procedures.

F. Histopathologic Analysis

The equatorial region of the hearts is routinely processed into paraffin, and 5-μm sections are stained with hematoxylin-eosin (H&E) and periodic acid-Schiff, and examined by light microscopy in a blinded fashion by a pathologist. Cardiac histopathology is assessed semi-quantitatively as follows. Arterial changes associated with hypertension (for example, media/adventitia hypertrophy, medial cell proliferation, fibrinoid and vacuolar degeneration, and periarterial and intramural inflammation) are graded based on severity and number of arteries affected. A scale from 1-4 is used to score the level of arterial. A score of “1” indicated that few arteries are affected, and mild changes are observed. A score of “2” indicated that few arteries are affected, and moderate changes are observed. A score of “3” indicated that most arteries are affected with moderate changes or few arteries are affected with severe changes. And a score of “4” indicated that most arteries are affected with moderate or severe changes. Myocardial damage (necrosis/loss of cardiomyocytes, interstitial inflammation, interstitial fibrosis, etc) is graded based on extent using a scale from 1-4. A score of “1” indicated that few, small, scattered foci are observed. A score of “2” indicated that scattered, moderately-sized foci are observed. A score of “3” indicated that frequent, large foci are observed. And a score of “4” indicated that extensive, coalescing areas are observed. Myocardial fibrosis is assessed as described in Heyen, J. R. R., et al.

G. Immunohistochemistry

Sections (5 μm) are immunostained following a standard procedure using a primary antibody for osteopontin (working dilution 1:100, University of Iowa, Iowa City, Iowa). Briefly, sections are deparaffinized, rehydrated in ethanol, and processed for antigen retrieval (Target Retrieval Solution, DAKO). Positive staining is detected using appropriate biotin-labeled secondary antibodies, horseradish peroxidase-conjugated streptavidin (DAKO), and incubating the sections in diaminobenzidine (DAKO). Nonspecific isotype-matched IgGs at similar concentrations are used as primary antibodies for negative controls, and tissues known to express these targets are used as positive controls.

H. Heart Weight and Samples

At the end of the experiment, each animal is anesthetized with pentobarbital (65 mg/kg i.p., Sigma Chemical, St. Louis, Mo.) and weighed with a Mettler PM6000 balance (Mettler-Toledo, Inc., Hightsown, N.J.). The abdominal cavity is opened to expose the abdominal aorta. An 18-guage needle is then inserted into the abdominal aorta, and the animals are exsanguinated. The resulting blood is immediately transferred into serum collection tubes (Terumo Medical Corp., Elkton, Md.), and placed on wet ice until sample collection is complete. The samples are then centrifuged for 15 min at 3,000 rev/min at 4° C. to form a serum that was, in turn, collected and frozen at −80° C. until further analysis.

Following exsanguination, the heart is isolated, removed, rinsed in cold PBS (Gibco, Gaithersburg, Md.), blotted dry, and weighed. Tibia also are removed (documented by X-ray analysis), and the length is determined using calipers. The observed heart weight is then normalized to tibial length (HW/TL). A 6-mm section is cut transversely through the middle of the heart and placed into 10% neutral-buffered formalin for 24 hr, followed by 70% alcohol until embedded into paraffin. The remaining apical portion of the heart is snap frozen in liquid nitrogen and stored at −80° C. for molecular analysis.

I. Molecular Biology

After RNA is extracted from the frozen hearts, TaqMan quantitative reverse-transcription polymerase chain reaction is performed as follows.

i) Principles of TaqMan Analysis

The fluorogenic 5′-nuclease assay (TaqMan PCR) using the 7700 Sequence Detection System (Applied Biosystems, Foster City, Calif.) allowed for real time detection/quantitation of a specific gene by monitoring the increase in fluorescence of a gene-specific, dye-labeled oligonucleotide probe. Probes for target and reference genes are labeled at the 5′-end with a 6-carboxyfluorescein (6FAM) reporter dye and at the 3′-end with a 6-carboxy-N,N,N′,N′-tetramethylrhodamine (TAMRA) quencher dye. When the probe is annealed to the target gene, fluorescence of 6FAM is prevented by the close proximity of TAMRA. The exonuclease activity of Taq polymerase released the dyes from the oligonucleotide probe by displacing the probe from the target sequence resulting in fluorescence excitation in direct proportion to the amount of target message present. Data analysis is performed using the Sequence Detection System software from Applied Biosystems.

ii) TaqMan primers and probes: MMP-2, MMP-3, MMP-13, MMP-14, TIMP-1, TIMP-2, TIMP4, MHCα, and MHCβ

All primers and probes are designed from known rat sequences using Primer Express software supplied with the 7700 Sequence Detection System and synthesized by Applied Biosystems. Standard curves using 5-fold dilutions of total RNA (from 200 ng to 320 pg) are performed to determine the efficiency of each primer/probe set in the TaqMan reaction before the analysis of the experimental samples. All target gene results are normalized to the reference gene cyclophilin. All samples are analyzed in duplicate. Suitable TaqMan RT-PCR gene marker primer/probe sets include, for example, those shown in Table 21:

TABLE 21

| | | | |
|---|---|---|---|
| Gene | Forward Primer | Reverse Primer | Probe |

| | | | |
|---|---|---|---|
| Matrix | CGAAGCTCAT | GGTTCTCCAACTT | CCTGATAACCTGGA |
| metalloprotease-2 | CGCAGACTCC | CAGGTAATAAGCA | TGCAGTCGTGGACC |
| (MMP-2) | | | |
| Matrix | TCCCAGGAAAAT | GAAACCCAAAT | TCCACCTTTGTG |
| metalloprotease-3 | AGCTGAGAACTT | GCTTCAAAGACA | CCAATGCCTGG |
| (MMP-3) | | | |
| Matrix | CCTGCCCCT | TTCAGGATTC | TGCAGAGCACTACTTGAA |
| metalloprotease-13 | TCCCTATGG | CCGCAAGAGT | ATCATACTACCATCCTGT |
| (MMP-13) | | | |
| Matrix | AGCCTTCCGAG | CTCCCGGATG | ACGCCACTGCG |
| metalloprotease-14 | TATGGGAGAGT | TAGGCATAGG | CTTCCGAGAAGT |
| (MMP-14) | | | |
| Tissue inhibitor | AAGGGCTACC | GGTATTGCCA | TTTGCCTGCCT |
| matrix | AGAGCGATCA | GGTGCACAAA | GCCACGGAATC |
| metalloprotease-1 | | | |
| (TIMP-1) | | | |
| Tissue inhibitor | CCCTATGATCC | GGTGCCCATT | CTGTGACCCAGTC |
| matrix | CATGCTACATCT | GATGCTCTTC | CATCCAGAGGCA |
| metalloprotease-2 | | | |
| (TIMP-2) | | | |
| Tissue inhibitor | CCCAGCACTA | CGTATTCCTTC | CCTCGGTACCAGCT |
| matrix | TGTCTGCATGA | CGGAGGTGTAG | ACAGATGCCATCAA |
| metalloprotease-4 | | | |
| (TIMP-4) | | | |
| Myosin heavy | GCCAAGGCTA | CGGGTGAGGT | TCCTCAGCCTTGCT |
| chain-beta (MHCα) | ACCTGGAGAAG | CATTGACAGA | CCGGTGTTCATTCAT |
| Myosin heavy | ACCTGGAGAAC | GGGCCTGCTC | AGGAAAAGCTCAAGAAGA |
| chain-alpha (MHCβ) | GACAAGCTTCA | GTCCTCTATT | AAGAGTTTGACATCAGTC |
| Cyclophilin | AGAGAAATTTGAG | TTGTGTTTGGT | AAGCATACAGGTCC |
| | GATGAGAACTTCAT | CCAGCATTTG | TGGCATCTTGTCCAT |

All oligonucleotides in Table 21 are written 5′-3′

iii) RNA isolation: MMP-2, MMP-3, MMP-13, MMP-14, TIMP-1, TIMP-2, TIMP-4, MHCα, and MHCβ

RNA is extracted from the frozen hearts using the RNeasy Midi Kit (Qiagen, Inc., Valencia, Calif.). More specifically, the tissue is crushed and homogenized at room temperature in RLT buffer (50% guanidium isothiocyanate/ethanol). 80 mAU of Qiagen Proteinase K is added, and the samples are incubated at 55° C. for 20 min. 0.5 vol ethanol is then added, and the samples are purified using RNeasy spin columns according to the manufacturer's (Qiagen, Inc.'s) instructions. RNA is eluted with 150 μl (×2) RNase-free water, frozen at −80° C. for 2 hr, thawed on wet ice, diluted, and analyzed spectrophotometrically for concentration and purity.

v) TaqMan analysis: MMP-2, MMP-3, MMP-13, MMP-14, TIMP-1, TIMP-2, TIMP-4, MHCα, and MHCβ

TaqMan reactions are performed as follows. 10 μL (200 ng) of DNased RNA is added to 15 μL of an RT-PCR reaction mix containing 12.5 μL of 2× One-Step PCR Master Mix without uracil-N-glycosylate (contains AmpliTaq Gold DNA Polymerase, dNTPs with dUTP, passive reference, and optimized buffer components), 0.625 μL of a 40× MultiScribe and RNAse Inhibitor Mix, 0.625 μL of 20 μM forward primer, 0.625 μL of 20 μM reverse primer, 0.5 μL of 5 μM TaqMan probe, and 0.125 μL of DNAse/RNAase-free water. Reactions are set up in duplicate in MicroAmp optical 96-well reaction plates with MicroAmp adhesive covers (Applied Biosystems), and loaded into the 7700 Sequence Detector. The following protocol is applied to all reactions: 30 min at 48° C. (reverse transcription), 10 min at 95° C. (inactivation of reverse transcriptase), 40 cycles of 15 sec at 95° C., and 1 min at 60° C. (PCR).

J. Urinary Proteinuria

Urinary proteinuria is determined by using the Bio-Rad protein dye reagent (Hercules, Calif.). The assay is modified to a 96-well plate format according to the manufacturer's instructions.

K. Detection of MMP Activity in Heart Tissue

Matrix metalloproteinase-2 and -9 (MMP-2 and MMP-9) activity is examined by zymography in heart extracts. Briefly, left ventricular tissue samples are homogenized in 25 ml ice-cold extraction buffer containing 1% Triton X-100, 25 mM HEPES, 0.15 M NaCl, 2 mM EDTA, and a complete protease inhibitor cocktail (Roche; Indianapolis, Ind.). The homogenates are centrifuged (4° C., 8,000 g, 20 min). Protein concentrations are then assessed using a bicinchoninic acid assay (Pierce; Rockford, Ill.), and equivalent amounts are separated by SDS-PAGE. After electrophoresis, gels are washed and allowed to renature for 1 hr. The gels are then incubated at 37° C. for 16-18 hr in developing buffer containing 1 mM Tris base, 40 mM Tris HCl, 200 nM NaCl, 5 mM CaCl₂, and 0.2% Brij 35, and stained with Coomassie blue. Proteases are visualized by the absence of staining indicating substrate cleavage.

L. Detection of p38 Activity in Heart Tissue

Anti-Hsp25 antibody is generated in rabbits by Quality Control Biochemicals, Inc. (Hopkinton, Mass.). The antigen peptide, conjugated to keyhole limpet hemocyanin (KLH), is as follows: YSRAL[pS]RQL(pS]S, with pS]denoting phosphorylated serine. Verification of antibody specificity is achieved using Western blotting techniques with competing, diphosphorylated peptide. Hsp-27 is a selective downstream target for p38 kinase. Thus, the level of phospholylation of Hsp27 in myocardium is directly correlated with cardiac activity of p38 MAPK.

M. Statistical Analysis

Data are analyzed using 1-way analysis of variance (ANOVA). Statistical analysis is performed on the rank transforms of the raw data (nonparametric analysis) to account for any inequality of variance. Statistical analysis on echocardiography data is performed on the change from baseline values. The p=0.05 level of significance is used for planned comparisons between the means. The Least Significant Differences (LSD) method is used for planned comparisons between groups. Data are analyzed using PROC GLM in the SAS statistical software package (SAS PC, version 6.12, SAS Institute, Cary, N.C.). All data are reported as mean±SEM.

III. Observations

During this experiment, the groups of rats are compared with respect to, for example, systolic blood pressure, ejection fraction, stroke volume, left ventricular end diastolic area, left ventricular end systolic area, left ventricular end diastolic volume, left ventricular end systolic volume, urinary protein, TNFα in the serum, TNFα in the heart tissue, left ventricular mass (absolute and normalized to tibial length), plasma osteopontin, cardiac p38 kinase activity, and MMP levels and activity.

### Example 3

### Spontaneously Hypertensive Heart Failure (SHHF) Rat Model to Evaluate a Combination Therapy of a p38 Kinase Inhibitor with a Diuretic

The SHHF rat model also may be used to evaluate a combination therapy of a p38 kinase inhibitor with a diuretic.

I. Experimental Protocol

This study is conducted using lean, male spontaneously SHHF rats (Genetic Models Inc., Indianapolis, Ind.), and age-matched Sprague-Dawley (SD) rats (Charles River Labs, Raleigh, N.C.) as controls. All the animals are housed in a room lighted 12 hours per day at an ambient temperature of 22±1° C. The animals are allowed 3 weeks to adjust after arrival, and are given free access to rodent diet (Purina 5002; Ralston Purina, St. Louis, Mo.) and tap water ad libitum. At the initiation of the study, all the animals are 15 months of age.

The study is conducted over 12 weeks, with measurements and samples taken at baseline, and after 4, 8, and 12 weeks of treatment (termination of study). Following acclimation, baseline measurements are performed, and 1 week later, the rats are assigned to one of the following treatment groups: (1) rats receiving no treatment; (2) rats receiving a diuretic of interest at a dose of interest, (3) rats receiving a p38 kinase inhibitor at a dose of interest, and (4) rats receiving a co-administration of the diuretic at a dosing of interest and the p38 inhibitor at a dosing of interest.

II. Assays and Analyses

The assays and analysis used here include those described above in Example 2.

III. Observations

During this experiment, the groups of rats are compared with respect to, for example, systolic blood pressure, ejection fraction, stroke volume, left ventricular end diastolic area, left ventricular end systolic area, left ventricular end diastolic volume, left ventricular end systolic volume, urinary protein, TNFα in the serum, TNFα in the heart tissue, left ventricular mass (absolute and normalized to tibial length), plasma osteopontin, cardiac p38 kinase activity, and MMP levels and activity.

### Example 4

### Volume Expanded Hypertensive Rat Model to Evaluate a Combination Therapy of a p38 Kinase Inhibitor with an Aldosterone Antagonist

The volume expanded hypertensive rat model (also known as the aldosterone/salt rat model) has been described in the art. See, e.g., Rocha, R., et al., “Aldosterone induces a vascular inflammatory phenotype in the rat heart”, Am. J. Physiol. Heart Circ. Physiol., vol. 283, pp. H1802-H1810 (2002) (incorporated by reference into this patent). See also, Blasi, E. R., et al., “Aldosterone/salt induces renal inflammation and fibrosis in hypertensive rats”, Kidney International, vol. 63, pp. 1791-1800 (2003) (incorporated by reference into this patent). See also, PCT Patent Publication No. WO 01/95893 (incorporated by reference into this patent). This model may be used to evaluate a combination therapy of a p38 kinase inhibitor with an aldosterone antagonist. An example using this model for such a purpose is described below.

Following acclimation, unnephrectomized rats are given 1% NaCl drinking water and infused subcutaneously with aldosterone (0.5 g/kg/hr) via an Alza osmotic pump, Model 2002. These rats are assigned to one of the following treatment groups: (1) rats receiving no treatment; (2) rats receiving an aldosterone antagonist of interest at a dosing of interest, (3) rats receiving a p38 kinase inhibitor of interest at a dosing of interest, and (4) rats receiving a co-administration of the aldosterone antagonist at a dosing of interest and the p38 inhibitor at a dosing of interest. The treatments continued for 3 weeks. Over that period, blood pressure and heart rate are evaluated continuously by telemetry via an implanted transmitter connected to a pressure transducer cannulated to the abdominal aorta. The blood pressure and heart rate data is averaged over 24-hour periods.

During this experiment, the groups of rats are compared with respect to, for example, changes in average blood pressure and average heart rate, levels of inflammation markers, organ damage, and vascular damage.

### Example 5

### Volume Expanded Hypertensive Rat Model to Evaluate a Combination Therapy of a p38 Kinase Inhibitor with a Diuretic

The volume expanded hypertensive rat model also may be used to evaluate combination therapy of a p38 kinase inhibitor with a diuretic.

Following acclimation, unnephrectomized rats are given 1% NaCl drinking water and infused subcutaneously with aldosterone (0.5 g/kg/hr) via an Alza osmotic pump, Model 2002. These rats are assigned to one of the following treatment groups: (1) rats receiving no treatment; (2) rats receiving an diuretic of interest at a dosing of interest, (3) rats receiving a p38 kinase inhibitor of interest at a dosing of interest, and (4) rats receiving a co-administration of the diuretic at a dosing of interest and the p38 inhibitor at a dosing of interest. The treatments are continued for 3 weeks. Over that period, blood pressure and heart rate are evaluated continuously by telemetry via an implanted transmitter connected to a pressure transducer cannulated to the abdominal aorta. The blood pressure and heart rate data is averaged over 24-hour periods.

During this experiment, the groups of rats are compared with respect to, for example, changes in average blood pressure and average heart rate, levels of inflammation markers, organ damage, and vascular damage.

### Example 6

### Stroke Prone Spontaneously Hypertensive Rat (SHR-SP) Model to Evaluate a Combination Therapy of a p38 Kinase Inhibitor with an Aldosterone Antagonist

The stroke prone spontaneously hypertensive rat (SHR-SP) model has been described in the art. See, e.g., Rocha, R., et al., “Pathophysiological effects of aldosterone in cardiovascular tissues”, Trends in Endocrin. & Met., vol. 12(7), pp. 308-314 (September 2001) (incorporated by reference into this patent). This model may be used to evaluate a combination therapy of a p38 kinase inhibitor with an aldosterone antagonist. Examples using the SHR-SP model for such a purpose are described below.

I. Animals

A study using the SHR-SP model may, for example, be conducted in accordance with institutional guidelines using male SHRSP/A3N rats bred from NIH stock and derived from the SHRSP/A3N substrain described in Okamoto, et al, Circ. Res., 34 and 35 (suppl. I-143 to I-153). Typically, these rats are housed in a room maintained on a 12:12-hr light:dark-cycle and an ambient temperature of 22±1° C. The rats are weaned at 4 weeks of age, and allowed free access to Purina Lab Chow 5001 (Ralston Purina, St. Louis, Mo.) and tap water until the initiation of the experimental protocols. One source of SHR-SP rats is the Animal Care Facility at New York Medical College.

II. Effects on Blood Pressure

A. Experimental Protocol

SHR-SP rats are maintained on normal rat chow and non-saline drinking water (i.e., tap water). At the age of 13 weeks, the rats are assigned to one of the following treatment groups: (1) rats receiving no treatment (the control); (2) rats receiving an aldosterone antagonist of interest at a dosing of interest, (3) rats receiving a p38 kinase inhibitor of interest at a dosing of interest, and (4) rats receiving a co-administration of the aldosterone antagonist at a dosing of interest and the p38 inhibitor at a dosing of interest. These treatments are conducted over a 3-week period. Indirect measurements of systolic blood are assessed by tail cuff plethylsmography.

B. Observations

During this experiment, the groups of rats are compared with respect to, for example, changes in systolic blood pressure.

III. Prevention of Stroke and Cerebrovascular Damage

A. Experimental Protocol

Saline-drinking SHR-SP rats at the age of 9 weeks are assigned to one of the following treatment groups: (1) rats receiving no treatment (the control); (2) rats receiving an aldosterone antagonist of interest at a dosing of interest, (3) rats receiving a p38 kinase inhibitor of interest at a dosing of interest, and (4) rats receiving a co-administration of the aldosterone antagonist at a dosing of interest and the p38 inhibitor at a dosing of interest. These treatments are conducted up to 9.5 weeks (to the extent the rats survived the entire period). At the end of this period, the surviving rats are sacrificed for further evaluation.

B. Observations

During this experiment, the groups of rats are compared with respect to, for example, signs of stroke, development of proteinuria, and severity of hypertension. Histopathic analysis of the brains of the sacrificed rats also is conducted to determine the effect of the treatments with respect to the development of liquofactive neorosis associated with fibrinoid necrotic lesions in cerebral arteries and arterioles with focal hemorrhages.

IV. Vascular Protective Effects

A. Experimental Protocol

i) First Protocol

SHR-SP rats are given 1% NaCl to drink ad libitum, and are fed Stroke-Prone Rodent Diet (#39-288, Zeigler Bros., Inc., Gardners, Pa.) starting at 8.1 weeks of age. This diet is lower in potassium (0.7% v 1.2% by weight) and protein (17% v 22% by weight) than the standard diet, and induces a higher incidence of stroke in SHR-SP rats (see, e.g., Stier, C. T., et al, Hypertension, vol. 13, pp. 115-121 (1989) (incorporated by reference into this patent)). At 8.4 weeks of age, the rats are assigned to one of the following treatment groups: (1) rats receiving no treatment; (2) rats receiving an aldosterone antagonist of interest at a dosing of interest, (3) rats receiving a p38 kinase inhibitor of interest at a dosing of interest, and (4) rats receiving a co-administration of the aldosterone antagonist at a dosing of interest and the p38 inhibitor at a dosing of interest. These procedures are carried out for 5 weeks. The rats are housed individually in metabolic cages so that measurements of 24-hr urine output and protein excretion can be made. Animals are examined daily for signs of stroke. Systolic arterial pressure and heart rate are measured each week in awake rats. At the end of the weeks, trunk blood is collected into chilled EDTA tubes following rapid decapitation of the animals between 10:00 am and 12:00 pm. Blood is stored at 20° C. for later measurement of plasma aldosterone levels. The kidneys are rapidly removed, weighed, and preserved in fixative for later histologic examination.

ii) Second Protocol

SHR-SP rats are given 1% NaCl to drink ad libitum and are fed Stroke-Prone Rodent Diet (#39-288, Zeigler Bros., Inc., Gardners, Pa.) starting at 8.3 weeks of age. To provide a consistent background suppression of endogenous angiotensin II levels among the animals, captopril (Sigma Chemical Col, St. Louis, Mo.) is added to the drinking solution of all animals to provide a dose of 50 mg/kg/day. This dose of captopril, in the absence of angiotensin II infusion, will prevent the development of renal and cerebrovascular lesions in saline-drinking SHR-SP rats (see Rocha, R., et al., Hypertension, vol. 33, pp. 232-237 (incorporated by reference into this patent)). At 9.3 weeks of age, Alzet osmotic mini-pumps (Model 2002, Alza Co., Palo Alto, Calif.), containing angiotensin II (human type, American Peptide Inc., Sunnyvale, Calif.) or its vehicle (sterile 0.9% NaCl) are implanted beneath the skin at the nape of the necks in SHR-SP rats receiving inhalatory anesthesia with isofluorane (Ohmeda Caribe, Inc., Guayama, PR). The rats are housed in individual metabolic cages and assigned to one of the following treatment groups: (1) rats receiving an infusion of the vehicle (the first control); (2) rats receiving angiotensin II infusion (25 ng/min, subcutaneously) (second control); (3) rats receiving an aldosterone antagonist of interest at a dosing of interest and angiotensin II infusion (25 ng/min, subcutaneously); (4) rats receiving a p38 kinase inhibitor of interest at a dosing of interest and angiotensin II infusion (25 ng/min, subcutaneously); and (5) rats receiving a co-administration of the aldosterone antagonist at a dosing of interest and the p38 inhibitor at a dosing of interest and angiotensin II infusion (25 ng/min, subcutaneously). It has been reported that a dose of 25 ng/min of angiotensin II could reverse the vascular protective effect of ACE inhibitor treatment with enalapril in saline-drinking SHR-SP rats. See WO 01/95893.

These above treatments are conducted for 2 weeks. During this period, the animals are handled and weighed daily, urine samples are collected for the assessment of proteinuria, and systolic blood pressure and heart rate are measured each week. At the end of the two weeks, the animals are decapitated. Trunk blood is collected into chilled EDTA tubes, and the kidneys are removed, blotted dried, and weighed. Coronal sections of kidney are fixed and later processed for light microscopic evaluation.

Assays and Analysis

i) Measurement of Blood Pressure, Heart Rate, Urine Volume, Urinary Protein Concentration, and Plasma Aldosterone

Systolic blood pressure and heart rate of awake animals are measured by tail-cuff plethysmography using a Natsume KN-210 manometer and tachometer (Peninsula Laboratories Inc., Belmont, Calif.). Rats are warmed at 37° C. for 10 min and allowed to rest quietly in a Lucite chamber before measurement of blood pressure. Measurements of urine volume are made gravimetrically. Urinary protein concentration is determined by the sulfosalicylic acid turbidity method. Plasma aldosterone is measured by radioimmunoassay using ¹²⁵I-aldosterone as a tracer (Coat-a Count Aldosterone, Diagnostic Products Co., Los Angeles, Calif.).

ii) Histology

The kidneys are preserved in 10% phosphate-buffered formalin. Coronal sections (2-3 μm) are stained with hematoxylin and eosin, and examined by light microscopy in a blinded fashion as described in Stier, C. T., et al., J. Pharmacol. Exp. Ther., vol. 269, pp. 1410-1415 (1992) (incorporated by reference into this patent). Glomerular damage is categorized as ischemic or thrombotic. Ischemic lesions are defined as retraction of glomerular capillary tufts with or without appreciable mesangiolysis. Glomerular thrombotic lesions are defined as any one of a combination of the following: segmental to global fibrinoid necrosis, focal thrombosis of glomerular capillaries, swelling and proliferation of intra-capillary (endothelial and mesangial) and/or extra-capillary cells (crescents), and expansion of reticulated mesangial matrix with or without significant hypercellularity. The number of glomeruli exhibiting lesions in either category is enumerated from each kidney, and is expressed as a percentage of the total number of glomeruli present per mid-coronal section. Vascular thrombotic lesions are defined as any one or a combination of the following: mural fibrinoid necrosis, extravasation and fragmentation of red blood cells, and luminal and/or mural thrombosis. Proliferative arteriopathy is characterized by proliferation of markedly swollen myointimal cells with swollen round to ovoid vesicular nuclei surrounded by mucinous extracellular matrix (“onion skinning”) often resulting in nodular thickening. Vascular damage is expressed as the number of arteries and arterioles with lesions per 100 glomeruli. The presence of casts and tubular (ischemic) retraction and simplification is assessed semi-quantitatively.

ii) Statistical Analysis

Significant effects with respect to treatment and time are determined by two-way analysis of variance. Data with only one grouping variable are analyzed statistically by Student's impaired t tests. When more than two groups are compared, one-way analysis of variance is performed, followed by the post-hoc Newman-Keul's multiple comparison test. Data is analyzed using version 2.01 of the GraphPad Prism statistical software package (GraphPad Software Inc., San Diego, Calif.). P<0.05 is considered statistically significant. Data is reported as mean±SEM.

C. Observations

During this experiment, the groups of rats are compared with respect to, for example, changes in body weight, changes in systolic blood pressure and heart rate, changes in urinary protein excretion, development of renal lesions, development of cardiac damage, development of cerebral damage, kidney weight (absolute and normalized with body weight), development of vascular lesions, development of signs of stroke, and changes in aldosterone levels. Analysis of renal lesions includes, for example, analysis for glomerular damage (ischemic and thrombotic damage), renal arteriopathy (thrombotic and proliferative damage in the small arteries and arterioles), malignant nephrosclerosis, ischemic retraction, thrombonecrosis of capillary tufts, arteriolar fibrinoid necrosis with fragmented and extravasated erythrocytes, concentric proliferative arteriopathy, simplification of tubules, dilation of tubules with protein casts, inflammatory cell filtration, and mortality.

### Example 7

### Stroke Prone Spontaneously Hypertensive Rat (SHR-SP) Model to Evaluate a Combination Therapy of a p38 Kinase Inhibitor with a Diuretic

The SHR-SP model discussed above also may be used to evaluate a combination therapy of a p38 kinase inhibitor with a diuretic. Examples using the SHR-SP model for such a purpose are described below.

I. Animals

The animals used here include those described above in Part I of Example 6.

II. Effects on Blood Pressure

A. Experimental Protocol

SHR-SP rats are maintained on normal rat chow and non-saline drinking water (i.e., tap water). At the age of 13 weeks, the rats are assigned to one of the following treatment groups: (1) rats receiving no treatment (the control); (2) rats receiving an diuretic of interest at a dosing of interest, (3) rats receiving a p38 kinase inhibitor of interest at a dosing of interest, and (4) rats receiving a co-administration of the diuretic at a dosing of interest and the p38 inhibitor at a dosing of interest. These treatments are conducted over a 3-week period. Indirect measurements of systolic blood are assessed by tail cuff plethylsmography.

B. Observations

During this experiment, the groups of rats are compared with respect to, for example, changes in systolic blood pressure.

III. Prevention of Stroke and Cerebrovascular Damage

A. Experimental Protocol

Saline-drinking SHR-SP rats at the age of 9 weeks are assigned to one of the following treatment groups: (1) rats receiving no treatment (the control); (2) rats receiving an diuretic of interest at a dosing of interest, (3) rats receiving a p38 kinase inhibitor of interest at a dosing of interest, and (4) rats receiving a co-administration of the diuretic at a dosing of interest and the p38 inhibitor at a dosing of interest. These treatments are conducted up to 9.5 weeks (to the extent the rats survived the entire period). At the end of this period, the surviving rats are sacrificed for further evaluation.

B. Observations

During this experiment, the each group of rats is compared with the other groups in its protocol with respect to, for example, signs of stroke, development of proteinuria, and severity of hypertension. Histopathic analysis of the brains of the sacrificed rats also is conducted to determine the effect of the treatments with respect to the development of liquofactive neorosis associated with fibrinoid necrotic lesions in cerebral arteries and arterioles with focal hemorrhages.

IV. Vascular Protective Effects

A. Experimental Protocol

i) First Protocol

SHR-SP rats are given 1% NaCl to drink ad libitum, and are fed Stroke-Prone Rodent Diet (#39-288, Zeigler Bros., Inc., Gardners, Pa.) starting at 8.1 weeks of age. This diet is lower in potassium (0.7% v 1.2% by weight) and protein (17% v 22% by weight) than the standard diet, and induces a higher incidence of stroke in SHR-SP rats (see, e.g., Stier, C. T., et al, Hypertension, vol. 13, pp. 115-121 (1989)). At 8.4 weeks of age, the rats are assigned to one of the following treatment groups: (1) rats receiving no treatment; (2) rats receiving an diuretic of interest at a dosing of interest, (3) rats receiving a p38 kinase inhibitor of interest at a dosing of interest, and (4) rats receiving a co-administration of the diuretic at a dosing of interest and the p38 inhibitor at a dosing of interest. These procedures are carried out for 5 weeks. The rats are housed individually in metabolic cages so that measurements of 24-hr urine output and protein excretion can be made. Animals are examined daily for signs of stroke. Systolic arterial pressure and heart rate are measured each week in awake rats. At the end of the weeks, trunk blood is collected into chilled EDTA tubes following rapid decapitation of the animals between 10:00 am and 12:00 pm. Blood is stored at 20° C. for later measurement of plasma aldosterone levels. The kidneys are rapidly removed, weighed, and preserved in fixative for later histologic examination.

ii) Second Protocol

SHR-SP rats are given 1% NaCl to drink ad libitum and are fed Stroke-Prone Rodent Diet (#39-288, Zeigler Bros., Inc., Gardners, Pa.) starting at 8.3 weeks of age. To provide a consistent background suppression of endogenous angiotensin II levels among the animals, captopril (Sigma Chemical Col, St. Louis, Mo.) is added to the drinking solution of all animals to provide a dose of 50 mg/kg/day. This dose of captopril, in the absence of angiotensin II infusion, will prevent the development of renal and cerebrovascular lesions in saline-drinking SHR-SP rats (see Rocha, R., et al., Hypertension, vol. 33, pp. 232-237). At 9.3 weeks of age, Alzet osmotic mini-pumps (Model 2002, Alza Co., Palo Alto, Calif.), containing angiotensin II (human type, American Peptide Inc., Sunnyvale, Calif.) or its vehicle (sterile 0.9% NaCl) are implanted beneath the skin at the nape of the necks in SHR-SP rats receiving inhalatory anesthesia with isofluorane (Ohmeda Caribe, Inc., Guayama, PR). The rats are housed in individual metabolic cages and assigned to one of the following treatment groups: (1) rats receiving an infusion of the vehicle (the first control); (2) rats receiving angiotensin II infusion (25 ng/min, subcutaneously) (second control); (3) rats receiving an diuretic of interest at a dosing of interest and angiotensin II infusion (25 ng/min, subcutaneously); (4) rats receiving a p38 kinase inhibitor of interest at a dosing of interest and angiotensin II infusion (25 ng/min, subcutaneously); and (5) rats receiving a co-administration of the diuretic at a dosing of interest and the p38 inhibitor at a dosing of interest and angiotensin II infusion (25 ng/min, subcutaneously).

The above treatments are conducted for 2 weeks. During this period, the animals are handled and weighed daily, urine samples are collected for the assessment of proteinuria, and systolic blood pressure and heart rate are measured each week. At the end of the two weeks, the animals are decapitated. Trunk blood is collected into chilled EDTA tubes, and the kidneys are removed, blotted dried, and weighed. Coronal sections of kidney are fixed and later processed for light microscopic evaluation.

B. Assays and Analysis

The assays and analysis used here include those described above in Part III (B) of Example 6.

C. Observations

During this experiment, the each group of rats is compared with the other groups in its protocol with respect to, for example, changes in body weight, changes in systolic blood pressure and heart rate, changes in urinary protein excretion, development of renal lesions, development of cardiac damage, development of cerebral damage, kidney weight (absolute and normalized with body weight), development of vascular lesions, development of signs of stroke, and changes in aldosterone levels. Analysis of renal lesions includes, for example, analysis for glomerular damage (ischemic and thrombotic damage), renal arteriopathy (thrombotic and proliferative damage in the small arteries and arterioles), malignant nephrosclerosis, ischemic retraction, thrombonecrosis of capillary tufts, arteriolar fibrinoid necrosis with fragmented and extravasated erythrocytes, concentric proliferative arteriopathy, simplification of tubules, dilation of tubules with protein casts, inflammatory cell filtration, and mortality.

### Example 8

### Chronic Heart Failure Dog Model to Evaluate a Combination Therapy of a p38 Kinase Inhibitor with an Aldosterone Antagonist

A canine model of chronic heart failure has been described in the art. See, e.g., Suzuki, G., “Effects of Long-Term Monotherapy With Eplerenone, a Novel Aldosterone Blocker, on Progression of Left Ventricular Dysfunction and Remodeling in Dogs with heart failure”, Circulation, vol. 106, pp. 2967-2972 (Dec. 3, 2002) (incorporated by reference into this patent). See also, Sabbah, H. N., et al., “A canine model of chronic heart failure produced by multiple sequential coronary microembolizations”, Am. J. Physiol., vol. 260, pp. H1379-H1384 (1991) (incorporated by reference into this patent). This model may be used to evaluate a combination therapy of a p38 kinase inhibitor with an aldosterone antagonist. An example using this model for such a purpose is described below.

Study Protocol

In this study, mongrel dogs undergo serial coronary microembolizations to produce heart failure. Embolizations are performed 1 to 3 weeks apart, and are discontinued when left ventricular ejection fraction is 30% to 40%. Microembolizations are performed during cardiac catheterization under general anesthesia and sterile conditions. Anesthesia consists of a combination of intravenous injections of oxymorphone (0.22 mg/kg), diazepam (0.17 mg/kg), and sodium pentobarbital (150 to 250 mg to effect).

Two weeks after the last microembolization, the dogs undergo a pre-randomization left and right heart catheterization. One day later, the dogs are randomized, and then assigned to one of the following treatment groups: (1) dogs receiving no treatment; (2) dogs receiving an aldosterone antagonist of interest at a dosing of interest, (3) dogs receiving a p38 kinase inhibitor of interest at a dosing of interest, and (4) dogs receiving a co-administration of the aldosterone antagonist at a dosing of interest and the p38 inhibitor at a dosing of interest. This treatment is continued for 3 months. Final hemodynamic and angiographic measurements are made at the end of the 3 months. While under anesthesia, the each dog's chest is opened, the heart is removed, and tissue is prepared for biochemical and histological evaluations.

II. Assays and Analysis

A. Hemodynamic and Angiographic Measurements

Hemodynamic and angiographic measurements are made during cardiac catheterizations at baseline, 1 day before initiation of therapy, and at the end of 3 months of therapy. Aortic and left ventricular pressures are measured with catheter-tip micromanometers (Millar Instruments). Mean pulmonary artery pressure is measured with a fluid-filled catheter in conjunction with a Perceptor DT pressure transducer (Boston Scientific). Peak left ventricular rate of change in pressure during isovolumic contraction (+dP/dt) and relaxation (−dP/dt) and end-diastolic pressure are measured from the left ventricular pressure waveform. The time constant of isovolumic relaxation, τ, is calculated as described in Weiss, J. L., et al., “Hemodynamic determinants of the time-course of fall in canine left ventricular pressure”, J. Clin. Invest., vol. 58, pp. 751-760 (1976) (incorporated by reference into this patent).

Left ventriculograms are obtained after completion of the hemodynamic measurements, with each dog placed on its right side, and recorded on 35-mm cine film at 30 frames/second during the injection of 20 mL of contrast material (RENO-M-60, Squibb). Correction for image magnification is made with a radiopaque calibrated grid placed at the level of the left ventricle. Left ventricular end-diastolic volume, end-systolic volume, and ejection fraction are calculated as described in Sabbah, H. N., et al. Global indexes of left ventricular shape are used to quantify changes in chamber sphericity. Left ventricular shape is quantified from angiographic silhouettes as the ratio of the major to minor axes at end diastole and end systole. Venous blood samples are obtained before and 3 months after initiation of therapy for measurement of plasma concentrations of Na⁺, K⁺, blood urea nitrogen (BUN), and creatinine.

B. Echocardiographic Measurements

Echocardiograms are performed with a Hewlett-Packard model 77020A ultrasound system with a 3.5-MHz transducer, and recorded on a VHS recorder. The thickness of the intraventricular septum and left ventricular posterior wall is determined by M-mode echocardiography, summed, and averaged to obtain a single representative measure of left ventricular wall thickness. The end-diastolic left ventricular major and minor semiaxes at the midwall are measured from 2D echocardiograms with the apical 4-chamber view. Left ventricular end-diastolic circumferential wall stress is calculated as described in Grossman, W., “Pressure Measurement”, Cardiac Catheterization, Angiography, and Intervention, p. 123 (ed: Grossman, W., et al., Lea & Feiger, Philadelphia, Pa. (1991)).

C. Histological and Morphometric Assessments

From each heart, 3 transverse slices (=3 mm thick, 1 each from the basal, middle, and apical thirds of the left ventricular) are obtained. For comparison, tissue samples from normal dogs also are prepared in an identical manner. From each slice, transmural tissue blocks are obtained and embedded in paraffin blocks. From each block, 6-μm-thick sections are prepared and stained with Gomori trichrome to identify fibrous tissue. The volume fraction of replacement fibrosis, namely, the proportion of scar tissue to viable tissue in all 3 transverse left ventricular slices, is calculated as the percent total surface area occupied by fibrous tissue by use of computer-based video densitometry (MOCHA, Jandel Scientific). Left ventricular free-wall tissue blocks are obtained from a second midventricular transverse slice, mounted on cork with Tissue-Tek embedding medium (Sakura), and rapidly frozen in isopentane (pre-cooled in liquid nitrogen) and stored at −70° C. until used. Cryostat sections are prepared and stained with fluorescein-labeled peanut agglutinin (Vector Laboratories Inc.) after pretreatment with 3.3 U/mL neuraminidase type V (Sigma Chemical Co.) to delineate the myocyte border and the interstitial space, including capillaries. Sections are double stained with rhodamine-labeled Griffonia Simplicifolia lectin I (GSL-I) to identify capillaries. Ten radially oriented microscopic fields (magnification ×100, objective ×40, and ocular 2.5) are selected at random from each section for analysis. Fields that contain scar tissue (infarcts) are excluded. Average myocyte cross-sectional area is calculated by computer-assisted planimetry. Volume fraction of interstitial fibrosis is calculated as the percent total surface area occupied by interstitial space minus the percent total area occupied by capillaries. Capillary density is calculated as the number of capillaries per square millimeter.

D. TaqMan Analysis and Zymography

RNA is extracted and purified from frozen left ventricular tissue with the RNeasy Midi Kit (Qiagen, Inc), followed by DNA removal with DNAse (Qiagen, Inc). Primers and probes for basic fibroblast growth factor are designed with Primer Express software supplied with the 7700 Sequence Detection System and synthesized by Applied Biosystems. Target gene results are normalized to the housekeeping gene cyclophilin. Purified RNA (200 ng of total) is added to a reverse transcription-polymerase chain reaction mix that contained the following: 12.5 μL of 2× One-Step PCR Master Mix without uracil-N-glycosylase, 0.625 μL of a 40× MultiScribe and RNAse Inhibitor Mix, 0.625 μL of 20 μmol/L forward primer, 0.625 μL of 20 μmol/L reverse primer, 0.5 μL of 5 μmol/L TaqMan probe, and 0.125 μL of DNAse/RNAse-free water. Reactions are analyzed in duplicate in the 7700-Sequence Detector with the following protocol: 30 min at 48° C. (reverse transcription), 10 min at 95° C. (inactivation of reverse transcriptase and polymerase activation), 40 cycles of 15 sec at 95° C. (denaturation), and 1 min at 60° C. (annealing). Zymography is performed as described in Sabbah, H. N., et al. Gelatinase activity is analyzed by densitometry, and activity is represented as optical density.

E. Data Analysis

Intra-group comparisons are made between measurements obtained before initiation of therapy and measurements made after 3 months of therapy. For these comparisons, a Student's paired t test is used, and a probability ≦0.05 is considered significant. To ensure that all study measures are similar at baseline and at the time of randomization, inter-group comparisons are made with a t statistic for 2 means. To assess treatment effect, the change in each measure from before treatment to after treatment is calculated for each group. To determine whether significant differences are present between groups, a t statistic for 2 means is used, with P ≦0.05 considered significant. Differences in electrolytes, BUN, creatinine, bFGF, gelatinase activity, and histomorphometric measures are examined with ANOVA, with oset at 0.05, and pair-wise comparisons are made with the Student-Neuman-Keuls test, with P ≦0.05 considered significant. All data are reported as mean±SEM.

III. Observations

During this experiment, the groups of dogs are compared with respect to, for example, changes in left ventricular ejection fraction; end-diastolic volume; end-systolic volume; peak left ventricular +dP/dt; peak left ventricular −dP/dt; pulmonary artery pressure; the time constant of isovolumic relaxation, r, left ventricular end-diastolic and end-systolic axes ratios (which, in turn, indicate changes in left ventricular chamber sphericity); left ventricular end-diastolic wall stress; body weight; heart weight (normalized with body weight); left ventricular wall thickness; Na⁺, K⁺, BUN, and creatinine; mean aortic pressure; and heart rate. Comparisons also are made with respect to, for example, cardiac myocyte cross-sectional area (which, in turn, is a measure of cell hypertrophy), volume fraction of interstitial fibrosis, and volume fraction of replacement fibrosis, and capillary density, gelatinase activity, and transcription of basic fibroblast growth factor.

### Example 9

### Chronic Heart Failure Dog Model to Evaluate a Combination Therapy of a p38 Kinase Inhibitor with a Diuretic

The chronic heart failure dog model discussed above also may be used to evaluate a combination therapy of a p38 kinase inhibitor with a diuretic. An example using this model for such a purpose is described below.

I. Study Protocol

In this study, mongrel dogs undergo serial coronary microembolizations to produce heart failure. Embolizations are performed 1 to 3 weeks apart, and are discontinued when left ventricular ejection fraction is 30% to 40%. Microembolizations are performed during cardiac catheterization under general anesthesia and sterile conditions. Anesthesia consists of a combination of intravenous injections of oxymorphone (0.22 mg/kg), diazepam (0.17 mg/kg), and sodium pentobarbital (150 to 250 mg to effect).

Two weeks after the last microembolization, the dogs undergo a pre-randomization left and right heart catheterization. One day later, the dogs are randomized, and then assigned to one of the following treatment groups: (1) dogs receiving no treatment; (2) dogs receiving an diuretic of interest at a dosing of interest, (3) dogs receiving a p38 kinase inhibitor of interest at a dosing of interest, and (4) dogs receiving a co-administration of the diuretic at a dosing of interest and the p38 inhibitor at a dosing of interest. This treatment is continued for 3 months. Final hemodynamic and angiographic measurements are made at the end of the 3 months. While under anesthesia, the each dog's chest is opened, the heart is removed, and tissue is prepared for biochemical and histological evaluations.

II. Assays and Analysis

Assays and analysis used here include those described above in Example 8.

III. Observations

During this experiment, the groups of dogs are compared with respect to, for example, changes in left ventricular ejection fraction; end-diastolic volume; end-systolic volume; peak left ventricular +dP/dt; peak left ventricular −dP/dt; pulmonary artery pressure; the time constant of isovolumic relaxation, τ; left ventricular end-diastolic and end-systolic axes ratios (which, in turn, indicate changes in left ventricular chamber sphericity); left ventricular end-diastolic wall stress; body weight; heart weight (normalized with body weight); left ventricular wall thickness; Na⁺, K⁺, BUN, and creatinine; mean aortic pressure; and heart rate. Comparisons also are made with respect to, for example, cardiac myocyte cross-sectional area (which, in turn, is a measure of cell hypertrophy), volume fraction of interstitial fibrosis, and volume fraction of replacement fibrosis, and capillary density, gelatinase activity, and transcription of basic fibroblast growth factor.

Several other animal models are available that are appropriate for evaluating combinations of p38-kinase inhibitors with ACE inhibitors to treat cardiovascular conditions and other associated conditions. Appropriate models may include, for example, those disclosed in PCT Patent Publication No. WO 02/09759. Appropriate models also may include, for example, those disclosed in PCT Patent Publication No. WO 01/95893. These references are incorporated by reference into this patent.

The above detailed description of preferred embodiments is intended only to acquaint others skilled in the art with the invention, its principles, and its practical application so that others skilled in the art may adapt and apply the invention in its numerous forms, as they may be best suited to the requirements of a particular use. This invention, therefore, is not limited to the above embodiments, and may be variously modified.

1. A method for treating a pathological condition in a mammal, wherein:
the method comprises administering to the mammal:
a first amount of a compound that comprises a substituted-pyrazole p38-kinase inhibitor, and
a second amount of a compound that comprises an aldosterone antagonist or a diuretic; and
the first and second amounts of the compounds together comprise a therapeutically-effective amount of the compounds.

2. A method according to claim 1, wherein the pathological condition comprises a cardiovascular disease, renal dysfunction, cerebrovascular disease, vascular disease, retinopathy, neuropathy, edema, endothelial dysfunction, or insulinopathy.

3. A method according to claim 2, wherein the pathological condition comprises a cardiovascular disease.

4. A method according to claim 3, wherein the cardiovascular disease comprises hypertension, vascular inflammation in the heart, coronary angioplasty, coronary thrombosis, cardiac lesions, myocarditis, coronary artery disease, heart failure, arrhythmia, diastolic dysfunction, systolic dysfunction, ischemia, cardiomyopathy, sudden cardiac death, myocardial fibrosis, vascular fibrosis, impaired arterial compliance, myocardial necrotic lesions, vascular damage in the heart, myocardial infarction, left ventricular hypertrophy, decreased ejection fraction, vascular wall hypertrophy in the heart, or endothelial thickening.

5. A method according to claim 4, wherein the cardiovascular disease comprises fibrinoid necrosis of coronary arteries, congestive heart failure, chronic heart failure, acute heart failure, left ventricular diastolic dysfunction, diastolic heart failure, impaired diastolic filling, myocardial ischemia, hypertrophic cardiomyopathy, dilated cardiomyopathy, an acute post-myocardial-infarction condition, or a chronic post-myocardial-infarction condition.

6. A method according to claim 4, wherein the cardiovascular disease comprises hypertension.

7. A method according to claim 4, wherein the cardiovascular disease comprises heart failure.

8. A method according to claim 2, wherein the pathological condition comprises a renal dysfunction.

9. A method according to claim 8, wherein the renal dysfunction comprises glomerulosclerosis, end-stage renal disease, acute renal failure, diabetic nephropathy, reduced renal blood flow, increased glomerular filtration fraction, proteinuria, decreased glomerular filtration rate, decreased creatine clearance, microalbuminuria, renal arteriopathy, ischemic lesions, vascular damage in the kidney, vascular inflammation in the kidney, or malignant nephrosclerosis.

10. A method according to claim 2, wherein the second amount comprises an aldosterone antagonist.

11. A method according to claim 10, wherein the aldosterone antagonist comprises an epoxy-steroidal aldosterone antagonist.

12. A method according to claim 11, wherein the aldosterone receptor antagonist comprises eplerenone.

13. A method according to claim 12, wherein the pathological condition comprises heart failure.

14. A method according to claim 13, wherein the mammal is a dog.

15. A method according to claim 10, wherein the aldosterone antagonist comprises an non-epoxy-steroidal aldosterone antagonist.

16. A method according to claim 15, wherein the aldosterone receptor antagonist comprises spironolactone.

17. A method according to claim 10, wherein the method further comprises a third amount of a compound comprising a diuretic.

18. A method according to claim 2, wherein the second amount comprises a diuretic.

19. A method according to claim 18, wherein the diuretic comprises amanozine, amiloride, arbutin, chlorazanil, ethacrynic acid, etozolin, hydracarbazine, isosorbide, mannitol, metochalcone, muzolimine, perhexiline, ticrynafen, triamterene, urea, amiloride, bumetamide, chlorothiazide, ethacrynic acid, furosemide, hydrochlorothiazide, triamterene, a benzothiadiazine derivative, a sulfonamide derivative, an organic mercurial diuretic, a loop diuretic, or a potassium-sparing diuretic.

20. A method according to claim 19, wherein the diuretic comprises althiazide, bendroflumethiazide, benzthiazide, benzylhydrochlorothiazide, buthiazide, chlorothiazide, chlorthalidone, cyclopenthiazide, cyclothiazide, epithiazide, ethiazide, fenquizone, hydrochlorothiazide, hydroflumethiazide, indapamide, methyclothiazide, meticrane, metolazone, paraflutizide, polythiazide, quinethazone, teclothiazide, trichlormethiazide, acetazolamide, ambuside, azosemide, bumetamide, butazolamide, chloraminophenamide, clofenamide, clopamide, clorexolone, disulfamide, ethoxolamide, furosemide, mefruside, methazolamide, piretamide, torasemide, tripamide, xipamide, mercaptomerin sodium, merethoxylline, procaine, or mersalyl with thiophylline.

21. A method according to claim 2, wherein the first amount comprises a compound corresponding in structure to a formula selected from the group consisting of the following (or is a tautomer of any such compound, or a pharmaceutically-acceptable salt any such compound or tautomer):

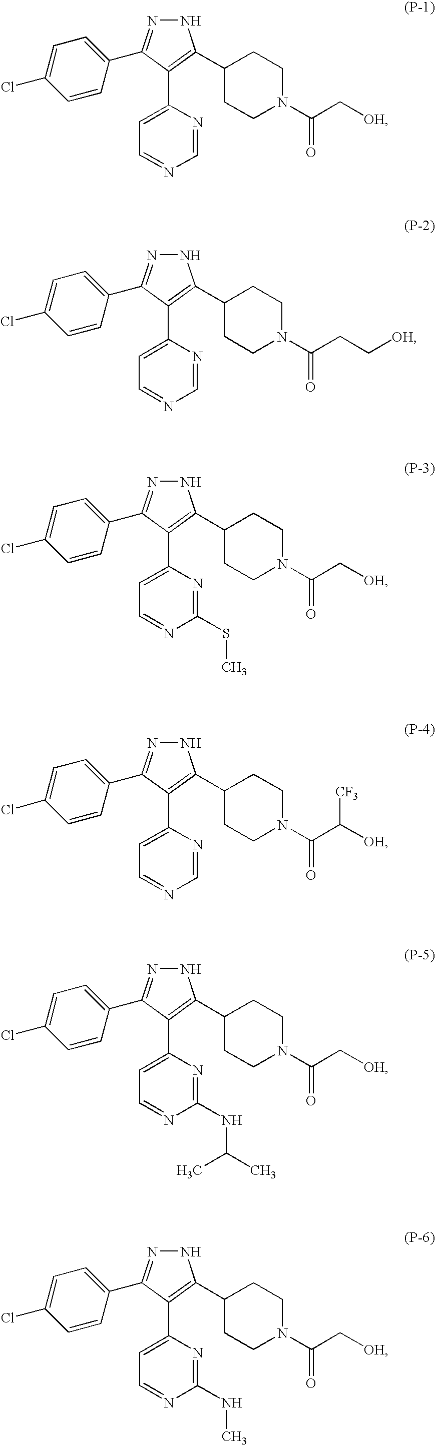

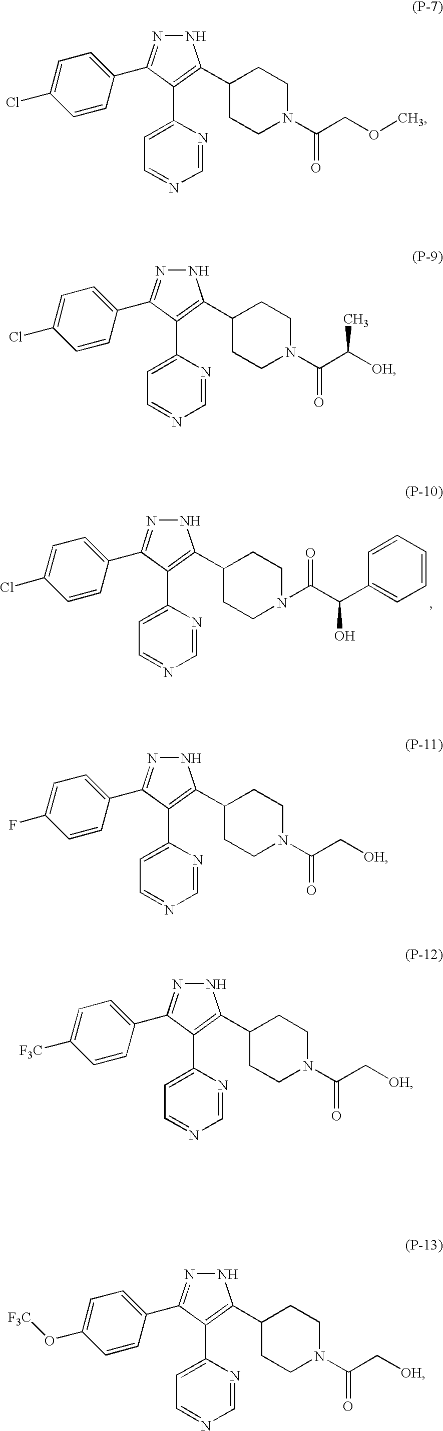

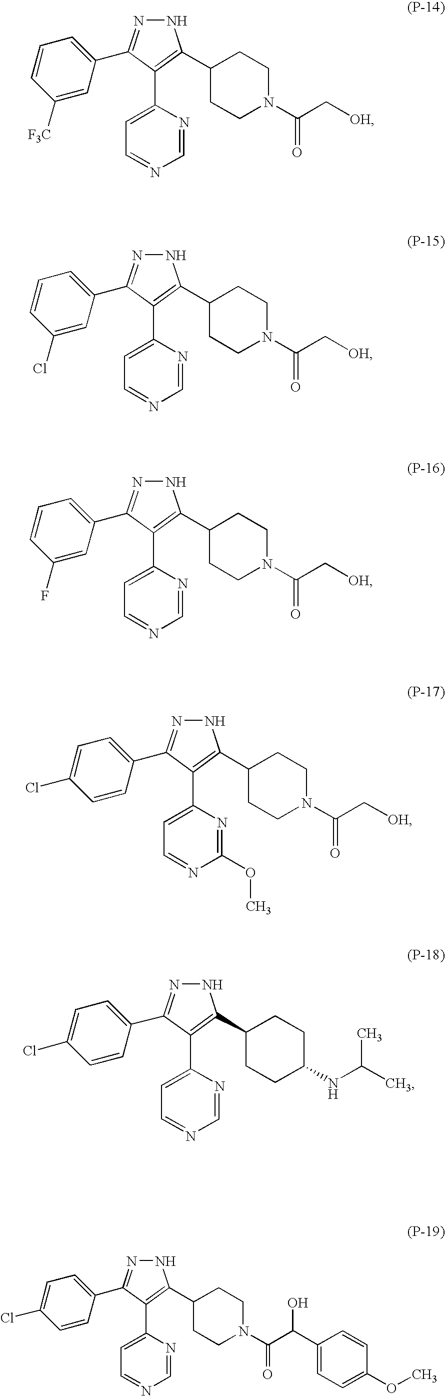

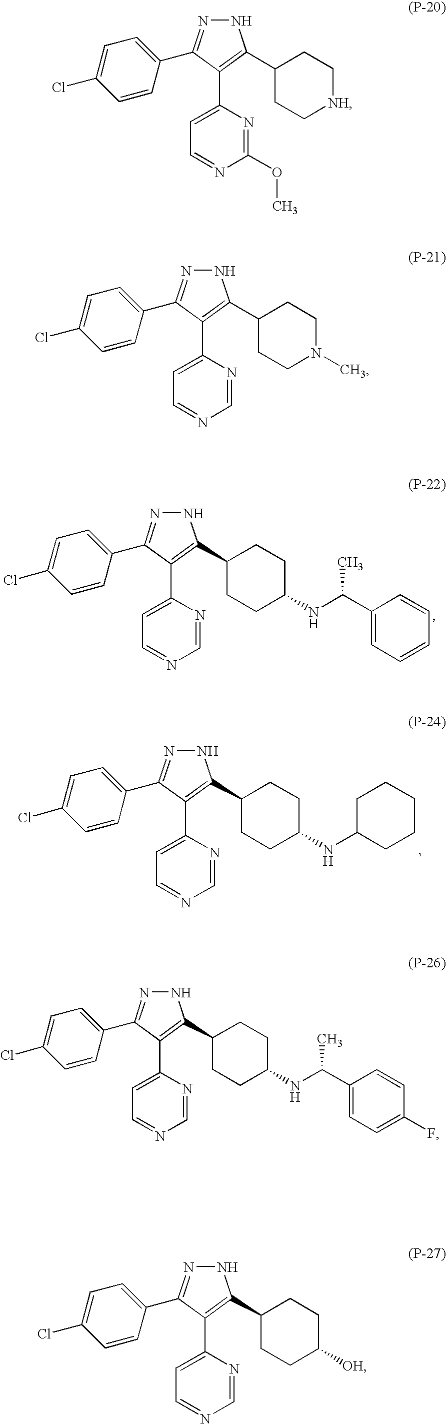

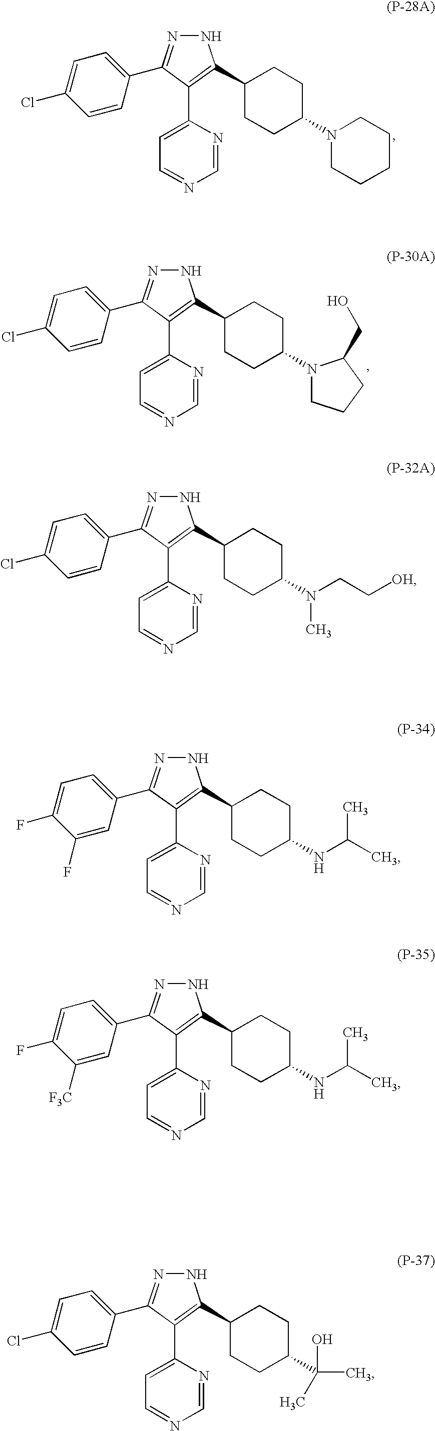

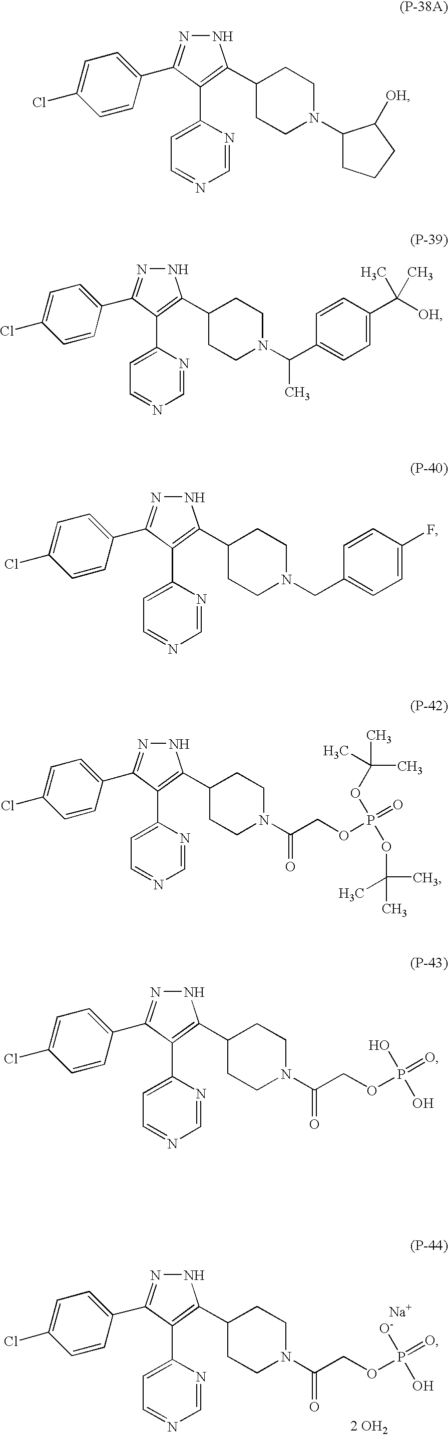

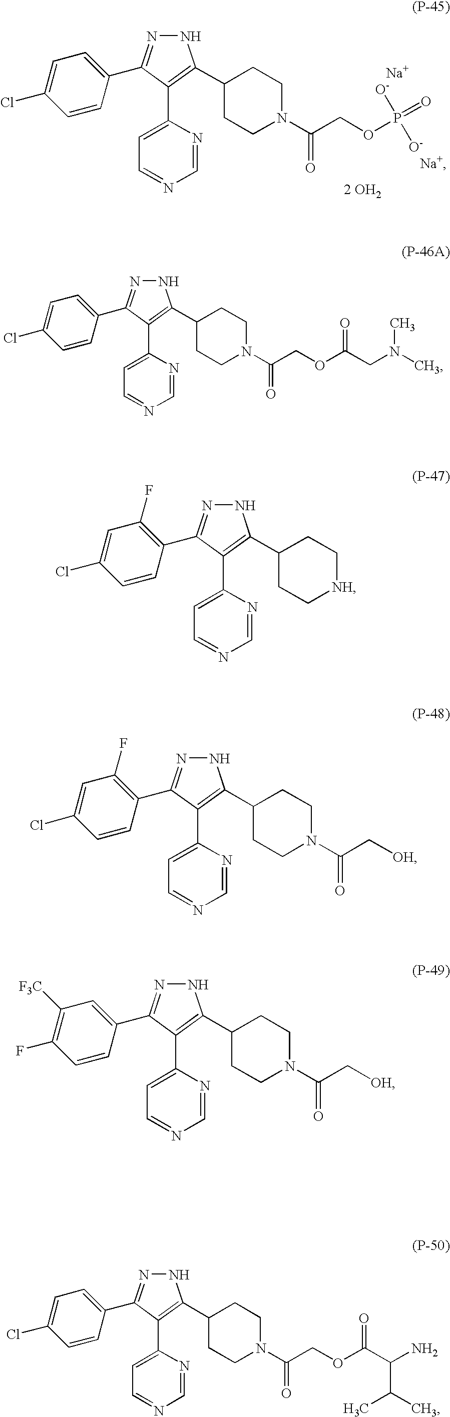

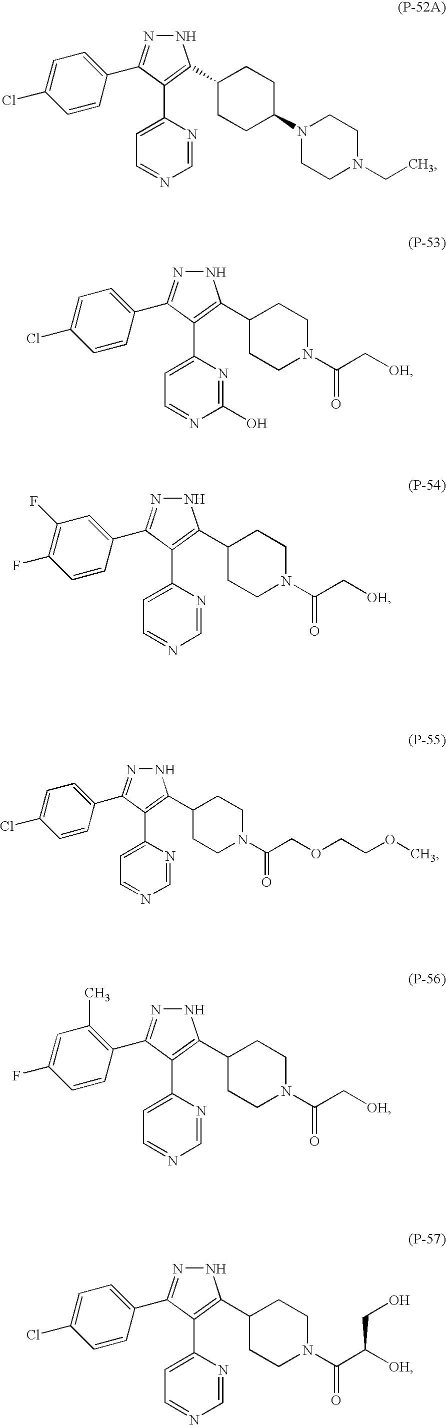

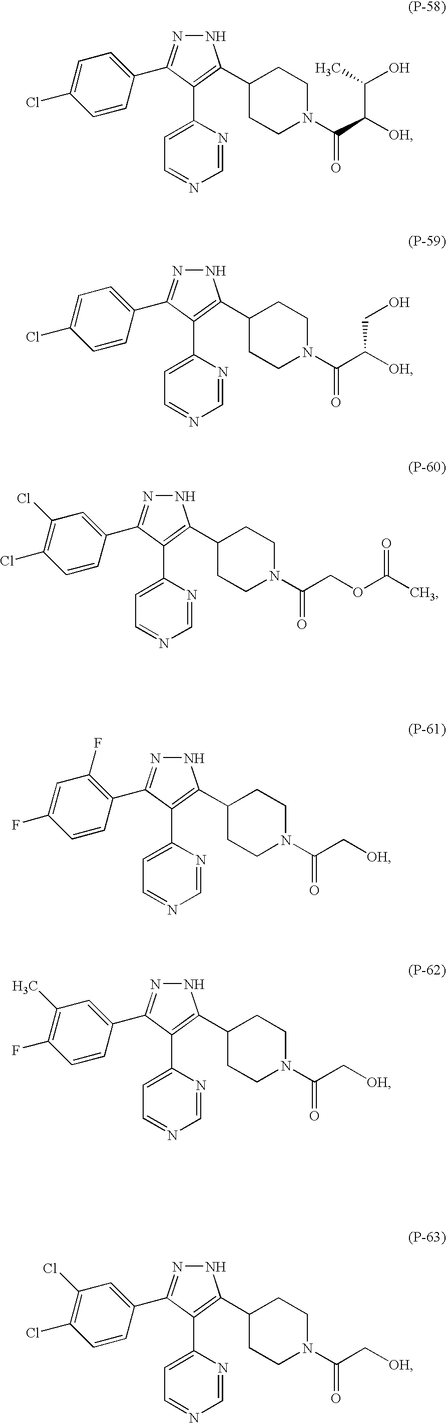

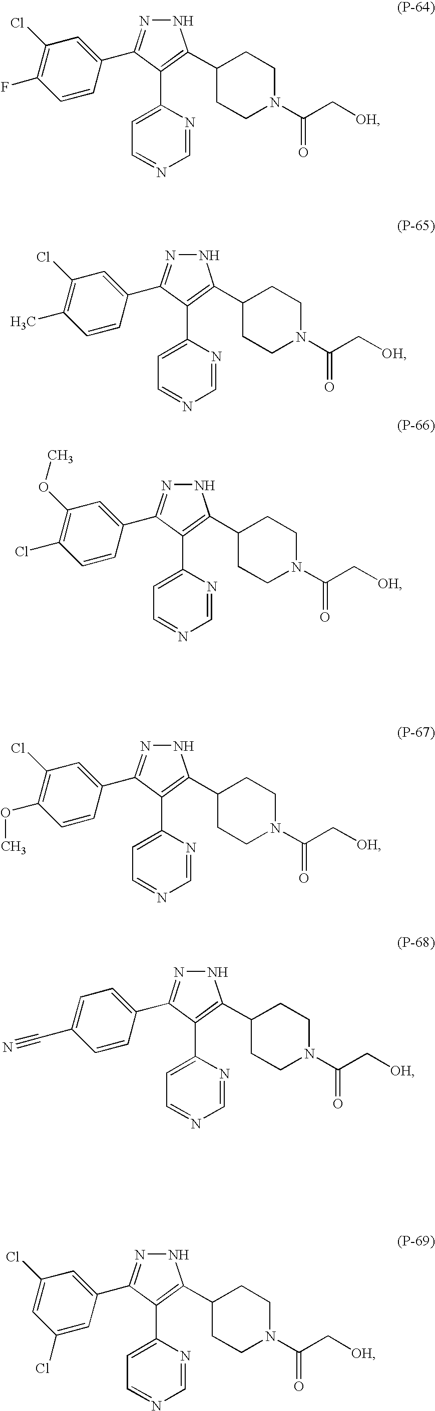

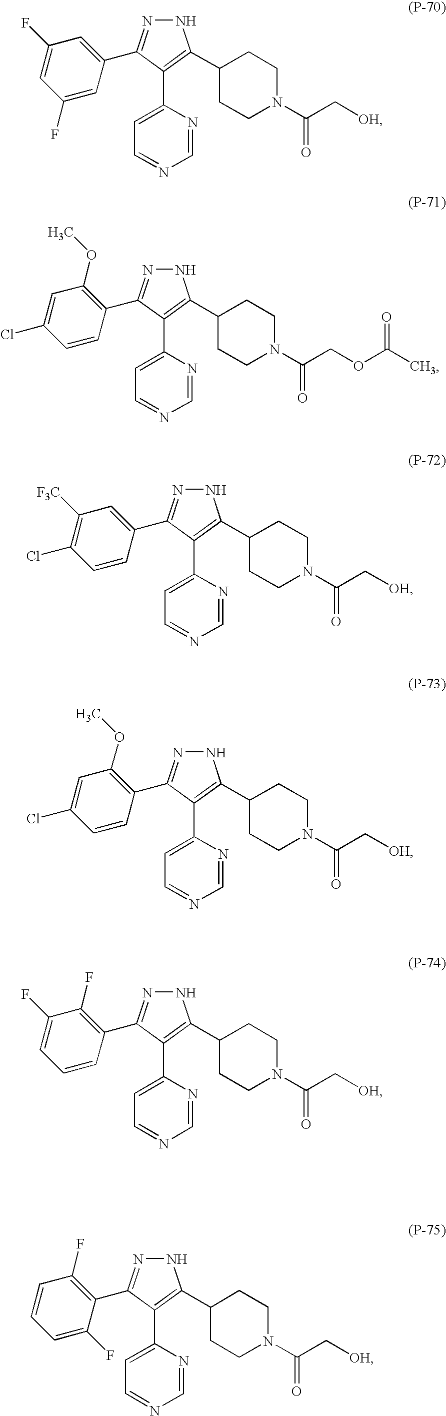

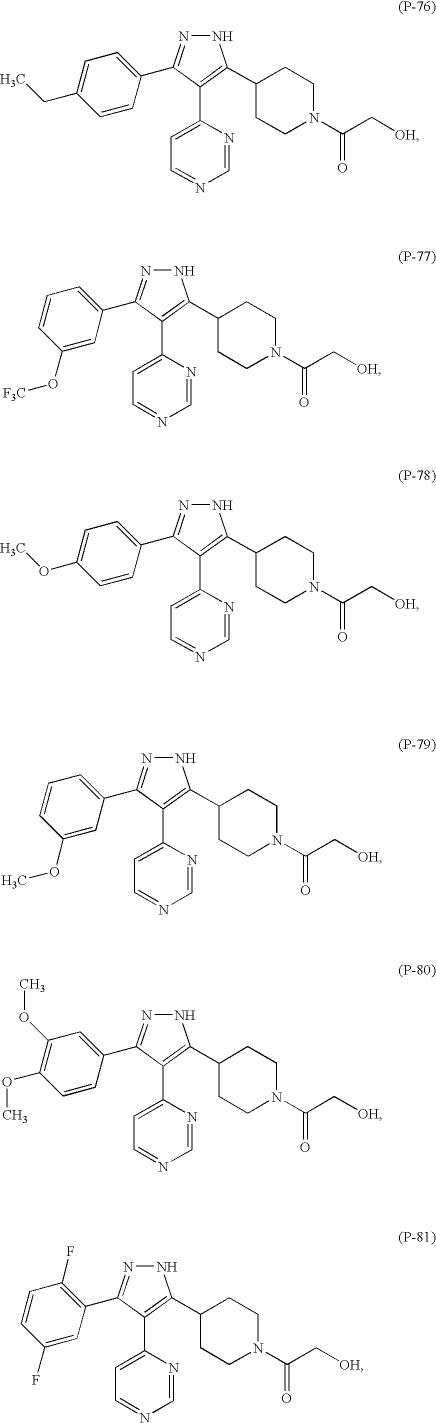

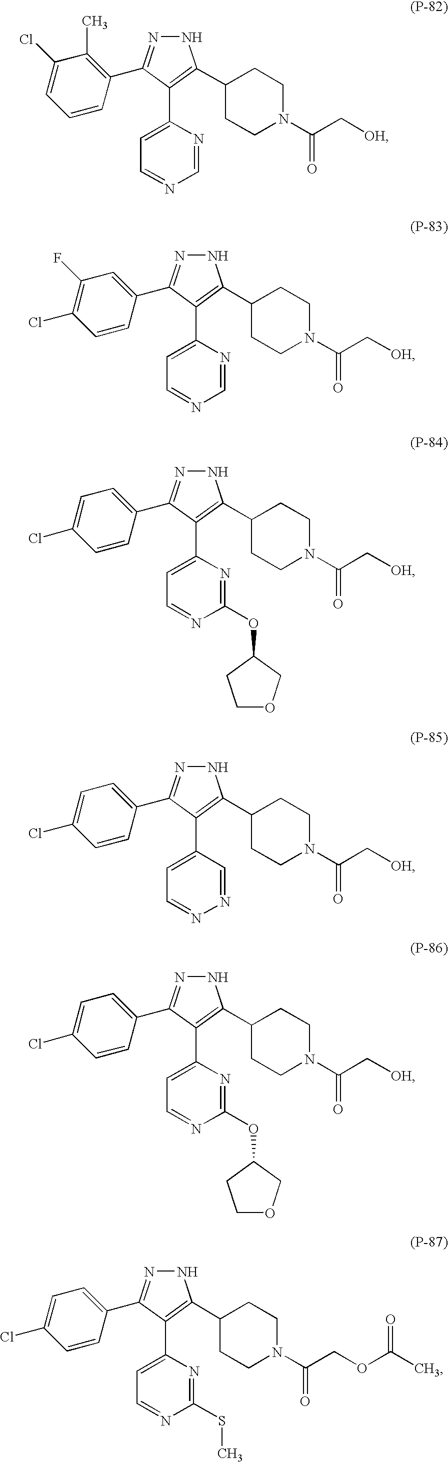

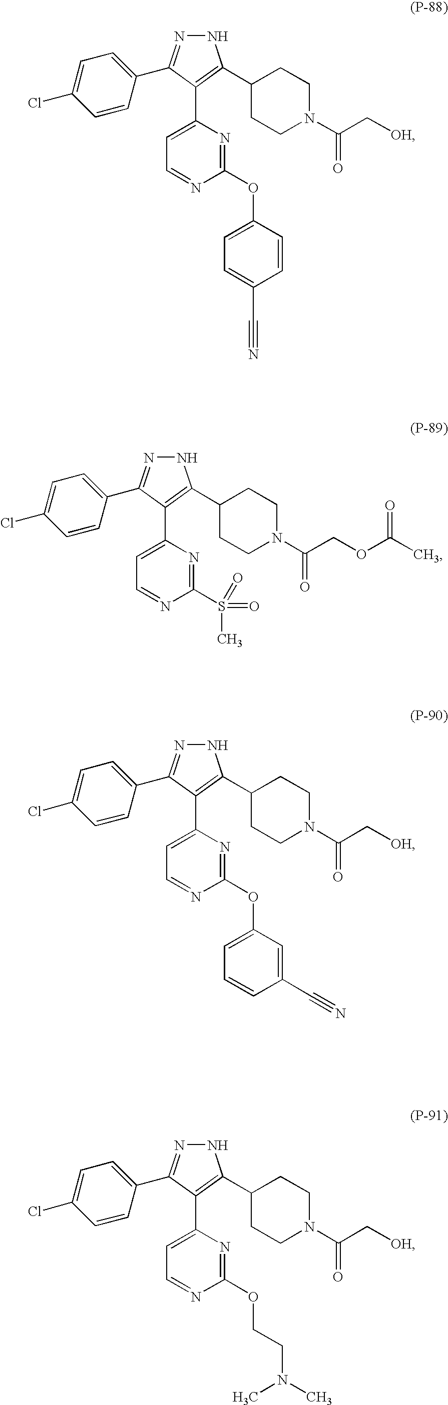

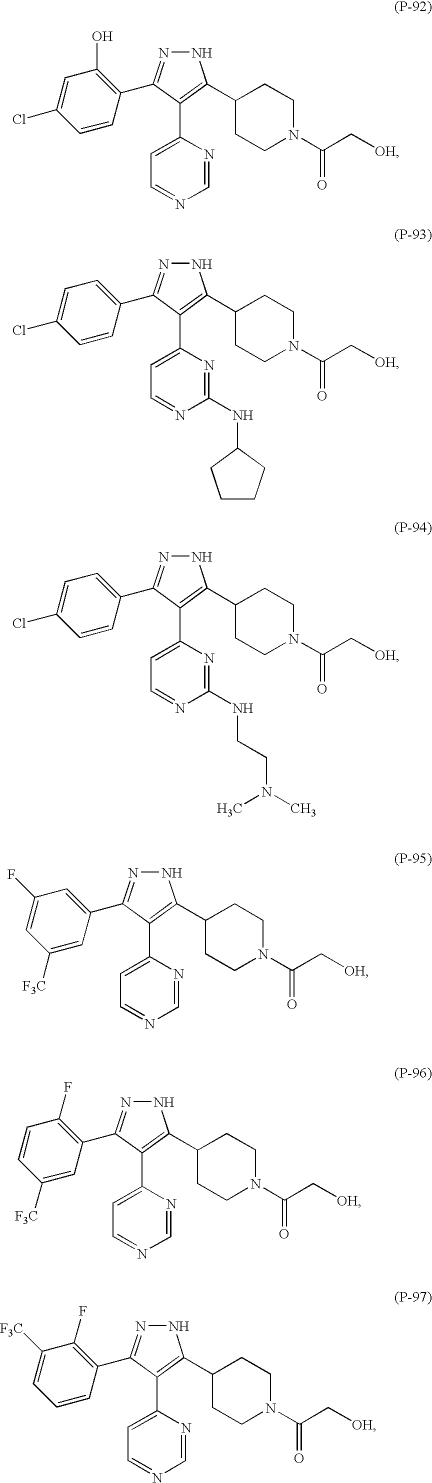

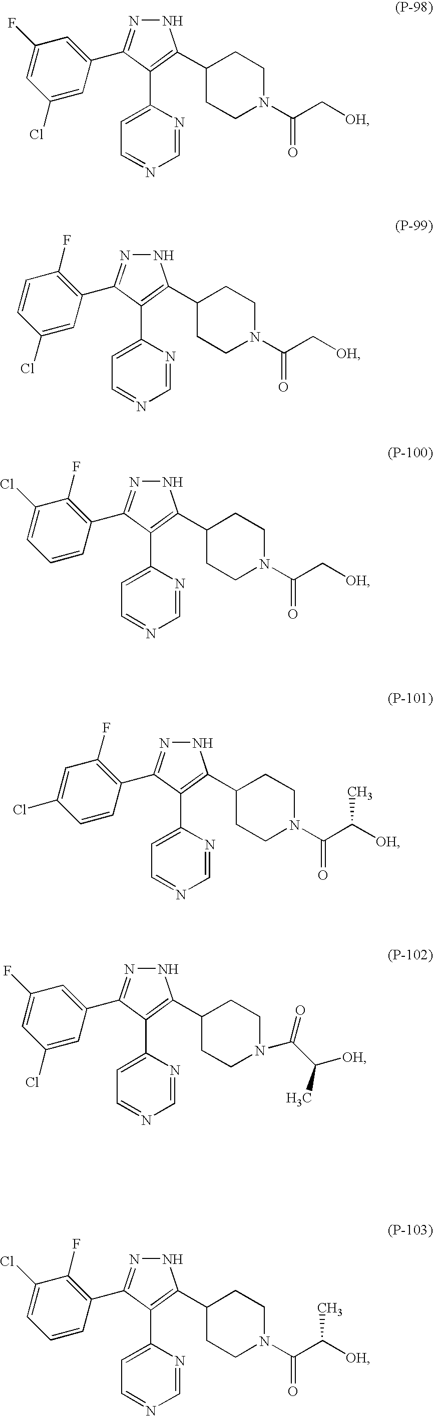

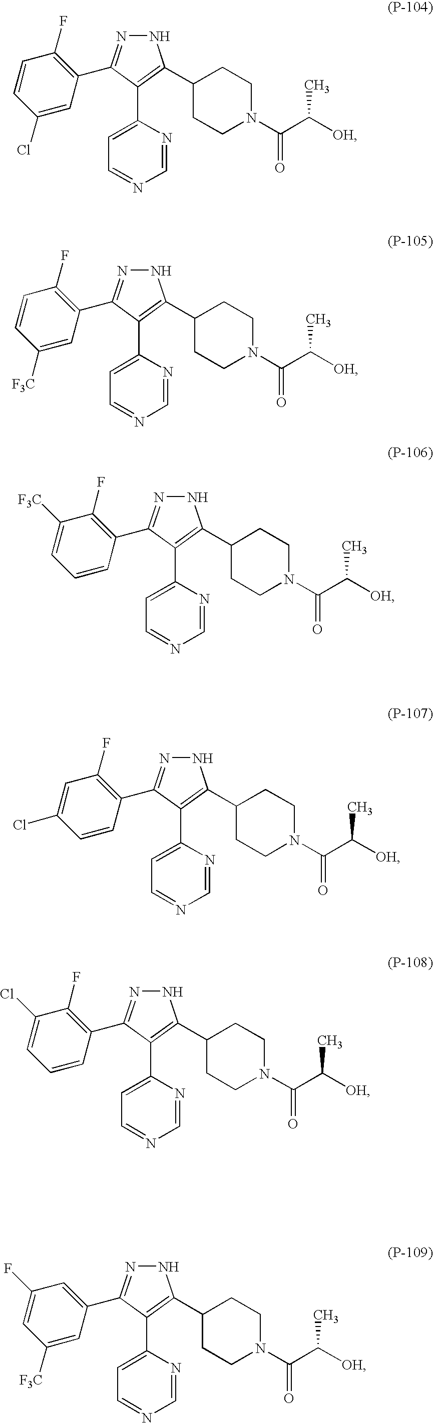

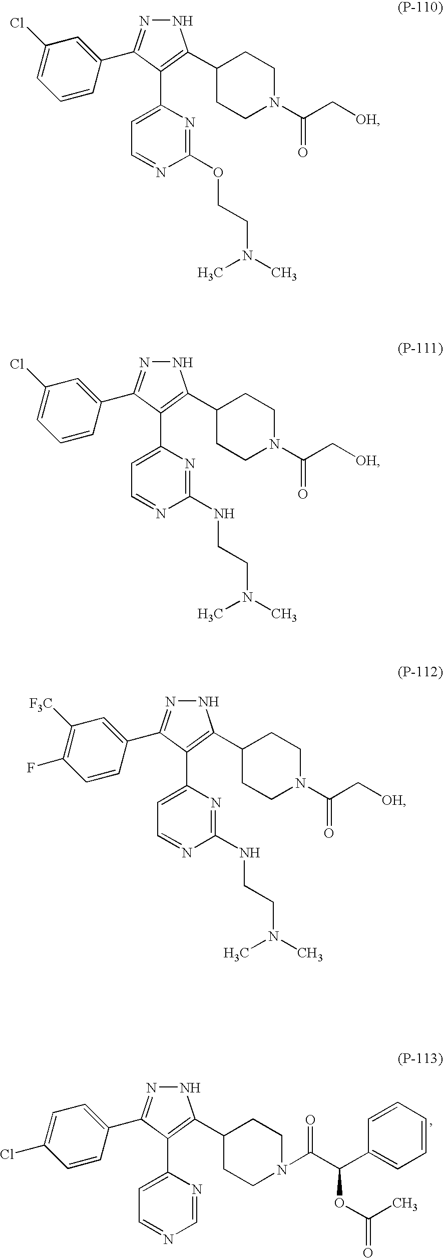

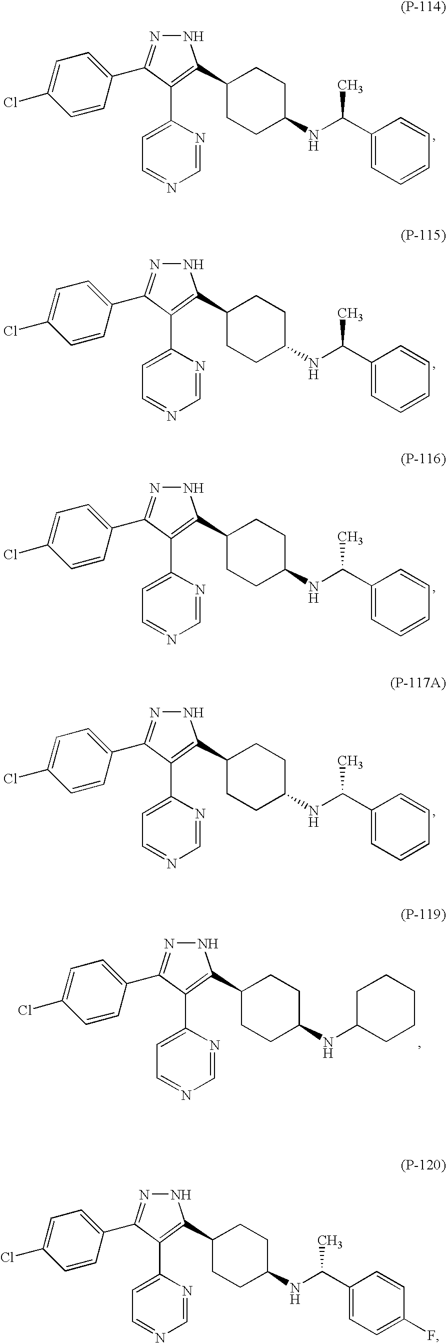

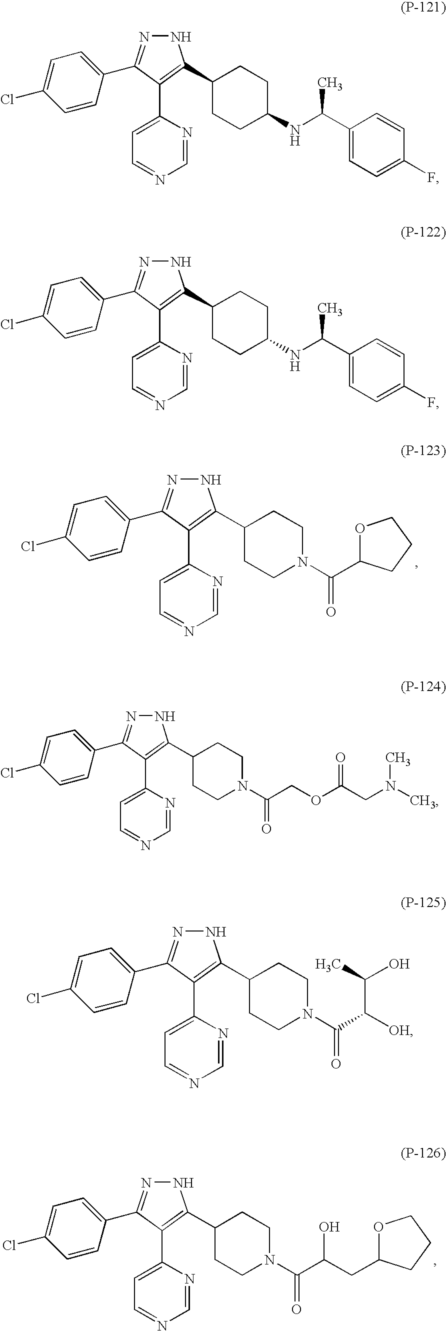

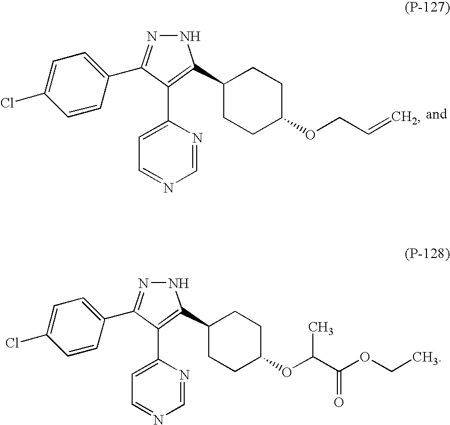

22. A method according to claim 2, wherein the first amount comprises a compound corresponding in structure to a formula selected from the group consisting of the following (or is a tautomer of any such compound, or a pharmaceutically-acceptable salt any such compound or tautomer):

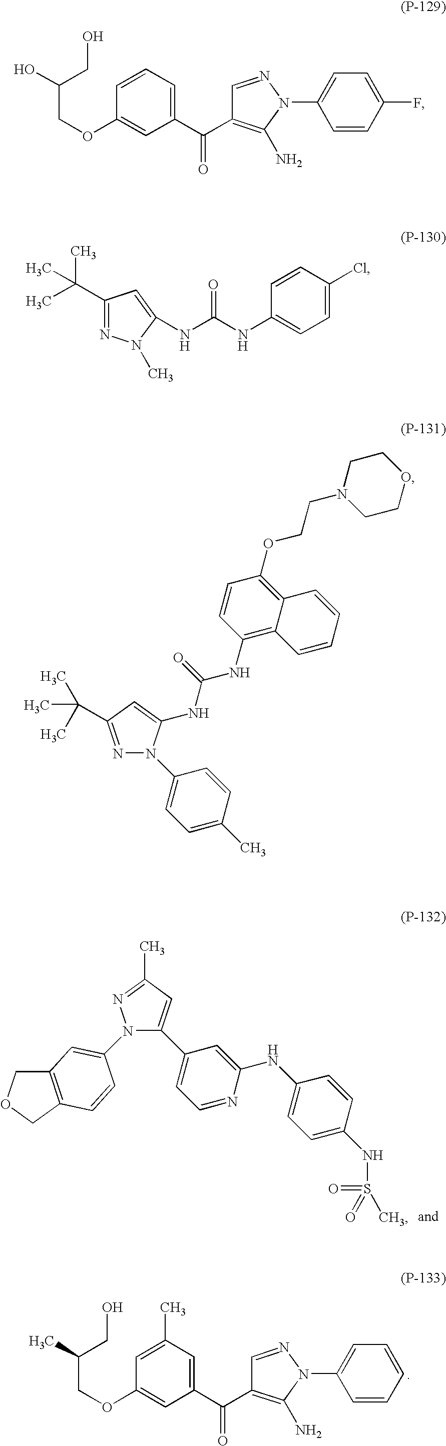

23. A method for treating a pathological condition in a mammal, wherein:
the method comprises administering to the mammal:
a first amount of a compound that comprises a p38-kinase inhibitor, and
a second amount of a compound that comprises an aldosterone antagonist or diuretic; and
the first and second amounts of the compounds together comprise a therapeutically-effective amount of the compounds; and
the pathological condition comprises a cardiovascular disease, glomerulosclerosis, end-stage renal disease, acute renal failure, diabetic nephropathy, reduced renal blood flow, increased glomerular filtration fraction, decreased glomerular filtration rate, decreased creatine clearance, renal arteriopathy, ischemic renal lesions, vascular damage in the kidney, vascular inflammation in the kidney, malignant nephrosclerosis, thrombotic vascular disease, proliferative arteriopathy, atherosclerosis, decreased vascular compliance, retinopathy, neuropathy, edema, or insulinopathy.

24. A method according to claim 23, wherein the pathological condition comprises ischemic renal retraction, thrombonecrosis of renal capillary tufts, renal arteriolar fibrinoid necrosis, thrombotic microangiopathic lesions affecting renal glomeruli or microvessels, atherosclerosis, mural fibrinoid necrosis, extravasation of red blood cells, fragmentation of red blood cells, luminal thrombosis, mural thrombosis, swollen myointimal cells surrounded by mucinous extracellular matrix or nodular thickening, pathological vascular stiffness or reduced ventricular compliance, or retinopathy.

25. A method according to claim 23, wherein the pathological condition comprises a cardiovascular disease.

26. A method according to claim 25, wherein the cardiovascular disease comprises hypertension, vascular inflammation in the heart, coronary angioplasty, coronary thrombosis, cardiac lesions, myocarditis, coronary artery disease, heart failure, arrhythmia, diastolic dysfunction, systolic dysfunction, ischemia, cardiomyopathy, sudden cardiac death, myocardial fibrosis, vascular fibrosis, impaired arterial compliance, myocardial necrotic lesions, vascular damage in the heart, myocardial infarction, left ventricular hypertrophy, decreased ejection fraction, vascular wall hypertrophy in the heart, or endothelial thickening

27. A method according to claim 26, wherein the cardiovascular disease comprises fibrinoid necrosis of coronary arteries, congestive heart failure, chronic heart failure, acute heart failure, left ventricular diastolic dysfunction, diastolic heart failure, impaired diastolic filling, myocardial ischemia, hypertrophic cardiomyopathy, dilated cardiomyopathy, an acute post-myocardial-infarction condition, or a chronic post-myocardial-infarction condition.

28. A method according to claim 26, wherein the cardiovascular disease comprises hypertension.

29. A method according to claim 26, wherein the cardiovascular disease comprises heart failure.

30. A method according to claim 23, wherein the p38-kinase inhibiting compound comprises a substituted imidazole.

31. A method according to claim 30, wherein the first amount comprises a compound corresponding in structure to a formula selected from the group consisting of the following (or is a tautomer of any such compound, or a pharmaceutically-acceptable salt any such compound or tautomer):

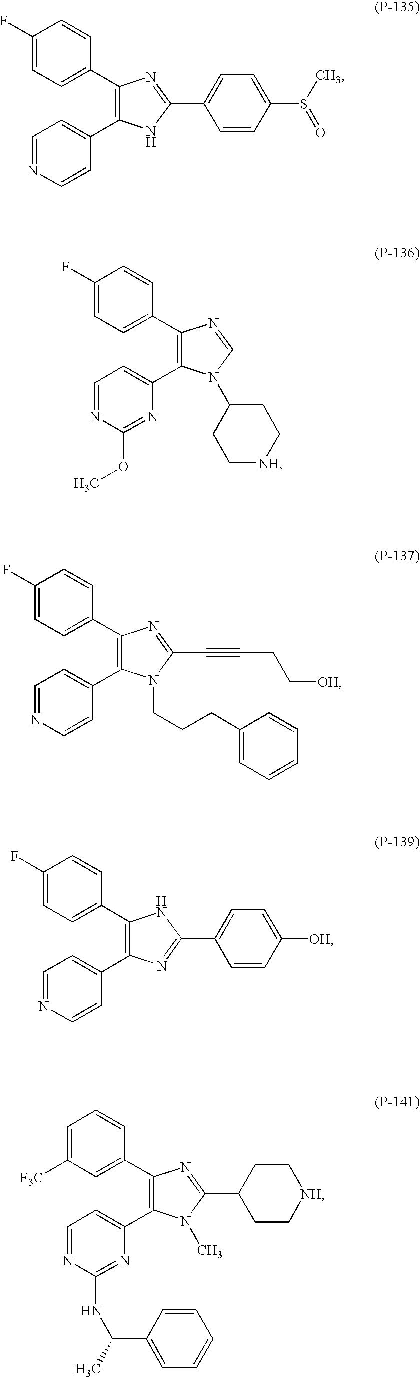

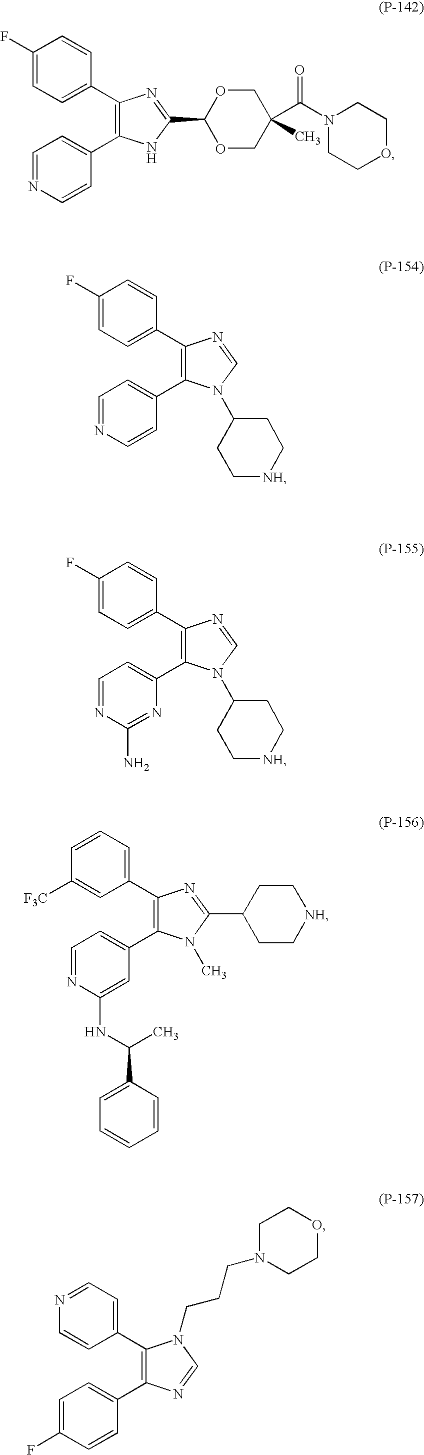

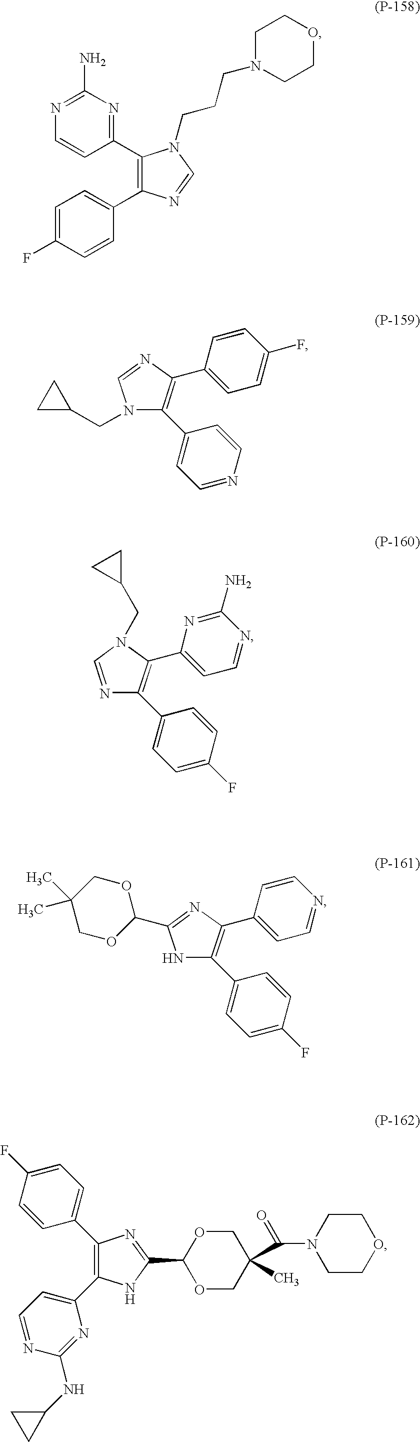

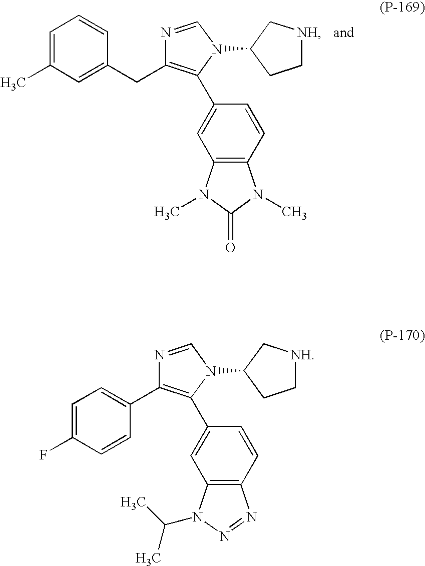

32. A method according to claim 23, wherein the p38-kinase inhibiting compound comprises a substituted pyrazole.

33. A method according to claim 32, wherein the first amount comprises a compound corresponding in structure to a formula selected from the group consisting of the following (or is a tautomer of any such compound, or a pharmaceutically-acceptable salt any such compound or tautomer):

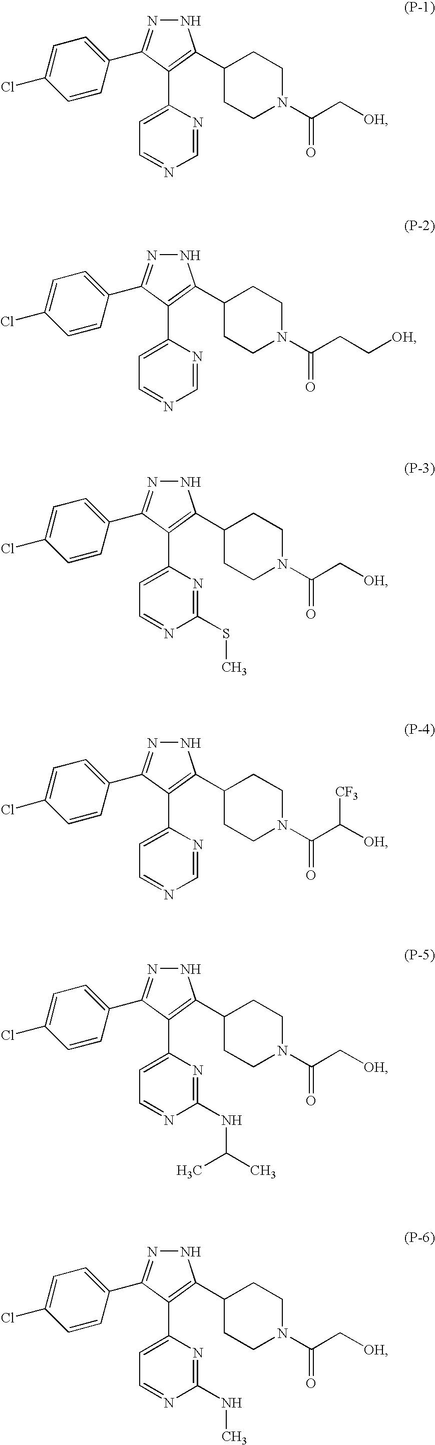

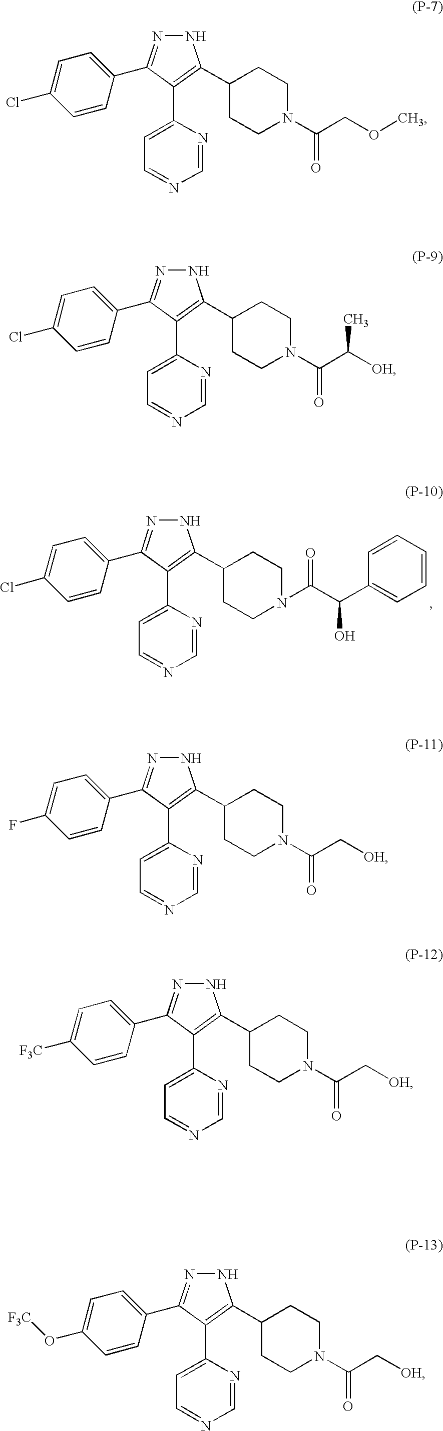

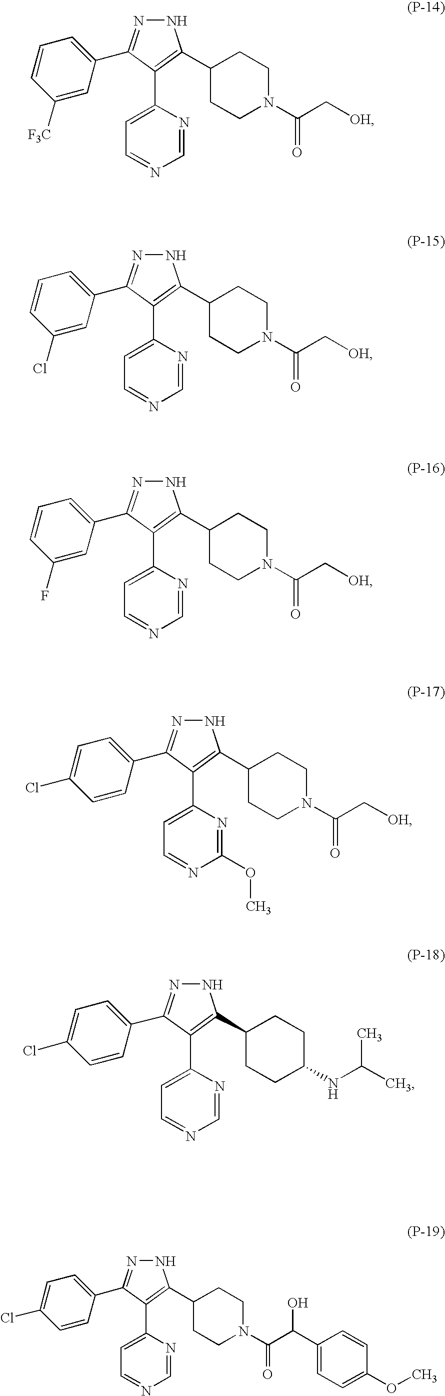

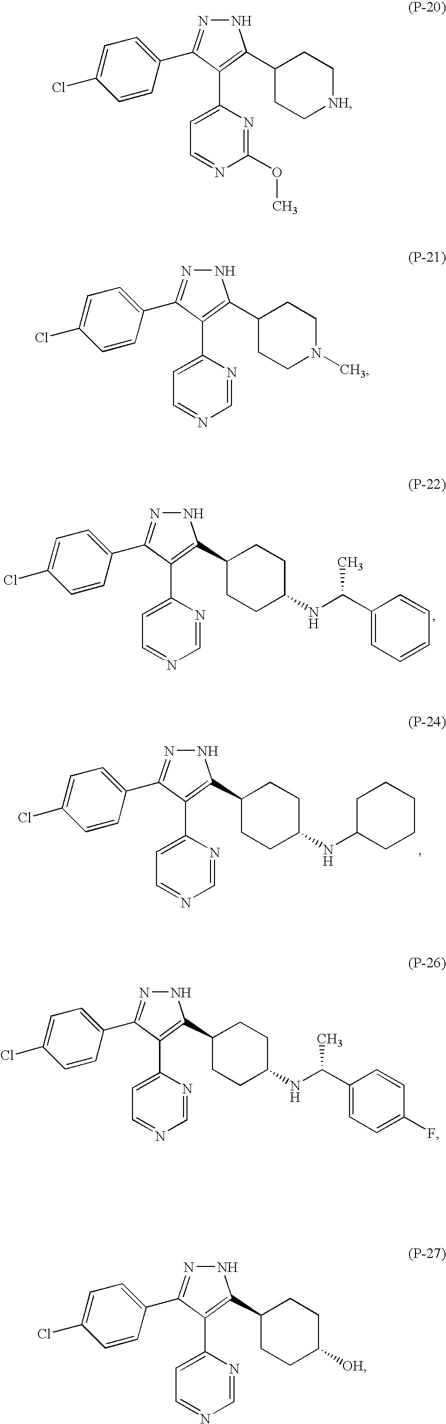

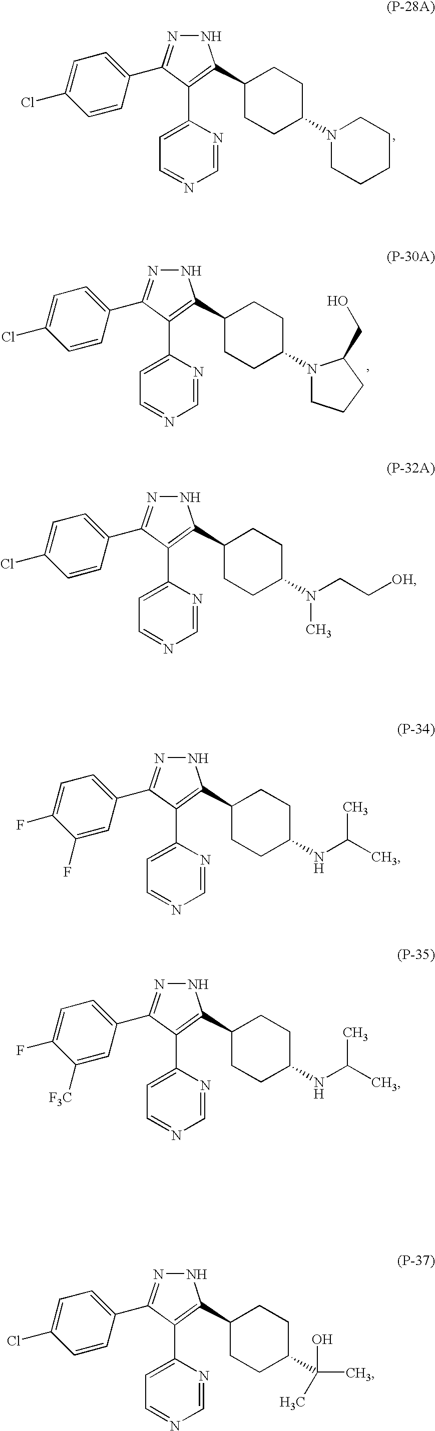

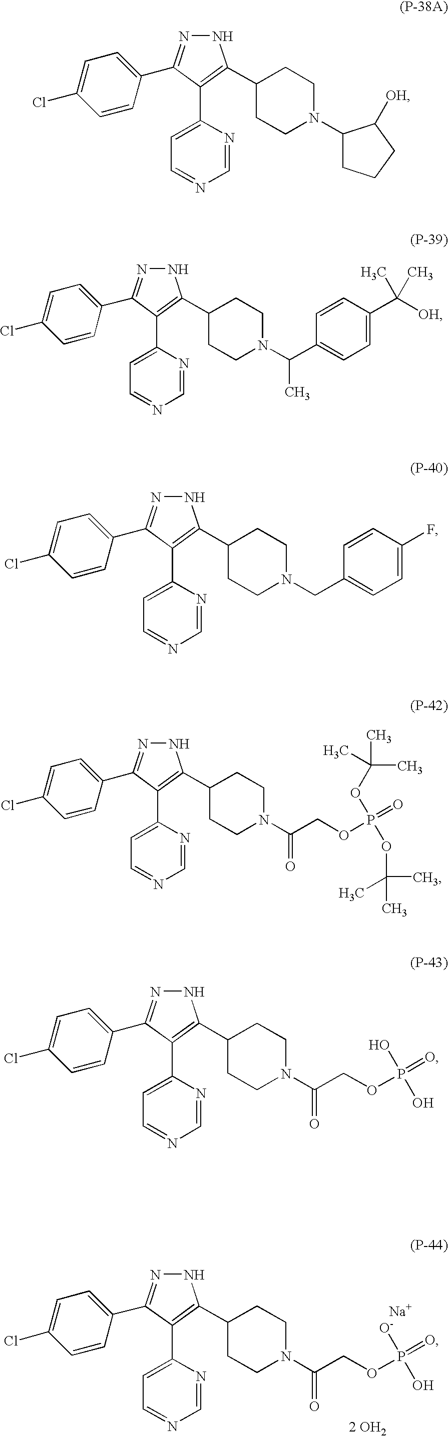

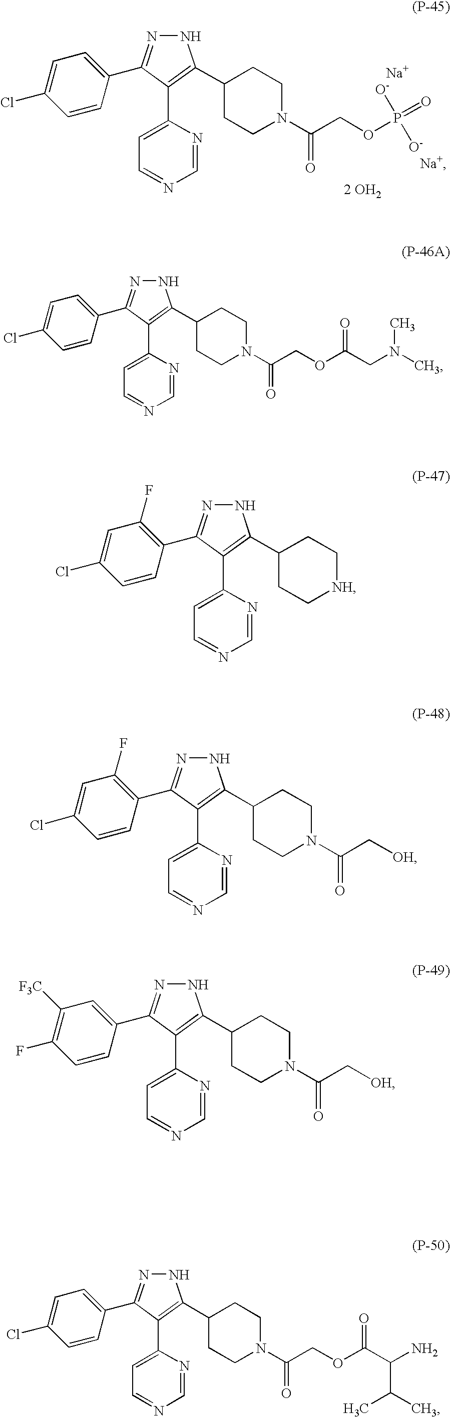

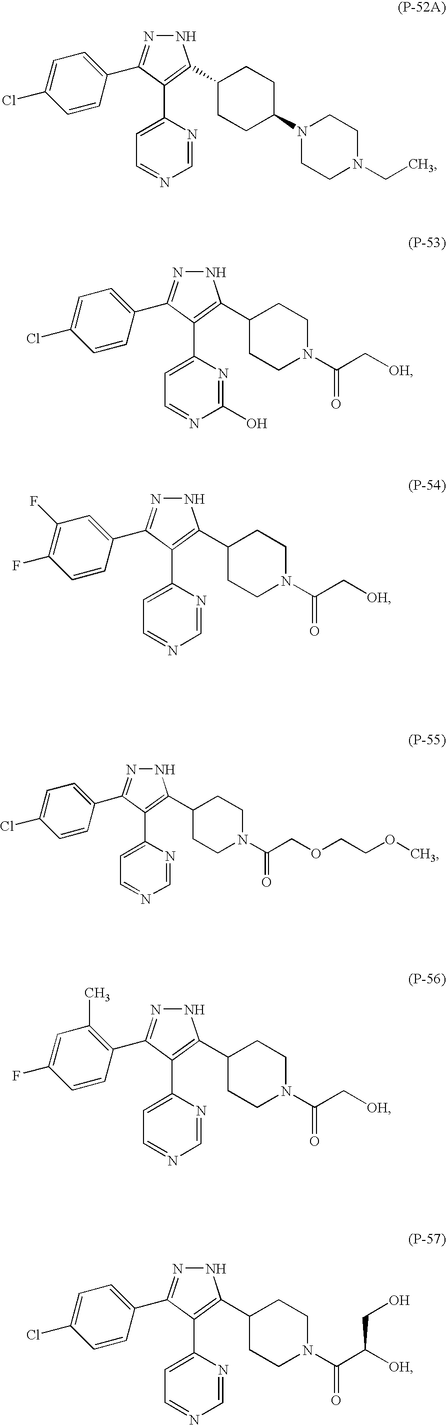

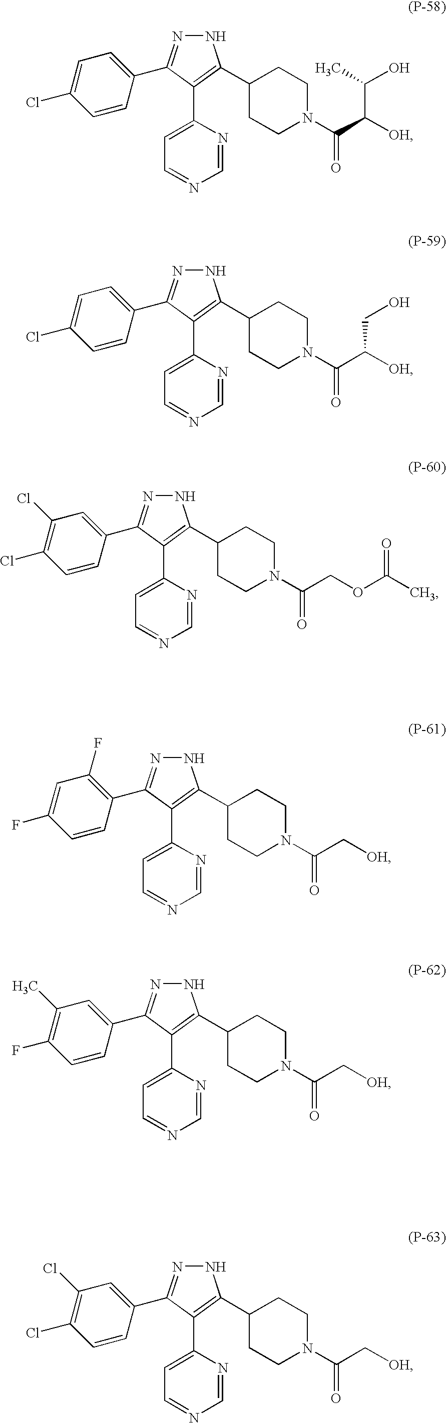

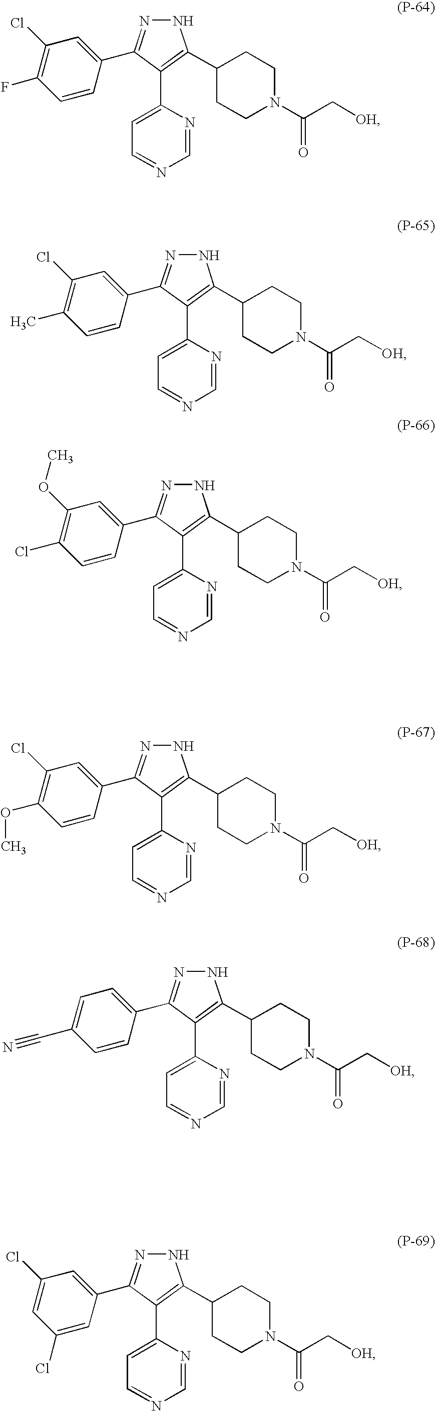

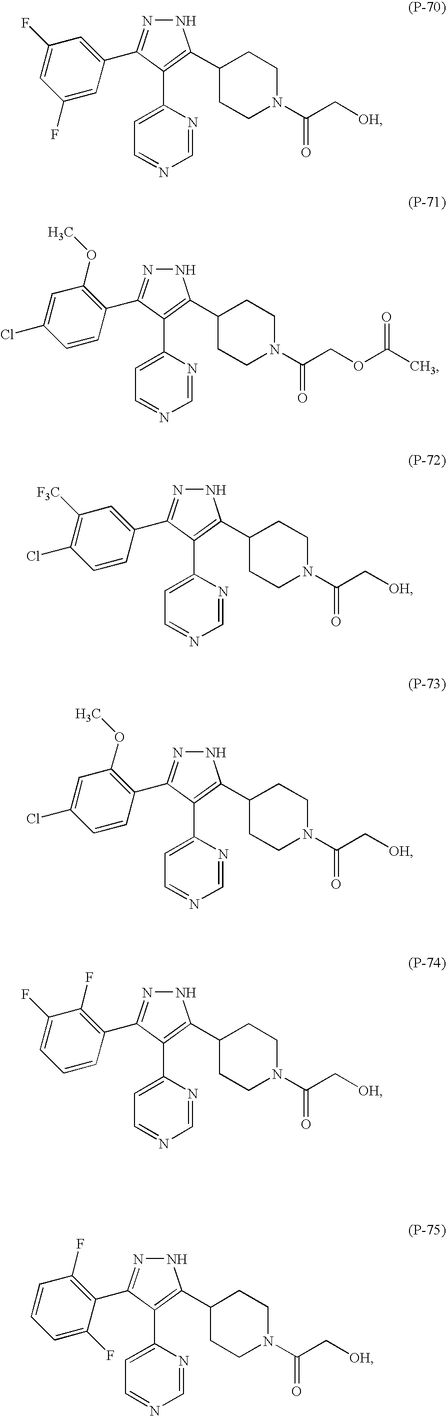

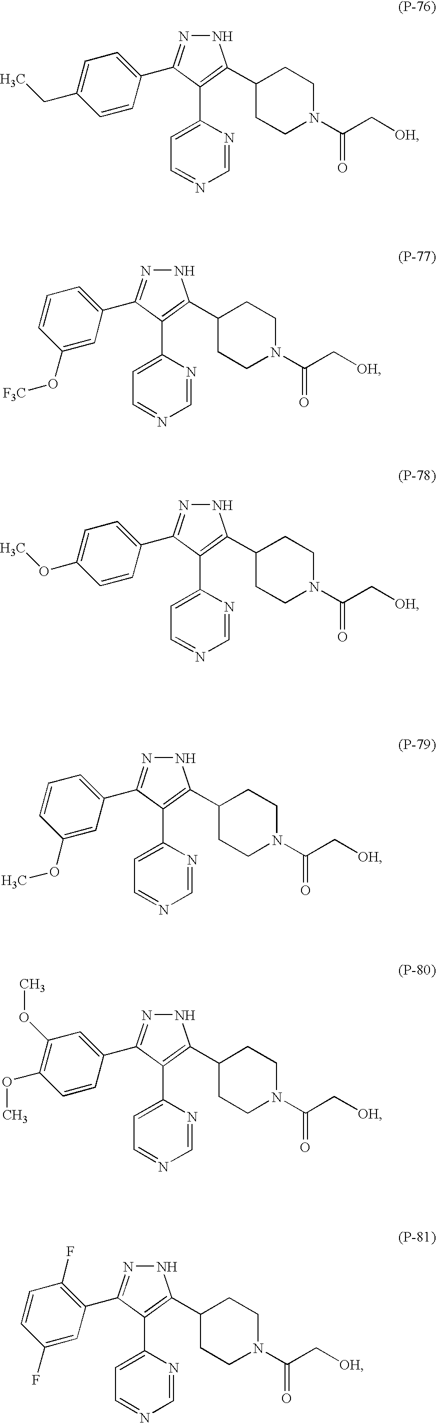

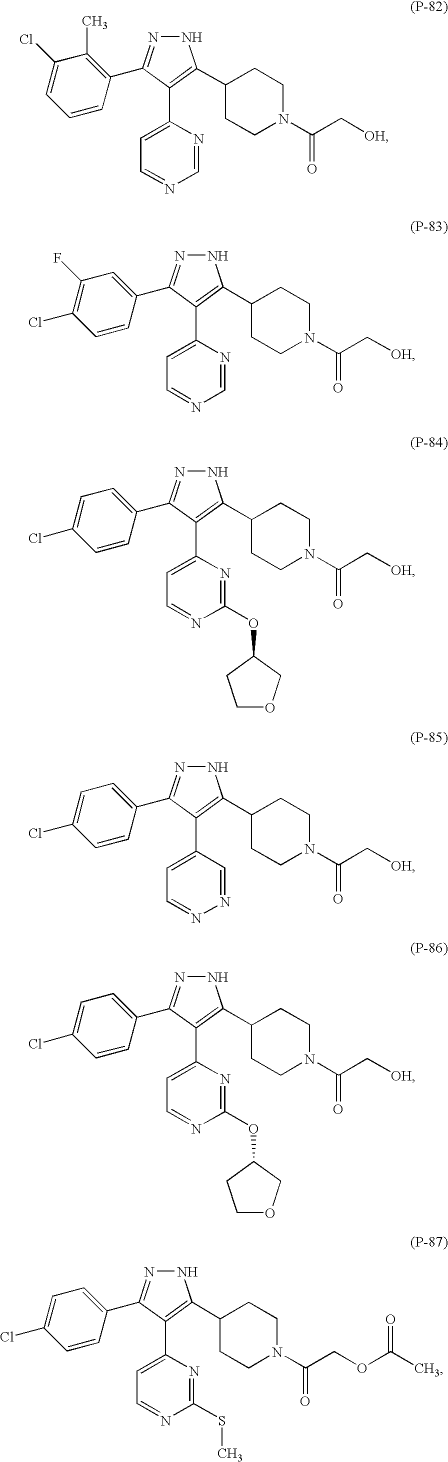

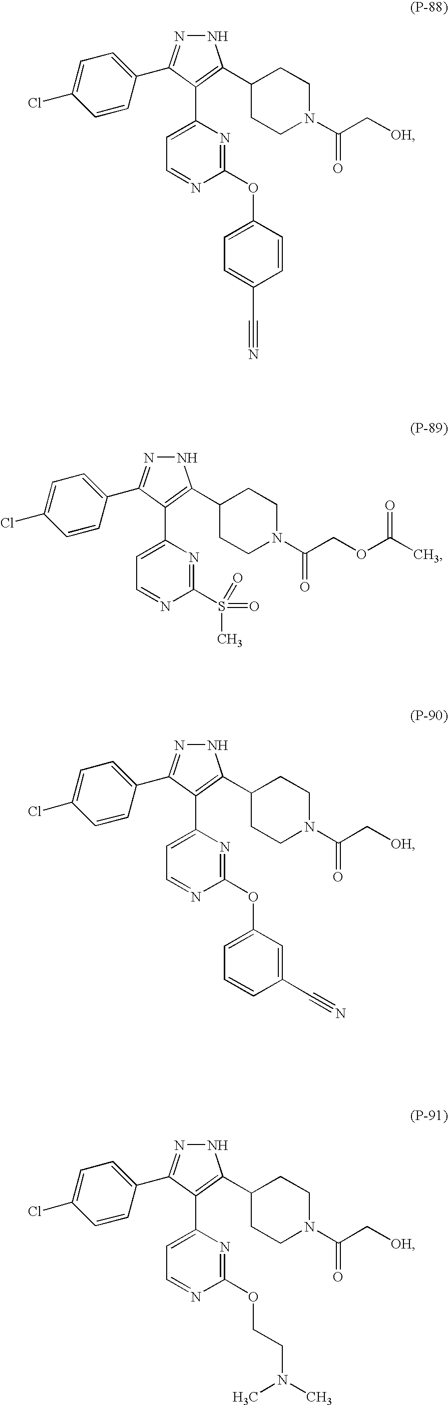

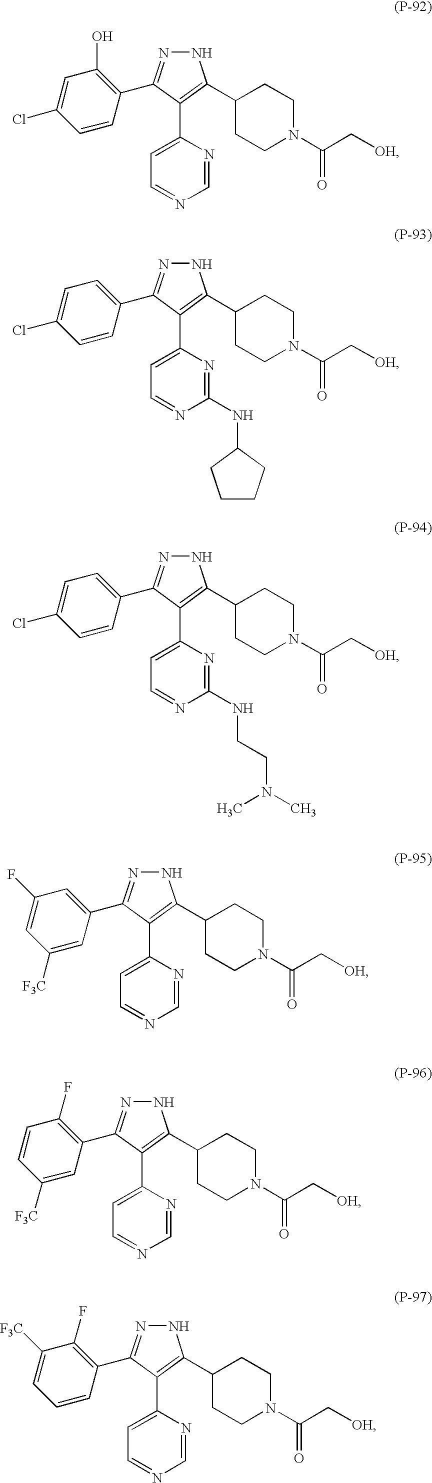

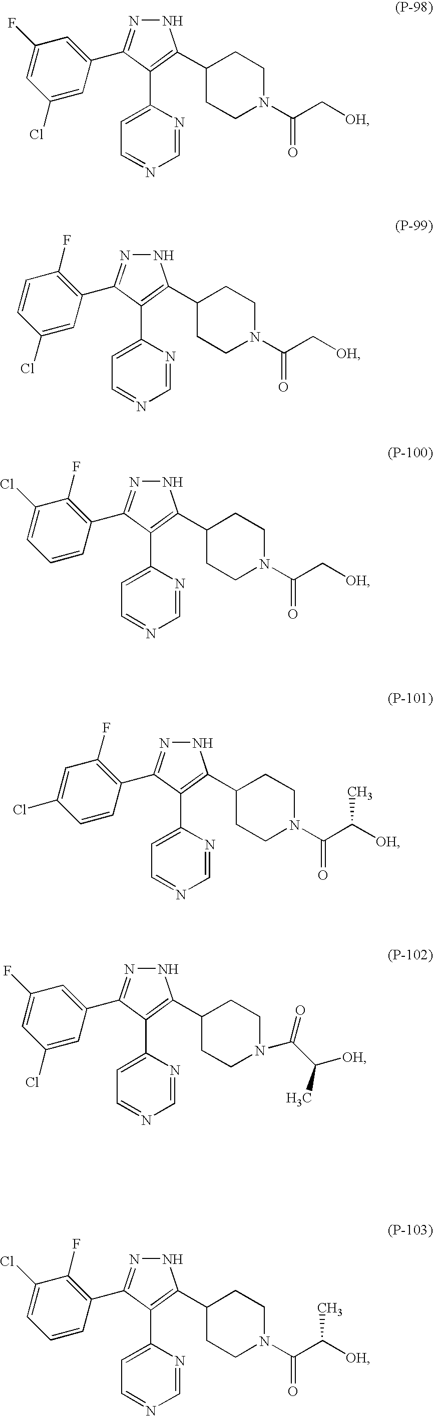

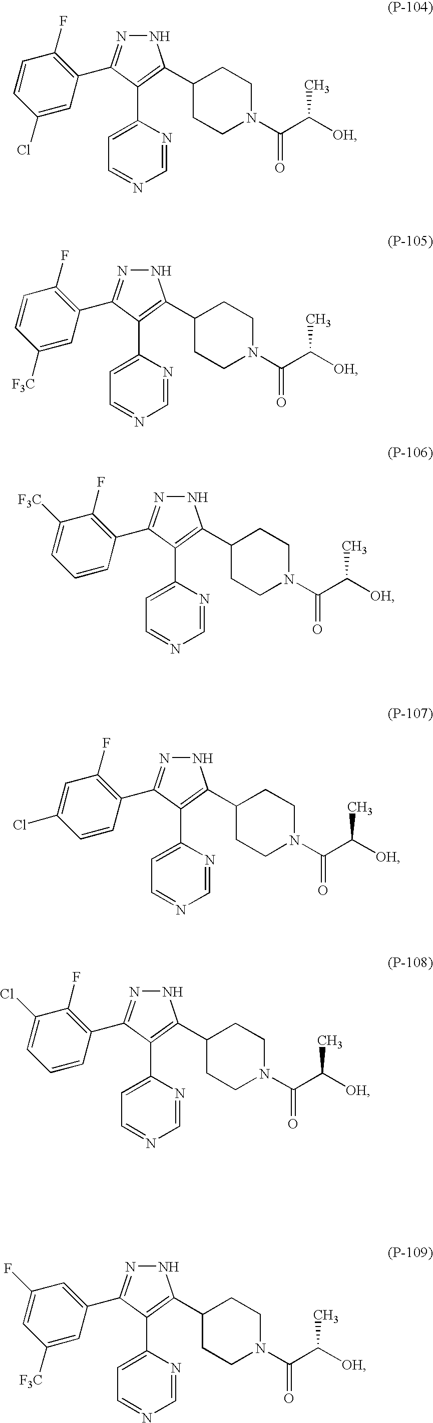

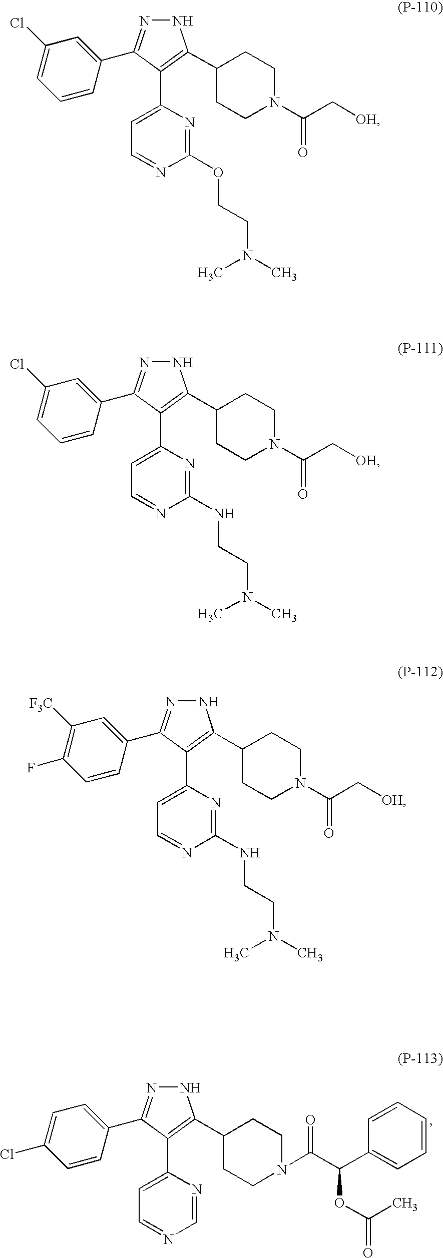

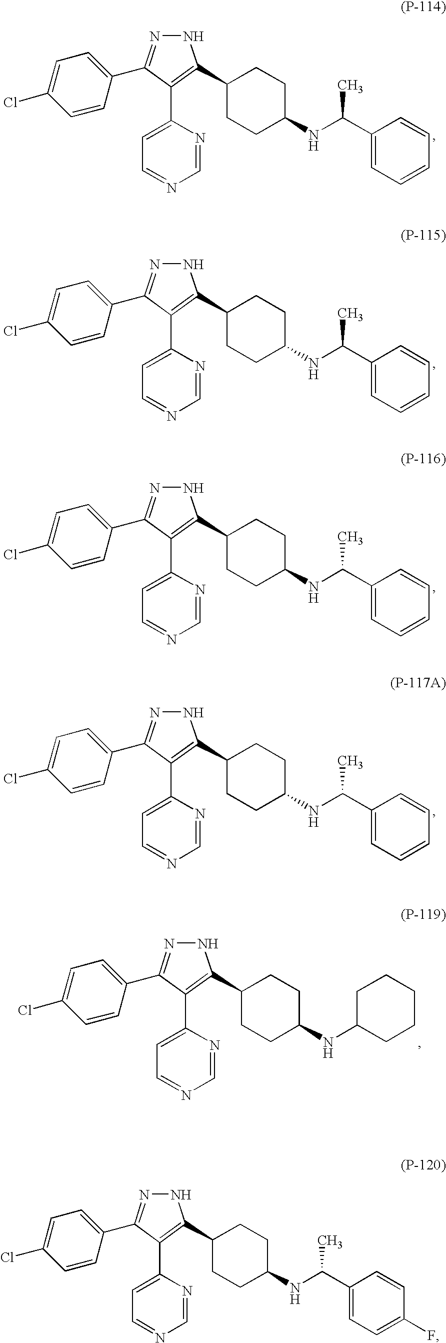

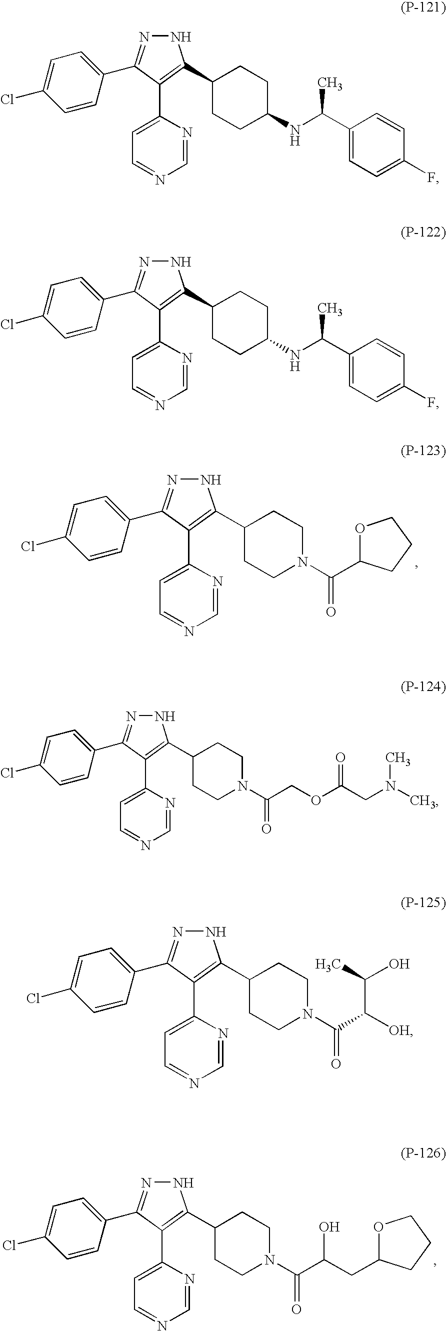

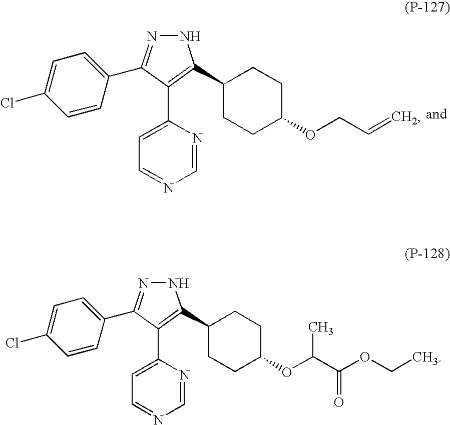

34. A method according to claim 32, wherein the first amount comprises a compound corresponding in structure to a formula selected from the group consisting of the following (or is a tautomer of any such compound, or a pharmaceutically-acceptable salt any such compound or tautomer):

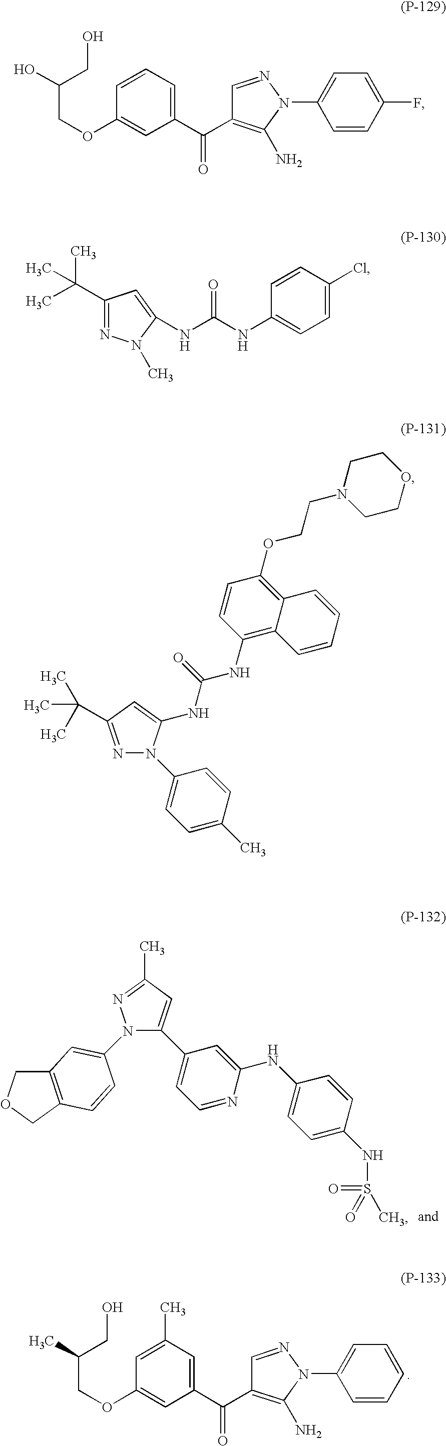

35. A method according to claim 23, wherein the first amount comprises a compound corresponding in structure to a formula selected from the group consisting of the following (or is a tautomer of any such compound, or a pharmaceutically-acceptable salt any such compound or tautomer):

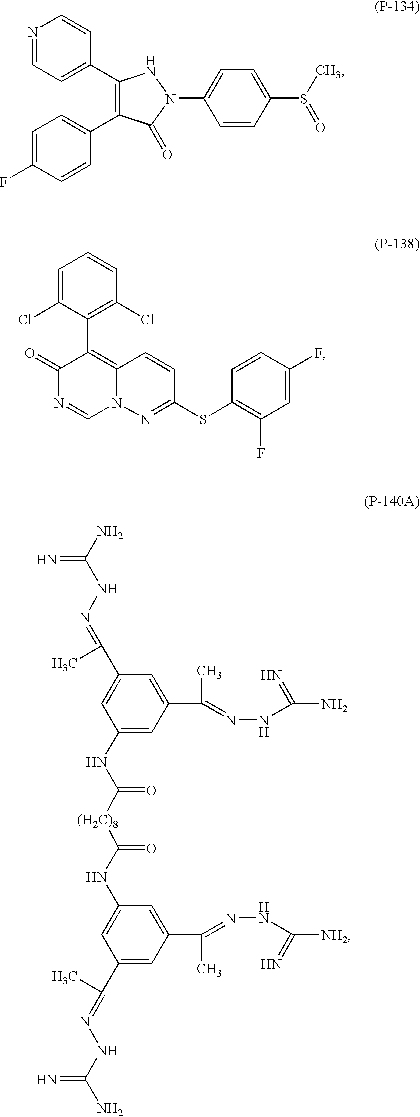

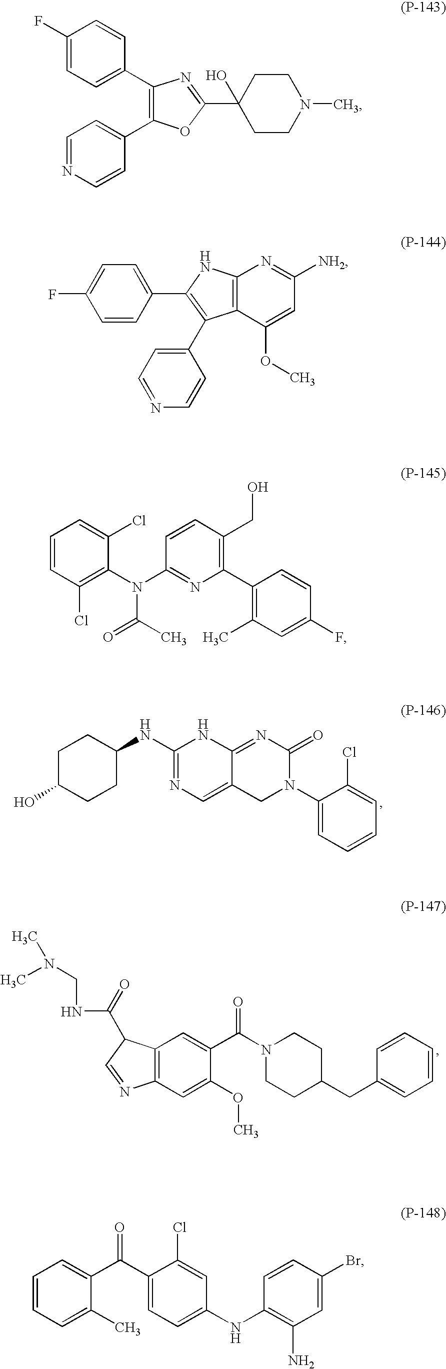

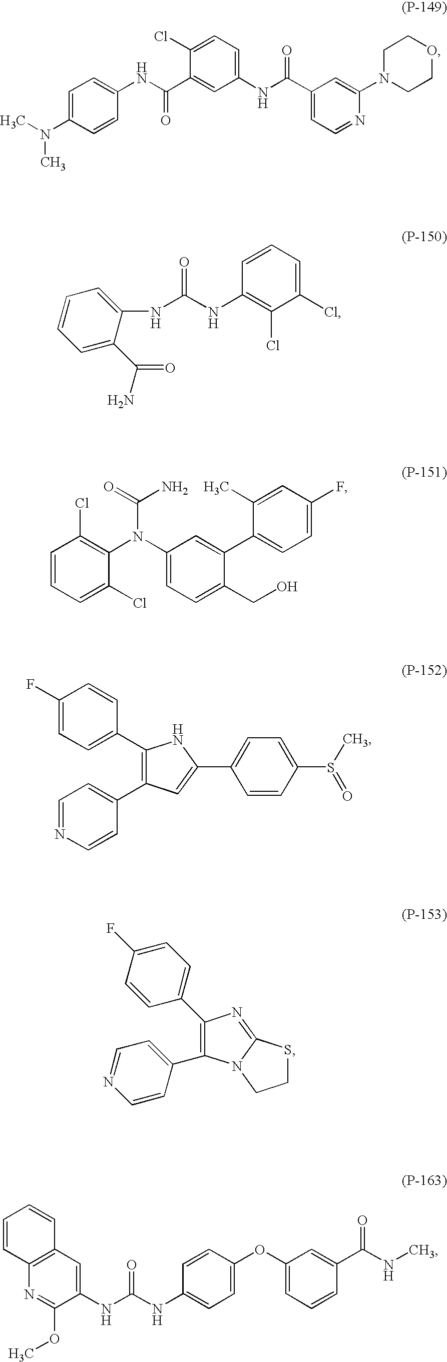

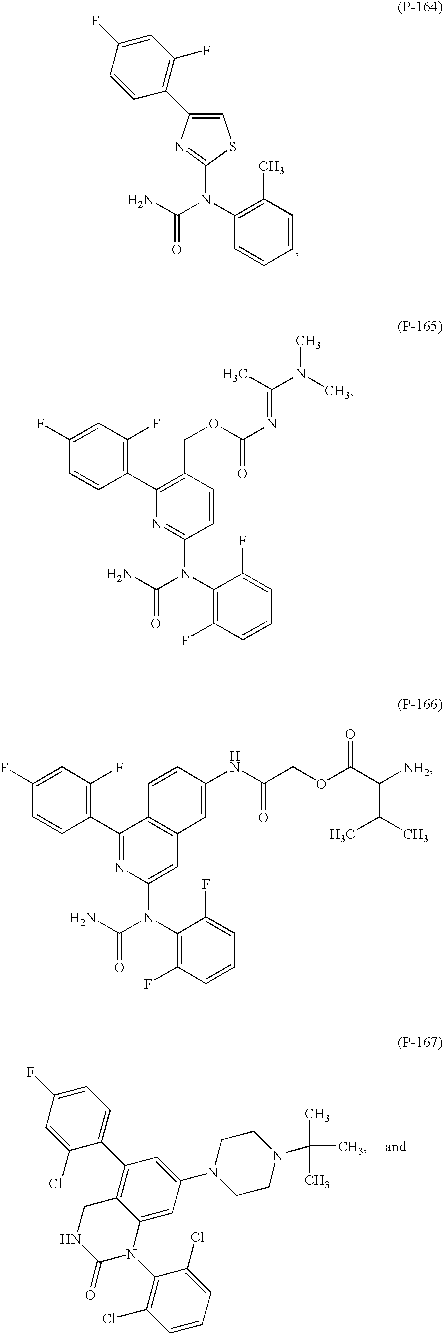

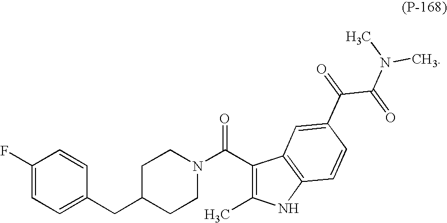

36. A method according to claim 23, wherein the second amount comprises an aldosterone antagonist.

37. A method according to claim 36, wherein the aldosterone antagonist comprises an epoxy-steroidal aldosterone antagonist.

38. A method according to claim 37, wherein the aldosterone receptor antagonist comprises eplerenone.

39. A method according to claim 38, wherein the pathological condition comprises heart failure.

40. A method according to claim 39, wherein the mammal is a dog.

41. A method according to claim 36, wherein the aldosterone antagonist comprises an non-epoxy-steroidal aldosterone antagonist.

42. A method according to claim 41, wherein the aldosterone receptor antagonist comprises spironolactone.

43. A method according to claim 36, wherein the method further comprises a third amount of a compound comprising a diuretic.

44. A method according to claim 23, wherein the second amount comprises a diuretic.

45. A method according to claim 44, wherein the diuretic comprises amanozine, amiloride, arbutin, chlorazanil, ethacrynic acid, etozolin, hydracarbazine, isosorbide, mannitol, metochalcone, muzolimine, perhexiline, ticrynafen, triamterene, urea, amiloride, bumetamide, chlorothiazide, ethacrynic acid, furosemide, hydrochlorothiazide, triamterene, a benzothiadiazine derivative, a sulfonamide derivative, an organic mercurial diuretic, a loop diuretic, or a potassium-sparing diuretic.

46. A method according to claim 45, wherein the diuretic comprises althiazide, bendroflumethiazide, benzthiazide, benzylhydrochlorothiazide, buthiazide, chlorothiazide, chlorthalidone, cyclopenthiazide, cyclothiazide, epithiazide, ethiazide, fenquizone, hydrochlorothiazide, hydroflumethiazide, indapamide, methyclothiazide, meticrane, metolazone, paraflutizide, polythiazide, quinethazone, teclothiazide, trichlormethiazide, acetazolamide, ambuside, azosemide, bumetamide, butazolamide, chloraminophenamide, clofenamide, clopamide, clorexolone, disulfamide, ethoxolamide, furosemide, mefruside, methazolamide, piretanide, torasemide, tripamide, xipamide, or mercaptomerin sodium, merethoxylline, procaine, or mersalyl with thiophylline.

47. A composition, wherein the composition comprises:
a first amount of a compound that comprises a p38-kinase inhibitor, and
a second amount of a compound that comprises an aldosterone antagonist or diuretic.

48. A kit, wherein the kit comprises:
a first dosage form comprising a compound that comprises a p38-kinase inhibitor, and
a second dosage form comprising an aldosterone antagonist or diuretic.


SEQUENCE LISTING (34 sequences)

SEQ ID NO: 1 - DNA, 31 bp, Artificial Sequence
  primer
gatcgaggattcatgtctcaggagaggccca

SEQ ID NO: 2 - DNA, 30 bp, Artificial Sequence
  primer
gatcgaggattctcaggactccatctcttc

SEQ ID NO: 3 - DNA, 17 bp, Artificial Sequence
  primer
atgaatgctgtgcagtc

SEQ ID NO: 4 - DNA, 18 bp, Artificial Sequence
  primer
aaggttcttccattcaat

SEQ ID NO: 5 - DNA, 20 bp, Artificial Sequence
  primer
cgaagctcatcgcagactcc

SEQ ID NO: 6 - DNA, 26 bp, Artificial Sequence
  primer
ggttctccaacttcaggtaataagca

SEQ ID NO: 7 - DNA, 28 bp, Artificial Sequence
  probe
cctgataacctggatgcagtcgtggacc

SEQ ID NO: 8 - DNA, 24 bp, Artificial Sequence
  primer
tcccaggaaaatagctgagaactt

SEQ ID NO: 9 - DNA, 23 bp, Artificial Sequence
  primer
gaaacccaaatgcttcaaagaca

SEQ ID NO: 10 - DNA, 23 bp, Artificial Sequence
  probe
tccacctttgtgccaatgcctgg

SEQ ID NO: 11 - DNA, 18 bp, Artificial Sequence
  primer
cctgccccttccctatgg

SEQ ID NO: 12 - DNA, 20 bp, Artificial Sequence
  primer
ttcaggattcccgcaagagt

SEQ ID NO: 13 - DNA, 36 bp, Artificial Sequence
  probe
tgcagagcactacttgaaatcatactaccatcctgt

SEQ ID NO: 14 - DNA, 22 bp, Artificial Sequence
  primer
agccttccgagtatgggagagt

SEQ ID NO: 15 - DNA, 20 bp, Artificial Sequence
  primer
ctcccggatgtaggcatagg

SEQ ID NO: 16 - DNA, 23 bp, Artificial Sequence
  probe
acgccactgcgcttccgagaagt

SEQ ID NO: 17 - DNA, 20 bp, Artificial Sequence
  primer
aagggctaccagagcgatca

SEQ ID NO: 18 - DNA, 20 bp, Artificial Sequence
  primer
ggtattgccaggtgcacaaa

SEQ ID NO: 19 - DNA, 22 bp, Artificial Sequence
  probe
tttgcctgcctgccacggaatc

SEQ ID NO: 20 - DNA, 23 bp, Artificial Sequence
  primer
ccctatgatcccatgctacatct

SEQ ID NO: 21 - DNA, 20 bp, Artificial Sequence
  primer
ggtgcccattgatgctcttc

SEQ ID NO: 22 - DNA, 25 bp, Artificial Sequence
  probe
ctgtgacccagtccatccagaggca

SEQ ID NO: 23 - DNA, 21 bp, Artificial Sequence
  primer
cccagcactatgtctgcatga

SEQ ID NO: 24 - DNA, 22 bp, Artificial Sequence
  primer
cgtattccttccggaggtgtag

SEQ ID NO: 25 - DNA, 28 bp, Artificial Sequence
  probe
cctcggtaccagctacagatgccatcaa

SEQ ID NO: 26 - DNA, 21 bp, Artificial Sequence
  primer
gccaaggctaacctggagaag

SEQ ID NO: 27 - DNA, 20 bp, Artificial Sequence
  primer
cgggtgaggtcattgacaga

SEQ ID NO: 28 - DNA, 29 bp, Artificial Sequence
  probe
tcctcagccttgctccggtgttcattcat

SEQ ID NO: 29 - DNA, 22 bp, Artificial Sequence
  primer
acctggagaacgacaagcttca

SEQ ID NO: 30 - DNA, 20 bp, Artificial Sequence
  primer
gggcctgctcgtcctctatt

SEQ ID NO: 31 - DNA, 36 bp, Artificial Sequence
  probe
aggaaaagctcaagaagaaagagtttgacatcagtc

SEQ ID NO: 32 - DNA, 27 bp, Artificial Sequence
  primer
agagaaatttgaggatgagaacttcat

SEQ ID NO: 33 - DNA, 21 bp, Artificial Sequence
  primer
ttgtgtttggtccagcatttg

SEQ ID NO: 34 - DNA, 29 bp, Artificial Sequence
  probe
aagcatacaggtcctggcatcttgtccat